(19) United States
(12) Patent Application Publication
(10) Pub. No.: US 2020/0291018 A1
(43) Pub. Date: Sep. 17, 2020

(54) TAM KINASE INHIBITORS

(71) Applicant: SYROS PHARMACEUTICALS, INC., Cambridge, MA (US)

(72) Inventors: Yi ZHANG, Belmont, MA (US); Claudio Edmundo CHUAQUE, Arlington, MA (US); Goran MALOJCIC, Boston, MA (US); William SINKO, Roslindale, MA (US); Stephane CIBLAT, Montreal (CA); Clint JAMES, Laval (CA); Huiping Amy GUAN, Acton, MA (US); Jason J. MARINEAU, Franklin, MA (US); David MOEBIUS, Westwood, MA (US)

(73) Assignee: Syros Pharmaceuticals, Inc., Cambridge, MA (US)

(21) Appl. No.: 16/630,059

(22) PCT Filed: Jul. 13, 2018
(86) PCT No.: PCT/US2018/041928

§ 371 Date: Jan. 10, 2020

Related U.S. Application Data
(60) Provisional application No. 62/532,135, filed on Jul. 13, 2017.

(51) Int. Cl. C07D 471/04

(52) U.S. Cl. CPC C07D 471/04

(57) ABSTRACT

Described herein are compounds, methods of making such compounds, pharmaceutical compositions, and medicaments comprising such compounds, and methods of using such compounds to treat diseases, such as cancer.

### CROSS-REFERENCE TO RELATED APPLICATIONS

This application is a U.S. National Phase Application under 35 U.S.C. § 371 of International Application No. PCT/US2018/041928, filed Jul. 13, 2018, which claims the benefit of the filing date of U.S. Provisional Application No. 62/532,135, filed Jul. 13, 2017, the entire content of each of which is hereby incorporated by reference herein.

### BACKGROUND

The TAM receptor tyrosine kinases (TYRO3, AXL and MERTK; the “TAM kinases”) constitute a family of receptor tyrosine kinases (RTKs) that play several important roles in normal macrophage physiology, including regulation of cytokine secretion and clearance of apoptotic cells. Modulation of TAM kinases has been shown to be useful in the treatment of a variety of diseases.

### SUMMARY

The present disclosure provides compounds represented by structural Formula I:

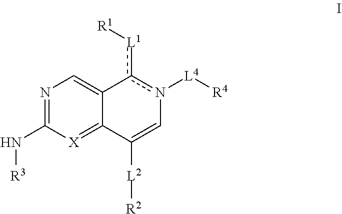

a prodrug thereof, or a pharmaceutically acceptable salt thereof, wherein:
each [symbol] represents a single or a double bond, provided only one [symbol] is a single bond;

X is N or C(R);

R is hydrogen, deuterium, halogen, —CN, —S(O)₂R⁶, —S(O)₂N(R⁵)R⁶, —C(O)N(R⁵)R⁶, C(O)₂R⁵, P═O(R⁶)₂, N(R⁵)R⁶, OR⁵, optionally substituted C₁-C₆ alkyl, optionally substituted aryl, optionally substituted carbocyclyl, optionally substituted heteroaryl, or optionally substituted heterocyclyl;

R⁵ is hydrogen, C₁-C₆ alkyl, aryl, C₃-C₈ cycloalkyl, heteroaryl, or heterocyclyl, wherein R⁵ is optionally substituted when other than hydrogen;

each R⁶ is independently C₁-C₆ alkyl, aryl, C₃-C₈ cycloalkyl, heteroaryl, or heterocycloalkyl, wherein R⁶ is optionally substituted; or

R⁵ and R⁶ are optionally taken together to form an optionally substituted heterocyclyl;

L¹ is a bond, ═O, —C₁-C₆ alkylene, —O—(C₀-C₆ alkylene)-†, —NH—(C₀-C₆ alkylene)-†, or —N(C₁-C₆ alkyl)-(C₀-C₆ alkylene)-†, wherein “†” represents a portion of L¹ bound to R¹, and the alkylene portion of L¹, if present, is optionally substituted;

when L¹ is ═O, R¹ is absent, or

when L¹ is a bond, —C₁-C₆ alkylene, —O—(C₀-C₆ alkylene)-†, —NH—(C₀-C₆ alkylene)-†, or —N(C₁-C₆ alkyl)-(C₀-C₆ alkylene)-†, R¹ is C₁-C₆ alkyl, aryl, heteroaryl, heterocyclyl, or carbocyclyl, wherein R¹ is optionally substituted with up to four independently selected substituents, or

L¹ and R¹ are taken together to form an optionally substituted, N-linked saturated heterocyclyl;

L² is a bond, —O—(C₀-C₆ alkylene)-*, —NH—(C₀-C₆ alkylene)-*, or —N(C₁-C₆ alkyl)-(C₀-C₆ alkylene)-*, wherein “*” represents a portion of L² bound to R², and the alkylene or alkyl portion of L², if present, is optionally substituted;

R² is C₁-C₆ alkyl, aryl, heteroaryl, heterocyclyl or carbocyclyl, wherein R² is optionally substituted with up to four different substituents, and R² is additionally hydrogen when L² is other than a bond;

R³ is optionally substituted —C₁-C₆ alkyl, —C₂-C₆ alkenyl, —C₂-C₆ alkynyl, —(C₂-C₆ alkylene)-O—(C₁-C₆ alkyl), —(C₂-C₆ alkylene)-O-aryl, —(C₂-C₆ alkylene)-O-heteroaryl, —(C₀-C₆ alkylene)-aryl, —(C₀-C₆ alkylene)-carbocyclyl, —(C₀-C₆ alkylene)-heterocyclyl, or —(C₀-C₆ alkylene)-heteroaryl, wherein each substituent on an alkyl or alkylene portion of R³ is a monovalent substituent, and wherein R³ is other than 2-aminocyclohexyl or 2-(t-butyloxycarbonylamino)cyclohexyl;

when L¹ is other than ═O, L⁴-R⁴ is absent and

when L¹ is ═O, L⁴ is a bond, or —(C₁-C₆ alkylene)-‡, wherein “‡” represents a portion of L⁴ bound to R⁴, and the alkylene portion of L⁴, if present, is optionally substituted and

R⁴ is C₁-C₆ alkyl, —OR⁶, —N(R⁵)R⁶, —S(O)₂N(R⁵)R⁶, —S(O)₂—R⁶, —N(R⁵)—S(O)₂—R⁶, —N(R⁵)—C(O)—R⁶, —N(R⁵)—C(O)—N(R⁵)R⁶, —C(O)—N(R⁵)R⁶, aryl, heteroaryl, heterocyclyl or carbocyclyl, wherein R⁴ is optionally substituted with up to four different substituents, and R⁴ is additionally hydrogen when L⁴ is other than a bond, wherein:

when X is N and L¹ is ═O, R³ is other than optionally substituted phenyl.

In each instance where a moiety is described as “optionally substituted,” it may be substituted or unsubstituted. For example, “optionally substituted phenyl” encompasses “substituted phenyl” and “unsubstituted phenyl.”

A compound described herein, a pharmaceutically acceptable salt thereof, and/or a pharmaceutical composition containing the compound or its salt may be used to inhibit one or moreTAM kinases, at least at a site of interest (e.g., in a tissue, in a cell, or in a subcellular location, any of which may be located in vivo or in vitro). In some embodiments, a provided compound and/or composition containing it (e.g., a pharmaceutical composition) may be used to inhibit cell proliferation, particularly cell proliferation that is uncontrolled, excessive and/or detrimental to a patient. Without limiting the invention to compounds that exert an effect by any particular mechanism, a provided compound and/or composition containing it may inhibit a kinase activity of any one or more of the TAM kinases (e.g., MERTK, AXL and TYRO3) and may have an increased specificity for at least one TAM kinase relative to FLT3. A provided compound and/or composition containing it may inhibit a TAM kinase without inhibiting FLT3.

For ease of reading, we will not refer to both a compound of the invention and a pharmaceutically acceptable salt thereof when describing each and every composition, method, and use within the scope of the invention. It is to be understood that where a compound of the invention can be used, a pharmaceutically acceptable salt thereof may also be useful, and that determination is well within the ability of one of ordinary skill in the art. For example, a compound of the invention or a pharmaceutically acceptable salt thereof may have an increased specificity for at least one TAM kinase; may exhibit that specificity relative to FLT3; and so forth as described herein.

A provided compound and/or a composition containing it (e.g., a pharmaceutical composition) can be contacted with and/or administered to cells, such as cancer cells, in vitro or in vivo. Accordingly, the invention features methods of treating or preventing a disease described herein (e.g., a cancer) by administering a therapeutically effective amount of a compound or composition described herein to a patient in need thereof. Each therapeutic or prophylactic method may also be expressed in terms of use. For example, the invention encompasses the use of a compound or composition described herein for the treatment of a disease described herein (e.g., cancer); a compound or composition for use in treating or preventing a disease (e.g., cancer); and the use of the compound or composition for the preparation of a medicament for treating a disease described herein (e.g., cancer). The cancer a patient has been diagnosed as having and/or the cancer cells contacted with a compound or composition can be of the following type: a blood cancer, a bone cancer, a breast cancer (e.g., a triple-negative breast cancer (TNBC)), an endocrine cancer (e.g., cancer of the thyroid or parathyroid gland), a gastrointestinal cancer (e.g., a gastric cancer or colorectal cancer), a genitourinary cancer (e.g., cancer of the bladder, kidney, prostate, cervix, or uterus (e.g., an endometrial cancer)), a head and neck cancer (e.g., cancer of the larynx), a liver cancer, a lung cancer (e.g., non-small cell lung cancer (NSCLC)), melanoma (e.g., a skin cancer or a cutaneous or intraocular melanoma), a nervous system or brain cancer (e.g., glioblastoma), an oral cancer (e.g., a cancer of the mouth or throat), an ovarian cancer, a pancreatic cancer (e.g., pancreatic ductal adenocarcinoma), a plasma cell neoplasm or myeloma (typically referred to as a plasmacytoma when plasma cells form a single tumor in bone or soft tissue or multiple myeloma when multiple tumors are formed), or rhabdomyosarcoma. The blood cancer, which may also be referred to as a hematopoietic or hematological cancer or malignancy, can be a leukemia such as acute lymphocytic leukemia (ALL; e.g., B cell ALL or T cell ALL), acute myelocytic leukemia (AML; e.g., B cell AML, or T cell AML), chronic myelocytic leukemia (CIVIL; e.g., B cell CML or T cell CML), or chronic lymphocytic leukemia (CLL; e.g., B cell CLL (e.g., harry cell leukemia) or T cell CLL). The blood cancer can also be a lymphoma such as Hodgkin lymphoma (HL; e.g., B cell HL or T cell HL), non-Hodgkin lymphoma (NHL; e.g., B cell NHL or T cell NHL), follicular lymphoma, chronic lymphocytic leukemia/small lymphocytic lymphoma (CLL/SLL), mantle cell lymphoma (MCL), a marginal zone B cell lymphoma (e.g., splenic marginal zone B cell lymphoma), primary mediastinal B cell lymphoma, Burkitt lymphoma, lymphoplasmacytic lymphoma (i.e., Waldenstrom's macroglobulinemia), immunoblastic large cell lymphoma, precursor B-lymphoblastic lymphoma, or primary central nervous system (CNS) lymphoma. The B cell NHL can be diffuse large cell lymphoma (DLCL; e.g., diffuse large B cell lymphoma), and the T cell NHL can be precursor T lymphoblastic lymphoma or a peripheral T cell lymphoma (PTCL). In turn, the PTCL can be a cutaneous T cell lymphoma (CTCL) such as mycosis fungoides or Sezary syndrome, angioimmunoblastic T cell lymphoma, extranodal natural killer T cell lymphoma, enteropathy type T cell lymphoma, subcutaneous anniculitis-like T cell lymphoma, or anaplastic large cell lymphoma. While the invention is not limited to treating or preventing blood cancers having any particular cause or presentation, stem cells within the bone marrow may proliferate, thereby becoming a dominant cell type within the bone marrow and a target for the present compounds. Leukemic cells can accumulate in the blood and infiltrate organs such as the lymph nodes, spleen, liver, and kidney.

The methods of the invention that concern treating or preventing a disease described herein (e.g., a cancer (or use of a compound for such purposes)) may specifically exclude any one or more of the types of cancers described herein (e.g., above). For example, the invention features methods of treating or preventing cancer by administering a compound as described herein with the proviso that the cancer is not a breast cancer; with the proviso that the cancer is not breast cancer or leukemia; with the proviso that the cancer is not breast cancer, a leukemia, or a lymphoma; with the proviso that the cancer is not breast cancer, a leukemia, a lymphoma, gastric cancer, prostate cancer, a pituitary adenoma, NSCLC, melanoma, glioblastoma, ovarian cancer or rhabdomyosarcoma; and so forth, with exclusions selected from any of the diseases described herein and with the same notion of variable exclusion from lists of elements relevant to other aspects of the invention (e.g., chemical substituents of a compound described herein or components of pharmaceutical compositions).

A provided compound and/or a composition containing it (e.g., a pharmaceutical composition) can be administered to cancer cells or a cancer patient, including to cells or a patient having any of the types of cancer described above, and such administration can occur in the event the cancer is resistant to a checkpoint inhibitor (i.e., a method or use described herein may be applied to a patient who has received, is receiving, or is scheduled to receive a checkpoint inhibitor). Checkpoint inhibitors include, but are not limited to, PD-1 inhibitors (e.g., avelumab, nivolumab, and pembrolizumab), PD-L1 inhibitors (e.g., atezolizumab and durvalumab), and CTLA4 inhibitors (e.g., ipilimumab). In some embodiments, a compound and/or pharmaceutical composition described herein is administered to a patient who has a cancer associated with elevated myeloid infiltration. In some embodiments, a compound and/or pharmaceutical composition described herein is brought into contact with cells (e.g., by administration to a patient) affected by a hematologic disorder, such as myelodysplastic syndrome (MDS) or myeloproliferative disease (MPS), in which precursor cells in the bone marrow (e.g., stem cells) do not mature properly. Some types of MDS may develop into AML. Patients who have been diagnosed with MDS or MPS that then transforms to AML may be referred to as having AML or AML with myelodysplasia-related changes. A provided compound and/or composition containing it (e.g., a pharmaceutical composition) can also be administered to cells or to a patient who has a benign lesion such as a papilloma or adenoma (e.g., a pituitary adenoma). Although hematologic disorders and benign lesions generally have far less serious consequences for a patient than cancer, we may refer to any of these conditions as a “disease” and any are amenable to treatment and preventative care as described herein.

### BRIEF DESCRIPTION OF THE DRAWINGS

FIG. 1 is a table describing physiochemical properties of exemplary Compound Nos. 101-218, 220-257, and 259-437 and the protocol numbers by which they were made.

### DETAILED DESCRIPTION

The TAM receptors (TYRO3, AXL, and MERTK) comprise a family of receptor tyrosine kinases (RTKs) that play several important roles in normal macrophage physiology, including regulation of cytokine secretion and clearance of apoptotic cells. Expression of the TAM kinases is heterogeneous among macrophage subsets, being mostly restricted to anti-inflammatory M2 macrophages, which contribute significantly to the immunosuppression present in the tumor microenvironment. By inhibiting TAM kinases on tumor-infiltrating macrophages, the immunosuppressive environment can be reduced by repolarizing M2 macrophages, which can increase effector killer immune cell function and promote tumor regression. Current CD8+ T cell- and NK cell-directed immunotherapies have shown promise but only in a limited percentage of patients. The reason for this limitation is not well understood, but it may be that the immunosuppressive environment is inhibiting efficacy. Since M2 macrophages contribute significantly to this environment, reversal of the M2 phenotype may increase the number of patients who will respond to CD8+T cell- and NK cell-directed immunotherapies. Therefore, pan-TAM kinase inhibitors can be used to treat cancers where checkpoint inhibitors have shown limited efficacy and have high myeloid infiltration, such as pancreatic ductal adenocarcinoma, ovarian cancer, TNBC, glioblastoma, and colorectal cancer.

In addition, abnormally increased MERTK expression has been reported in multiple human cancer types including leukemias, lymphomas, gastric cancer, prostate cancer, breast cancer, pituitary adenoma, NSCLC, melanoma, glioblastoma, ovarian cancer and rhabdomyosarcoma. The overexpression of MERTK in at least some cancer cells results in increased survival and resistance to apoptosis, resulting in oncogenesis. In some embodiments, compounds of the present invention inhibit all three of the known TAM kinases. While treatment with, or use of, the compounds and compositions described herein is not so limited, that treatment or use is expected to lower tumor burden and/or increase patient survival.

Compounds previously reported in the literature to have potent inhibitory activity against one or more of the TAM kinases have limitations. For example, some of these inhibitors may be potent against only one or two TAM kinases. Such partial activity may be sufficient for a direct anti-cancer effect in a cancer that is known to be associated with a specific TAM kinase. However, given the redundancy of TAM kinases in driving the M2 immunosuppressive functional state, the uninhibited kinase(s) may compensate for the inhibited kinase(s).

When CD14⁺ cells are isolated from patient tumors, heterogeneity is observed in the expression of the three TAM kinases amongst patients, but in the majority of patients studied, all three TAM kinases are expressed. Many compounds have undesirable off-target effects. At least some compounds that reportedly inhibit TAM kinases also potently inhibit kinases that are required for immune system maintenance and renewal (e.g., RET, KIT, MET, FLT3, and others). Inhibiting these kinases causes cytopenia and inhibits immune system function in patients, thereby negating the use of these compounds in treatments that depend upon an immune-based mechanism of action.

As noted, the compounds and compositions described herein are intended for the treatment and prevention of cancer, and in some embodiments, the cancer may be a disease in which a TAM kinase is overexpressed or overly active. Accordingly, any of the treatment or prophylactic methods described herein (whether expressed as a method per se or expressed in terms of the use of a compound or composition) may include a step of determining whether the cancer is one in which a TAM kinase is overexpressed or overly active by, for example, exceeding a pre-determined threshold level. Making that determination can be done by obtaining a sample from a patient (e.g., a biological sample comprising cancer cells) and using any number of routine techniques to assess the level of expression or activity of a TAM kinase. Alternatively, the information concerning TAM kinase expression or activity may have been previously determined, in which case the methods or uses of the compounds and compositions described herein may include receiving that information. Thus, in embodiments in which one or more TAM kinases are assessed, the assessment can require determining, having determined, or receiving information concerning an expression level, activity, and/or threshold level useful as a reference for one or more of the TAM kinases (e.g., TYRO3, AXL, and MERTK).

Compounds of this invention include those described herein, including the compounds generally described above and those further illustrated by the classes, subclasses, and species disclosed below. The invention also encompasses methods of making the disclosed compounds, methods of administering the compounds to patients and/or to cells (e.g., cancer cells from or within the patient and/or cancer cell lines), and any disclosed compound or pharmaceutical composition for use in treating a patient or as a medicament for use in the treatment of a disease disclosed herein (e.g., a cancer). For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed. Eds. M. B. Smith, and J. March, John Wiley & Sons, New York: 2001, the entire contents of which are hereby incorporated by reference herein.

The following definitions shall apply unless otherwise indicated.

The terms “aliphatic” and “aliphatic group,” as used herein, mean a branched or unbranched (i.e., straight-chain), substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclyl,” “cycloaliphatic,” or “cycloalkyl”) and that has a single point of attachment to the rest of the molecule (i.e., compound). Unless otherwise specified, aliphatic groups contain 1-6 aliphatic carbon atoms (“C₁-C₆”). Aliphatic groups in the present compounds can contain 1-5 aliphatic carbon atoms (“C₁-C₅”); 1-4 aliphatic carbon atoms (“C₁-C₄”); 1-3 aliphatic carbon atoms (“C₁-C₃”); or 1-2 aliphatic carbon atoms (“C₁-C₂”). In some embodiments, “cycloaliphatic” (or “carbocycle” or “cycloalkyl”) refers to a monocyclic hydrocarbon containing 3-6 aliphatic carbon atoms (“C₃-C₆”) that is completely saturated or that contains one or more units of unsaturation, but which is not aromatic and that has a single point of attachment to the rest of the molecule (i.e., compound). Suitable aliphatic groups within the present compounds include, but are not limited to, branched or unbranched (i.e., straight-chain), substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl,” as used herein, means abranched or an unbranched (i.e., straight) chain, saturated, monovalent hydrocarbon residue containing 1 to 10 carbon atoms. Suitable alkyl groups include methyl, ethyl, n- and iso-propyl, n-, sec-, iso- and tert-butyl, and neopentyl.

The term “alkenyl,” as used herein, means a monovalent branched or unbranched (i.e., straight) chain of, unless otherwise specified, from 2 to 10 carbon atoms (“C₂-C₁₀”) containing one or more carbon-carbon double bonds and is exemplified by ethenyl, propenyl, butenyl, pentenyl, and hexenyl.

The term “alkynyl,” as used herein, means a monovalent branched or unbranched (i.e., straight) chain from 2 to 10 carbon atoms (“C₂-C₁₀”) containing at least one carbon-carbon triple bond. Suitable alkynyl groups include ethynyl, propynyl, butynyl, pentynyl, and hexynyl.

The term “alkylene,” as used herein, means a branched or unbranched (i.e., straight) bivalent alkyl group. Exemplary alkylenes include —CH₂—, —CH₂CH₂—, —CH(CH₃)—, —CH₂CH(CH₃)—, —CH(CH₃)CH₂—, etc. In some embodiments, an “alkylene chain” is a polymethylene group, i.e., —(CH₂)ₙ—, wherein n is a positive integer, preferably from 1 to 6 (e.g., 1-4, 1-3, 1-2, or 2-3). A substituted alkylene chain is a bivalent alkyl group in which one or more hydrogen atoms are replaced with a substituent. Suitable substituents include those described below for a substituted aliphatic group.

The term “alkenylene,” as used herein, means a bivalent alkenyl group. A substituted alkenylene chain is a bivalent alkenyl group containing at least one double bond in which one or more hydrogen atoms are optionally replaced with a substituent. Suitable substituents include those described below for a substituted aliphatic group.

The terms “aryl” and “aryl ring,” as used herein, mean monocyclic, bicyclic and tricyclic ring systems having a total of six to fourteen ring atoms, with each ring atom being carbon, at least one ring in the system being aromatic, and each ring in the system containing three to seven ring atoms. In certain embodiments, “aryl” refers to an aromatic ring system that includes, but is not limited to, phenyl, biphenyl, naphthyl, and anthracyl, which may bear one or more substituents. As used herein, “aryl” may also refer to a group in which an aromatic ring is fused to one or more non-aromatic rings, such as indanyl, naphthimidyl, or tetrahydronaphthyl.

The term “bivalent C₂₋₈ (or C₂₋₆) unsaturated, branched or unbranched, hydrocarbon chain,” as used herein, means bivalent alkenylene and alkynylene chains that are branched or unbranched (i.e., straight) as defined herein and have one or more units of unsaturation.

The references herein to a “compound” are references to a chemical compound (e.g., a compound represented by structural Formula I, a sub-genus thereof, or a species thereof). Any given compound may be biologically active and/or therapeutically active (e.g., when incorporated into a pharmaceutical composition in a therapeutically effect amount) and can be provided and/or utilized (e.g., used in a biological assay, administered to a patient, incorporated into a medicament, or otherwise used as described herein) in any of a variety of forms. Unless the context clearly indicates otherwise, the references herein to a “compound” encompass the compound per se, a hydrate thereof, a stereoisomeric form thereof (e.g., an optical and/or structural isomer), or an isotopic form thereof. One of ordinary skill in the art will appreciate that certain compounds have structures that can exist in one or more stereoisomeric or tautomeric forms, and such compounds may be utilized as described herein. For example, the compounds can exist and be utilized as described when in the form of an individual enantiomer, diastereomer or geometric isomer, or when in the form of a mixture of stereoisomers (e.g., in a racemic mixture). The compounds may also have one or more isotopic substitutions (e.g., ²H or ³H for H; ¹¹C; ¹³C or ¹⁴C for ¹²C; ¹³N or ¹⁵N for ¹⁴N; ¹⁷O or ¹⁸O for ¹⁶O; ³⁶Cl for ³⁵C; ¹⁸F for ¹⁹F; ¹³¹I for ¹²⁷I; etc.). Thus, unless otherwise indicated, the chemical structures depicted herein describe and encompass all stereoisomers of the depicted structure (i.e., enantiomers and diastereomers (e.g., cis/trans isomers, and conformational isomers)). These include the R and S configurations for each asymmetric center, Z and E double bond isomers, and Z and E conformational isomers. Compositions containing single stereochemical isomers as well as enantiomeric, diastereomeric, and geometric (or conformational) mixtures of the present compounds are within the scope of the invention, as are all tautomeric forms of the compounds. Thus, it is to be understood that a chemical structure depicted herein (e.g., Formula I, a sub-genus thereof, or a species thereof) also describes compounds that differ from the depicted structures only by virtue of containing one or more isotopically variant (e.g., enriched) atoms. For example, the presented structures equally well describe compounds in which an illustrated hydrogen is replaced by deuterium or tritium, and those in which ¹²C is replaced by ¹³C- or ¹⁴C-enriched carbon. Such compounds have use, for example, as analytical tools, as probes in biological assays, and/or as therapeutic or prophylactic agents for use in accordance with the present invention. In any embodiment, a compound may be a specific form of a compound. In some embodiments, the R¹ group of Formula I comprises one or more deuterium atoms.

Should a compound be one that exists in nature, that compound may be provided and/or utilized as described herein in a form different from that in which it exists or is found in nature (i.e., any compound described herein can be in a non-naturally occurring form (e.g., in a non-naturally occurring racemic mixture or provided as a non-naturally occurring isotopic form or tautomer)). A composition of the invention (e.g., a pharmaceutical composition) can contain a different level, amount, concentration or ratio of one or more individual compounds or forms thereof than a reference composition or source (e.g., a natural source) of the compound and would, therefore, be a non-naturally occurring composition. For example, in some embodiments, a compound is “substantially pure” by virtue of being substantially free of other, distinct chemical compounds (e.g., a preparation of a compound of the invention can contain less than about 25%, 20%, 15%, 10%, 9%, 8%, 7%, 6%, 5%, 4%, 3%, 2%, 1%, 0.5% or 0.1%, by weight, of other distinct chemical compounds). In case of doubt, and with regard to natural occurrence, a composition containing a single stereoisomer of a compound differs from a composition containing a racemic mixture of that compound; a particular salt of a compound differs from other salt forms of the compound; compounds having one conformational isomer ((Z) or (E)) of a double bond differ from compounds having the other conformational isomer ((E) or (Z)) of the double bond; and compounds in which one or more atoms are of a different isotope than is present in compounds of a reference preparation differ from that reference preparation.

The term “halogen,” as used herein, means F, Cl, Br, or I.

The term “heteroalkyl,” as used herein, refers to an alkyl group in which one or more methylene groups is replaced with oxygen, sulfur, S═O, SO₂ or NR where R in this instance is H, alkyl, or substituted alkyl.

The terms “heteroaryl” and “heteroar-”, used alone or as part of a larger term such as “heteroaralkyl” or “heteroaralkoxy,” refer to groups having 5 to 14 ring atoms (e.g., 5, 6, or 9 ring atoms); 6, 10, or 14 n electrons shared in a cyclic array; and, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. When used in reference to a ring atom of a heteroaryl, the term “nitrogen” includes a substituted nitrogen. As an example, in a heteroaryl ring having 0-3 heteroatoms selected from oxygen, sulfur or nitrogen, nitrogen may be N (as in pyridinyl-

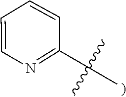 [SMILES: *c1ccccn1]

or ⁺NR\hat{ } (as in N-substituted pyridinyl-

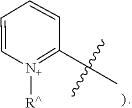

Heteroaryl groups may be mono-, bi- or tricyclic. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, and pteridinyl. “Heteroaryl” and “heteroar-” may also be used to refer to groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, 5,6,7,8-tetrahydroquinolinyl, 5,6,7,8-tetrahydroisoquinolinyl, and pyrido[2,3-b]-1,4-oxazin-3(4H)-one. When a heteroaryl ring is fused to an aryl ring, the term “heteroaro” is used to refer to the heteroaryl ring that is fused to the aryl ring. The term “heteroaryl” may be used interchangeably with “heteroaryl ring,” “heteroaryl group,” or “heteroaromatic,” and any of such rings can be optionally substituted.

As used herein, the terms “heterocycle,” “heterocyclyl,” “heterocyclic radical,” and “heterocyclic ring” are used interchangeably and refer to a stable 4- to 7-membered monocyclic, 7-11-membered bicyclic, or 10-16-membered tricyclic heterocyclic moiety that is either saturated or partially unsaturated, and that has, in addition to carbon atoms, one or more (e.g., 1-4) heteroatoms as defined above. When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen may be N (as in 3,4-dihydro-2H-pyrrolyl-

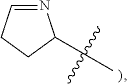 [SMILES: *C1CCC=N1]

NH (as in pyrrolidinyl-

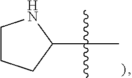 [SMILES: *C1CCCN1]

NR\hat{ } (as in N-substituted 2-pyrrolidinyl-

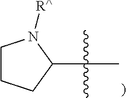

or ⁺NR\hat{ } (as in N-substituted 1-pyrrolidinyl-

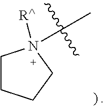

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” and “heterocyclic group” also refer to groups in which a heterocyclyl ring is fused to one or more aryl, heterocyclyl, or cycloaliphatic rings, such as indolinyl, 3H-indolyl, chromanyl, 1,2,3,4-tetrahydroisoquinolinyl or 1,2,3,4-tetrahydroquinolinyl. For purposes of clarity, a “heterocyclic” ring system includes a saturated or partially unsaturated, but not aromatic, ring having one or more heteroatoms, wherein the ring is either monocyclic or fused to one or more aryl, heterocyclyl or cycloaliphatic rings. When a heterocyclic ring is fused to an aryl ring, the term “heterocyclo” is used to refer to the heterocyclic ring that is fused to the aryl ring. A “saturated heterocyclic ring” refers to a saturated ring having one or more heteroatoms, wherein the ring is monocyclic or fused to one or more saturated cycloaliphatic rings.

The term “heteroatom,” as used herein, means one or more of oxygen, sulfur, nitrogen, phosphorus, or silicon (including any oxidized forms thereof) and the quaternized form of any basic nitrogen or a substitutable nitrogen of a heterocyclic ring, for example N (as in 3,4-dihydro-2H-pyrrolyl-

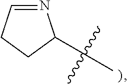 [SMILES: *C1CCC=N1]

NH (as in pyrrolidinyl-

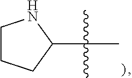 [SMILES: *C1CCCN1]

NR\hat{ } (as in N-substituted 2-pyrrolidinyl-

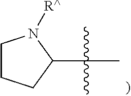

or ⁺NR\hat{ } (as in N-substituted 1-pyrrolidinyl-

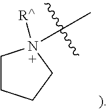

As used herein, the term “partially unsaturated” refers to a ring moiety that includes at least one double or triple bond and is intended to encompass rings having multiple sites of unsaturation (but not aryl or heteroaryl moieties, as herein defined).

As described herein, a disclosed compound may contain one or more “optionally substituted” moieties. In general, the term “substituted,” whether preceded by the term “optionally” or not, means that one or more hydrogens of the designated moiety are replaced with a suitable substituent. Unless otherwise indicated, an “optionally substituted” moiety may have a suitable substituent at each substitutable position of the moiety, and when more than one position is substituted with more than one substituent selected from a specified group, the substituent may be either the same or different at every position. The combination of substituents may result in the formation of stable and/or chemically feasible compounds. A “stable” compound is one that is not substantially altered when subjected to conditions that allow for its production, detection, or formulation and, when relevant, its recovery, purification, and use for one or more of the purposes disclosed herein.

Suitable monovalent substituents on a substitutable carbon atom of an “optionally substituted” group may be, independently, deuterium, halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)_{0- 4}Ph, which may be substituted with R^{∘}; —(CH₂)₀₋₄O(CH₂)₀₋₁Ph, which may be substituted with R^{∘}; —CH═CHPh, which may be substituted with R^{∘}; —(CH₂)₀₋₄O(CH₂)₀₋₁-pyridyl, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘}C(S)R^{∘}; —(CH₂)₀₋₄N(R^{∘})C(O)NR^{∘}₂; —N(R^{∘})C(S)NR^{∘}₂; —(CH₂)₀₋₄N(R^{∘}C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(O)R^{∘}₂; —OP(O)R^{∘}₂; —OP(O)(OR^{∘}₂; —OP(O)(OR^{∘})R^{∘}, —SiR^{∘}₃; —(C₁₋₄ branched or unbranched)alkylene)O—N(R^{∘)}₂; or —(C₁₋₄ branched or unbranched) alkylene)C(O)O—N(R^{∘)}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘}(or the ring formed by taking two independent occurrences of R^{∘} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —C(O)SR^{●}, —(C₁₋₄ branched or unbranched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁-6 aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

As used herein, “ [symbol] ” appearing on a structure and joining a functional group to the structure in the position of a bond generally indicates a mixture of, or either of, the possible isomers, e.g., containing (R)- and (S)-stereochemistry.

As used herein, “ [symbol] ” appearing across or at the end of a bond indicates a point of attachment between two atoms. For example:

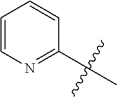 [SMILES: *c1ccccn1]

means that the pyridine (Py) ring above is bound through the indicated ring carbon atom to an undepicted structure on which it is a substituent.

As used herein, the term “pharmaceutically acceptable salt” refers to a salt that is, within the scope of sound medical judgment, suitable for use in contact with the tissues of humans and lower animals without unacceptable toxicity, irritation, allergic response and the like, and that can be used in a manner commensurate with a reasonable benefit/risk ratio. Pharmaceutically acceptable salts are well known in the art. For example, Berge et al. (J. Pharm. Sci. 66:1-19, 1977; incorporated herein by reference) describe pharmaceutically acceptable salts in detail. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “pharmaceutical composition” refers to a composition in which an active agent (e.g., any one or more of the compounds described herein) is formulated together with one or more pharmaceutically acceptable carriers. The active agent can be present in a unit dose amount appropriate for administration in a therapeutic regimen that shows a statistically significant probability of achieving a predetermined therapeutic effect when administered to a relevant population of patients. The pharmaceutical composition may be specially formulated for administration in solid, semi-solid, or liquid form, including formulations adapted for oral or parenteral administration. For example, oral preparations can be formulated as drenches (aqueous or non-aqueous solutions or suspensions) or as a tablet or capsule. Compositions formulated for parenteral administration can be prepared for subcutaneous, intramuscular, intravenous or epidural injection as, for example, a sterile solution or suspension; for topical or transdermal application as, for example, a cream or ointment, or within a patch or spray applied to the skin; for intravaginal or intrarectal administration, for example, as a pessary, suppository, cream, or foam; for application to another mucosal surface (e.g., buccal, sublingual, intranasal or pulmonary administration) as, for example, a bolus, powder, granular formulation, paste, or nasal spray); or ocularly, for example, within eye drops. Any of these formulations can be prepared for sustained- or controlled release. Any of the compounds described herein and pharmaceutical compositions containing them can also be referred to as a “medicament.”

As used herein, the term “pharmaceutically acceptable,” as applied to a composition or to the carrier, diluent, or excipient used to formulate a pharmaceutical composition as described herein means that the composition is not unacceptably deleterious to a population of patients for whom it is intended and that the carrier, diluent, or excipient is compatible with the other ingredients of the composition.

The term “unsaturated,” as used herein, means that a moiety has one or more units of unsaturation. A unit of unsaturation can be a carbon-carbon double bond (i.e., —C═C—) or a carbon-carbon triple bond (i.e., —C≡C).

### Other Definitions

With regard to certain values, the term “about” is used to describe standard variation as would be understood by one of ordinary skill in the art or a range within plus-or-minus 10% (e.g., plus- or minus 1%, 2%, or 5%) of the stated value. For example, “about 2% by weight” means 1.8-2.2% by weight. In case of doubt, “about X” can be “X” (e.g., about 80% can be 80%).

As used herein, the term “administration” typically refers to the administration of a compound described herein or a composition containing it to a subject (e.g., a human patient) or system. One of ordinary skill in the art will be aware of a variety of routes that may, in appropriate circumstances, be utilized for administration to a subject. For example, the route of administration may be bronchial (e.g., by bronchial instillation), buccal, dermal (which may be or comprise topical application to the dermis or intradermal, interdermal, or transdermal administration), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, or vitreal. Administration may involve dosing that is intermittent (e.g., a plurality of doses separated in time) and/or periodic (e.g., individual doses separated by a common period of time). In other embodiments, administration may involve continuous dosing (e.g., perfusion) for at least a selected period of time. In some embodiments, administration of a compound may be achieved by administration of a composition that achieves delivery of the compound (e.g., administration of a composition that includes a prodrug or other variant of the compound that is metabolized to the compound upon administration of the composition). It is to be understood that where a compound of the invention is useful, a prodrug that provides that compound is also useful. Accordingly, the treatments, uses, and methods of use described herein can be carried out with a compound described herein or a prodrug thereof.

Two events or entities are “associated” with one another, as that term is used herein, if the presence, level and/or form of one is correlated with that of the other. For example, a particular entity (e.g., polypeptide (e.g., a TAM kinase), genetic signature, metabolite, microbe, or event (e.g., myeloid infiltration)) is considered to be associated with a particular disease (e.g., a particular cancer) if its presence, level and/or form correlates with the incidence of and/or susceptibility to the disease (e.g., across a relevant population). Two or more entities can be physically “associated” with one another if they interact, directly or indirectly, so that they are and/or remain in physical proximity with one another. Two or more entities that are physically associated with one another can be covalently linked to one another or non-covalently associated, for example by means of hydrogen bonds, van der Waals interaction, hydrophobic interactions, magnetism, or combinations thereof.

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts, including where interacting entities or moieties are studied in isolation or in the context of more complex systems (e.g., while covalently or otherwise associated with a carrier entity and/or in a biological system or cell (e.g., in culture)).

As used herein, the term “biologically active” refers to an observable biological effect or result achieved by an agent or entity of interest (e.g., a compound described herein). For example, a specific binding interaction can be a biological activity. In some embodiments, modulation (e.g., induction, enhancement, or inhibition) of a biological pathway or event is a biological activity. The presence or extent of a biological activity is assessed through detection of a direct or indirect product produced by a biological pathway or event of interest.

As used herein, the term “biological sample” typically refers to a sample obtained or derived from a biological source (e.g., a tissue or organism (e.g., an animal or human patient) or cell culture) of interest. The biological sample can be or can comprise a biological tissue or fluid. For example, a biological sample can be or can comprise bone marrow; blood; blood cells; ascites; tissue or fine needle biopsy samples; cell-containing body fluids; free floating nucleic acids; sputum; saliva; urine; cerebrospinal fluid (CSF), peritoneal fluid; pleural fluid; feces; lymph; gynecological fluids; tissue swabbed from the skin or a mucus membrane (e.g., in the nose, mouth, or vagina); washings or lavages such as a ductal lavages or broncheoalveolar lavages; aspirates; scrapings; tissue biopsy specimens; surgical specimens; or other body fluids, secretions, and/or excretions and/or cells therefrom. In some embodiments, obtained cells are or include cells from an individual from whom the sample is obtained or for whom a treatment is intended. A sample can be a “primary sample” obtained directly from a source of interest by any appropriate means (e.g., by biopsy (e.g., fine needle aspiration or tissue biopsy), surgery, collection of body fluid (e.g., blood, lymph, feces etc.)). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

As used herein, the term “biomarker” refers to an entity whose presence, level, or form, correlates with a particular biological event or state of interest, so that it is considered to be a “marker” of that event or state. For example, a biomarker may be or may comprise a marker for a particular disease (e.g., cancer or a particular type of cancer, MDS, MPS, or a benign lesion), disease state (e.g., stage or grade of cancer), or for the likelihood that a particular disease may develop. In some embodiments, a biomarker may be or comprise a marker for a particular disease or therapeutic outcome, or likelihood thereof. Thus, a biomarker can be predictive, prognostic, or diagnostic, of a biological event or state of interest. A biomarker may be an entity of any chemical class. For example, a biomarker may be or comprise a nucleic acid, polypeptide, lipid, carbohydrate, small molecule, inorganic agent (e.g., a metal or ion), or a combination thereof. A biomarker can be a cell surface marker, an intracellular moiety, or found outside of cells (e.g., it can be secreted or is otherwise generated or present outside of cells, e.g., in a body fluid such as blood, plasma, urine, tears, saliva, CSF, etc.).

As used herein, the term “cancer” refers to a disease in which cells exhibit relatively abnormal, uncontrolled, and/or autonomous growth, resulting in an aberrant growth phenotype characterized by loss of control of cell proliferation to an extent detrimental to the patient having the disease. Intrinsic factors (e.g., a genetic mutation) and/or extrinsic factors (e.g., exposure to a pathogen or carcinogen) may have contributed to a patient's cancer. Further, the cancer can be classified by the type of tissue in which it originated (histological type) and/or by the primary site in the body in which the cancer first developed. Based on histological type, cancers are generally grouped into six major categories: carcinomas; sarcomas; myelomas; leukemias; lymphomas; and mixed types. A cancer treated as described herein may be of any one of these types and may comprise cells that are precancerous (e.g., benign), malignant, pre-metastatic, metastatic, and/or non-metastatic. A patient who has a malignancy or malignant lesion has a cancer. The present disclosure specifically identifies certain cancers to which its teachings may be particularly relevant, and one or more of these cancers may be associated with overexpression of one or more TAM kinases. A relevant cancer may be characterized by a solid tumor or by a hematologic tumor, which may also be known as a blood cancer (e.g., a type described above).

As used herein, the term “carrier” refers to a diluent, excipient, or vehicle with which a composition (e.g., a compound disclosed herein) is administered. Carriers include sterile or sterilizable liquids, including water (e.g., water for injection; WFI) and oils, including oils of petroleum, animal, vegetable or synthetic origin (e.g., peanut oil, soybean oil, mineral oil, and sesame oil). Carriers can be liquids, solids, or a mixture thereof (e.g., liquid carriers can include one or more solid carriers).

As used herein, the term “comparable” refers to two or more agents, entities, situations, sets of conditions, etc., that are not be identical to one another but are sufficiently similar to permit comparison therebetween so that one of ordinary skill in the art will appreciate that conclusions may reasonably be drawn based on differences or similarities observed. In some embodiments, comparable sets of conditions, circumstances, individuals, or populations are characterized by a plurality of substantially identical features and one or a small number of varied features. One of ordinary skill in the art will understand, in context, what degree of identity is required in any given circumstance for two or more such agents, entities, situations, sets of conditions, etc. to be considered comparable. For example, one of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “combination therapy” refers to situations in which a subject is simultaneously exposed to two or more therapeutic regimens (e.g., two or more therapeutic agents, including one or more compounds described herein). The two or more regimens may be administered simultaneously or sequentially (e.g., with sequential administration, all doses of a first regimen are administered prior to administration of any doses of a second regimen). In other embodiments, such compounds are administered in overlapping dosing regimens. “Administration” of a combination therapy may involve administration of one or more compounds to a subject receiving the other compound(s) in the combination. For clarity, combination therapy does not require that individual compounds be administered together in a single composition (or even necessarily at the same time), although two or more compounds may be administered together in a combination composition, or even in a combination compound (e.g., as part of a single chemical complex or covalent entity).

As used herein, the terms “dosage form” or “unit dosage form” refer to a physically discrete unit of an active agent (e.g., a therapeutic or diagnostic agent (e.g., a compound described herein)) for administration to a subject. Typically, each such unit contains a predetermined quantity of active agent, which may be a unit dosage amount (or a whole fraction thereof) appropriate for administration in accordance with a dosing regimen that has been determined to correlate with a desired or beneficial outcome when administered to a relevant population (i.e., with a therapeutic dosing regimen). One of ordinary skill in the art would appreciate that the total amount of a compound or pharmaceutical composition administered to a particular subject is determined by one or more attending physicians and may involve administration of multiple dosage forms.

As used herein, the term “dosing regimen” refers to a set of unit doses (typically more than one) that is administered individually to a subject, typically separated by periods of time. In some embodiments, a given therapeutic agent (e.g., compound) has a recommended dosing regimen comprising a plurality of doses, each of which is separated in time. Individual doses may be separated from one another by a time period of the same length. In other embodiments, at least two different time periods separate individual doses. Doses within a dosing regimen may be of the same unit dose amount or may contain at least two different unit dose amounts (e.g., a dosing regimen can comprise a first dose in a first dose amount, followed by one or more additional doses in a second dose amount that is the same as or different from the first dose amount). Where a dosing regimen correlates with a desired or beneficial outcome when administered across a relevant population, it may be referred to as a therapeutic dosing regimen.

As used herein, the term “inhibitor” refers to an agent (e.g., a compound or composition described herein), condition, or event whose presence, level, degree, type, or form correlates with a decreased level or activity of another agent (i.e., the inhibited agent, or target (e.g., a TAM kinase)). In general, an inhibitor may be or include an agent of any chemical class including, for example, small molecules (including any compound described herein), polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that the level of expression and/or activity of the target is reduced).

As used herein, the term “patient” or “subject” refers to any organism to which a compound or composition, as described herein, is administered. The compound and/or composition can be administered or provided for use in experimental, diagnostic, prophylactic, and/or therapeutic methods. Typical subjects include animals (e.g., mammals such as mice, rats, rabbits, dogs, cats, non-human primates, and humans; insects; etc.). A patient or subject can be suffering from a disease (e.g., cancer) or disorder, as described herein.

As used herein, the terms “prevent,” “prevention,” and “preventing,” when used in connection with the occurrence of a disease refer to reducing the risk of developing the disease and/or to delaying the onset of a sign or symptom of the disease. Prevention may be considered complete when onset of the disease has been delayed for a predefined period of time.

As used herein, the term “reference” describes a standard or control relative to which a comparison is performed. For example, an agent (e.g., a compound or composition), animal, cell, individual, population, sample, sequence or value of interest is compared with a reference or control agent (e.g., a compound or composition), animal, cell, individual, population, sample, sequence or value. A reference can be tested and/or determined substantially simultaneously with the testing or determination of the item of interest or it may be a historical reference or control, optionally embodied in a tangible medium. Typically, as would be understood by one or ordinary skill in the art, a reference is determined or characterized under comparable conditions or circumstances to those under assessment. One of ordinary skill in the art will appreciate when sufficient similarities are present to justify reliance on and/or comparison to a particular possible reference.

With respect to methods of treating or preventing a disease described herein and uses of the compounds and compositions described herein, the term “response” refers to any beneficial alteration in a subject's condition that occurs as a result of, or that correlates with, administration of a compound or composition described herein. Such alteration may include stabilization of the condition (e.g., inhibiting deterioration that would have been expected to take place in the absence of the treatment, amelioration of signs or symptoms of the condition, and/or improvement in the prospects for cure of the condition. The alteration may refer to a subject's response or to a tumor's response. Response may be measured according to a wide variety of criteria, including clinical criteria and objective criteria. Techniques for assessing response include, but are not limited to, assay assessment, clinical examination, positron emission tomography (PET), X-ray, computed tomography (CT) scan, magnetic resonance imaging (MRI), ultrasound, endoscopy, laparoscopy, the presence or level of tumor markers in a sample obtained from a subject (biomarkers), cytology, and/or histology. Regarding a tumor's response, methods and guidelines for assessment are discussed in Therasse et al. (J. Natl. Cancer Inst., 92(3):205-216, 2000). The exact response criteria can be selected in any appropriate manner, provided that when comparing groups of patients and/or tumors, the groups to be compared are assessed based on the same or comparable criteria for determining response rate. One of ordinary skill in the art will be able to select appropriate criteria.

As used herein, the term “substantially” refers to the qualitative condition of exhibiting a characteristic or property of interest to a total or near total extent or degree. One of ordinary skill in the art will understand that biological and chemical phenomena rarely, if ever, go to completion and/or proceed to completeness or achieve or avoid an absolute result. The term “substantially” is therefore used herein to describe the potential lack of completeness inherent in many biological and chemical phenomena.

As used herein, an individual who is “susceptible to” a disease is at risk for developing the disease. In some embodiments, such an individual has not been diagnosed with the disease. An individual who is susceptible to a disease can be an individual who has been exposed to conditions associated with development of the disease (e.g., an individual who is susceptible to cancer may have been exposed to high levels of radiation or carcinogens). In some embodiments, a risk of developing a disease is a population-based risk (e.g., family members of individuals suffering from the disease may be susceptible to, or have an elevated risk of developing, the disease).

As used herein, “symptoms are reduced” when one or more symptoms of a particular disease is reduced in magnitude (e.g., intensity, severity, etc.) and/or frequency. The same is true for a sign of the disease; a sign is reduced when reduced in magnitude and/or frequency.

As used herein, terms such as “improve,” “heighten,” “increase,” “decrease,” “reduce,” and grammatical equivalents thereof may refer to changes in a value relative to a reference, such as a measurement obtained from the same subject prior to initiation of a treatment or preventative method described herein or from a control individual (or multiple control individuals (i.e., populations may be assessed)) in the absence of the treatment described herein. A “control individual” can be an individual afflicted with the same disease as an individual being treated or can be a individual who is healthy by virture of not being afflicted with that disease.

As used herein, a “therapeutic regimen” is a dosing regimen whose administration across a relevant population is correlated with a desired or beneficial therapeutic outcome.

As used herein, a “therapeutically effective amount” refers to an amount (of, for example, a compound or composition described herein) that produces or is expected to produce the desired effect for which it is administered. In some embodiments, the term refers to an amount that is sufficient, when administered to a population suffering from or susceptible to a disease in accordance with a therapeutic dosing regimen, to treat the disease. In some embodiments, a therapeutically effective amount is one that reduces the incidence and/or severity of one or more signs or symptoms of the disease. One of ordinary skill in the art will appreciate that the term “therapeutically effective amount” does not in fact require successful treatment be achieved in any particular individual. Rather, a therapeutically effective amount is that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease) or fluids (e.g., blood, saliva, serum, sweat, tears, urine, etc.). In some embodiments, a therapeutically effective amount of a compound or composition described herein may be formulated and/or administered in a single dose; in other embodiments, the therapeutically effective amount will be administered in a plurality of doses, for example, as part of a dosing regimen.

As used herein, the term “treatment” (also “treat” or “treating”) refers to any administration of a therapy that partially or completely alleviates, ameliorates, relives, inhibits, reduces the severity of, and/or reduces the incidence of one or more symptoms, features, and/or causes of a particular disease; for example, with treatment, a patient's symptoms are reduced. Treatment may be of a subject who exhibits only early signs or symptoms of the disease. Alternatively or additionally, treatment may be of a subject who exhibits one or more established signs or symptoms of the relevant disease, and the subject may have been diagnosed as suffering from the relevant disease. The term “treatment,” as used herein, is distinguished from “prophylaxis,” which relates to prevention of a disease.

As described above, in certain embodiments provided compounds are of formula I:

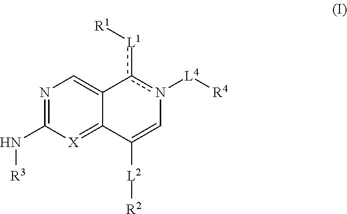

a prodrug thereof, or a pharmaceutically acceptable salt thereof, wherein each [symbol] and each of X, L¹, R¹, L², R², R³, L⁴, and R⁴ is as defined above and described in classes and subclasses herein, both singly and in combination.

In some embodiments of Formula I, R¹ is additionally selected from halo.

In some embodiments of Formula I, when L¹ is a bond, R¹ is C₂-C₆ alkyl, aryl, heteroaryl, heterocyclyl or carbocyclyl, wherein R¹ is optionally substituted with up to four different substituents; in some embodiments, when L¹ is a bond, R¹ is other than —CH₃ or —CHF₂.

In some embodiments of Formula I, when L² is a bond, R² is C₁-C₆ alkyl, monocyclic aryl, monocyclic heteroaryl, monocyclic heterocyclyl or monocyclic carbocyclyl, wherein R² is optionally substituted with up to four different substituents. In some embodiments of Formula I, when L² is a bond, R² is other than optionally substituted 1H-indolyl. In some embodiments of Formula I, when L² is a bond, R² is other than optionally substituted 1H-indol-3-yl. In some embodiments of Formula I, when L² is a bond, R² is other than trifluoromethyl-substituted 1H-indol-3-yl or cyano-substituted 1H-indol-3-yl.

In some embodiments of Formula I, when L¹ is a bond, —C₁-C₆ alkylene, —O—(C₀-C₆ alkylene)-†, —NH—(C₀-C₆ alkylene-†, or —N(C₁-C₆alkyl)-(C₀-C₆ alkylene-†, and R¹ is C₁-C₆ alkyl (or C₂-C₆ alkyl), any alkyl or alkylene portion of L¹ is optionally and independently substituted with one or more monovalent substitutents; and R¹ is optionally substituted with up to four different monovalent substituents.

In some embodiments of Formula I, when L² is a bond, —O—(C₀-C₆ alkylene)-*, —NH—(C₀-C₆ alkylene)-*, or —N(C₀-C₆ alkyl)-(C₀-C₆ alkylene)-*, and R² is C₁-C₆ alkyl, any alkyl or alkylene portion of L² is optionally substituted with one or more monovalent substitutents; and R² is optionally substituted with up to four different monovalent substituents.

For the purpose of clarity, the term monvalent substituent means a substituent that is bound by a single bond. Thus, the term monovalent substituent specifically excludes carbon substituents, such as ═O, ═S and ═NR.

It should be understood that compounds of the invention where L¹ and R¹ are taken together to form an optionally substituted, N-linked saturated heterocyclyl are equivalent to compounds where L¹ is a bond and R¹ is optionally substituted, nitrogen-containing heterocyclyl, wherein a ring nitrogen in R¹ is directly bound to the depicted bicyclic ring in Formula I.

As used herein, unless otherwise stated, references to formula I also include all subgenera of formula I defined and described herein (e.g., formulae Ia, Ib, Ic and Id).

In some embodiments, L¹, if present, is —O—, —O—CH₂-†, —O—CH₂—CH₂†, —NH—, —N(CH₃)—, or —NH—CH₂-†; or L¹ and R¹ are taken together to form an optionally substituted azetidinyl, pyrrolidinyl, or piperidinyl (i.e., L¹ is a bond; and R¹ is an optionally substituted azetidin-1-yl, pyrrolidin-1-yl or piperidin-1-yl). In some embodiments, L¹ is additionally selected from a bond, and —CH₂—.

In some embodiments, R¹, if present, is morpholinyl, cyclopropyl, cyclohexyl, cyclobutyl, tetrahydropyranyl, tetrahydrofuranyl, piperidinyl, thiopyranyl, pyrrolidinyl, oxabicyclo[3.2.1]octanyl, pyridinyl, pyrazolyl, or phenyl, wherein R¹ is optionally substituted with up to 5 substituents independently selected from halo, —OH, —NH₂, —C₁-C₄ alkyl, —O—C₁-C₄ alkyl, —C₁-C₄ alkylene-O—C₁-C₄ alkyl, —S(O)₂—C₁-C₄ alkyl, optionally substituted heterocyclyl, optionally substituted heteroaryl, —C(O)—C₁-C₄ alkyl, —NH—C(O)—C₁-C₄ alkyl, —C(O)—C₁-C₄ alkylene-O—C₁-C₄ alkyl, and —NH—C(O)—O—C₁-C₄ alkyl.

In some embodiments, R¹, if present, is C₁-C₄ alkyl, or —C₁-C₄ alkylene-O—C₁-C₄ alkyl.

In some embodiments, R¹, if present, is halo and L¹ is a bond.

In some embodiments, R¹, if present, is chloro or fluoro; and L¹ is a bond. In some aspects of these embodiments, R¹ is chloro.

In some embodiments, R¹, if present, is —CH₃, —CH₂CHOCH₃, morpholin-4-yl, 4,4-difluorocyclohexyl, 4-methoxycyclohexyl, 4-hydroxycyclohexyl, 4-hydroxycyclobutyl, tetrahydropyran-4-yl, piperidin-4-yl, 1-trifluoromethylcarbonylpiperidin-4-yl, 1-methylpiperidin-4-yl, 1-methyl sulfonylpiperidin-4-yl, 1-(2-dimethylaminocarbonylethyl)piperidin-4-yl, 1-(dimethylaminocarbonylmethyl)piperidin-4-yl, 1-(2-methoxyethyl)piperidin-4-yl, 1,1-dioxotetrahydro-2H-thiopyran-4-yl, 1,2,2,6,6-pentamethylpiperidin-4-yl, 8-oxabicyclo[3.2.1]-octan-3-yl, 4-fluorophenyl; or wherein L¹ and R¹ are taken together to form 3-dimethylamino-pyrrolidin-1-yl, 3-dimethylaminopiperidin-1-yl, or 3-dimethylaminoazetidin-1-yl (i.e., wherein L¹ is a bond and R¹ is 3-dimethylaminopiperidin-1-yl, or 3-dimethylaminoazetidin-1-yl).

In some embodiments, R¹, if present, is —CH₂CH₃, —CH₂CH₂N(CH₃)₂, —CH₂CF₃, —CH(CH₂OH)₂, —CF₃, 1-(2-methoxyethyl)-2,2,6,6-tetrafluoro-piperidin-4-yl, 1-(2-methylpyrimidin-4-yl)piperidin-4-yl, 1-(2-methoxyethyl)-2,2,6,6-tetramethyl-piperidin-4-yl, 1-(methoxymethylcarbonyl)-2,2,6,6-tetramethylpiperidin-4-yl, 1-(tetrahydrofuran-2-ylmethyl)-2,2,6,6-tetramethylpiperidin-4-yl, 1,2,2-trimethylpiperidin-4-yl, 1,2,6-trimethylpiperidin-4-yl, 1-acetyl-2,2,6,6-tetramethyl-piperidin-4-yl, 1-ethyl-2,2,6,6-tetramethylpiperidin-4-yl, 1-methylpiperidin-3-yl, 1-methyl-pyridin-4-yl, 1-methylpyrrolidin-3-yl, 1-tetrahydrofuran-3-ylpiperidin-4-yl, 2-methylpyridin-4-yl, 2,2-dimethyl-3-hydroxycyclobutyl, 2,6-dimethyltetrahydrpyran-4-yl, 2,2,6,6-tetramethylpiperidin-4-yl, 3-(4-methylpiperazin-1-ylcarbonyl)cyclobutyl, 3-(methoxycarbonylamino)cyclobutyl, 3-(methylcarbonylamino)cyclobutyl, 3-(morpholin-4-yl)cyclobutyl, 3-(morpholin-4-ylmethyl)cyclobutyl, 3,3-difluoropiperidin-4-yl, 3-dimethylaminocyclopentyl, 3-hydroxycyclobutyl, 3-methoxycyclobutyl, 3-methyl-3-hydroxycyclobutyl, 4-aminocyclohexyl, 4-methyl-4-hydroxycyclohexyl, 4-(1-methyl-1-hydroxyethyl)cyclohexyl, 4-hydroxybicyclo[2.2.2]octanyl, 6-methylpyridin-3-yl, 8-methyl-8-azabicyclo[3.2.1]octan-3-yl, cyclopropyl, pyridin-3-yl, pyridin-4-yl, tetrahydrofuran-3-yl, tetrahydropyran-4-yl, 3-methoxycyclobutyl, 3-acetylaminocyclobutyl, 3-methoxycarbonyl-aminocyclobutyl, 3-(morpholin-4-yl)cyclobutyl, 8-(2-methoxyethyl)-8-azabicyclo[3.2.1]octan-3-yl, 8-(oxetan-3-yl)-8-azabicyclo[3.2.1]octan-3-yl, 8-methylcarbonyl-8-azabicyclo[3.2.1]octan-3-yl, 8-(tetrahydrofuran-2-ylmethyl)-8-azabicyclo[3.2.1]octan-3-yl, 8-(tetrahydrofuran-3-yl)-8-azabicyclo[3.2.1]octan-3-yl, 8-(tetrahydrofuran-3-ylmethyl)-8-azabicyclo[3.2.1]octan-3-yl, 8-(2,2,2-trifluoroethyl)-8-azabicyclo[3.2.1]octan-3-yl, and 1-(1-(morpholin-4-yl)carbonyl-1-methyl-ethyl)pyrazol-4-yl.

In some embodiments, R, if present, is hydrogen.

In some embodiments, L⁴, if present, is a bond, —CH₂—, or —CH₂CH₂—.

In some embodiments, R⁴, if present, is amino-substituted C₁-C₆ alkyl, optionally substituted phenyl or optionally substituted saturated heterocyclyl.

In some embodiments, wherein R⁴, if present, is —CH₂N(CH₃)₂, morpholin-4-yl, 1-methylpiperidin-4-yl, 1-isopropylpiperidin-4-yl, 1-methylsulfonylpiperidin-4-yl, 1-acetylpiperidin-4-yl, 1-(dimethylaminocarbonylmethyl)piperidin-4-yl, 1-(dimethylaminomethylcarbonyl)piperidin-4-yl, 1-(2-dimethylaminocarbonylethyl)piperidin-4-yl, 1-(2-methoxyethyl)piperidin-4-yl, 4-(morpholin-4-ylmethyl)phenyl, 4-fluorophenyl, or tetrahydropyran-4-yl.

In some embodiments, R⁴, if present, is additionally selected from —CH(CH₃)₂, 1-methyl-3-fluoropiperidin-4-yl, 1-methylpiperidin-3-yl, 1-(oxetan-3-yl)piperidin-4-yl, 4-hydroxybi-cyclo[2.2.2]octanyl, 4-aminobicyclo[2.2.2]octanyl, 4-dimethylaminobicyclo[2.2.2]octanyl, 4-methylmorpholin-3-yl, 3-dimethylaminocyclopentyl, 4-aminophenyl, and 4-aminocyclohexyl.

In some embodiments, L² is a bond. In some embodiments, L² is —CH₂—.

In some embodiments, R² is phenyl, cyclohexyl, 1H-pyrazolyl, piperidinyl, pyridinyl, pyridazinyl, or 4,5,6,7-tetrahydro-1H-indazolyl wherein R² is optionally substituted with up to 3 substituents independently selected from halo, —OH, —NH₂, —NH(C₁-C₄ alkyl), —N—(C₁-C₄ alkyl)₂, —S(O)₂—C₁-C₄ alkyl, —C₁-C₄ alkyl optionally substituted with one or more substituent selected from —CN, —OH and halo, —C(O)—NH₂, —C(O)—NH(C₁-C₄ alkyl), —C(O)—N(C₁-C₄ alkyl)₂, —S(O)₂—NH₂, —S(O)₂—NH(C₁-C₄ alkyl), —S(O)₂—N(C₁-C₄ alkyl)₂, —NH—C(O)—C₁-C₄ alkyl, —NH—S(O)₂—C₁-C₄ alkyl, —NH—C(O)—NH—C₁-C₄ alkyl, —NH—S(O)₂—NH—C₁-C₄ alkyl, and optionally substituted pyrrolidinyl.

In some embodiments, R² is selected from 1-(1-methylpyrrolidin-3-yl)pyrazol-4-yl, 1H-pyrazol-4-yl, 1-methylaminocarbonylpiperidin-4-yl, 1-methylaminosulfonylpiperidin-4-yl, 1-methylpiperidin-2-yl, 1-methylpiperidin-3-yl, 1-methylpiperidin-4-yl, 1-(t-butoxycarbonyl)piperidin-4-yl, 1,4-dioxaspiro[4.5]decan-8-yl, 1,4-dioxaspiro[4.5]dec-7-en-8-yl, 3-(azetidin-3-yl sulfonyl)phenyl, 3-(methylsulfonylamino)phenyl, 3-amino-carbonylphenyl, 3-aminosulfonylphenyl, 3-cyanophenyl, 3-cyanomethylphenyl, 3-dimethyl-aminosulfonylphenyl, 3-(hydroxycarbonylmethyl)phenyl, 3-isopropylsulfonylphenyl, 3-methyl sulfonylphenyl, 4-(1-cyanocyclopropyl)phenyl, 4,4-difluorocyclohexyl, 4-acetylaminocyclohexyl, 4-aminophenyl, 4-cyanophenyl, 4-cyanomethylphenyl, 4-dimethylaminocyclohexyl, 4-fluorophenyl, 4-hydroxycyclohexyl, 4-hydroxyphenyl, 4-hydroxymethylphenyl, 4-methylaminocarbonylamino-cyclohexyl, 4-methylaminosulfonyl-aminocyclohexyl, 4-(methylaminocarbonylamino)phenyl, 4-methyl sulfonylphenyl, 4-methyl sulfonylaminocyclohexyl, 4-oxocyclohexyl, 4-trifluoromethyl-4-hydroxycyclohexyl, 4-methyl-4-hydroxycyclohexyl, 4-(4-methylpiperazin-1-yl)phenyl, 4-(1-methylpiperidin-4-yl)phenyl, 6-aminopyridin-3-yl, piperidin-2-yl, piperidin-3-yl, piperidin-4-yl, pyridazin-4-yl, 4,5,6,7-tetrahydro-1H-indazol-5-yl, and 4,5,6,7-tetrahydro-1H-indazol-6-yl.

In some embodiments, R² is optionally substituted C₃-C₈ cycloalkyl.

In some embodiments, R² is 4-hydroxycyclohexyl or 4-dimethylaminocyclohexyl. In some aspects of these embodiments, R² is

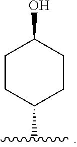 [SMILES: *[C@H]1CC[C@H](O)CC1]

In some embodiments, R³ is —C₃-C₆ alkyl, —(C₂-C₆ alkylene)-O—(C₁-C₆ alkyl), phenyl, C₃-C₆ cycloalkyl, saturated heterocyclyl, —(C₁-C₂ alkylene)-aryl or —(C₁-C₂ alkylene)-heteroaryl, wherein each R³ is optionally substituted with 1-2 substituents independently selected from halo, —OH, —C₁-C₄ alkyl, and —O—C₁-C₄ alkyl. In some embodiments, the 1-2 optional and independently selected substitutents on R³ are additionally selected from deuterium, —CN, —S(O)₂—C₁-C₄ alkyl, —C(O)OH, —C(O)O—C₁-C₄ alkyl, —C(O)NH₂, —C(O)NH—(C₁-C₄ alkyl), tetrazolyl, and oxo. In some embodiments, each R³ is optionally substituted with 3 substituents independently selected from halo and deuterium.

In some embodiments, R³ is n-butyl, isopropyl, butan-2-yl, heptan-2-yl, 1,3-dimethoxy-propan-2-yl, 3-methoxypropan-2-yl, pentan-2-yl, 4-methylpentan-2-yl, pentan-3-yl, 3-methyl-butan-2-yl, cyclopentyl, cyclohexyl, 4-chlorophenyl, tetrahydrofuran-3-yl, 3-hydroxypropan-2-yl, 3,3-dimethylcyclobutyl, 1-methyl-2-oxopyrrolidin-3-yl, 2-(2-methyl-1H-imidazol-1-yl)ethyl, 4,4-difluorocyclohexyl, 3-methylcyclobutyl, or 1-phenylethyl.

In some embodiments, R³ is 1-ethylcyclolpropyl, 1-methyl-5-oxopyrrolidin-3-yl, 1-methylcyclopropyl, 1-methylsulfonylpyrrolidin-3-yl, 2-(phenyl)ethan-2-yl, 2-(pyridin-4-yl)ethan-2-yl, 2-fluorophenyl, 2-methyl-4-chlorophenyl, 2-methyl-5-chlorophenyl, 2-methoxycyclopropanyl, 3-(tri-deuteromethoxy)propan-2-yl, 3-(difluoromethoxy)propan-2-yl, 3,3,3-trifluoropropyl, 3,3-difluorocyclopentyl, 3,3-difluoropropan-2-yl, 3,4-dichlorophenyl, 3-chlorophenyl, 3-cyanopropan-2-yl, 3-ethoxypropan-2-yl, 3-ethyl-4-chlorophenyl, 3-ethylphenyl, 3-fluorophenyl, 3-fluoropropan-2-yl, 3-methylphenyl, 4-(methoxy-carbonyl)phenyl, 4-(1,2,3,5-tetrazol-4-yl)phenyl, 4-(1,2,4,5-tetrazol-3-yl)phenyl, 4,4-difluorobutan-2-yl, 4,4,4-trifluorobutan-2-yl, 4-aminocarbonylphenyl, 4-cyanophenyl, 4-fluorobutan-2-yl, 4-fluorophenyl, 4-hydroxybutan-2-yl, 4-hydroxycarbonylcyclohexyl, 4-hydroxycarbonylphenyl, 4-methylaminocarbonylphenyl, 4-methoxybutan-2-yl, isobutyl, phenyl, propan-2-yl, pyrrolidin-3-yl, t-butyl, tetrahydropyran-3-yl, or tetrahydropyran-4-yl.

In some embodiments, provided compounds are of formula Ia:

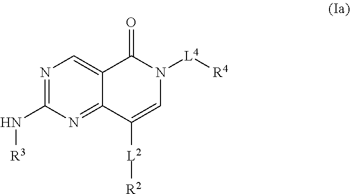

or a pharmaceutically acceptable salt thereof, wherein each of L², R², R³, L⁴ and R⁴ is as defined above and described in classes and subclasses herein, both singly and in combination.

In some embodiments, L⁴ is a bond and R⁴ is C₁-C₆ alkyl, heterocyclyl or carbocyclyl, wherein R⁴ is optionally substituted with up to four different substituents.

In some embodiments, R⁴ is amino-substituted C₁-C₆ alkyl, or optionally substituted saturated heterocyclyl. In some embodiments, R⁴ is —CH₂N(CH₃)₂, morpholin-4-yl, 1-methyl-piperidin-4-yl, 1-isopropylpiperidin-4-yl, 1-methylsulfonylpiperidin-4-yl, 1-acetylpiperidin-4-yl, 1-(dimethylaminocarbonylmethyl)piperidin-4-yl, 1-(dimethylaminomethylcarbonyl)piperidin-4-yl, 1-(2-dimethylaminocarbonylethyl)piperidin-4-yl, 1-(2-methoxyethyl)piperidin-4-yl, or tetra-hydropyran-4-yl. In some embodiments, R⁴ is —CH(CH₃)₂, 1-methyl-3-fluoropiperidin-4-yl, 1-methylpiperidin-3-yl, 1-(oxetan-3-yl)piperidin-4-yl, 4-hydroxybicyclo[2.2.2]octanyl, 4-amino-bicyclo[2.2.2]octanyl, 4-dimethylaminobicyclo[2.2.2]octanyl, 4-methylmorpholin-3-yl, 3-dimethylaminocyclopentyl, 4-aminophenyl, or 4-aminocyclohexyl.

In some embodiments, R³ is optionally substituted —C₁-C₆ alkyl, —C₂-C₆ alkenyl, —C₂-C₆ alkynyl, —(C₂-C₆ alkylene)-O—(C₁-C₆ alkyl), —(C₁-C₆ alkylene)-aryl, —(C₁-C₆ alkylene)-carbocyclyl, —(C₁-C₆ alkylene)-heterocyclyl, or —(C₁-C₆ alkylene)-heteroaryl, wherein each substituent on an alkyl or alkylene portion of R³ is a monovalent substituent. In some embodiments, R³ is —C₃-C₆ alkyl, —(C₂-C₆ alkylene)-O—(C₁-C₆ alkyl), —(C₁-C₂ alkylene)-aryl or —(C₁-C₂ alkylene)-heteroaryl, wherein each R³ is optionally substituted with 1-2 substituents independently selected from halo, —OH, —C₁-C₄ alkyl, and —O—C₁-C₄ alkyl. In some embodiments, R³ is n-butyl, isopropyl, butan-2-yl, heptan-2-yl, 1,3-dimethoxypropan-2-yl, 3-methoxypropan-2-yl, pentan-2-yl, 4-methylpentan-2-yl, pentan-3-yl, 3-methylbutan-2-yl, 3-hydroxypropan-2-yl, 2-(2-methyl-1H-imidazol-1-yl)ethyl, or 1-phenylethyl.

In some embodiments, provided compounds are of formula Ib:

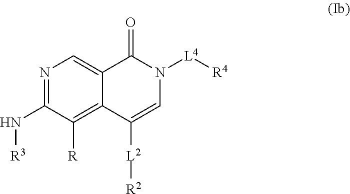

or a pharmaceutically acceptable salt thereof, wherein each of R, L², R², R³, L⁴ and R⁴ is as defined above and described in classes and subclasses herein, both singly and in combination.

In some embodiments, R³ is optionally substituted —C₁-C₆ alkyl, —C₂-C₆ alkenyl, —C₂-C₆ alkynyl, —(C₂-C₆ alkylene)-O—(C₁-C₆ alkyl), —(C₁-C₆ alkylene)-aryl, —(C₀-C₆ alkylene)-carbocyclyl, —(C₀-C₆ alkylene)-heterocyclyl, or —(C₁-C₆ alkylene)-heteroaryl, wherein each substituent on an alkyl or alkylene portion of R³ is a monovalent substituent. In some embodiments, R³ is —C₃-C₆ alkyl, —(C₂-C₆ alkylene)-O—(C₁-C₆ alkyl), C₃-C₆ cycloalkyl, saturated heterocyclyl, —(C₁-C₂ alkylene)-aryl or —(C₁-C₂ alkylene)-heteroaryl, wherein each R³ is optionally substituted with 1-2 substituents independently selected from halo, —OH, —C₁-C₄ alkyl, and —O—C₁-C₄ alkyl. In some embodiments, R³ is n-butyl, isopropyl, butan-2-yl, heptan-2-yl, 1,3-dimethoxypropan-2-yl, 3-methoxypropan-2-yl, pentan-2-yl, 4-methylpentan-2-yl, pentan-3-yl, 3-methylbutan-2-yl, cyclopentyl, cyclohexyl, tetrahydrofuran-3-yl, 3-hydroxypropan-2-yl, 3,3-dimethylcyclobutyl, 1-methyl-2-oxopyrrolidin-3-yl, 2(2-methyl-1H-imidazol-1-yl)ethyl, 4,4-difluorocyclohexyl, 3-methylcyclobutyl, or 1-phenylethyl.

In some embodiments, provided compounds are of formula Ic:

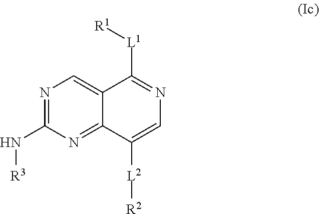

or a pharmaceutically acceptable salt thereof, wherein each of L¹, R¹, L², R² and R³ is as defined above and described in classes and subclasses herein, both singly and in combination.

In some embodiments of formula Ic, the compound is of formula Ic-1:

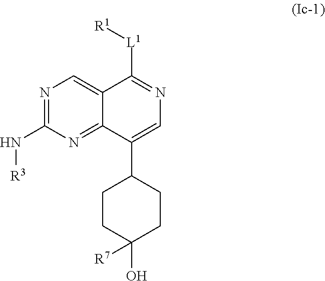

wherein each of L¹, R¹, and R³ is as defined above and described in classes and subclasses herein, both singly and in combination; and R⁷ is selected from hydrogen, or C₁-C₃ alkyl. In some embodiments of formula Ic-1, L¹ is —O—. In some embodiments of formula Ic-1, R¹ is C₃-C₆ cycloalkyl or heterocyclyl, wherein R¹ is substituted with 1 to 5 substituents independently selected from —OH, —CH₃, and —CH₂CH₃. In some embodiments of formula Ic-1, R¹ is C₃-C₆ cycloalkyl or heterocyclyl, wherein R¹ is substituted with 1 to 5 substituents independently selected from —OH, —CH₃, —CH₂CH₃, —CH₂CH₂OCH₃, —NHC(O)CH₃, tetrahydrofuran-2-ylmethyl, tetrahydrofuran-3-ylmethyl, tetrahydrofuran-3-yl, tetrahydrofuran-2-yl, morpholin-4-yl, and morpholin-4-ylmethyl. In some embodiments of formula Ic-1, R⁷ is hydrogen. In some embodiments of formula Ic-1, R⁷ is methyl. In more specific embodiments of formula Ic-1, L¹ is —O— and R¹ is selected from 3-hydroxycyclobutyl, 1-ethyl-2,2,6,6-tetramethylpiperidin-4-yl, 8-methyl-8-azabicyclo[3.2.1]octan-3-yl, 8-(2-methoxyethyl)-8-azabicyclo[3.2.1]octan-3-yl, 8-(oxetan-3yl)-8-azabicyclo[3.2.1]octan-3-yl, and 8-methylcarbonyl-8-azabicyclo[3.2.1]octan-3-yl. In some embodiments of formula Ic-1, R³ is a C₃-C₅ alkyl optionally substituted with up to 3 halo substituents. In some embodiments of formula Ic-1, R³ is selected from

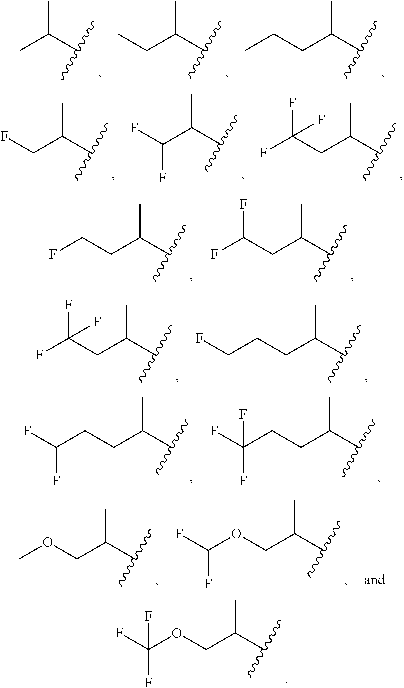

In some embodiments of formula Ic-1, R³ is

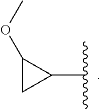 [SMILES: *C1CC1OC]

In some embodiments of formula Ic, the compound is of formula Ic-2:

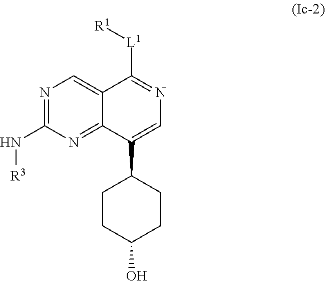

wherein each of L¹, R¹, and R³ is as defined above and described in classes and subclasses herein, both singly and in combination. In some embodiments of formula Ic-2, L¹ is —O—. In some embodiments of formula Ic-2, R¹ is C₃-C₆ cycloalkyl or heterocyclyl, wherein R¹ is substituted with 1 to 5 substituents independently selected from —OH, —CH₃, —CH₂CH₃, —CH₂CH₂OCH₃, —NHC(O)CH₃, tetrahydrofuran-2-ylmethyl, tetrahydrofuran-3-ylmethyl, tetrahydrofuran-3-yl, tetrahydrofuran-2-yl, morpholin-4-yl, and morpholin-4-ylmethyl In some more specific embodiments of Formula Ic-2, -L¹-R¹ is selected from:

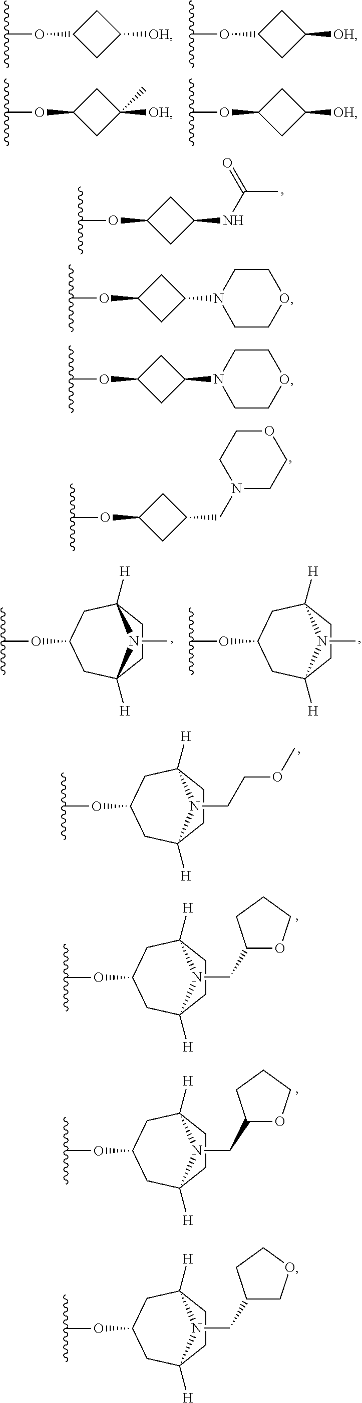

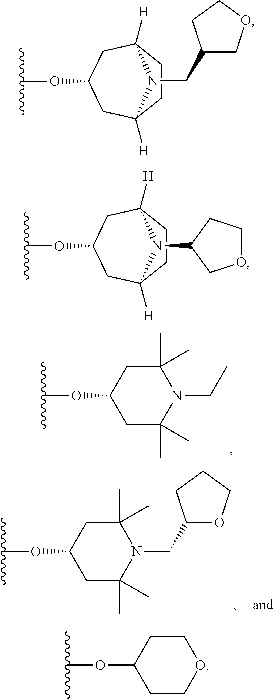

In some embodiments of formula Ic-2, R³ is a C₃-C₅ alkyl optionally substituted with up to 3 halo substituents. In some embodiments of formula Ic-2, R³ is selected from the following (S)-alkyl, (S)-haloalkyl, (S)-alkoxyalkyl, and (S)-alkoxyhaloalkyl groups:

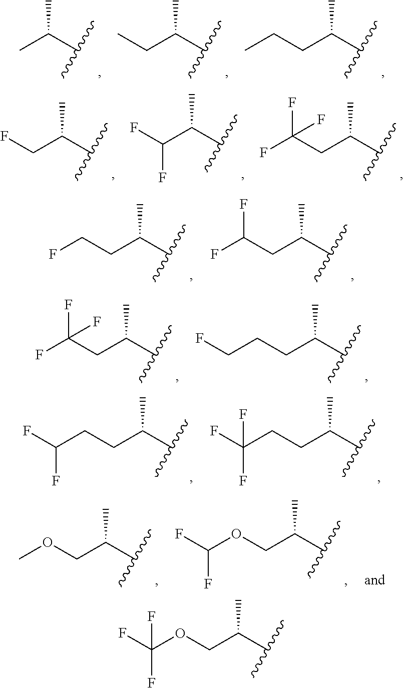

with the branched methyl in the S-configuration. In some embodiments of formula Ic-2, R³ is

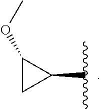 [SMILES: *[C@H]1C[C@@H]1OC]

In some embodiments of formula Ic, the compound is of formula Ic-3:

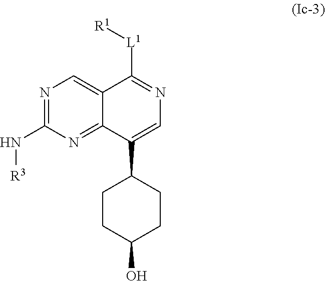

wherein each of L¹, R¹, and R³ is as defined above and described in classes and subclasses herein, both singly and in combination. In some embodiments of formula Ic-3, L¹ is —O—. In some embodiments of formula Ic-3, R¹ is C₃-C₆ cycloalkyl or heterocyclyl, wherein R¹ is substituted with 1 to 5 substituents independently selected from —OH, —CH₃, —CH₂CH₃, —CH₂CH₂OCH₃, —NHC(O)CH₃, tetrahydrofuran-2-ylmethyl, tetrahydrofuran-3-ylmethyl, tetrahydrofuran-3-yl, tetrahydrofuran-2-yl, morpholin-4-yl, and morpholin-4-ylmethyl. In some more specific embodiments of Formula Ic-3, -L¹-R¹ is selected from:

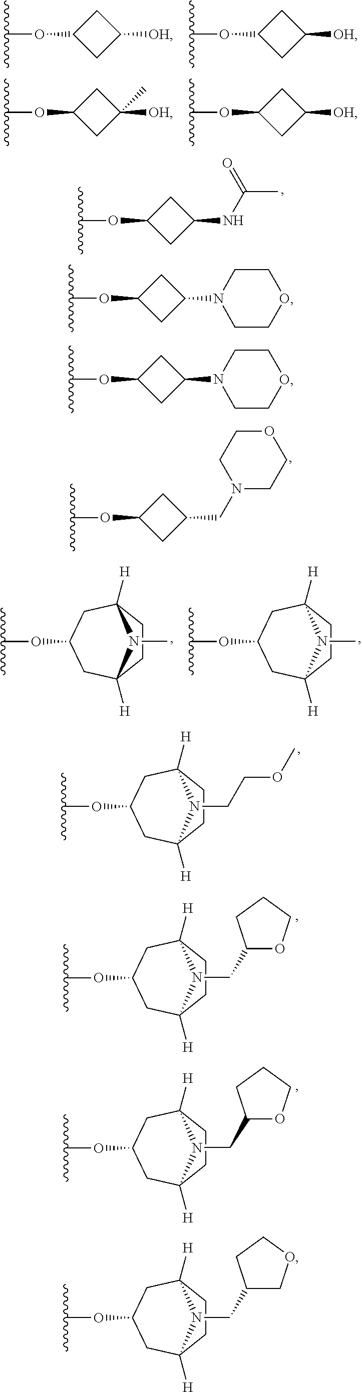

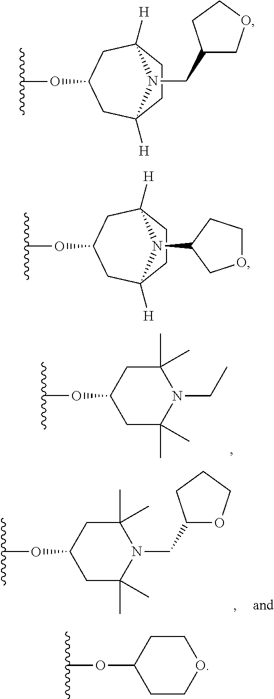

In some embodiments of formula Ic-3, R³ is a C₃-C₅ alkyl optionally substituted with up to 3 halo substituents. In some embodiments of formula Ic-3, R³ is selected from the following (S)-alkyl, (S)-haloalkyl, (5)-alkoxyalkyl, and (S)-alkoxyhaloalkyl groups:

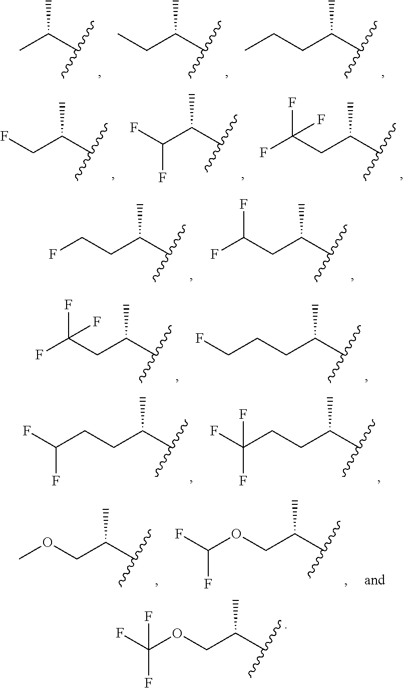

In some embodiments of formula Ic-3, R³ is

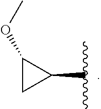 [SMILES: *[C@H]1C[C@@H]1OC]

In some embodiments, provided compounds are of formula Id:

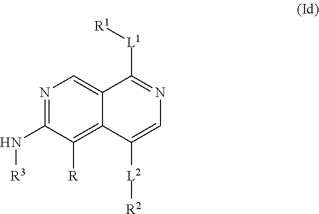

or a prodrug or pharmaceutically acceptable salt thereof, wherein each of R, L¹, R¹, L², R² and R³ is as defined above and described in classes and subclasses herein, both singly and in combination.

In some embodiments, a provided compound is a compound depicted in Table 1, or a prodrug or pharmaceutically acceptable salt thereof.

TABLE 1

| Compound | Structure |
|---|---|
| 101 | 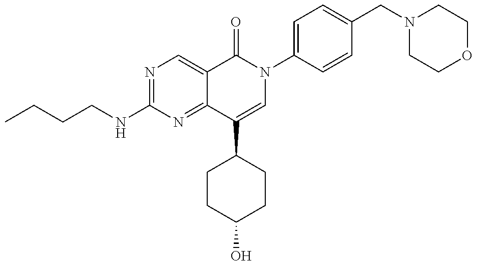 [SMILES: CCCCNc1ncc2c(=O)n(-c3ccc(CN4CCOCC4)cc3)cc([C@H]3CC[C@H](O)CC3)c2n1] |
| 102 | 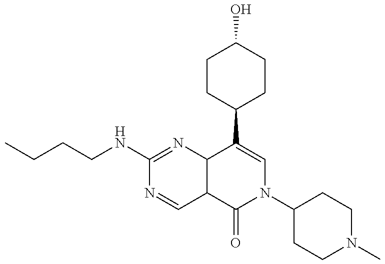 [SMILES: CCCCNC1=NC2C([C@H]3CC[C@H](O)CC3)=CN(C3CCN(C)CC3)C(=O)C2C=N1] |
| 103 | 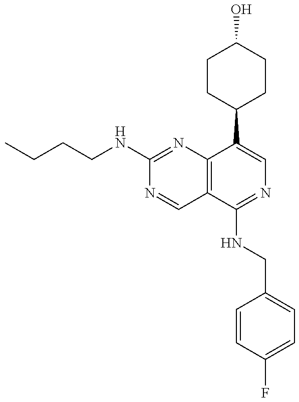 [SMILES: CCCCNc1ncc2c(NCc3ccc(F)cc3)ncc([C@H]3CC[C@H](O)CC3)c2n1] |
| 104 | 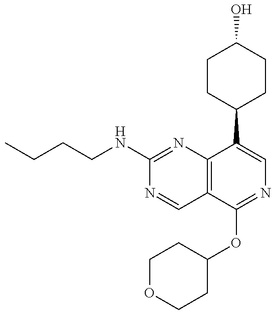 [SMILES: CCCCNc1ncc2c(OC3CCOCC3)ncc([C@H]3CC[C@H](O)CC3)c2n1] |
| 105 | 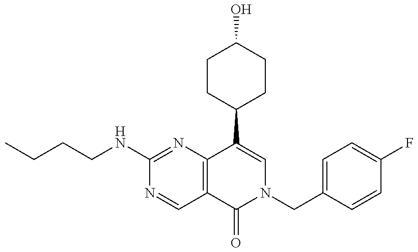 [SMILES: CCCCNc1ncc2c(=O)n(Cc3ccc(F)cc3)cc([C@H]3CC[C@H](O)CC3)c2n1] |
| 106 | 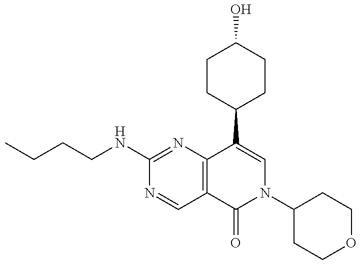 [SMILES: CCCCNc1ncc2c(=O)n(C3CCOCC3)cc([C@H]3CC[C@H](O)CC3)c2n1] |
| 107 | 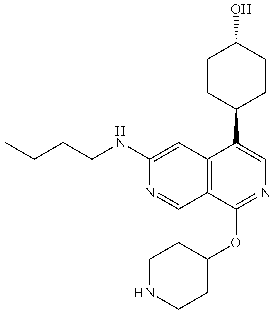 [SMILES: CCCCNc1cc2c(cn1)c(OC1CCNCC1)ncc2[C@H]1CC[C@H](O)CC1] |
| 108 | 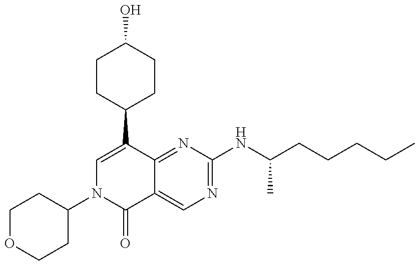 [SMILES: CCCCC[C@H](C)Nc1ncc2c(=O)n(C3CCOCC3)cc([C@H]3CC[C@H](O)CC3)c2n1] |
| 109 | 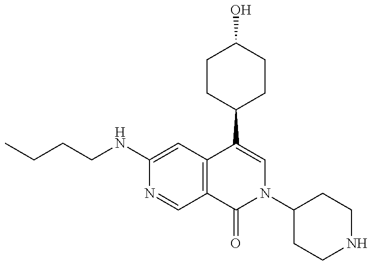 [SMILES: CCCCNc1cc2c(cn1)c(=O)n(C1CCNCC1)cc2[C@H]1CC[C@H](O)CC1] |
| 110 | 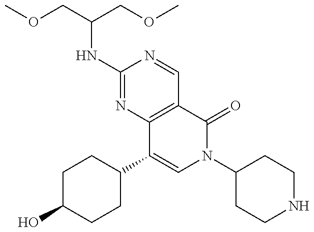 |
| 111 | 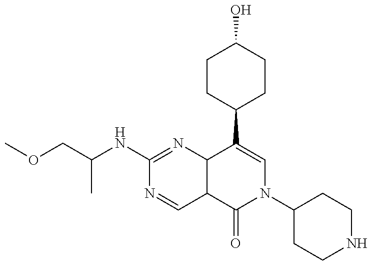 [SMILES: COCC(C)NC1=NC2C([C@H]3CC[C@H](O)CC3)=CN(C3CCNCC3)C(=O)C2C=N1] |
| 112 | 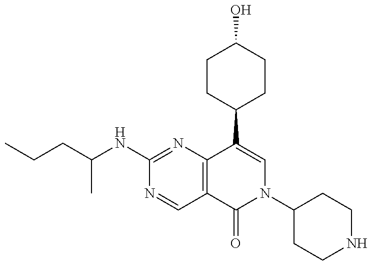 [SMILES: CCCC(C)Nc1ncc2c(=O)n(C3CCNCC3)cc([C@H]3CC[C@H](O)CC3)c2n1] |
| 113 | 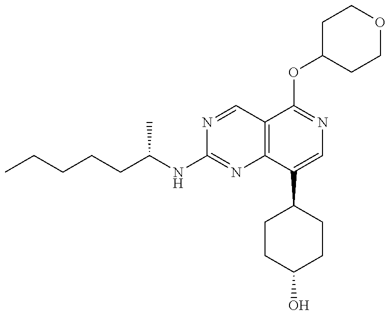 [SMILES: CCCCC[C@H](C)Nc1ncc2c(OC3CCOCC3)ncc([C@H]3CC[C@H](O)CC3)c2n1] |
| 114 | 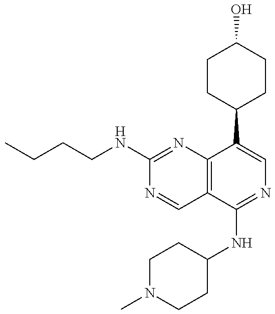 [SMILES: CCCCNc1ncc2c(NC3CCN(C)CC3)ncc([C@H]3CC[C@H](O)CC3)c2n1] |
| 115 | 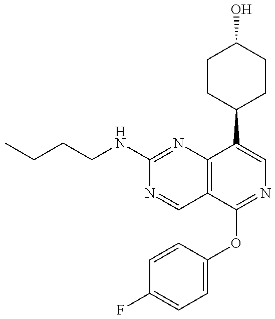 [SMILES: CCCCNc1ncc2c(Oc3ccc(F)cc3)ncc([C@H]3CC[C@H](O)CC3)c2n1] |
| 116 | 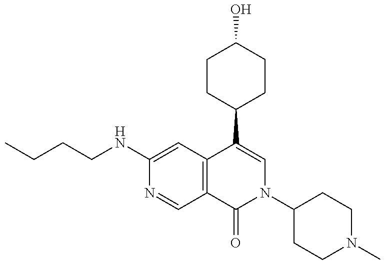 [SMILES: CCCCNc1cc2c(cn1)c(=O)n(C1CCN(C)CC1)cc2[C@H]1CC[C@H](O)CC1] |
| 117 | 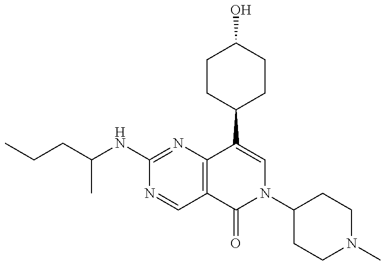 [SMILES: CCCC(C)Nc1ncc2c(=O)n(C3CCN(C)CC3)cc([C@H]3CC[C@H](O)CC3)c2n1] |
| 118 | 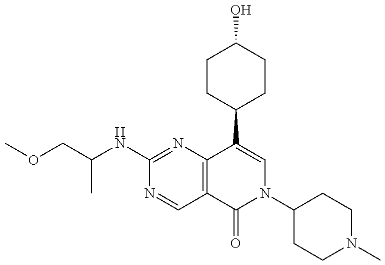 |
| 119 | 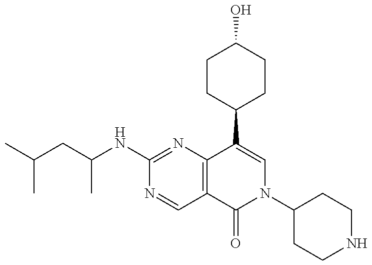 [SMILES: CC(C)CC(C)Nc1ncc2c(=O)n(C3CCNCC3)cc([C@H]3CC[C@H](O)CC3)c2n1] |
| 120 | 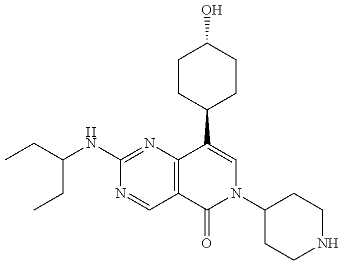 [SMILES: CCC(CC)Nc1ncc2c(=O)n(C3CCNCC3)cc([C@H]3CC[C@H](O)CC3)c2n1] |
| 121 | 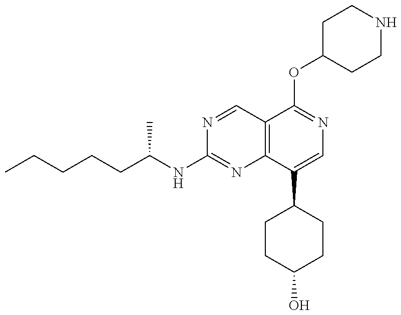 [SMILES: CCCCC[C@H](C)Nc1ncc2c(OC3CCNCC3)ncc([C@H]3CC[C@H](O)CC3)c2n1] |
| 122 | 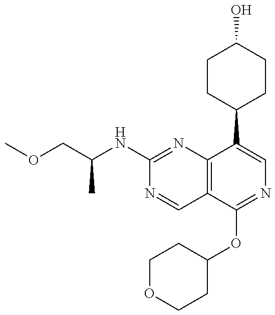 [SMILES: COC[C@H](C)Nc1ncc2c(OC3CCOCC3)ncc([C@H]3CC[C@H](O)CC3)c2n1] |
| 123 | 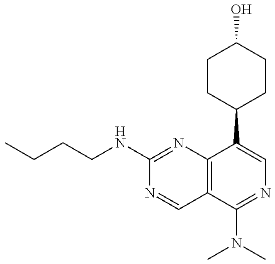 [SMILES: CCCCNc1ncc2c(N(C)C)ncc([C@H]3CC[C@H](O)CC3)c2n1] |
| 124 | 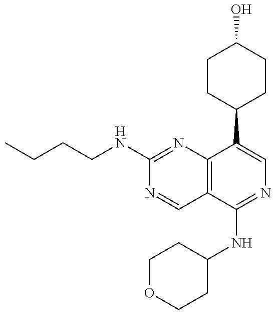 [SMILES: CCCCNc1ncc2c(NC3CCOCC3)ncc([C@H]3CC[C@H](O)CC3)c2n1] |
| 125 | 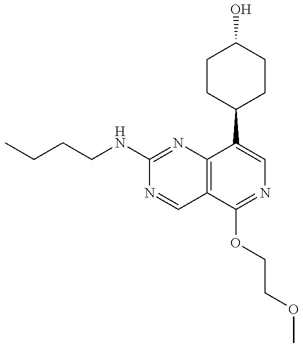 [SMILES: CCCCNc1ncc2c(OCCOC)ncc([C@H]3CC[C@H](O)CC3)c2n1] |
| 126 | 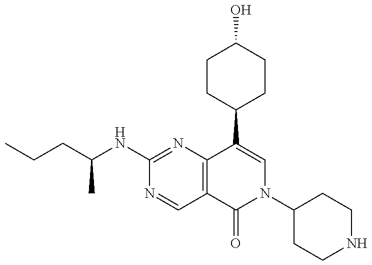 [SMILES: CCC[C@H](C)Nc1ncc2c(=O)n(C3CCNCC3)cc([C@H]3CC[C@H](O)CC3)c2n1] |
| 127 | 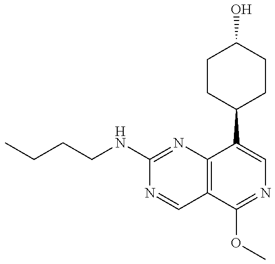 [SMILES: CCCCNc1ncc2c(OC)ncc([C@H]3CC[C@H](O)CC3)c2n1] |
| 128 | 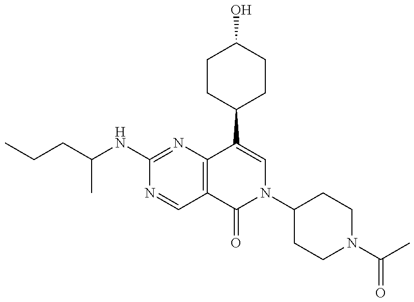 [SMILES: CCCC(C)Nc1ncc2c(=O)n(C3CCN(C(C)=O)CC3)cc([C@H]3CC[C@H](O)CC3)c2n1] |
| 129 | 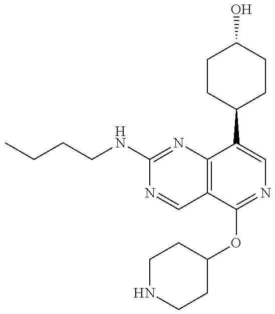 [SMILES: CCCCNc1ncc2c(OC3CCNCC3)ncc([C@H]3CC[C@H](O)CC3)c2n1] |
| 130 | 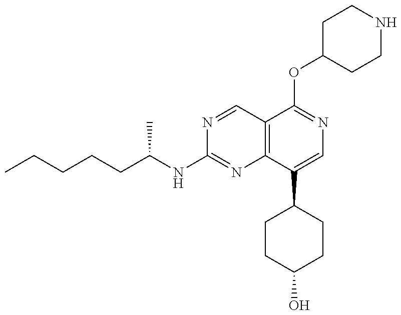 [SMILES: CCCCC[C@H](C)Nc1ncc2c(OC3CCNCC3)ncc([C@H]3CC[C@H](O)CC3)c2n1] |
| 131 | 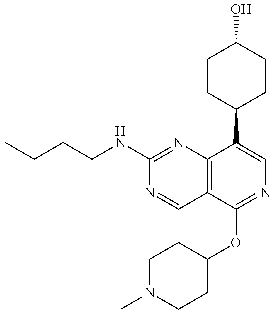 [SMILES: CCCCNc1ncc2c(OC3CCN(C)CC3)ncc([C@H]3CC[C@H](O)CC3)c2n1] |
| 132 | 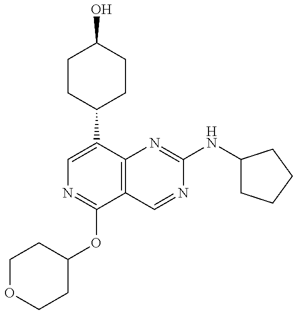 [SMILES: O[C@H]1CC[C@H](c2cnc(OC3CCOCC3)c3cnc(NC4CCCC4)nc32)CC1] |
| 133 | 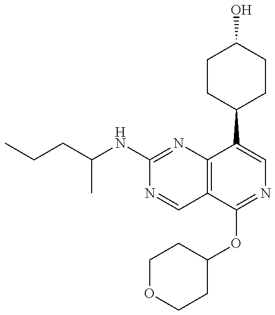 [SMILES: CCCC(C)Nc1ncc2c(OC3CCOCC3)ncc([C@H]3CC[C@H](O)CC3)c2n1] |
| 134 | 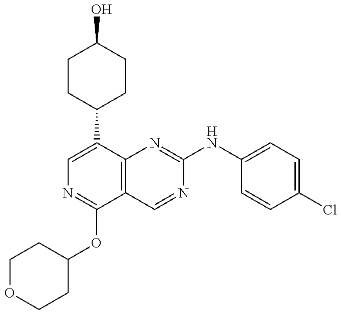 [SMILES: O[C@H]1CC[C@H](c2cnc(OC3CCOCC3)c3cnc(Nc4ccc(Cl)cc4)nc32)CC1] |
| 135 | 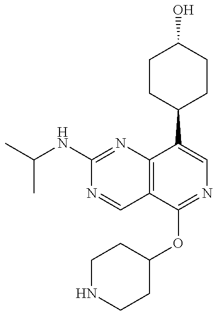 [SMILES: CC(C)Nc1ncc2c(OC3CCNCC3)ncc([C@H]3CC[C@H](O)CC3)c2n1] |
| 136 | 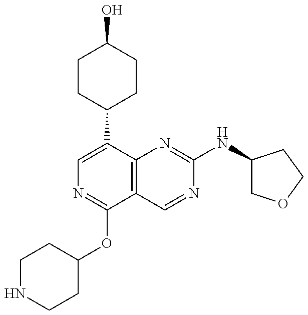 [SMILES: O[C@H]1CC[C@H](c2cnc(OC3CCNCC3)c3cnc(N[C@H]4CCOC4)nc32)CC1] |
| 137 | 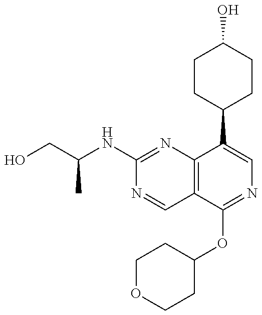 [SMILES: C[C@@H](CO)Nc1ncc2c(OC3CCOCC3)ncc([C@H]3CC[C@H](O)CC3)c2n1] |
| 138 | 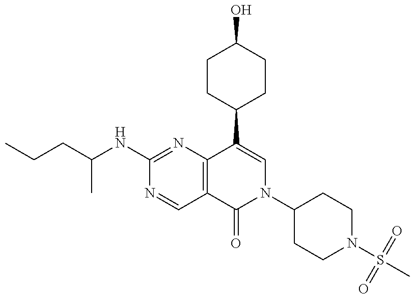 [SMILES: CCCC(C)Nc1ncc2c(=O)n(C3CCN(S(C)(=O)=O)CC3)cc([C@H]3CC[C@@H](O)CC3)c2n1] |
| 139 | 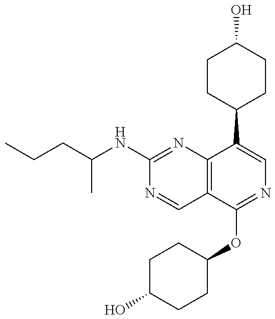 [SMILES: CCCC(C)Nc1ncc2c(O[C@H]3CC[C@H](O)CC3)ncc([C@H]3CC[C@H](O)CC3)c2n1] |
| 140 | 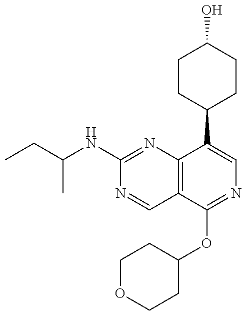 [SMILES: CCC(C)Nc1ncc2c(OC3CCOCC3)ncc([C@H]3CC[C@H](O)CC3)c2n1] |
| 141 | 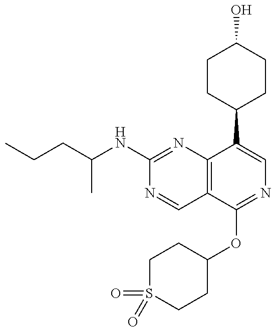 [SMILES: CCCC(C)Nc1ncc2c(OC3CCS(=O)(=O)CC3)ncc([C@H]3CC[C@H](O)CC3)c2n1] |
| 142 | 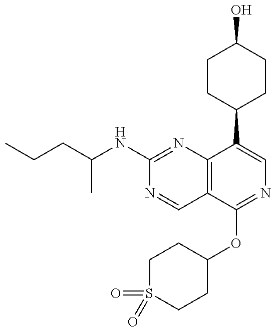 [SMILES: CCCC(C)Nc1ncc2c(OC3CCS(=O)(=O)CC3)ncc([C@H]3CC[C@@H](O)CC3)c2n1] |
| 143 | 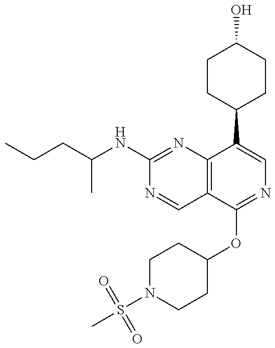 [SMILES: CCCC(C)Nc1ncc2c(OC3CCN(S(C)(=O)=O)CC3)ncc([C@H]3CC[C@H](O)CC3)c2n1] |
| 144 | 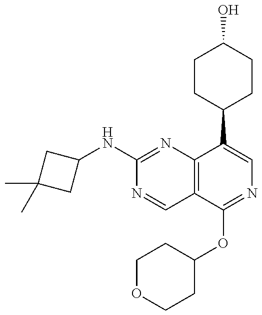 [SMILES: CC1(C)CC(Nc2ncc3c(OC4CCOCC4)ncc([C@H]4CC[C@H](O)CC4)c3n2)C1] |
| 145 | 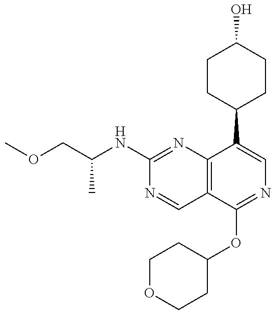 [SMILES: COC[C@@H](C)Nc1ncc2c(OC3CCOCC3)ncc([C@H]3CC[C@H](O)CC3)c2n1] |
| 146 | 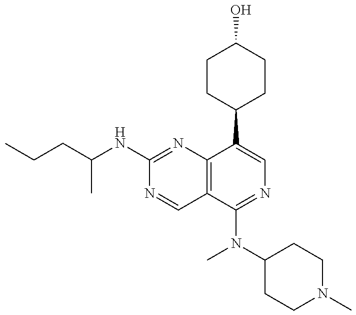 [SMILES: CCCC(C)Nc1ncc2c(N(C)C3CCN(C)CC3)ncc([C@H]3CC[C@H](O)CC3)c2n1] |
| 147 | 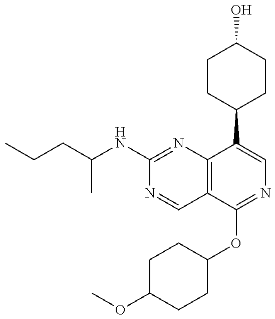 [SMILES: CCCC(C)Nc1ncc2c(OC3CCC(OC)CC3)ncc([C@H]3CC[C@H](O)CC3)c2n1] |
| 148 | 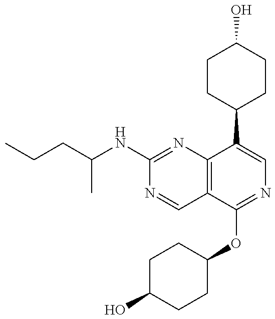 [SMILES: CCCC(C)Nc1ncc2c(O[C@H]3CC[C@@H](O)CC3)ncc([C@H]3CC[C@H](O)CC3)c2n1] |
| 149 | 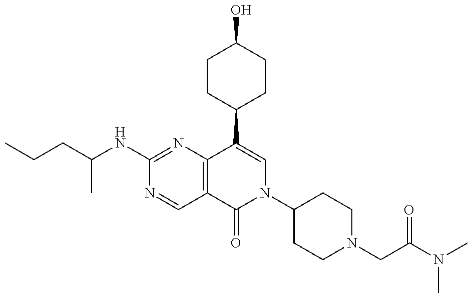 [SMILES: CCCC(C)Nc1ncc2c(=O)n(C3CCN(CC(=O)N(C)C)CC3)cc([C@H]3CC[C@@H](O)CC3)c2n1] |
| 150 | 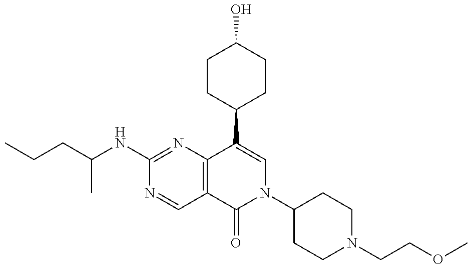 [SMILES: CCCC(C)Nc1ncc2c(=O)n(C3CCN(CCOC)CC3)cc([C@H]3CC[C@H](O)CC3)c2n1] |
| 151 | 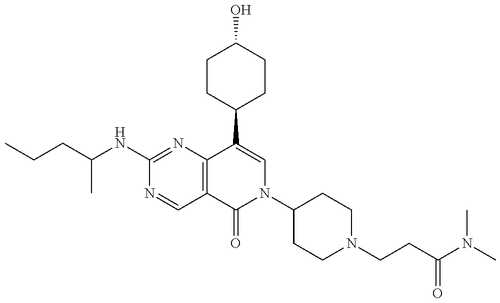 [SMILES: CCCC(C)Nc1ncc2c(=O)n(C3CCN(CCC(=O)N(C)C)CC3)cc([C@H]3CC[C@H](O)CC3)c2n1] |
| 152 | 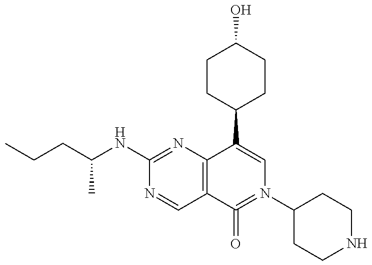 |
| 153 | 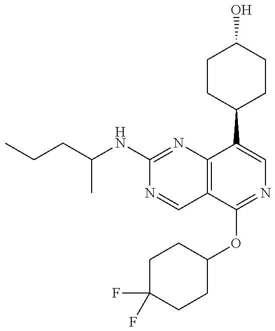 [SMILES: CCCC(C)Nc1ncc2c(OC3CCC(F)(F)CC3)ncc([C@H]3CC[C@H](O)CC3)c2n1] |
| 154 | 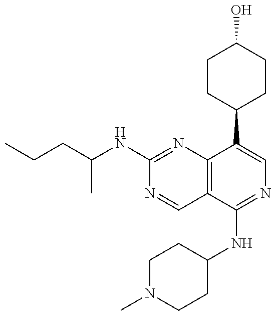 [SMILES: CCCC(C)Nc1ncc2c(NC3CCN(C)CC3)ncc([C@H]3CC[C@H](O)CC3)c2n1] |
| 155 | 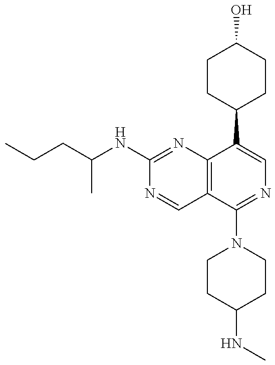 [SMILES: CCCC(C)Nc1ncc2c(N3CCC(NC)CC3)ncc([C@H]3CC[C@H](O)CC3)c2n1] |
| 156 | 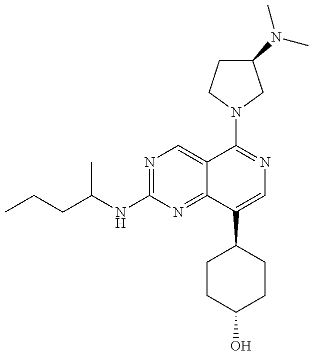 [SMILES: CCCC(C)Nc1ncc2c(N3CC[C@@H](N(C)C)C3)ncc([C@H]3CC[C@H](O)CC3)c2n1] |
| 157 | 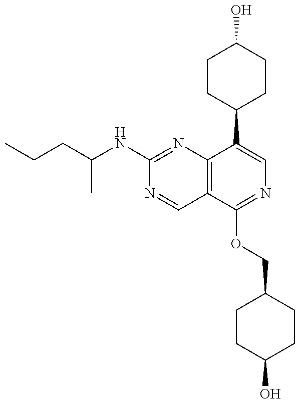 [SMILES: CCCC(C)Nc1ncc2c(OC[C@H]3CC[C@@H](O)CC3)ncc([C@H]3CC[C@H](O)CC3)c2n1] |
| 158 | 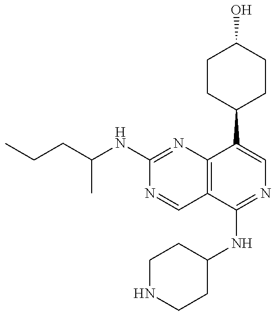 [SMILES: CCCC(C)Nc1ncc2c(NC3CCNCC3)ncc([C@H]3CC[C@H](O)CC3)c2n1] |
| 159 | 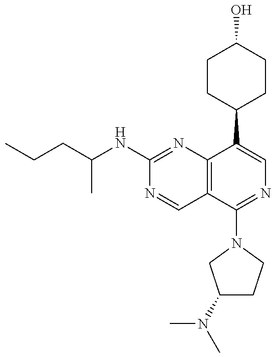 [SMILES: CCCC(C)Nc1ncc2c(N3CC[C@H](N(C)C)C3)ncc([C@H]3CC[C@H](O)CC3)c2n1] |
| 160 | 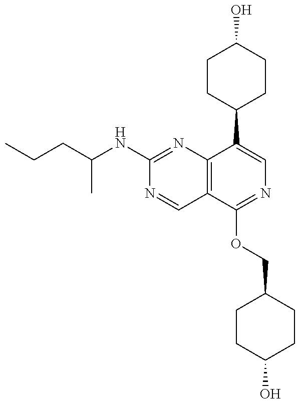 [SMILES: CCCC(C)Nc1ncc2c(OC[C@H]3CC[C@H](O)CC3)ncc([C@H]3CC[C@H](O)CC3)c2n1] |
| 161 | 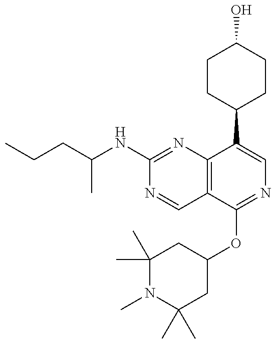 [SMILES: CCCC(C)Nc1ncc2c(OC3CC(C)(C)N(C)C(C)(C)C3)ncc([C@H]3CC[C@H](O)CC3)c2n1] |
| 162 | 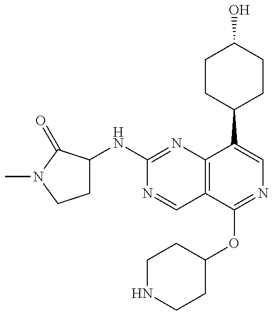 [SMILES: CN1CCC(Nc2ncc3c(OC4CCNCC4)ncc([C@H]4CC[C@H](O)CC4)c3n2)C1=O] |
| 163 | 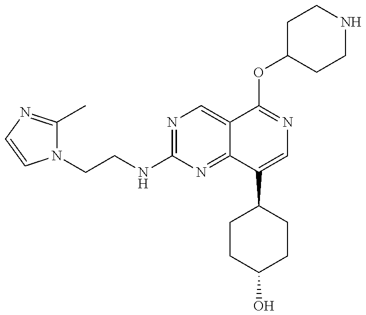 [SMILES: Cc1nccn1CCNc1ncc2c(OC3CCNCC3)ncc([C@H]3CC[C@H](O)CC3)c2n1] |
| 164 | 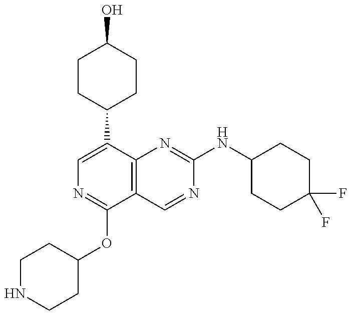 [SMILES: O[C@H]1CC[C@H](c2cnc(OC3CCNCC3)c3cnc(NC4CCC(F)(F)CC4)nc32)CC1] |
| 165 | 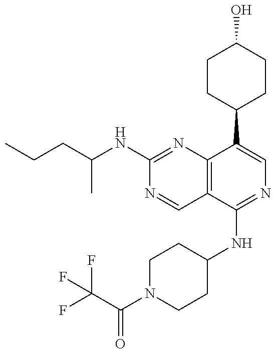 [SMILES: CCCC(C)Nc1ncc2c(NC3CCN(C(=O)C(F)(F)F)CC3)ncc([C@H]3CC[C@H](O)CC3)c2n1] |
| 166 | 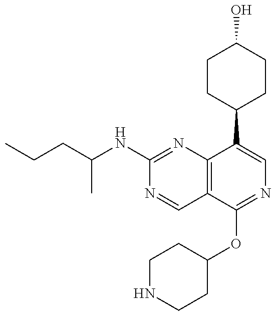 [SMILES: CCCC(C)Nc1ncc2c(OC3CCNCC3)ncc([C@H]3CC[C@H](O)CC3)c2n1] |
| 167 | 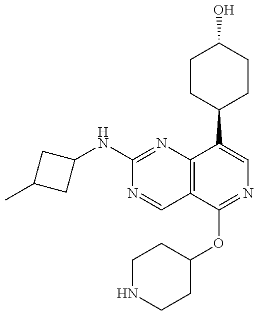 [SMILES: CC1CC(Nc2ncc3c(OC4CCNCC4)ncc([C@H]4CC[C@H](O)CC4)c3n2)C1] |
| 168 | 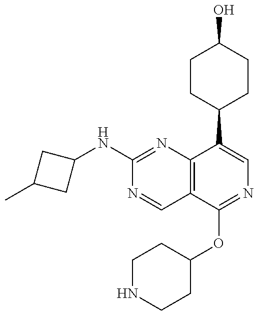 [SMILES: CC1CC(Nc2ncc3c(OC4CCNCC4)ncc([C@H]4CC[C@@H](O)CC4)c3n2)C1] |
| 169 | 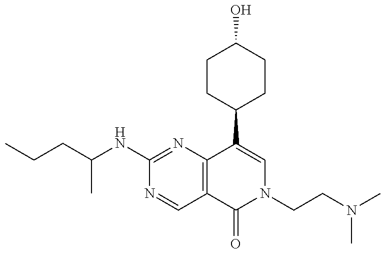 [SMILES: CCCC(C)Nc1ncc2c(=O)n(CCN(C)C)cc([C@H]3CC[C@H](O)CC3)c2n1] |
| 170 | 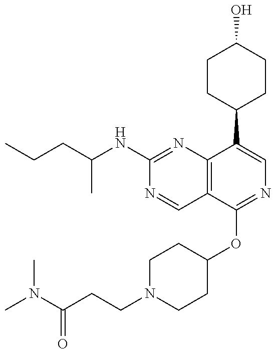 [SMILES: CCCC(C)Nc1ncc2c(OC3CCN(CCC(=O)N(C)C)CC3)ncc([C@H]3CC[C@H](O)CC3)c2n1] |
| 171 | 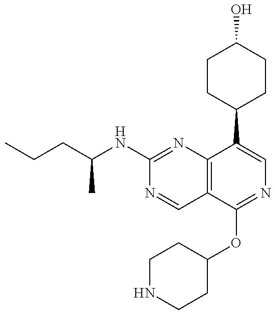 [SMILES: CCC[C@H](C)Nc1ncc2c(OC3CCNCC3)ncc([C@H]3CC[C@H](O)CC3)c2n1] |
| 172 | 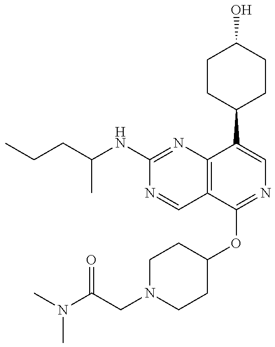 [SMILES: CCCC(C)Nc1ncc2c(OC3CCN(CC(=O)N(C)C)CC3)ncc([C@H]3CC[C@H](O)CC3)c2n1] |
| 173 | 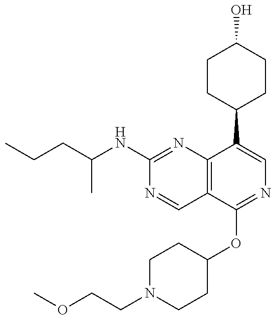 [SMILES: CCCC(C)Nc1ncc2c(OC3CCN(CCOC)CC3)ncc([C@H]3CC[C@H](O)CC3)c2n1] |
| 174 | 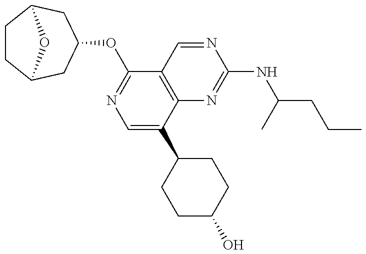 [SMILES: CCCC(C)Nc1ncc2c(O[C@@H]3C[C@H]4CC[C@@H](C3)O4)ncc([C@H]3CC[C@H](O)CC3)c2n1] |
| 175 | 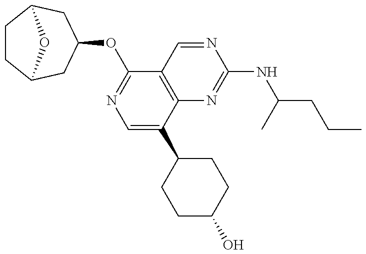 [SMILES: CCCC(C)Nc1ncc2c(O[C@H]3C[C@H]4CC[C@@H](C3)O4)ncc([C@H]3CC[C@H](O)CC3)c2n1] |
| 176 | 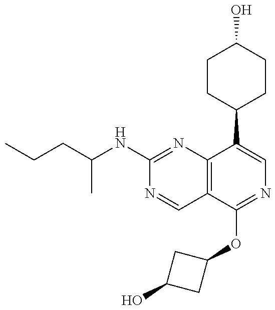 [SMILES: CCCC(C)Nc1ncc2c(O[C@H]3C[C@@H](O)C3)ncc([C@H]3CC[C@H](O)CC3)c2n1] |
| 177 | 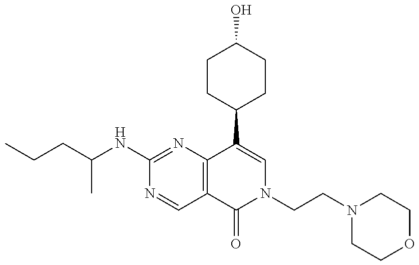 [SMILES: CCCC(C)Nc1ncc2c(=O)n(CCN3CCOCC3)cc([C@H]3CC[C@H](O)CC3)c2n1] |
| 178 | 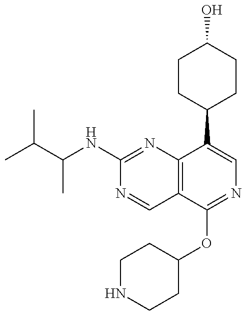 [SMILES: CC(C)C(C)Nc1ncc2c(OC3CCNCC3)ncc([C@H]3CC[C@H](O)CC3)c2n1] |
| 179 | 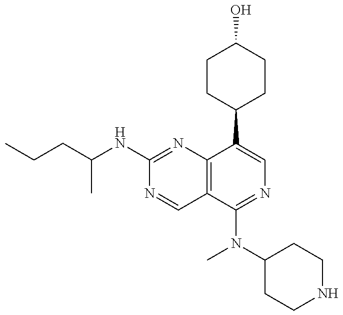 [SMILES: CCCC(C)Nc1ncc2c(N(C)C3CCNCC3)ncc([C@H]3CC[C@H](O)CC3)c2n1] |
| 180 | 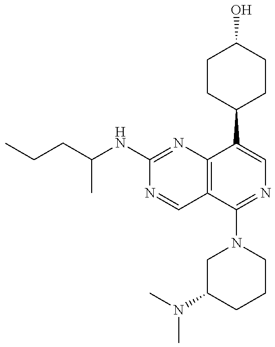 [SMILES: CCCC(C)Nc1ncc2c(N3CCC[C@H](N(C)C)C3)ncc([C@H]3CC[C@H](O)CC3)c2n1] |
| 181 | 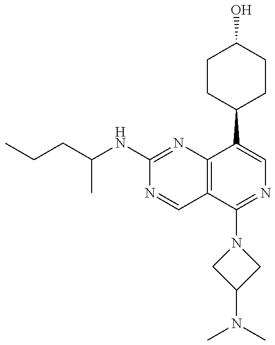 [SMILES: CCCC(C)Nc1ncc2c(N3CC(N(C)C)C3)ncc([C@H]3CC[C@H](O)CC3)c2n1] |
| 182 | 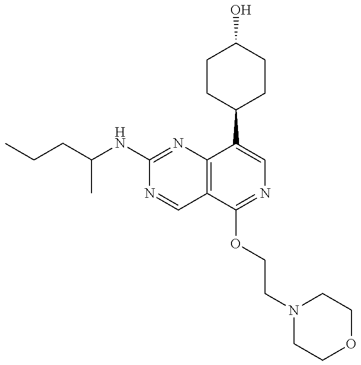 [SMILES: CCCC(C)Nc1ncc2c(OCCN3CCOCC3)ncc([C@H]3CC[C@H](O)CC3)c2n1] |
| 183 | 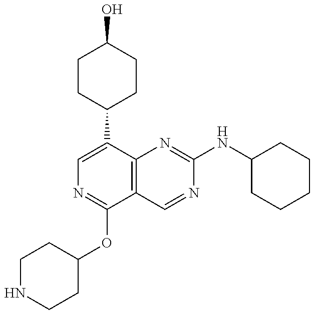 [SMILES: O[C@H]1CC[C@H](c2cnc(OC3CCNCC3)c3cnc(NC4CCCCC4)nc32)CC1] |
| 184 | 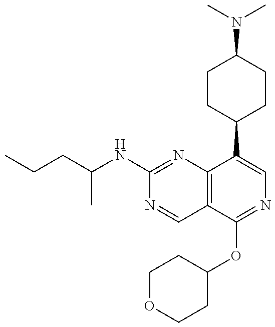 [SMILES: CCCC(C)Nc1ncc2c(OC3CCOCC3)ncc([C@H]3CC[C@@H](N(C)C)CC3)c2n1] |
| 185 | 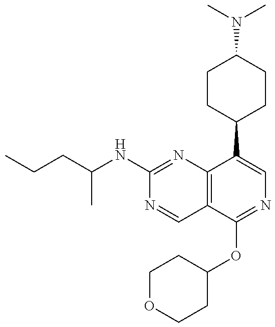 [SMILES: CCCC(C)Nc1ncc2c(OC3CCOCC3)ncc([C@H]3CC[C@H](N(C)C)CC3)c2n1] |
| 186 | 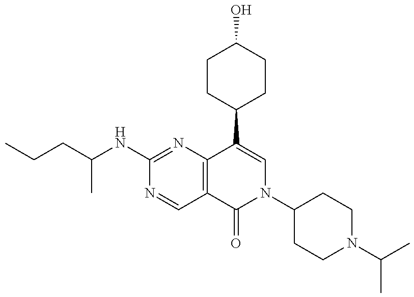 [SMILES: CCCC(C)Nc1ncc2c(=O)n(C3CCN(C(C)C)CC3)cc([C@H]3CC[C@H](O)CC3)c2n1] |
| 187 | 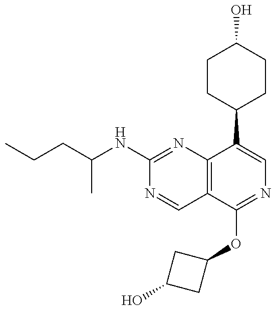 [SMILES: CCCC(C)Nc1ncc2c(O[C@H]3C[C@H](O)C3)ncc([C@H]3CC[C@H](O)CC3)c2n1] |
| 188 | 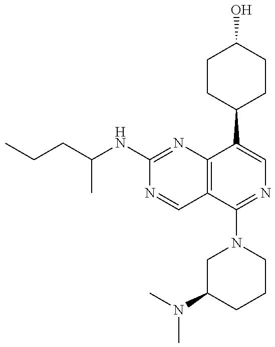 [SMILES: CCCC(C)Nc1ncc2c(N3CCC[C@@H](N(C)C)C3)ncc([C@H]3CC[C@H](O)CC3)c2n1] |
| 189 | 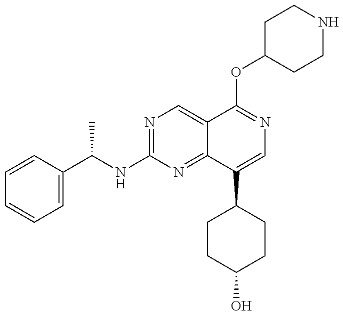 [SMILES: C[C@H](Nc1ncc2c(OC3CCNCC3)ncc([C@H]3CC[C@H](O)CC3)c2n1)c1ccccc1] |
| 190 | 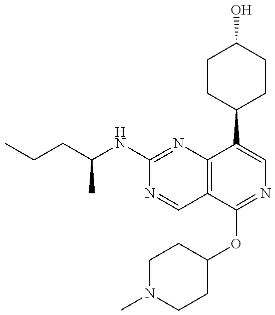 [SMILES: CCC[C@H](C)Nc1ncc2c(OC3CCN(C)CC3)ncc([C@H]3CC[C@H](O)CC3)c2n1] |
| 191 | 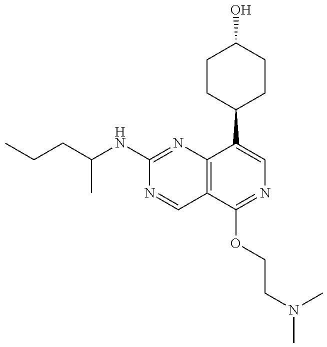 [SMILES: CCCC(C)Nc1ncc2c(OCCN(C)C)ncc([C@H]3CC[C@H](O)CC3)c2n1] |
| 192 | 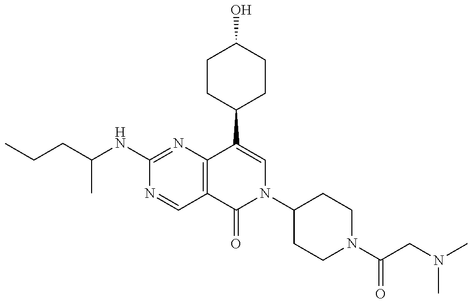 [SMILES: CCCC(C)Nc1ncc2c(=O)n(C3CCN(C(=O)CN(C)C)CC3)cc([C@H]3CC[C@H](O)CC3)c2n1] |
| 193 | 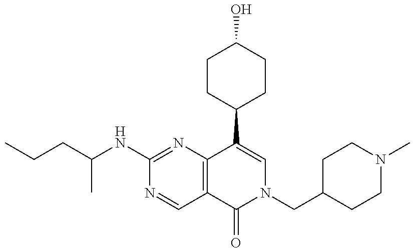 [SMILES: CCCC(C)Nc1ncc2c(=O)n(CC3CCN(C)CC3)cc([C@H]3CC[C@H](O)CC3)c2n1] |
| 194 | 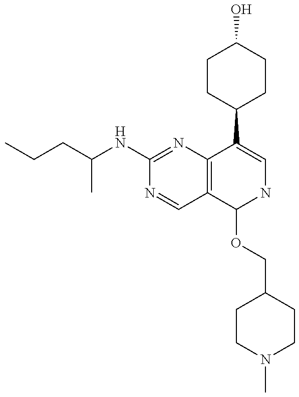 [SMILES: CCCC(C)Nc1ncc2c(n1)C([C@H]1CC[C@H](O)CC1)=CNC2OCC1CCN(C)CC1] |
| 195 | 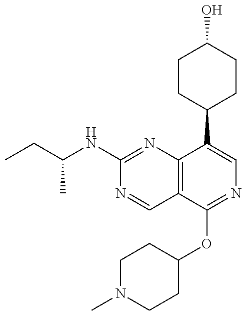 [SMILES: CC[C@@H](C)Nc1ncc2c(OC3CCN(C)CC3)ncc([C@H]3CC[C@H](O)CC3)c2n1] |
| 196 | 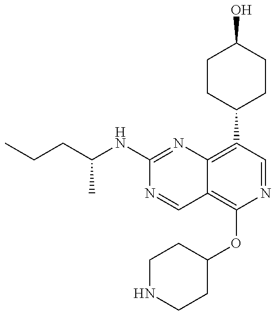 [SMILES: CCC[C@@H](C)Nc1ncc2c(OC3CCNCC3)ncc([C@H]3CC[C@H](O)CC3)c2n1] |
| 197 | 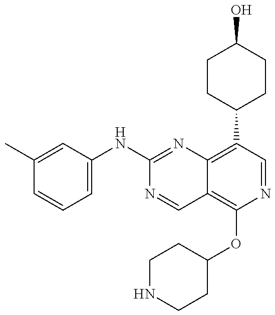 [SMILES: Cc1cccc(Nc2ncc3c(OC4CCNCC4)ncc([C@H]4CC[C@H](O)CC4)c3n2)c1] |
| 198 | 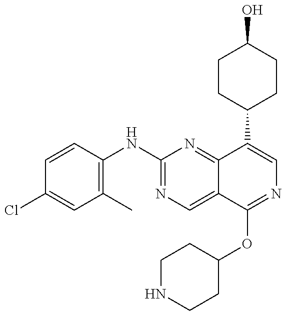 [SMILES: Cc1cc(Cl)ccc1Nc1ncc2c(OC3CCNCC3)ncc([C@H]3CC[C@H](O)CC3)c2n1] |
| 199 | 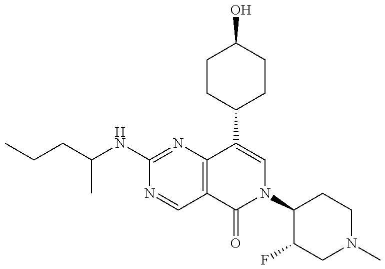 [SMILES: CCCC(C)Nc1ncc2c(=O)n([C@H]3CCN(C)C[C@@H]3F)cc([C@H]3CC[C@H](O)CC3)c2n1] |
| 200 | 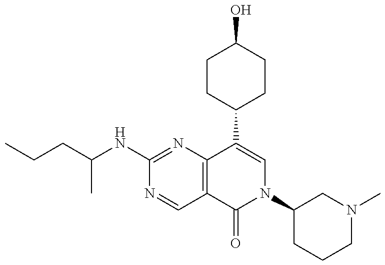 [SMILES: CCCC(C)Nc1ncc2c(=O)n([C@@H]3CCCN(C)C3)cc([C@H]3CC[C@H](O)CC3)c2n1] |
| 201 | 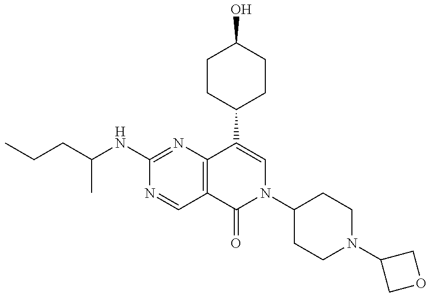 [SMILES: CCCC(C)Nc1ncc2c(=O)n(C3CCN(C4COC4)CC3)cc([C@H]3CC[C@H](O)CC3)c2n1] |
| 202 | 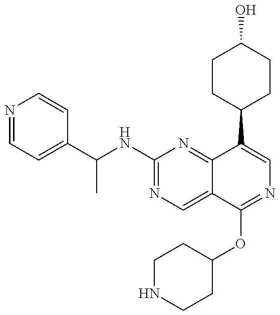 [SMILES: CC(Nc1ncc2c(OC3CCNCC3)ncc([C@H]3CC[C@H](O)CC3)c2n1)c1ccncc1] |
| 203 | 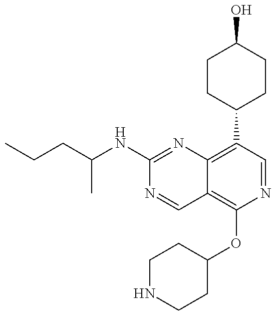 [SMILES: CCCC(C)Nc1ncc2c(OC3CCNCC3)ncc([C@H]3CC[C@H](O)CC3)c2n1] |
| 204 | 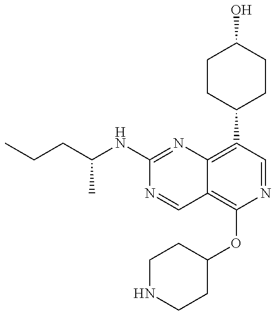 [SMILES: CCC[C@@H](C)Nc1ncc2c(OC3CCNCC3)ncc([C@H]3CC[C@@H](O)CC3)c2n1] |
| 205 | 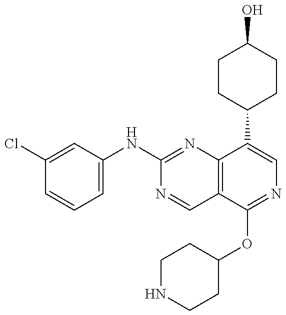 [SMILES: O[C@H]1CC[C@H](c2cnc(OC3CCNCC3)c3cnc(Nc4cccc(Cl)c4)nc32)CC1] |
| 206 | 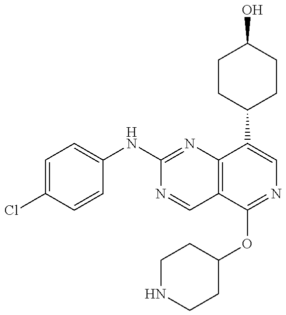 [SMILES: O[C@H]1CC[C@H](c2cnc(OC3CCNCC3)c3cnc(Nc4ccc(Cl)cc4)nc32)CC1] |
| 207 | 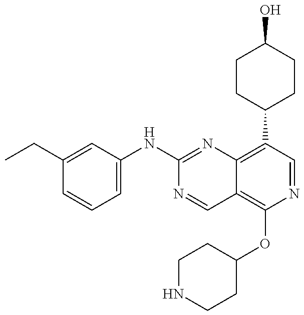 [SMILES: CCc1cccc(Nc2ncc3c(OC4CCNCC4)ncc([C@H]4CC[C@H](O)CC4)c3n2)c1] |
| 208 | 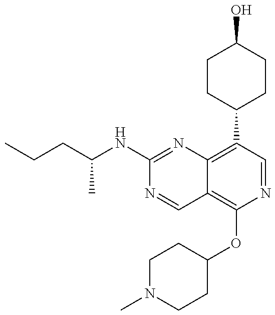 [SMILES: CCC[C@@H](C)Nc1ncc2c(OC3CCN(C)CC3)ncc([C@H]3CC[C@H](O)CC3)c2n1] |
| 209 | 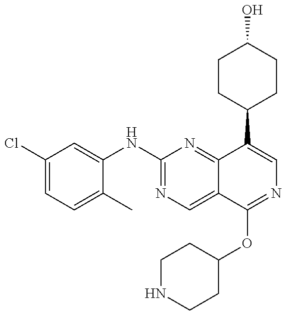 [SMILES: Cc1ccc(Cl)cc1Nc1ncc2c(OC3CCNCC3)ncc([C@H]3CC[C@H](O)CC3)c2n1] |
| 210 | 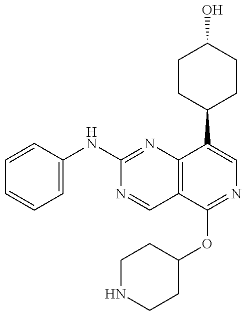 [SMILES: O[C@H]1CC[C@H](c2cnc(OC3CCNCC3)c3cnc(Nc4ccccc4)nc32)CC1] |
| 211 | 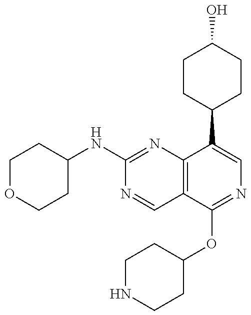 [SMILES: O[C@H]1CC[C@H](c2cnc(OC3CCNCC3)c3cnc(NC4CCOCC4)nc32)CC1] |
| 212 | 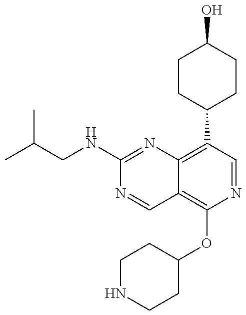 [SMILES: CC(C)CNc1ncc2c(OC3CCNCC3)ncc([C@H]3CC[C@H](O)CC3)c2n1] |
| 213 | 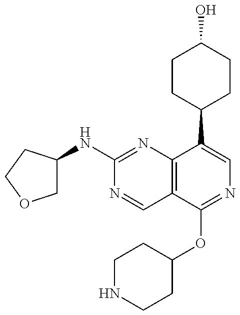 [SMILES: O[C@H]1CC[C@H](c2cnc(OC3CCNCC3)c3cnc(N[C@@H]4CCOC4)nc32)CC1] |
| 214 | 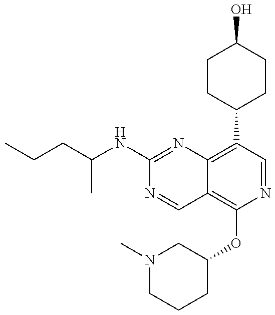 [SMILES: CCCC(C)Nc1ncc2c(O[C@@H]3CCCN(C)C3)ncc([C@H]3CC[C@H](O)CC3)c2n1] |
| 215 | 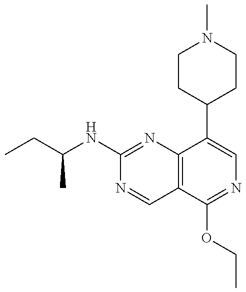 [SMILES: CCOc1ncc(C2CCN(C)CC2)c2nc(N[C@@H](C)CC)ncc12] |
| 216 | 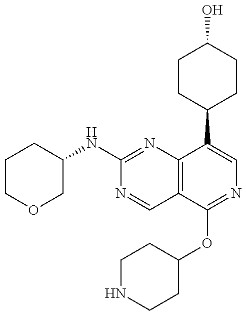 [SMILES: O[C@H]1CC[C@H](c2cnc(OC3CCNCC3)c3cnc(N[C@H]4CCCOC4)nc32)CC1] |
| 217 | 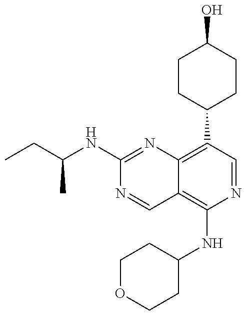 [SMILES: CC[C@H](C)Nc1ncc2c(NC3CCOCC3)ncc([C@H]3CC[C@H](O)CC3)c2n1] |
| 218 | 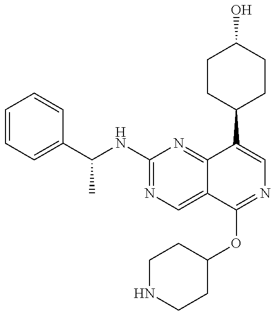 [SMILES: C[C@@H](Nc1ncc2c(OC3CCNCC3)ncc([C@H]3CC[C@H](O)CC3)c2n1)c1ccccc1] |
| 219 | Intentionally omitted |
| 220 | 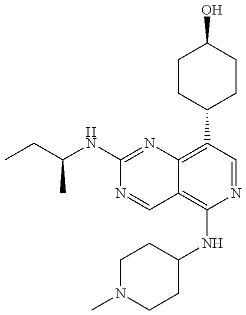 [SMILES: CC[C@H](C)Nc1ncc2c(NC3CCN(C)CC3)ncc([C@H]3CC[C@H](O)CC3)c2n1] |
| 221 | 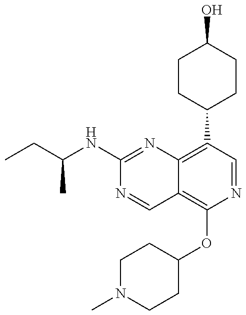 [SMILES: CC[C@H](C)Nc1ncc2c(OC3CCN(C)CC3)ncc([C@H]3CC[C@H](O)CC3)c2n1] |
| 222 | 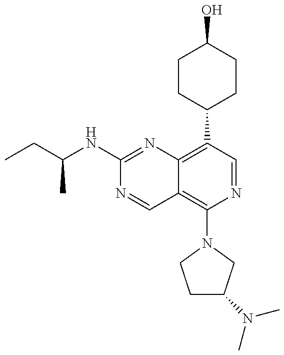 [SMILES: CC[C@H](C)Nc1ncc2c(N3CC[C@@H](N(C)C)C3)ncc([C@H]3CC[C@H](O)CC3)c2n1] |
| 223 | 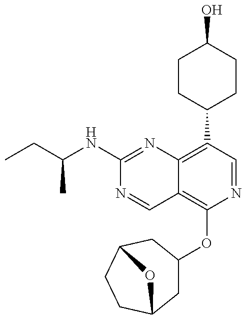 [SMILES: CC[C@H](C)Nc1ncc2c(OC3C[C@H]4CC[C@@H](C3)O4)ncc([C@H]3CC[C@H](O)CC3)c2n1] |
| 224 | 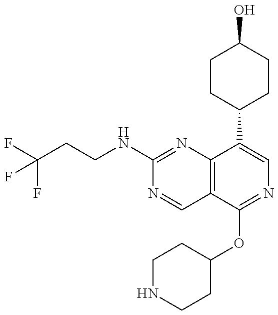 [SMILES: O[C@H]1CC[C@H](c2cnc(OC3CCNCC3)c3cnc(NCCC(F)(F)F)nc32)CC1] |
| 225 | 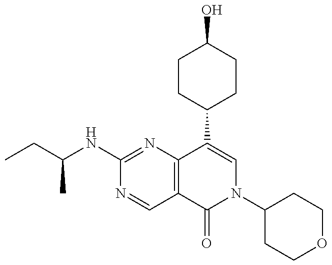 [SMILES: CC[C@H](C)Nc1ncc2c(=O)n(C3CCOCC3)cc([C@H]3CC[C@H](O)CC3)c2n1] |
| 226 | 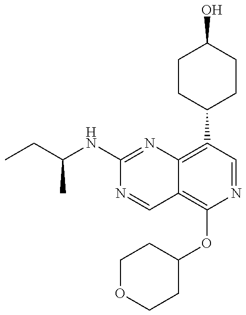 [SMILES: CC[C@H](C)Nc1ncc2c(OC3CCOCC3)ncc([C@H]3CC[C@H](O)CC3)c2n1] |
| 227 | 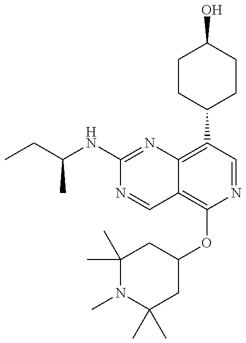 [SMILES: CC[C@H](C)Nc1ncc2c(OC3CC(C)(C)N(C)C(C)(C)C3)ncc([C@H]3CC[C@H](O)CC3)c2n1] |
| 228 | 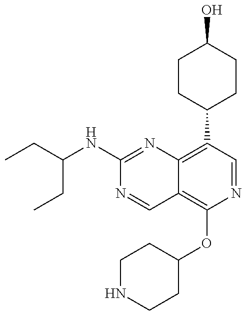 [SMILES: CCC(CC)Nc1ncc2c(OC3CCNCC3)ncc([C@H]3CC[C@H](O)CC3)c2n1] |
| 229 | 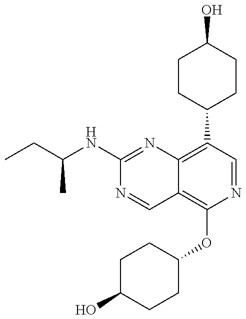 [SMILES: CC[C@H](C)Nc1ncc2c(O[C@H]3CC[C@H](O)CC3)ncc([C@H]3CC[C@H](O)CC3)c2n1] |
| 230 | 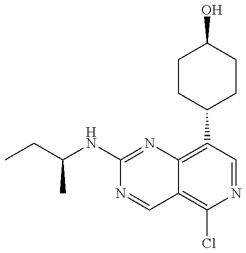 [SMILES: CC[C@H](C)Nc1ncc2c(Cl)ncc([C@H]3CC[C@H](O)CC3)c2n1] |
| 231 | 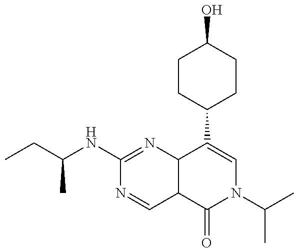 [SMILES: CC[C@H](C)NC1=NC2C([C@H]3CC[C@H](O)CC3)=CN(C(C)C)C(=O)C2C=N1] |
| 232 | 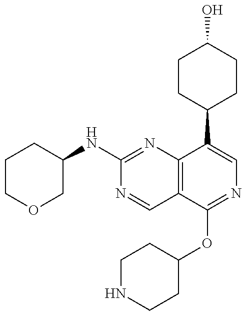 [SMILES: O[C@H]1CC[C@H](c2cnc(OC3CCNCC3)c3cnc(N[C@@H]4CCCOC4)nc32)CC1] |
| 233 | 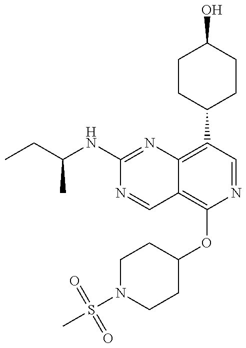 [SMILES: CC[C@H](C)Nc1ncc2c(OC3CCN(S(C)(=O)=O)CC3)ncc([C@H]3CC[C@H](O)CC3)c2n1] |
| 234 | 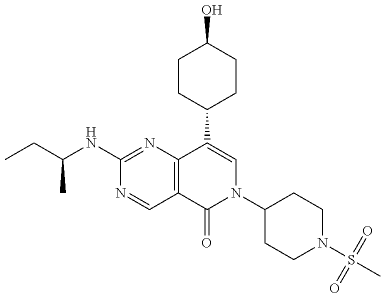 [SMILES: CC[C@H](C)Nc1ncc2c(=O)n(C3CCN(S(C)(=O)=O)CC3)cc([C@H]3CC[C@H](O)CC3)c2n1] |
| 235 | 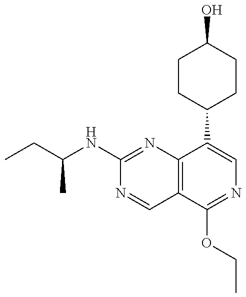 [SMILES: CCOc1ncc([C@H]2CC[C@H](O)CC2)c2nc(N[C@@H](C)CC)ncc12] |
| 236 | 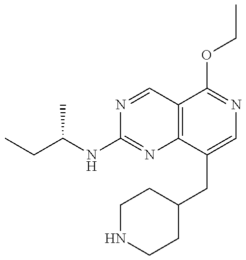 [SMILES: CCOc1ncc(CC2CCNCC2)c2nc(N[C@@H](C)CC)ncc12] |
| 237 | 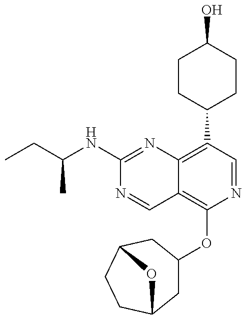 [SMILES: CC[C@H](C)Nc1ncc2c(OC3C[C@H]4CC[C@@H](C3)O4)ncc([C@H]3CC[C@H](O)CC3)c2n1] |
| 238 | 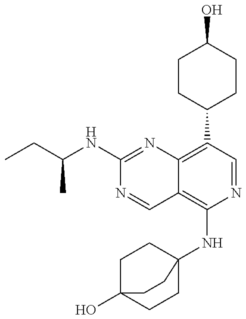 [SMILES: CC[C@H](C)Nc1ncc2c(NC34CCC(O)(CC3)CC4)ncc([C@H]3CC[C@H](O)CC3)c2n1] |
| 239 | 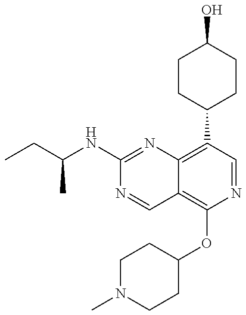 [SMILES: CC[C@H](C)Nc1ncc2c(OC3CCN(C)CC3)ncc([C@H]3CC[C@H](O)CC3)c2n1] |
| 240 | 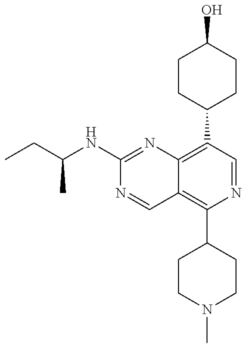 [SMILES: CC[C@H](C)Nc1ncc2c(C3CCN(C)CC3)ncc([C@H]3CC[C@H](O)CC3)c2n1] |
| 241 | 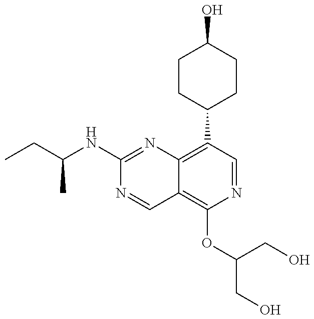 [SMILES: CC[C@H](C)Nc1ncc2c(OC(CO)CO)ncc([C@H]3CC[C@H](O)CC3)c2n1] |
| 242 | 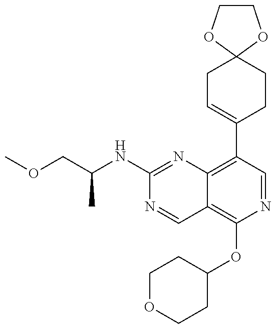 [SMILES: COC[C@H](C)Nc1ncc2c(OC3CCOCC3)ncc(C3=CCC4(CC3)OCCO4)c2n1] |
| 243 | 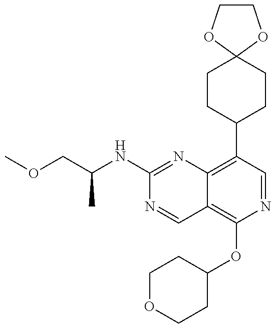 [SMILES: COC[C@H](C)Nc1ncc2c(OC3CCOCC3)ncc(C3CCC4(CC3)OCCO4)c2n1] |
| 244 | 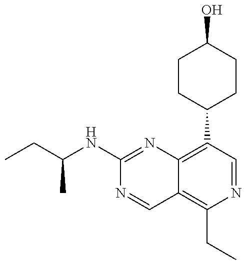 [SMILES: CCc1ncc([C@H]2CC[C@H](O)CC2)c2nc(N[C@@H](C)CC)ncc12] |
| 245 | 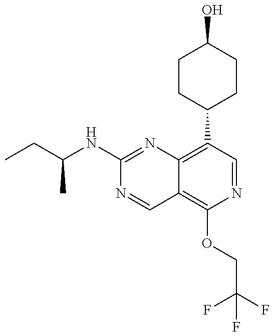 [SMILES: CC[C@H](C)Nc1ncc2c(OCC(F)(F)F)ncc([C@H]3CC[C@H](O)CC3)c2n1] |
| 246 | 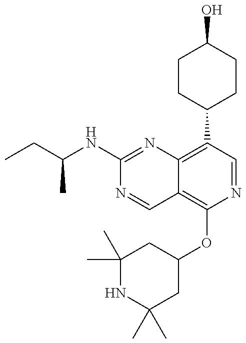 [SMILES: CC[C@H](C)Nc1ncc2c(OC3CC(C)(C)NC(C)(C)C3)ncc([C@H]3CC[C@H](O)CC3)c2n1] |
| 247 | 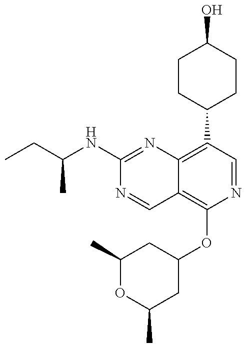 [SMILES: CC[C@H](C)Nc1ncc2c(OC3C[C@@H](C)O[C@@H](C)C3)ncc([C@H]3CC[C@H](O)CC3)c2n1] |
| 248 | 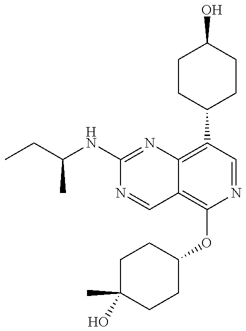 [SMILES: CC[C@H](C)Nc1ncc2c(O[C@H]3CC[C@@](C)(O)CC3)ncc([C@H]3CC[C@H](O)CC3)c2n1] |
| 249 | 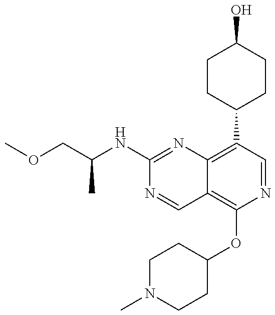 [SMILES: COC[C@H](C)Nc1ncc2c(OC3CCN(C)CC3)ncc([C@H]3CC[C@H](O)CC3)c2n1] |
| 250 | 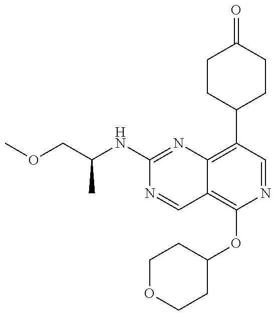 [SMILES: COC[C@H](C)Nc1ncc2c(OC3CCOCC3)ncc(C3CCC(=O)CC3)c2n1] |
| 251 | 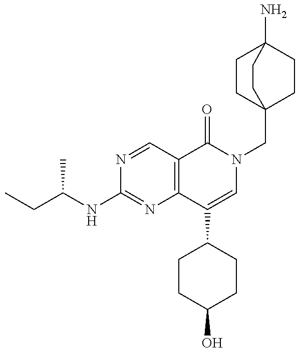 [SMILES: CC[C@H](C)Nc1ncc2c(=O)n(CC34CCC(N)(CC3)CC4)cc([C@H]3CC[C@H](O)CC3)c2n1] |
| 252 | 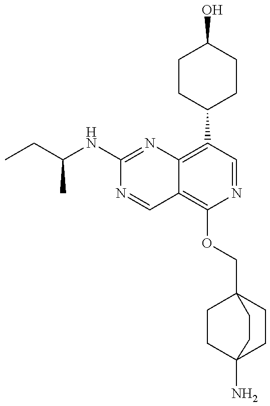 [SMILES: CC[C@H](C)Nc1ncc2c(OCC34CCC(N)(CC3)CC4)ncc([C@H]3CC[C@H](O)CC3)c2n1] |
| 253 | 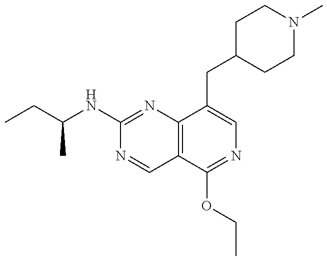 [SMILES: CCOc1ncc(CC2CCN(C)CC2)c2nc(N[C@@H](C)CC)ncc12] |
| 254 | 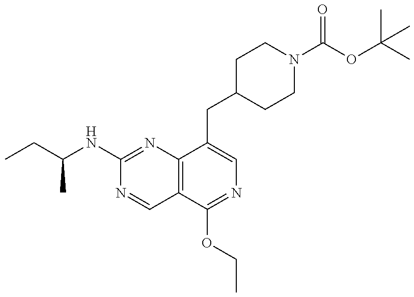 [SMILES: CCOc1ncc(CC2CCN(C(=O)OC(C)(C)C)CC2)c2nc(N[C@@H](C)CC)ncc12] |
| 255 | 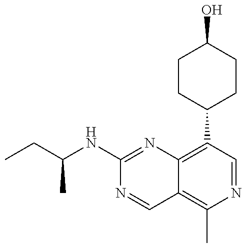 [SMILES: CC[C@H](C)Nc1ncc2c(C)ncc([C@H]3CC[C@H](O)CC3)c2n1] |
| 256 | 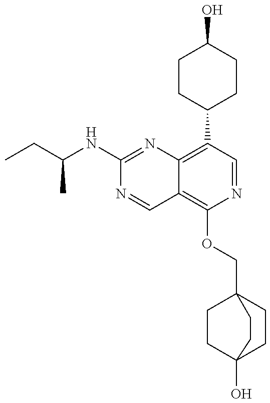 [SMILES: CC[C@H](C)Nc1ncc2c(OCC34CCC(O)(CC3)CC4)ncc([C@H]3CC[C@H](O)CC3)c2n1] |
| 257 | 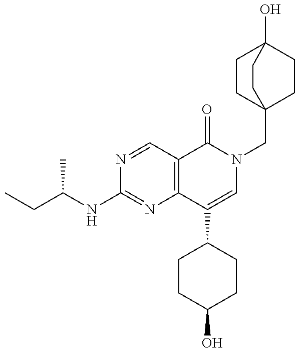 [SMILES: CC[C@H](C)Nc1ncc2c(=O)n(CC34CCC(O)(CC3)CC4)cc([C@H]3CC[C@H](O)CC3)c2n1] |
| 258 | Intentionally omitted |
| 259 | 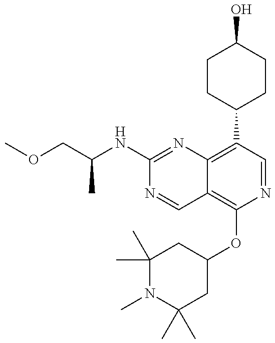 [SMILES: COC[C@H](C)Nc1ncc2c(OC3CC(C)(C)N(C)C(C)(C)C3)ncc([C@H]3CC[C@H](O)CC3)c2n1] |
| 260 | 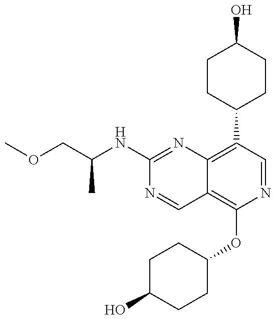 [SMILES: COC[C@H](C)Nc1ncc2c(O[C@H]3CC[C@H](O)CC3)ncc([C@H]3CC[C@H](O)CC3)c2n1] |
| 261 | 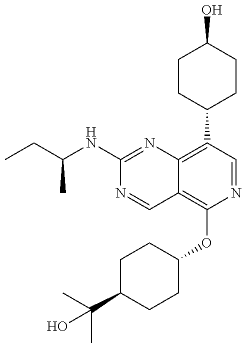 [SMILES: CC[C@H](C)Nc1ncc2c(O[C@H]3CC[C@H](C(C)(C)O)CC3)ncc([C@H]3CC[C@H](O)CC3)c2n1] |
| 262 | 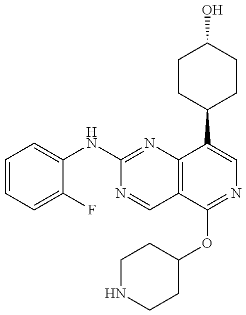 [SMILES: O[C@H]1CC[C@H](c2cnc(OC3CCNCC3)c3cnc(Nc4ccccc4F)nc32)CC1] |
| 263 | 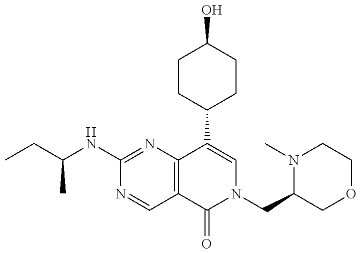 [SMILES: CC[C@H](C)Nc1ncc2c(=O)n(C[C@@H]3COCCN3C)cc([C@H]3CC[C@H](O)CC3)c2n1] |
| 264 | 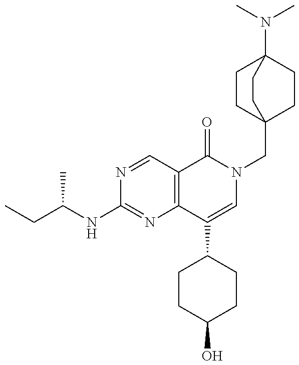 [SMILES: CC[C@H](C)Nc1ncc2c(=O)n(CC34CCC(N(C)C)(CC3)CC4)cc([C@H]3CC[C@H](O)CC3)c2n1] |
| 265 | 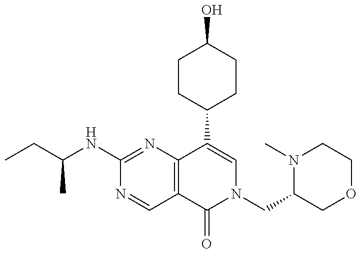 [SMILES: CC[C@H](C)Nc1ncc2c(=O)n(C[C@H]3COCCN3C)cc([C@H]3CC[C@H](O)CC3)c2n1] |
| 266 | 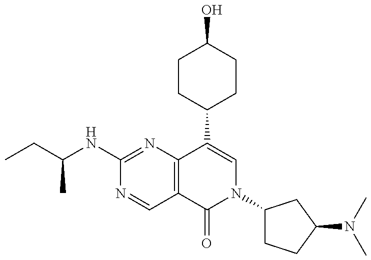 [SMILES: CC[C@H](C)Nc1ncc2c(=O)n([C@H]3CC[C@H](N(C)C)C3)cc([C@H]3CC[C@H](O)CC3)c2n1] |
| 267 | 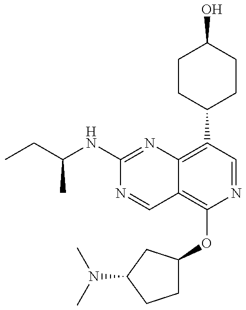 [SMILES: CC[C@H](C)Nc1ncc2c(O[C@H]3CC[C@H](N(C)C)C3)ncc([C@H]3CC[C@H](O)CC3)c2n1] |
| 268 | 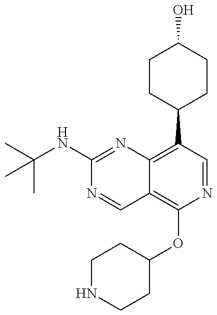 [SMILES: CC(C)(C)Nc1ncc2c(OC3CCNCC3)ncc([C@H]3CC[C@H](O)CC3)c2n1] |
| 269 | 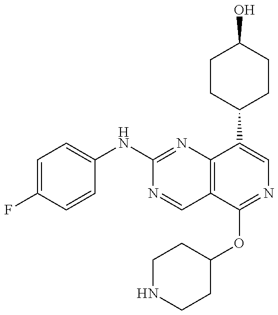 [SMILES: O[C@H]1CC[C@H](c2cnc(OC3CCNCC3)c3cnc(Nc4ccc(F)cc4)nc32)CC1] |
| 270 | 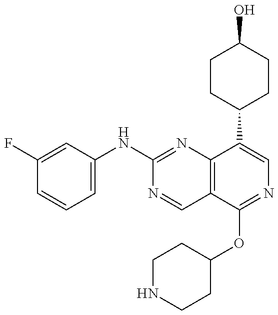 [SMILES: O[C@H]1CC[C@H](c2cnc(OC3CCNCC3)c3cnc(Nc4cccc(F)c4)nc32)CC1] |
| 271 | 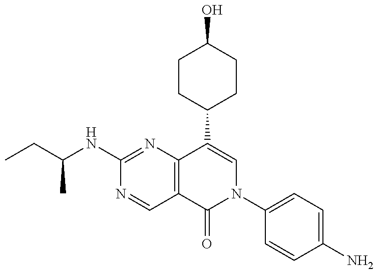 [SMILES: CC[C@H](C)Nc1ncc2c(=O)n(-c3ccc(N)cc3)cc([C@H]3CC[C@H](O)CC3)c2n1] |
| 272 | 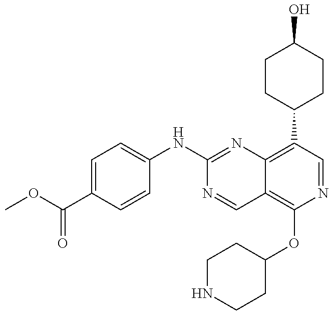 [SMILES: COC(=O)c1ccc(Nc2ncc3c(OC4CCNCC4)ncc([C@H]4CC[C@H](O)CC4)c3n2)cc1] |
| 273 | 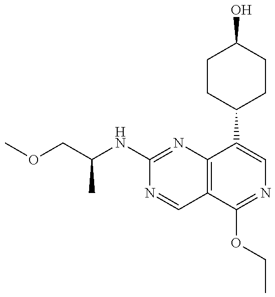 [SMILES: CCOc1ncc([C@H]2CC[C@H](O)CC2)c2nc(N[C@@H](C)COC)ncc12] |
| 274 | 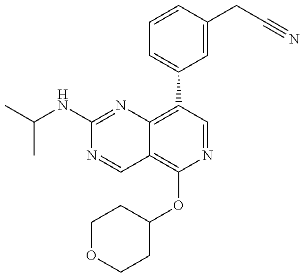 |
| 275 | 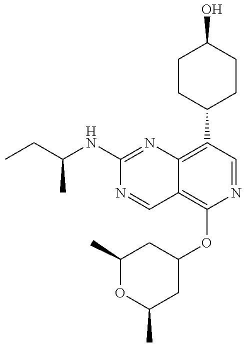 [SMILES: CC[C@H](C)Nc1ncc2c(OC3C[C@@H](C)O[C@@H](C)C3)ncc([C@H]3CC[C@H](O)CC3)c2n1] |
| 276 | 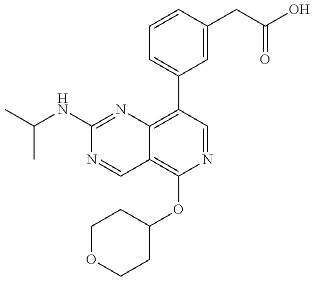 [SMILES: CC(C)Nc1ncc2c(OC3CCOCC3)ncc(-c3cccc(CC(=O)O)c3)c2n1] |
| 277 | 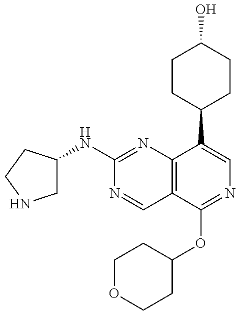 [SMILES: O[C@H]1CC[C@H](c2cnc(OC3CCOCC3)c3cnc(N[C@H]4CCNC4)nc32)CC1] |
| 278 | 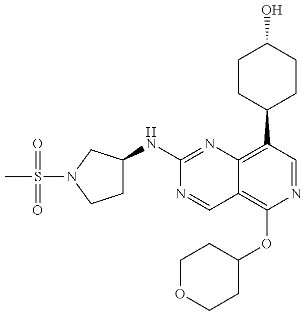 [SMILES: CS(=O)(=O)N1CC[C@H](Nc2ncc3c(OC4CCOCC4)ncc([C@H]4CC[C@H](O)CC4)c3n2)C1] |
| 279 | 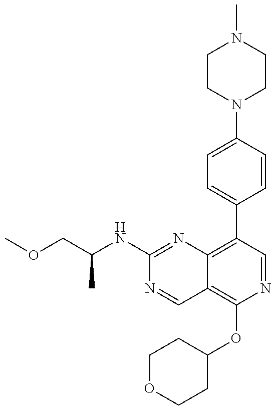 [SMILES: COC[C@H](C)Nc1ncc2c(OC3CCOCC3)ncc(-c3ccc(N4CCN(C)CC4)cc3)c2n1] |
| 280 | 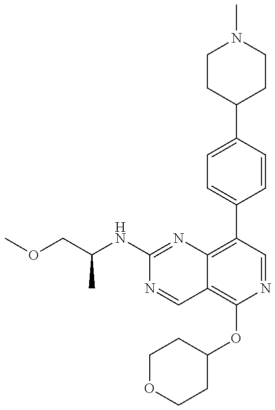 [SMILES: COC[C@H](C)Nc1ncc2c(OC3CCOCC3)ncc(-c3ccc(C4CCN(C)CC4)cc3)c2n1] |
| 281 | 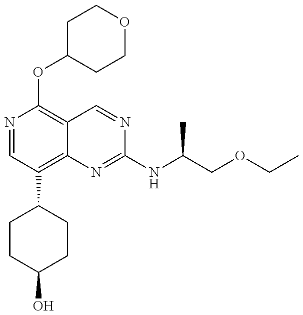 [SMILES: CCOC[C@H](C)Nc1ncc2c(OC3CCOCC3)ncc([C@H]3CC[C@H](O)CC3)c2n1] |
| 282 | 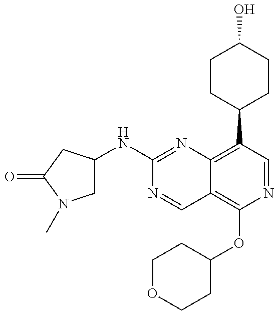 [SMILES: CN1CC(Nc2ncc3c(OC4CCOCC4)ncc([C@H]4CC[C@H](O)CC4)c3n2)CC1=O] |
| 283 | 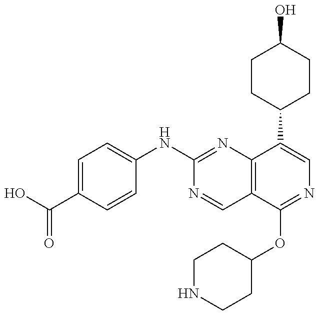 [SMILES: O=C(O)c1ccc(Nc2ncc3c(OC4CCNCC4)ncc([C@H]4CC[C@H](O)CC4)c3n2)cc1] |
| 284 | 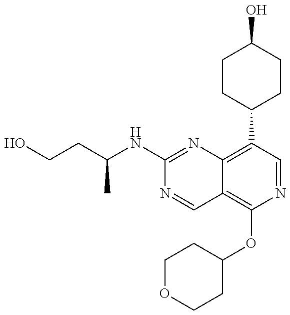 [SMILES: C[C@@H](CCO)Nc1ncc2c(OC3CCOCC3)ncc([C@H]3CC[C@H](O)CC3)c2n1] |
| 285 | 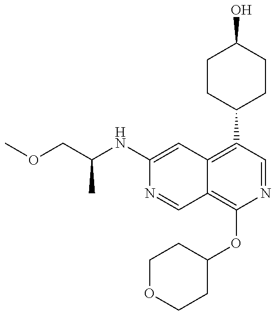 [SMILES: COC[C@H](C)Nc1cc2c(cn1)c(OC1CCOCC1)ncc2[C@H]1CC[C@H](O)CC1] |
| 286 | 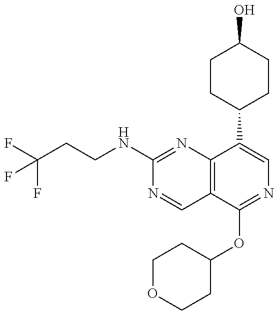 [SMILES: O[C@H]1CC[C@H](c2cnc(OC3CCOCC3)c3cnc(NCCC(F)(F)F)nc32)CC1] |
| 287 | 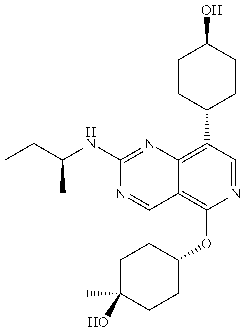 [SMILES: CC[C@H](C)Nc1ncc2c(O[C@H]3CC[C@](C)(O)CC3)ncc([C@H]3CC[C@H](O)CC3)c2n1] |
| 288 | 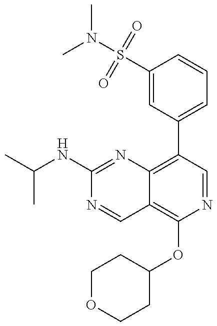 [SMILES: CC(C)Nc1ncc2c(OC3CCOCC3)ncc(-c3cccc(S(=O)(=O)N(C)C)c3)c2n1] |
| 289 | 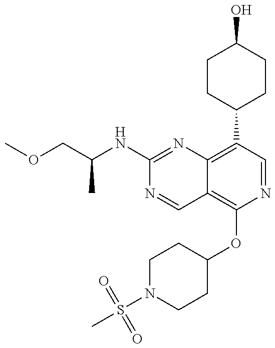 [SMILES: COC[C@H](C)Nc1ncc2c(OC3CCN(S(C)(=O)=O)CC3)ncc([C@H]3CC[C@H](O)CC3)c2n1] |
| 290 | 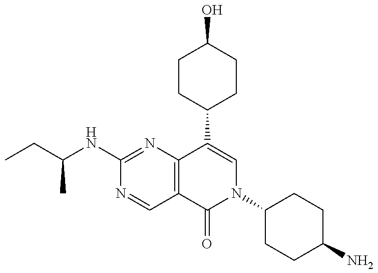 [SMILES: CC[C@H](C)Nc1ncc2c(=O)n([C@H]3CC[C@H](N)CC3)cc([C@H]3CC[C@H](O)CC3)c2n1] |
| 291 | 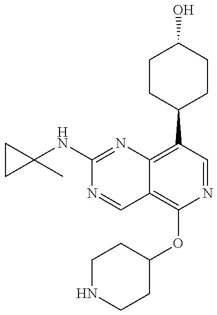 [SMILES: CC1(Nc2ncc3c(OC4CCNCC4)ncc([C@H]4CC[C@H](O)CC4)c3n2)CC1] |
| 292 | 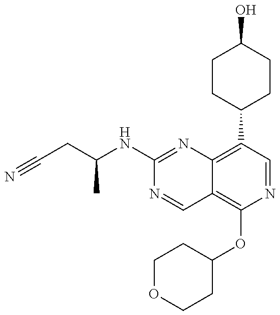 [SMILES: C[C@@H](CC#N)Nc1ncc2c(OC3CCOCC3)ncc([C@H]3CC[C@H](O)CC3)c2n1] |
| 293 | 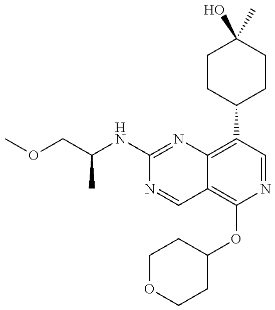 [SMILES: COC[C@H](C)Nc1ncc2c(OC3CCOCC3)ncc([C@H]3CC[C@](C)(O)CC3)c2n1] |
| 294 | 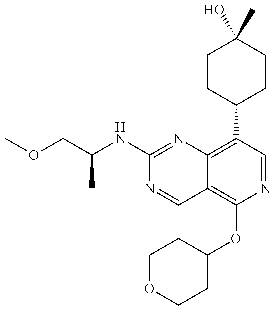 [SMILES: COC[C@H](C)Nc1ncc2c(OC3CCOCC3)ncc([C@H]3CC[C@@](C)(O)CC3)c2n1] |
| 295 | 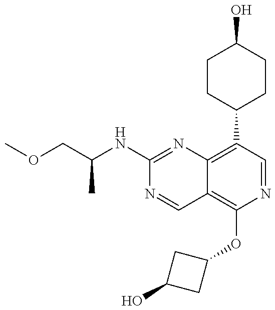 [SMILES: COC[C@H](C)Nc1ncc2c(O[C@H]3C[C@H](O)C3)ncc([C@H]3CC[C@H](O)CC3)c2n1] |
| 296 | 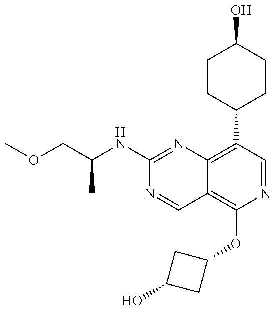 [SMILES: COC[C@H](C)Nc1ncc2c(O[C@H]3C[C@@H](O)C3)ncc([C@H]3CC[C@H](O)CC3)c2n1] |
| 297 | 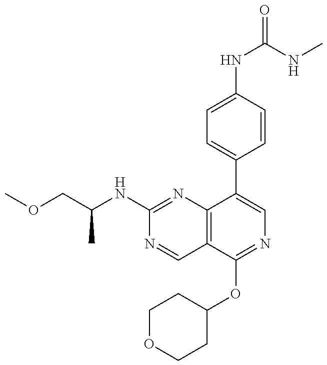 |
| 298 | 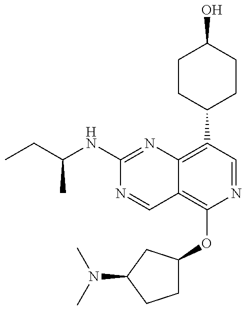 [SMILES: CC[C@H](C)Nc1ncc2c(O[C@H]3CC[C@@H](N(C)C)C3)ncc([C@H]3CC[C@H](O)CC3)c2n1] |
| 299 | 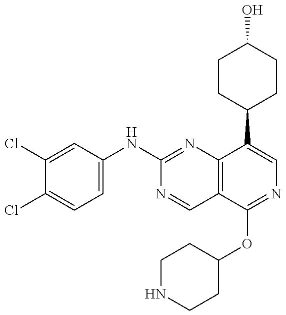 [SMILES: O[C@H]1CC[C@H](c2cnc(OC3CCNCC3)c3cnc(Nc4ccc(Cl)c(Cl)c4)nc32)CC1] |
| 300 | 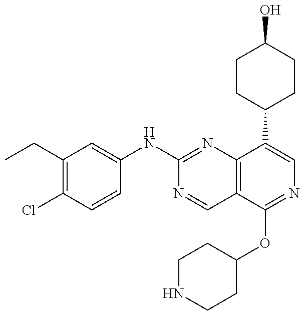 [SMILES: CCc1cc(Nc2ncc3c(OC4CCNCC4)ncc([C@H]4CC[C@H](O)CC4)c3n2)ccc1Cl] |
| 301 | 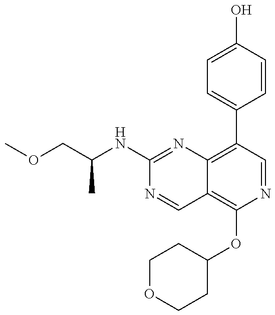 [SMILES: COC[C@H](C)Nc1ncc2c(OC3CCOCC3)ncc(-c3ccc(O)cc3)c2n1] |
| 302 | 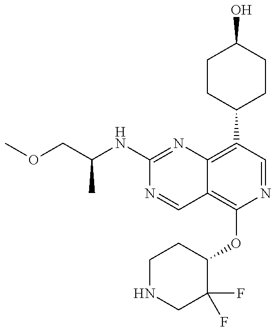 [SMILES: COC[C@H](C)Nc1ncc2c(O[C@H]3CCNCC3(F)F)ncc([C@H]3CC[C@H](O)CC3)c2n1] |
| 303 | 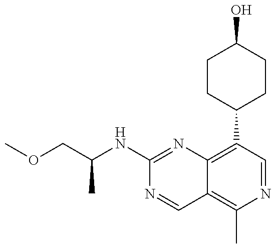 [SMILES: COC[C@H](C)Nc1ncc2c(C)ncc([C@H]3CC[C@H](O)CC3)c2n1] |
| 304 | 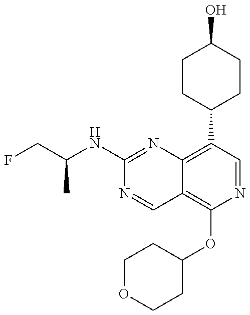 [SMILES: C[C@@H](CF)Nc1ncc2c(OC3CCOCC3)ncc([C@H]3CC[C@H](O)CC3)c2n1] |
| 305 | 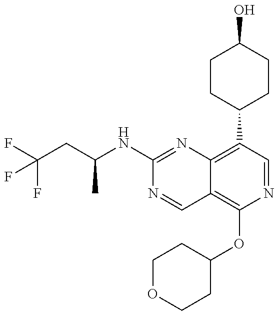 [SMILES: C[C@@H](CC(F)(F)F)Nc1ncc2c(OC3CCOCC3)ncc([C@H]3CC[C@H](O)CC3)c2n1] |
| 306 | 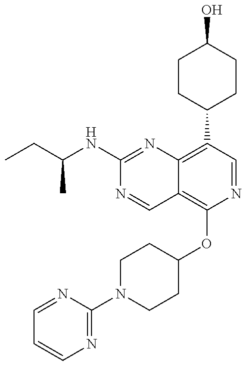 [SMILES: CC[C@H](C)Nc1ncc2c(OC3CCN(c4ncccn4)CC3)ncc([C@H]3CC[C@H](O)CC3)c2n1] |
| 307 | 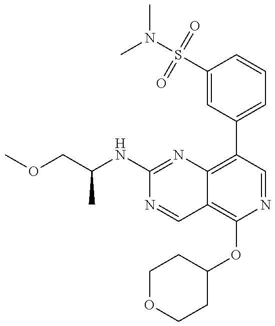 [SMILES: COC[C@H](C)Nc1ncc2c(OC3CCOCC3)ncc(-c3cccc(S(=O)(=O)N(C)C)c3)c2n1] |
| 308 | 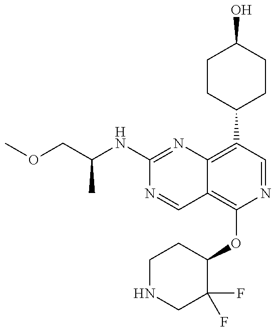 [SMILES: COC[C@H](C)Nc1ncc2c(O[C@@H]3CCNCC3(F)F)ncc([C@H]3CC[C@H](O)CC3)c2n1] |
| 309 | 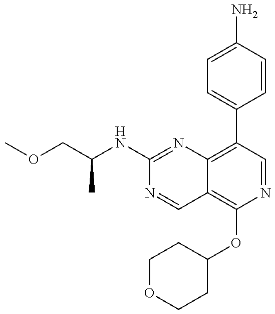 [SMILES: COC[C@H](C)Nc1ncc2c(OC3CCOCC3)ncc(-c3ccc(N)cc3)c2n1] |
| 310 | 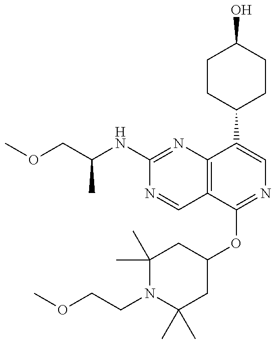 [SMILES: COCCN1C(C)(C)CC(Oc2ncc([C@H]3CC[C@H](O)CC3)c3nc(N[C@@H](C)COC)ncc23)CC1(C)C] |
| 311 | 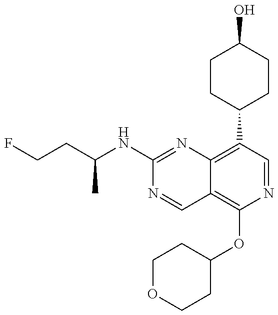 [SMILES: C[C@@H](CCF)Nc1ncc2c(OC3CCOCC3)ncc([C@H]3CC[C@H](O)CC3)c2n1] |
| 312 | 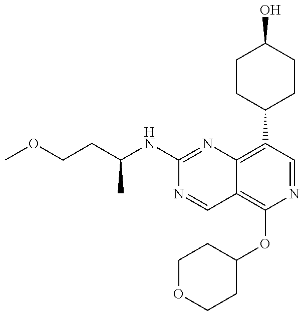 [SMILES: COCC[C@H](C)Nc1ncc2c(OC3CCOCC3)ncc([C@H]3CC[C@H](O)CC3)c2n1] |
| 313 | 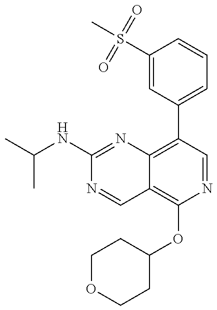 [SMILES: CC(C)Nc1ncc2c(OC3CCOCC3)ncc(-c3cccc(S(C)(=O)=O)c3)c2n1] |
| 314 | 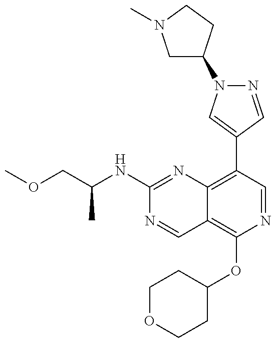 [SMILES: COC[C@H](C)Nc1ncc2c(OC3CCOCC3)ncc(-c3cnn([C@@H]4CCN(C)C4)c3)c2n1] |
| 315 | 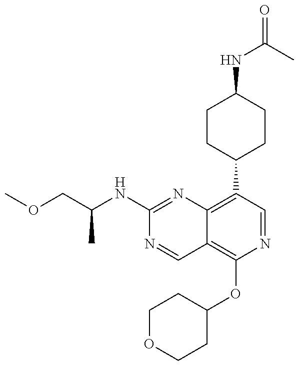 [SMILES: COC[C@H](C)Nc1ncc2c(OC3CCOCC3)ncc([C@H]3CC[C@H](NC(C)=O)CC3)c2n1] |
| 316 | 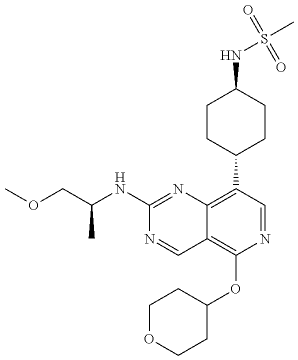 |
| 317 | 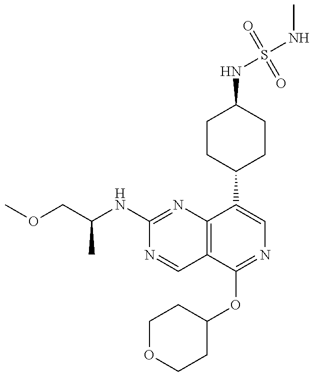 |
| 318 | 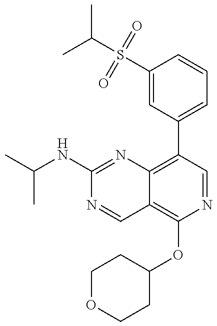 [SMILES: CC(C)Nc1ncc2c(OC3CCOCC3)ncc(-c3cccc(S(=O)(=O)C(C)C)c3)c2n1] |
| 319 | 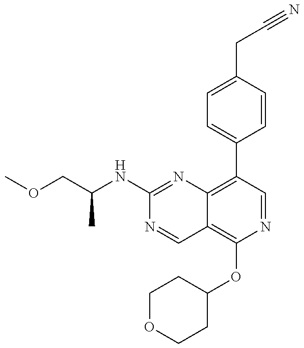 [SMILES: COC[C@H](C)Nc1ncc2c(OC3CCOCC3)ncc(-c3ccc(CC#N)cc3)c2n1] |
| 320 | 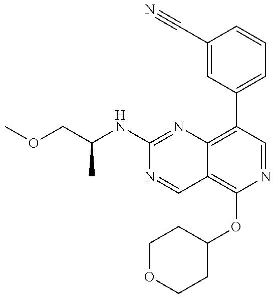 [SMILES: COC[C@H](C)Nc1ncc2c(OC3CCOCC3)ncc(-c3cccc(C#N)c3)c2n1] |
| 321 | 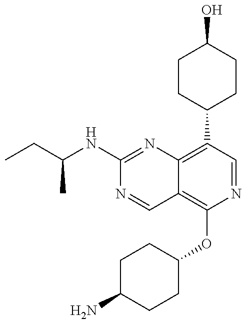 [SMILES: CC[C@H](C)Nc1ncc2c(O[C@H]3CC[C@H](N)CC3)ncc([C@H]3CC[C@H](O)CC3)c2n1] |
| 322 | 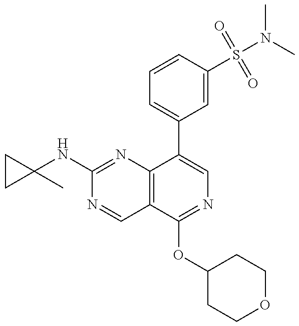 [SMILES: CN(C)S(=O)(=O)c1cccc(-c2cnc(OC3CCOCC3)c3cnc(NC4(C)CC4)nc23)c1] |
| 323 | 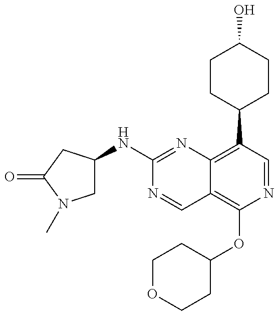 [SMILES: CN1C[C@H](Nc2ncc3c(OC4CCOCC4)ncc([C@H]4CC[C@H](O)CC4)c3n2)CC1=O] |
| 324 | 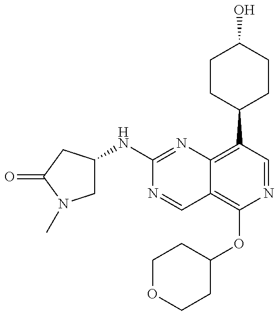 [SMILES: CN1C[C@@H](Nc2ncc3c(OC4CCOCC4)ncc([C@H]4CC[C@H](O)CC4)c3n2)CC1=O] |
| 325 | 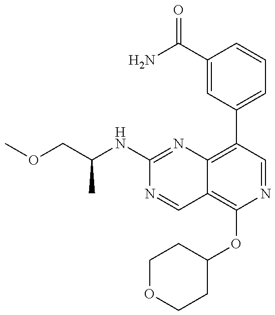 [SMILES: COC[C@H](C)Nc1ncc2c(OC3CCOCC3)ncc(-c3cccc(C(N)=O)c3)c2n1] |
| 326 | 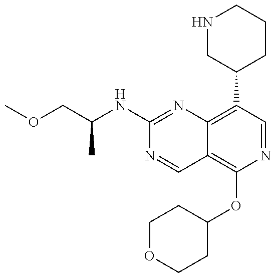 [SMILES: COC[C@H](C)Nc1ncc2c(OC3CCOCC3)ncc([C@H]3CCCNC3)c2n1] |
| 327 | 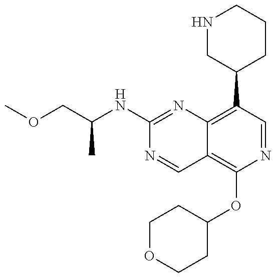 [SMILES: COC[C@H](C)Nc1ncc2c(OC3CCOCC3)ncc([C@@H]3CCCNC3)c2n1] |
| 328 | 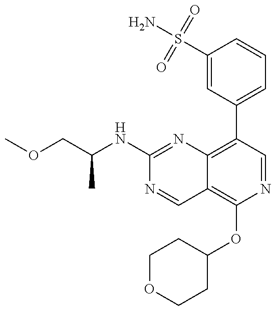 [SMILES: COC[C@H](C)Nc1ncc2c(OC3CCOCC3)ncc(-c3cccc(S(N)(=O)=O)c3)c2n1] |
| 329 | 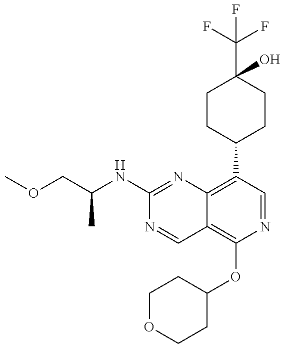 [SMILES: COC[C@H](C)Nc1ncc2c(OC3CCOCC3)ncc([C@H]3CC[C@@](O)(C(F)(F)F)CC3)c2n1] |
| 330 | 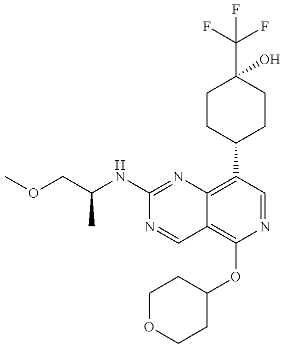 [SMILES: COC[C@H](C)Nc1ncc2c(OC3CCOCC3)ncc([C@H]3CC[C@](O)(C(F)(F)F)CC3)c2n1] |
| 331 | 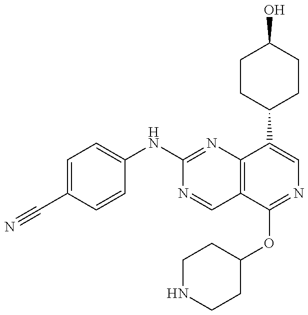 [SMILES: N#Cc1ccc(Nc2ncc3c(OC4CCNCC4)ncc([C@H]4CC[C@H](O)CC4)c3n2)cc1] |
| 332 | 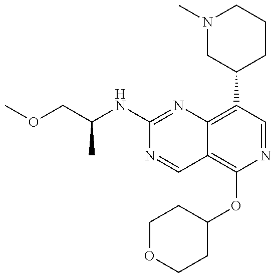 [SMILES: COC[C@H](C)Nc1ncc2c(OC3CCOCC3)ncc([C@H]3CCCN(C)C3)c2n1] |
| 333 | 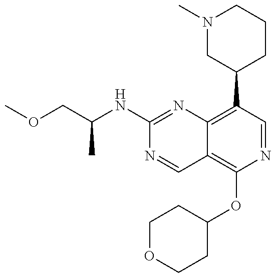 [SMILES: COC[C@H](C)Nc1ncc2c(OC3CCOCC3)ncc([C@@H]3CCCN(C)C3)c2n1] |
| 334 | 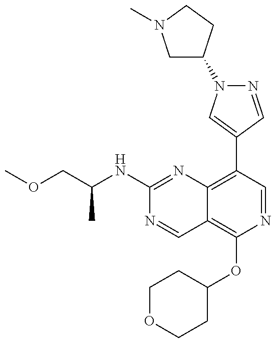 [SMILES: COC[C@H](C)Nc1ncc2c(OC3CCOCC3)ncc(-c3cnn([C@H]4CCN(C)C4)c3)c2n1] |
| 335 | 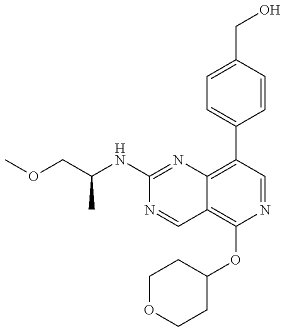 [SMILES: COC[C@H](C)Nc1ncc2c(OC3CCOCC3)ncc(-c3ccc(CO)cc3)c2n1] |
| 336 | 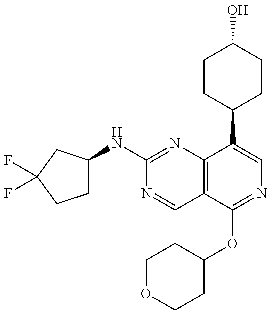 [SMILES: O[C@H]1CC[C@H](c2cnc(OC3CCOCC3)c3cnc(N[C@H]4CCC(F)(F)C4)nc32)CC1] |
| 337 | 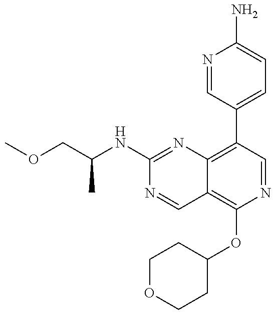 [SMILES: COC[C@H](C)Nc1ncc2c(OC3CCOCC3)ncc(-c3ccc(N)nc3)c2n1] |
| 338 | 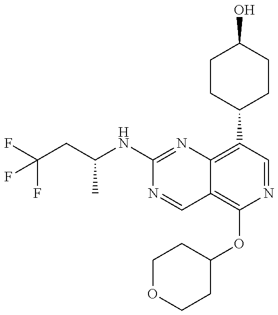 [SMILES: C[C@H](CC(F)(F)F)Nc1ncc2c(OC3CCOCC3)ncc([C@H]3CC[C@H](O)CC3)c2n1] |
| 339 | 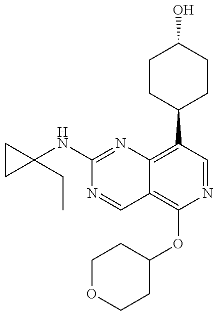 [SMILES: CCC1(Nc2ncc3c(OC4CCOCC4)ncc([C@H]4CC[C@H](O)CC4)c3n2)CC1] |
| 340 | 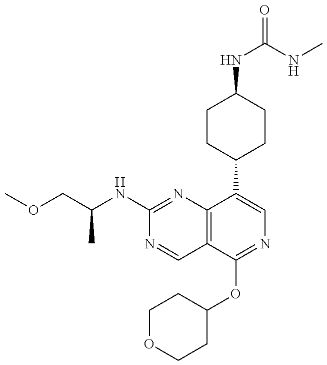 |
| 341 | 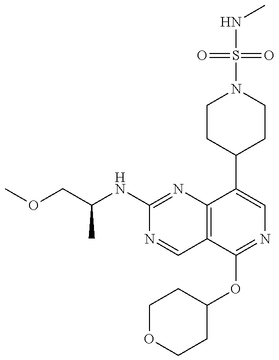 [SMILES: CNS(=O)(=O)N1CCC(c2cnc(OC3CCOCC3)c3cnc(N[C@@H](C)COC)nc23)CC1] |
| 342 | 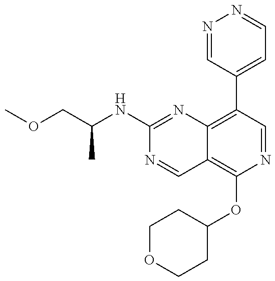 [SMILES: COC[C@H](C)Nc1ncc2c(OC3CCOCC3)ncc(-c3ccnnc3)c2n1] |
| 343 | 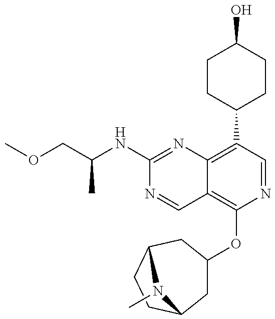 [SMILES: COC[C@H](C)Nc1ncc2c(OC3C[C@H]4CC[C@@H](C3)N4C)ncc([C@H]3CC[C@H](O)CC3)c2n1] |
| 344 | 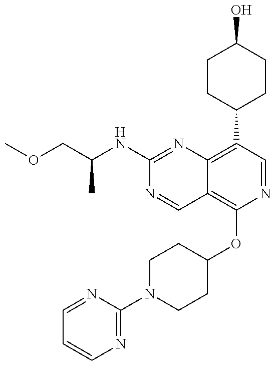 [SMILES: COC[C@H](C)Nc1ncc2c(OC3CCN(c4ncccn4)CC3)ncc([C@H]3CC[C@H](O)CC3)c2n1] |
| 345 | 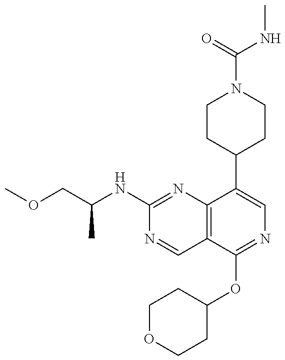 |
| 346 | 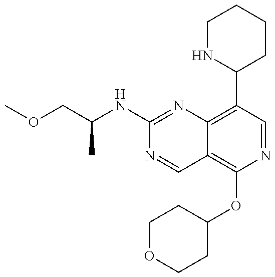 [SMILES: COC[C@H](C)Nc1ncc2c(OC3CCOCC3)ncc(C3CCCCN3)c2n1] |
| 347 | 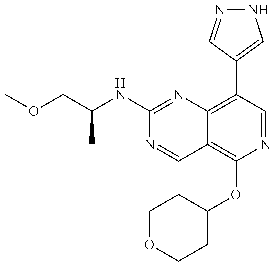 [SMILES: COC[C@H](C)Nc1ncc2c(OC3CCOCC3)ncc(-c3cn[nH]c3)c2n1] |
| 348 | 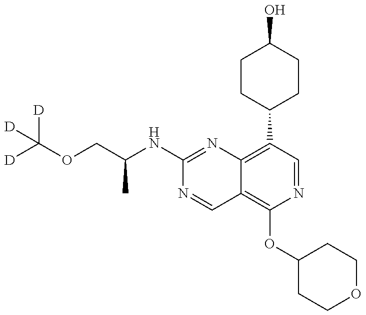 [SMILES: [2H]C([2H])([2H])OC[C@H](C)Nc1ncc2c(OC3CCOCC3)ncc([C@H]3CC[C@H](O)CC3)c2n1] |
| 349 | 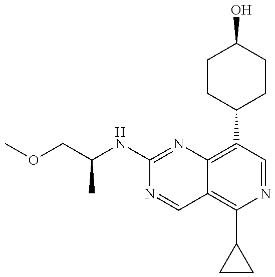 [SMILES: COC[C@H](C)Nc1ncc2c(C3CC3)ncc([C@H]3CC[C@H](O)CC3)c2n1] |
| 350 | 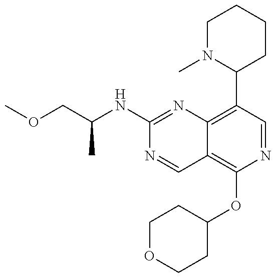 [SMILES: COC[C@H](C)Nc1ncc2c(OC3CCOCC3)ncc(C3CCCCN3C)c2n1] |
| 351 | 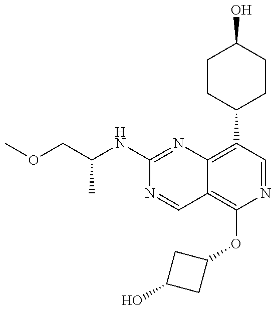 |
| 352 | 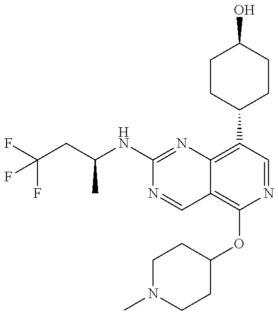 [SMILES: C[C@@H](CC(F)(F)F)Nc1ncc2c(OC3CCN(C)CC3)ncc([C@H]3CC[C@H](O)CC3)c2n1] |
| 353 | 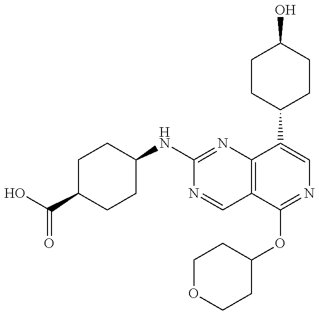 [SMILES: O=C(O)[C@H]1CC[C@@H](Nc2ncc3c(OC4CCOCC4)ncc([C@H]4CC[C@H](O)CC4)c3n2)CC1] |
| 354 | 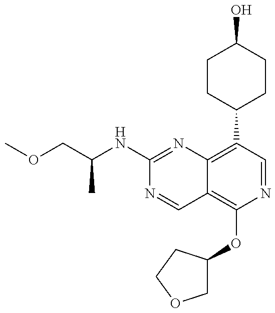 [SMILES: COC[C@H](C)Nc1ncc2c(O[C@@H]3CCOC3)ncc([C@H]3CC[C@H](O)CC3)c2n1] |
| 355 | 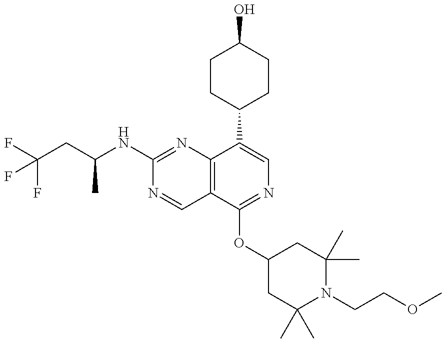 [SMILES: COCCN1C(C)(C)CC(Oc2ncc([C@H]3CC[C@H](O)CC3)c3nc(N[C@@H](C)CC(F)(F)F)ncc23)CC1(C)C] |
| 356 | 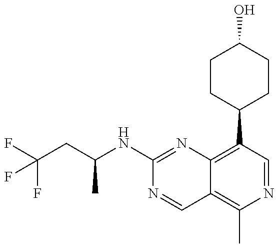 [SMILES: Cc1ncc([C@H]2CC[C@H](O)CC2)c2nc(N[C@@H](C)CC(F)(F)F)ncc12] |
| 357 | 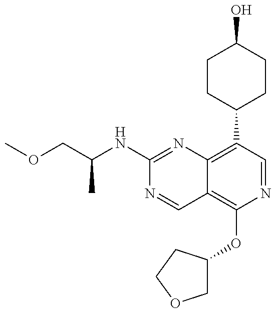 [SMILES: COC[C@H](C)Nc1ncc2c(O[C@H]3CCOC3)ncc([C@H]3CC[C@H](O)CC3)c2n1] |
| 358 | 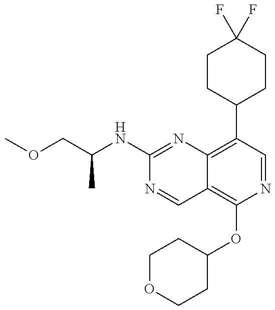 [SMILES: COC[C@H](C)Nc1ncc2c(OC3CCOCC3)ncc(C3CCC(F)(F)CC3)c2n1] |
| 359 | 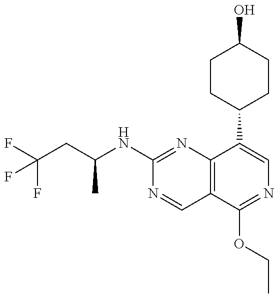 [SMILES: CCOc1ncc([C@H]2CC[C@H](O)CC2)c2nc(N[C@@H](C)CC(F)(F)F)ncc12] |
| 360 | 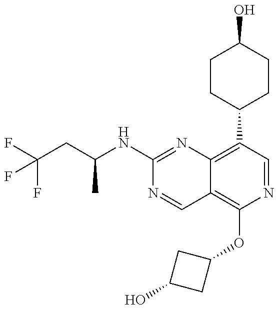 [SMILES: C[C@@H](CC(F)(F)F)Nc1ncc2c(O[C@H]3C[C@@H](O)C3)ncc([C@H]3CC[C@H](O)CC3)c2n1] |
| 361 | 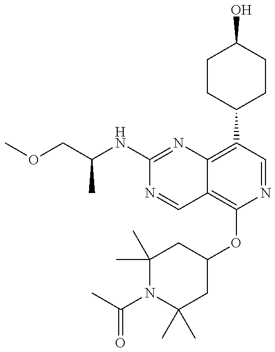 [SMILES: COC[C@H](C)Nc1ncc2c(OC3CC(C)(C)N(C(C)=O)C(C)(C)C3)ncc([C@H]3CC[C@H](O)CC3)c2n1] |
| 362 | 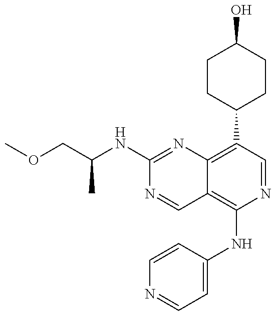 [SMILES: COC[C@H](C)Nc1ncc2c(Nc3ccncc3)ncc([C@H]3CC[C@H](O)CC3)c2n1] |
| 363 | 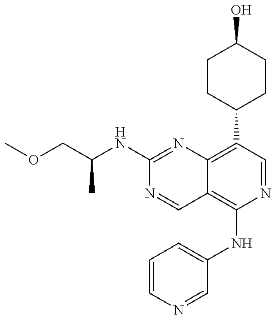 [SMILES: COC[C@H](C)Nc1ncc2c(Nc3cccnc3)ncc([C@H]3CC[C@H](O)CC3)c2n1] |
| 364 | 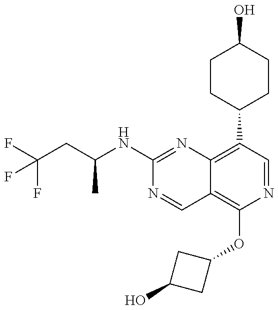 [SMILES: C[C@@H](CC(F)(F)F)Nc1ncc2c(O[C@H]3C[C@H](O)C3)ncc([C@H]3CC[C@H](O)CC3)c2n1] |
| 365 | 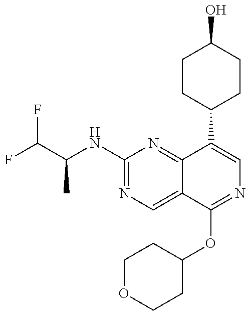 [SMILES: C[C@H](Nc1ncc2c(OC3CCOCC3)ncc([C@H]3CC[C@H](O)CC3)c2n1)C(F)F] |
| 366 | 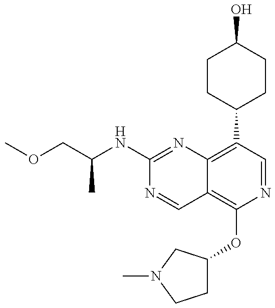 [SMILES: COC[C@H](C)Nc1ncc2c(O[C@@H]3CCN(C)C3)ncc([C@H]3CC[C@H](O)CC3)c2n1] |
| 367 | 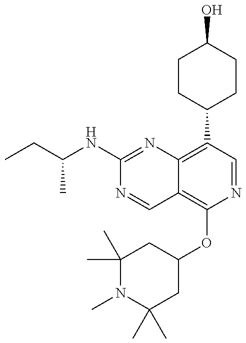 [SMILES: CC[C@@H](C)Nc1ncc2c(OC3CC(C)(C)N(C)C(C)(C)C3)ncc([C@H]3CC[C@H](O)CC3)c2n1] |
| 368 | 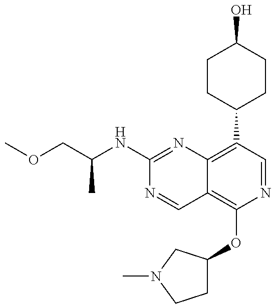 [SMILES: COC[C@H](C)Nc1ncc2c(O[C@H]3CCN(C)C3)ncc([C@H]3CC[C@H](O)CC3)c2n1] |
| 369 | 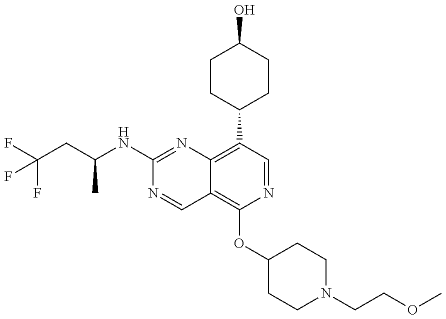 [SMILES: COCCN1CCC(Oc2ncc([C@H]3CC[C@H](O)CC3)c3nc(N[C@@H](C)CC(F)(F)F)ncc23)CC1] |
| 370 | 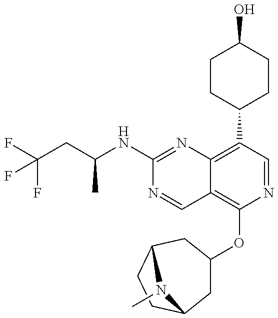 [SMILES: C[C@@H](CC(F)(F)F)Nc1ncc2c(OC3C[C@H]4CC[C@@H](C3)N4C)ncc([C@H]3CC[C@H](O)CC3)c2n1] |
| 371 | 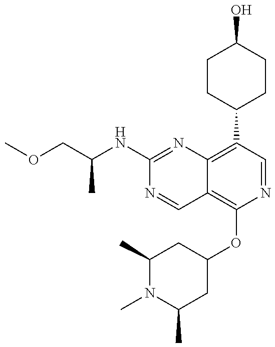 [SMILES: COC[C@H](C)Nc1ncc2c(OC3C[C@@H](C)N(C)[C@@H](C)C3)ncc([C@H]3CC[C@H](O)CC3)c2n1] |
| 372 | 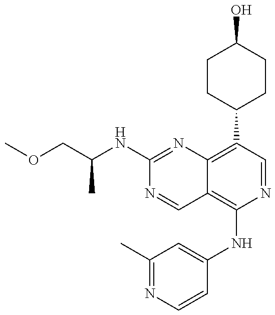 [SMILES: COC[C@H](C)Nc1ncc2c(Nc3ccnc(C)c3)ncc([C@H]3CC[C@H](O)CC3)c2n1] |
| 373 | 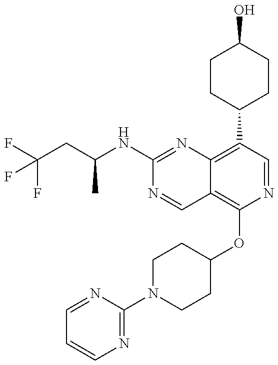 [SMILES: C[C@@H](CC(F)(F)F)Nc1ncc2c(OC3CCN(c4ncccn4)CC3)ncc([C@H]3CC[C@H](O)CC3)c2n1] |
| 374 | 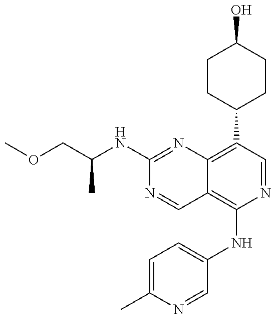 [SMILES: COC[C@H](C)Nc1ncc2c(Nc3ccc(C)nc3)ncc([C@H]3CC[C@H](O)CC3)c2n1] |
| 375 | 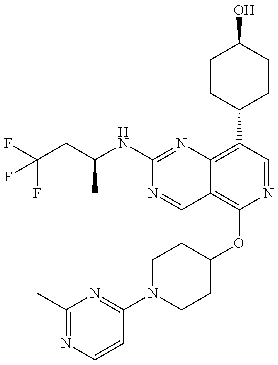 [SMILES: Cc1nccc(N2CCC(Oc3ncc([C@H]4CC[C@H](O)CC4)c4nc(N[C@@H](C)CC(F)(F)F)ncc34)CC2)n1] |
| 376 | 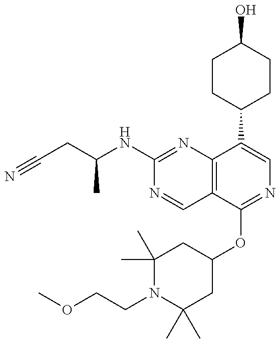 [SMILES: COCCN1C(C)(C)CC(Oc2ncc([C@H]3CC[C@H](O)CC3)c3nc(N[C@@H](C)CC#N)ncc23)CC1(C)C] |
| 377 | 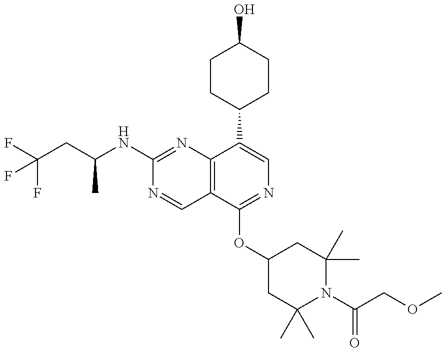 [SMILES: COCC(=O)N1C(C)(C)CC(Oc2ncc([C@H]3CC[C@H](O)CC3)c3nc(N[C@@H](C)CC(F)(F)F)ncc23)CC1(C)C] |
| 378 | 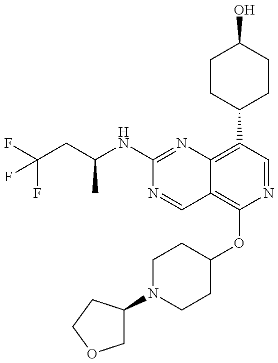 [SMILES: C[C@@H](CC(F)(F)F)Nc1ncc2c(OC3CCN([C@@H]4CCOC4)CC3)ncc([C@H]3CC[C@H](O)CC3)c2n1] |
| 379 | 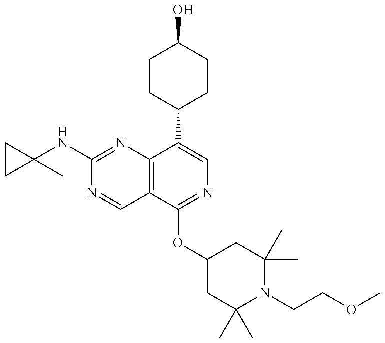 [SMILES: COCCN1C(C)(C)CC(Oc2ncc([C@H]3CC[C@H](O)CC3)c3nc(NC4(C)CC4)ncc23)CC1(C)C] |
| 380 | 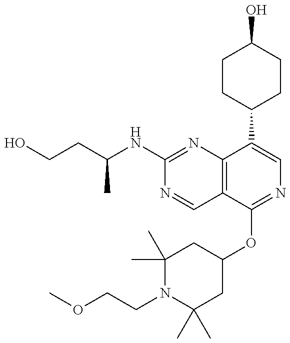 [SMILES: COCCN1C(C)(C)CC(Oc2ncc([C@H]3CC[C@H](O)CC3)c3nc(N[C@@H](C)CCO)ncc23)CC1(C)C] |
| 381 | 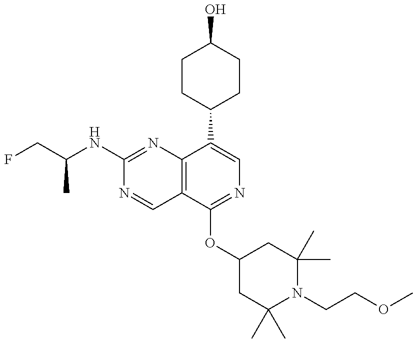 [SMILES: COCCN1C(C)(C)CC(Oc2ncc([C@H]3CC[C@H](O)CC3)c3nc(N[C@@H](C)CF)ncc23)CC1(C)C] |
| 382 | 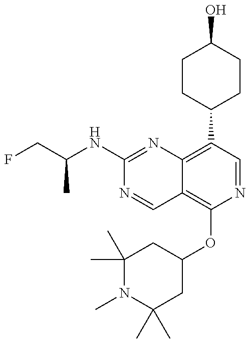 [SMILES: C[C@@H](CF)Nc1ncc2c(OC3CC(C)(C)N(C)C(C)(C)C3)ncc([C@H]3CC[C@H](O)CC3)c2n1] |
| 383 | 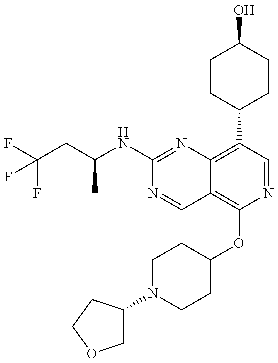 [SMILES: C[C@@H](CC(F)(F)F)Nc1ncc2c(OC3CCN([C@H]4CCOC4)CC3)ncc([C@H]3CC[C@H](O)CC3)c2n1] |
| 384 | 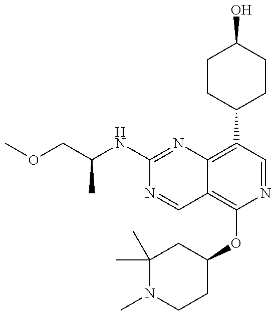 [SMILES: COC[C@H](C)Nc1ncc2c(O[C@H]3CCN(C)C(C)(C)C3)ncc([C@H]3CC[C@H](O)CC3)c2n1] |
| 385 | 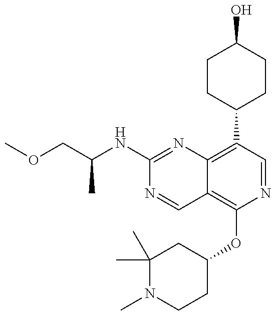 [SMILES: COC[C@H](C)Nc1ncc2c(O[C@@H]3CCN(C)C(C)(C)C3)ncc([C@H]3CC[C@H](O)CC3)c2n1] |
| 386 | 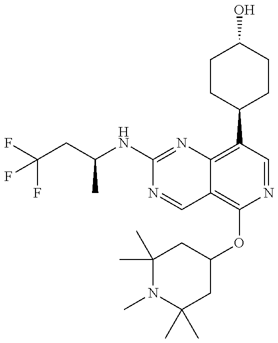 [SMILES: C[C@@H](CC(F)(F)F)Nc1ncc2c(OC3CC(C)(C)N(C)C(C)(C)C3)ncc([C@H]3CC[C@H](O)CC3)c2n1] |
| 387 | 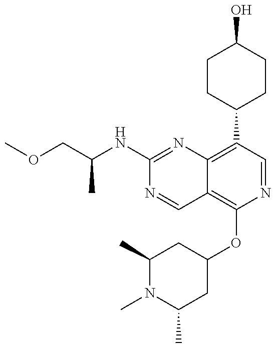 [SMILES: COC[C@H](C)Nc1ncc2c(OC3C[C@H](C)N(C)[C@@H](C)C3)ncc([C@H]3CC[C@H](O)CC3)c2n1] |
| 388 | 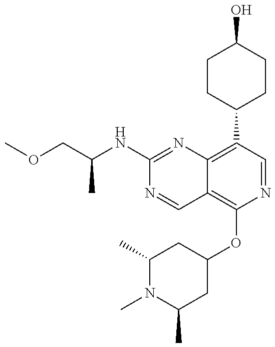 [SMILES: COC[C@H](C)Nc1ncc2c(OC3C[C@@H](C)N(C)[C@H](C)C3)ncc([C@H]3CC[C@H](O)CC3)c2n1] |
| 389 | 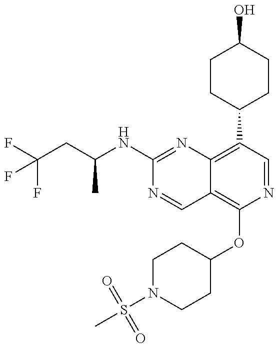 [SMILES: C[C@@H](CC(F)(F)F)Nc1ncc2c(OC3CCN(S(C)(=O)=O)CC3)ncc([C@H]3CC[C@H](O)CC3)c2n1] |
| 390 | 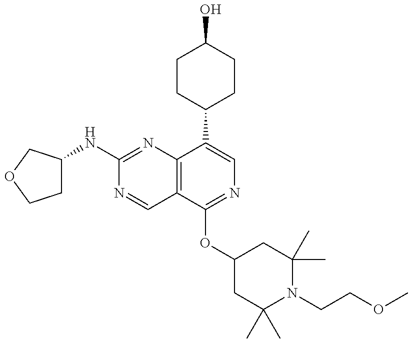 [SMILES: COCCN1C(C)(C)CC(Oc2ncc([C@H]3CC[C@H](O)CC3)c3nc(N[C@@H]4CCOC4)ncc23)CC1(C)C] |
| 391 | 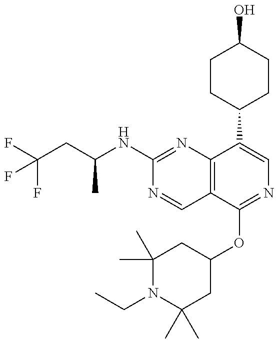 [SMILES: CCN1C(C)(C)CC(Oc2ncc([C@H]3CC[C@H](O)CC3)c3nc(N[C@@H](C)CC(F)(F)F)ncc23)CC1(C)C] |
| 392 | 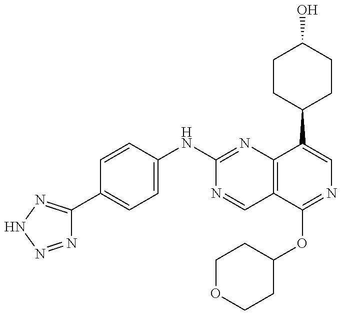 [SMILES: O[C@H]1CC[C@H](c2cnc(OC3CCOCC3)c3cnc(Nc4ccc(-c5nn[nH]n5)cc4)nc32)CC1] |
| 393 | 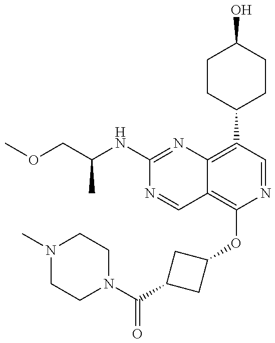 [SMILES: COC[C@H](C)Nc1ncc2c(O[C@H]3C[C@@H](C(=O)N4CCN(C)CC4)C3)ncc([C@H]3CC[C@H](O)CC3)c2n1] |
| 394 | 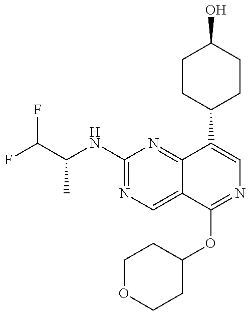 [SMILES: C[C@@H](Nc1ncc2c(OC3CCOCC3)ncc([C@H]3CC[C@H](O)CC3)c2n1)C(F)F] |
| 395 | 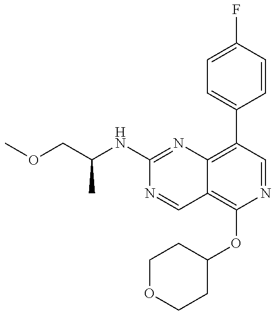 [SMILES: COC[C@H](C)Nc1ncc2c(OC3CCOCC3)ncc(-c3ccc(F)cc3)c2n1] |
| 396 | 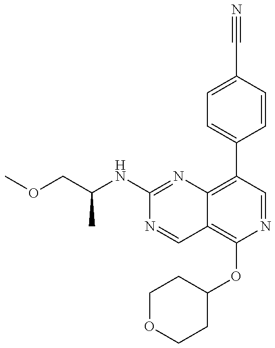 [SMILES: COC[C@H](C)Nc1ncc2c(OC3CCOCC3)ncc(-c3ccc(C#N)cc3)c2n1] |
| 397 | 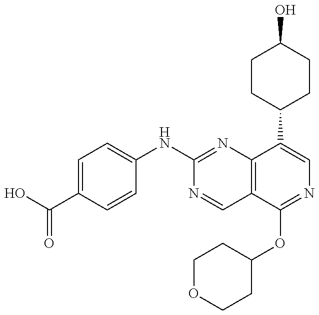 [SMILES: O=C(O)c1ccc(Nc2ncc3c(OC4CCOCC4)ncc([C@H]4CC[C@H](O)CC4)c3n2)cc1] |
| 398 | 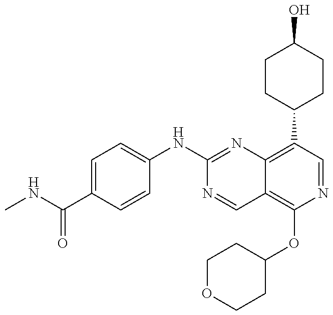 [SMILES: CNC(=O)c1ccc(Nc2ncc3c(OC4CCOCC4)ncc([C@H]4CC[C@H](O)CC4)c3n2)cc1] |
| 399 | 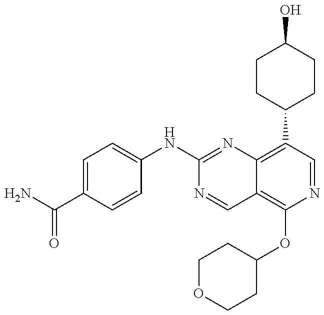 [SMILES: NC(=O)c1ccc(Nc2ncc3c(OC4CCOCC4)ncc([C@H]4CC[C@H](O)CC4)c3n2)cc1] |
| 400 | 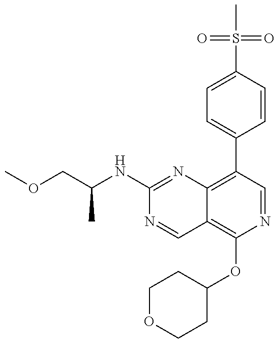 [SMILES: COC[C@H](C)Nc1ncc2c(OC3CCOCC3)ncc(-c3ccc(S(C)(=O)=O)cc3)c2n1] |
| 401 | 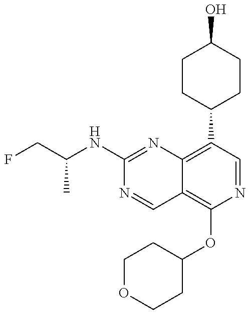 [SMILES: C[C@H](CF)Nc1ncc2c(OC3CCOCC3)ncc([C@H]3CC[C@H](O)CC3)c2n1] |
| 402 | 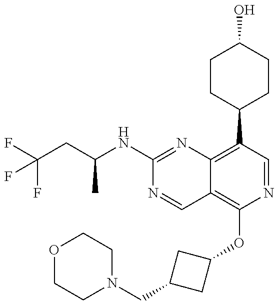 [SMILES: C[C@@H](CC(F)(F)F)Nc1ncc2c(O[C@H]3C[C@@H](CN4CCOCC4)C3)ncc([C@H]3CC[C@H](O)CC3)c2n1] |
| 403 | 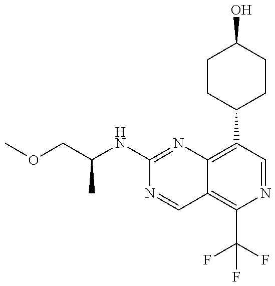 [SMILES: COC[C@H](C)Nc1ncc2c(C(F)(F)F)ncc([C@H]3CC[C@H](O)CC3)c2n1] |
| 404 | 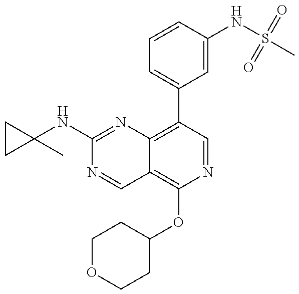 [SMILES: CC1(Nc2ncc3c(OC4CCOCC4)ncc(-c4cccc(NS(C)(=O)=O)c4)c3n2)CC1] |
| 405 | 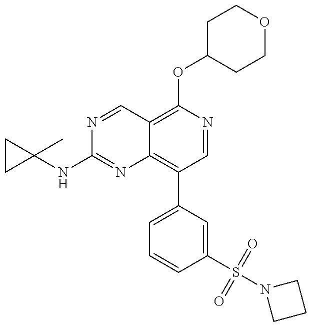 [SMILES: CC1(Nc2ncc3c(OC4CCOCC4)ncc(-c4cccc(S(=O)(=O)N5CCC5)c4)c3n2)CC1] |
| 406 | 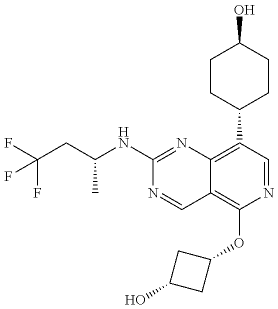 [SMILES: C[C@H](CC(F)(F)F)Nc1ncc2c(O[C@H]3C[C@@H](O)C3)ncc([C@H]3CC[C@H](O)CC3)c2n1] |
| 407 | 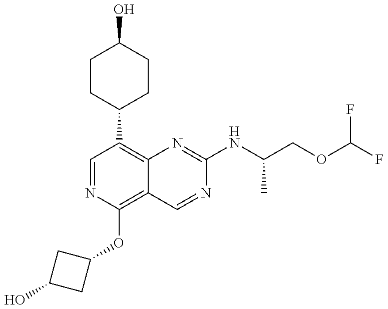 [SMILES: C[C@@H](COC(F)F)Nc1ncc2c(O[C@H]3C[C@@H](O)C3)ncc([C@H]3CC[C@H](O)CC3)c2n1] |
| 408 | 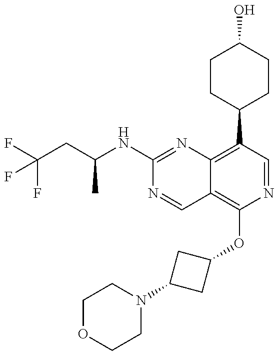 [SMILES: C[C@@H](CC(F)(F)F)Nc1ncc2c(O[C@H]3C[C@@H](N4CCOCC4)C3)ncc([C@H]3CC[C@H](O)CC3)c2n1] |
| 409 | 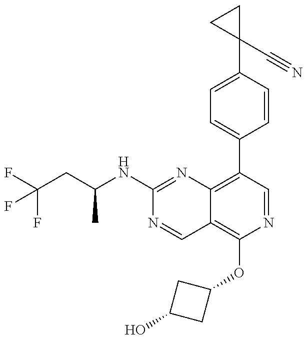 [SMILES: C[C@@H](CC(F)(F)F)Nc1ncc2c(O[C@H]3C[C@@H](O)C3)ncc(-c3ccc(C4(C#N)CC4)cc3)c2n1] |
| 410 | 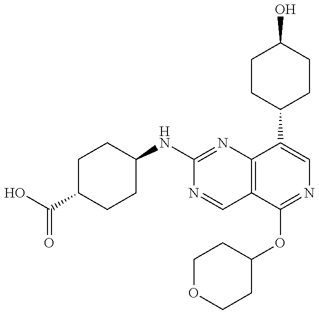 [SMILES: O=C(O)[C@H]1CC[C@H](Nc2ncc3c(OC4CCOCC4)ncc([C@H]4CC[C@H](O)CC4)c3n2)CC1] |
| 411 | 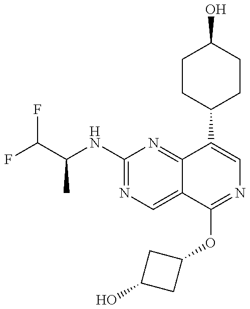 [SMILES: C[C@H](Nc1ncc2c(O[C@H]3C[C@@H](O)C3)ncc([C@H]3CC[C@H](O)CC3)c2n1)C(F)F] |
| 412 | 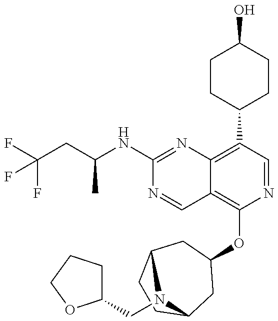 [SMILES: C[C@@H](CC(F)(F)F)Nc1ncc2c(O[C@@H]3C[C@H]4CC[C@@H](C3)N4C[C@H]3CCCO3)ncc([C@H]3CC[C@H](O)CC3)c2n1] |
| 413 | 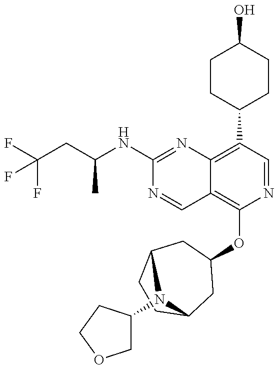 [SMILES: C[C@@H](CC(F)(F)F)Nc1ncc2c(O[C@@H]3C[C@H]4CC[C@@H](C3)N4[C@H]3CCOC3)ncc([C@H]3CC[C@H](O)CC3)c2n1] |
| 414 | 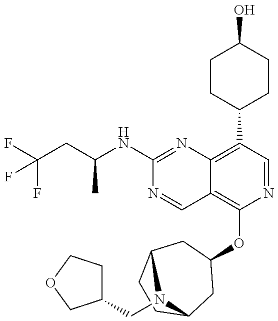 [SMILES: C[C@@H](CC(F)(F)F)Nc1ncc2c(O[C@@H]3C[C@H]4CC[C@@H](C3)N4C[C@H]3CCOC3)ncc([C@H]3CC[C@H](O)CC3)c2n1] |
| 415 | 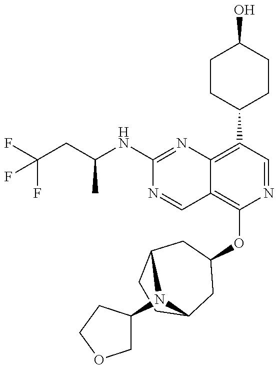 [SMILES: C[C@@H](CC(F)(F)F)Nc1ncc2c(O[C@@H]3C[C@H]4CC[C@@H](C3)N4[C@@H]3CCOC3)ncc([C@H]3CC[C@H](O)CC3)c2n1] |
| 416 | 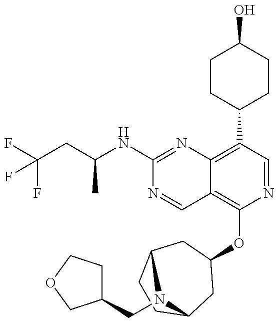 [SMILES: C[C@@H](CC(F)(F)F)Nc1ncc2c(O[C@@H]3C[C@H]4CC[C@@H](C3)N4C[C@@H]3CCOC3)ncc([C@H]3CC[C@H](O)CC3)c2n1] |
| 417 | 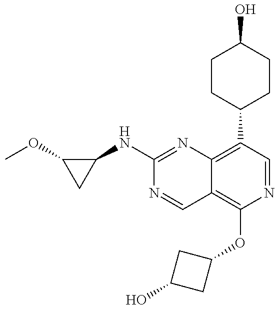 [SMILES: CO[C@H]1C[C@@H]1Nc1ncc2c(O[C@H]3C[C@@H](O)C3)ncc([C@H]3CC[C@H](O)CC3)c2n1] |
| 418 | 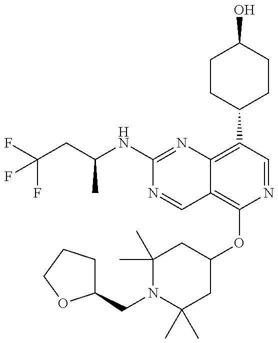 [SMILES: C[C@@H](CC(F)(F)F)Nc1ncc2c(OC3CC(C)(C)N(C[C@@H]4CCCO4)C(C)(C)C3)ncc([C@H]3CC[C@H](O)CC3)c2n1] |
| 419 | 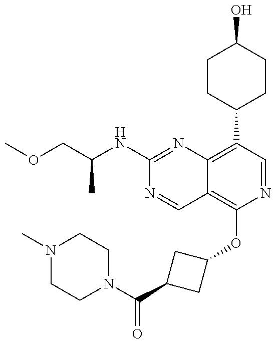 [SMILES: COC[C@H](C)Nc1ncc2c(O[C@H]3C[C@H](C(=O)N4CCN(C)CC4)C3)ncc([C@H]3CC[C@H](O)CC3)c2n1] |
| 420 | 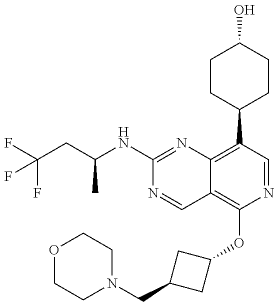 [SMILES: C[C@@H](CC(F)(F)F)Nc1ncc2c(O[C@H]3C[C@H](CN4CCOCC4)C3)ncc([C@H]3CC[C@H](O)CC3)c2n1] |
| 421 | 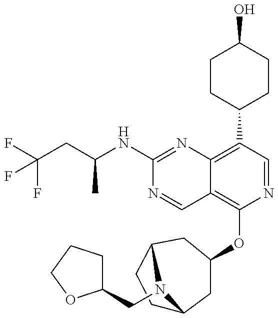 [SMILES: C[C@@H](CC(F)(F)F)Nc1ncc2c(O[C@@H]3C[C@H]4CC[C@@H](C3)N4C[C@@H]3CCCO3)ncc([C@H]3CC[C@H](O)CC3)c2n1] |
| 422 | 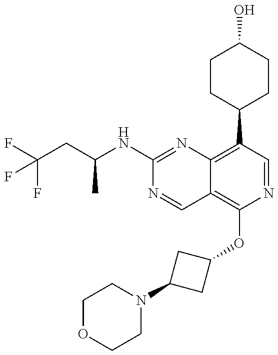 [SMILES: C[C@@H](CC(F)(F)F)Nc1ncc2c(O[C@H]3C[C@H](N4CCOCC4)C3)ncc([C@H]3CC[C@H](O)CC3)c2n1] |
| 423 | 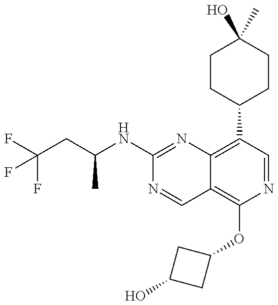 [SMILES: C[C@@H](CC(F)(F)F)Nc1ncc2c(O[C@H]3C[C@@H](O)C3)ncc([C@H]3CC[C@](C)(O)CC3)c2n1] |
| 424 | 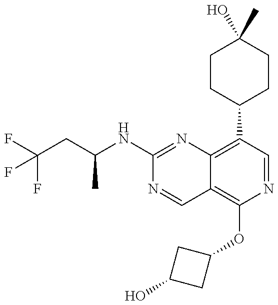 [SMILES: C[C@@H](CC(F)(F)F)Nc1ncc2c(O[C@H]3C[C@@H](O)C3)ncc([C@H]3CC[C@@](C)(O)CC3)c2n1] |
| 425 | 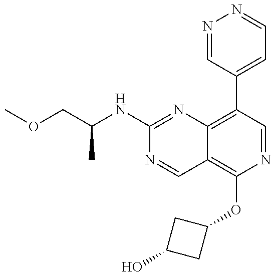 [SMILES: COC[C@H](C)Nc1ncc2c(O[C@H]3C[C@@H](O)C3)ncc(-c3ccnnc3)c2n1] |
| 426 | 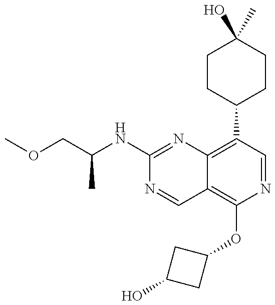 [SMILES: COC[C@H](C)Nc1ncc2c(O[C@H]3C[C@@H](O)C3)ncc([C@H]3CC[C@](C)(O)CC3)c2n1] |
| 427 | 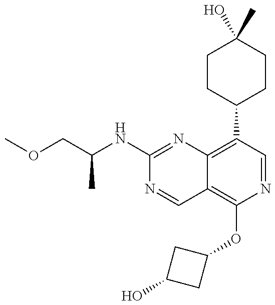 [SMILES: COC[C@H](C)Nc1ncc2c(O[C@H]3C[C@@H](O)C3)ncc([C@H]3CC[C@@](C)(O)CC3)c2n1] |
| 428 | 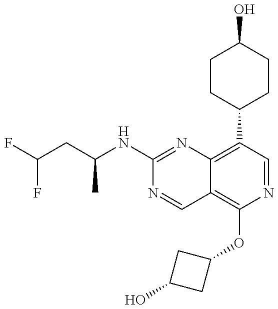 [SMILES: C[C@@H](CC(F)F)Nc1ncc2c(O[C@H]3C[C@@H](O)C3)ncc([C@H]3CC[C@H](O)CC3)c2n1] |
| 429 | 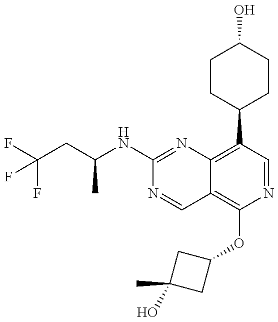 [SMILES: C[C@@H](CC(F)(F)F)Nc1ncc2c(O[C@H]3C[C@@](C)(O)C3)ncc([C@H]3CC[C@H](O)CC3)c2n1] |
| 430 | 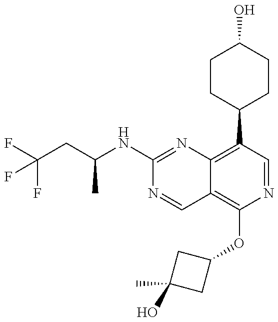 [SMILES: C[C@@H](CC(F)(F)F)Nc1ncc2c(O[C@H]3C[C@](C)(O)C3)ncc([C@H]3CC[C@H](O)CC3)c2n1] |
| 431 | 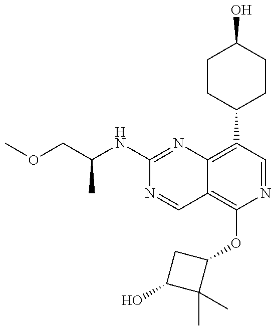 [SMILES: COC[C@H](C)Nc1ncc2c(O[C@H]3C[C@@H](O)C3(C)C)ncc([C@H]3CC[C@H](O)CC3)c2n1] |
| 432 | 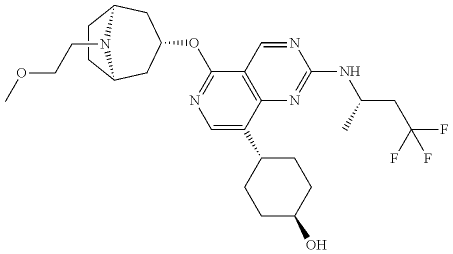 [SMILES: COCCN1[C@@H]2CC[C@H]1C[C@H](Oc1ncc([C@H]3CC[C@H](O)CC3)c3nc(N[C@@H](C)CC(F)(F)F)ncc13)C2] |
| 433 | 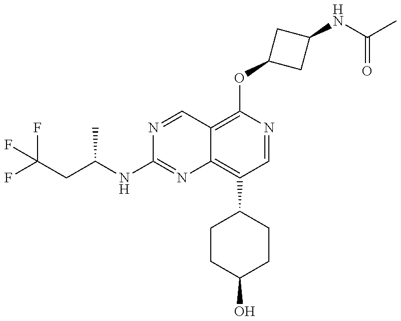 [SMILES: CC(=O)N[C@H]1C[C@@H](Oc2ncc([C@H]3CC[C@H](O)CC3)c3nc(N[C@@H](C)CC(F)(F)F)ncc23)C1] |
| 434 | 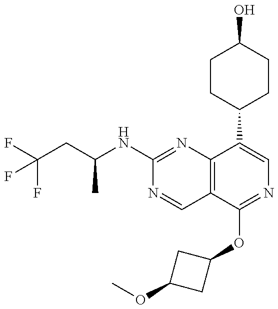 [SMILES: CO[C@H]1C[C@@H](Oc2ncc([C@H]3CC[C@H](O)CC3)c3nc(N[C@@H](C)CC(F)(F)F)ncc23)C1] |
| 435 | 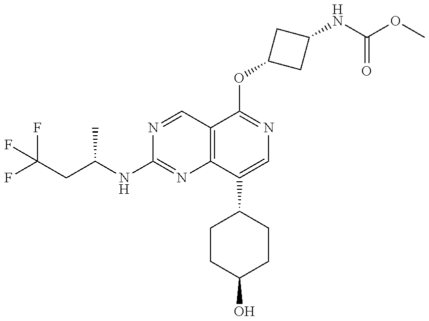 [SMILES: COC(=O)N[C@H]1C[C@@H](Oc2ncc([C@H]3CC[C@H](O)CC3)c3nc(N[C@@H](C)CC(F)(F)F)ncc23)C1] |
| 436 | 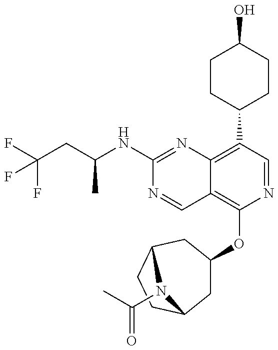 [SMILES: CC(=O)N1[C@@H]2CC[C@H]1C[C@H](Oc1ncc([C@H]3CC[C@H](O)CC3)c3nc(N[C@@H](C)CC(F)(F)F)ncc13)C2] |
| 437 | 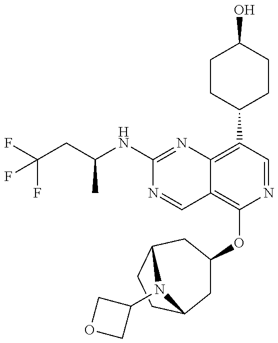 [SMILES: C[C@@H](CC(F)(F)F)Nc1ncc2c(O[C@@H]3C[C@H]4CC[C@@H](C3)N4C3COC3)ncc([C@H]3CC[C@H](O)CC3)c2n1] |

Compounds described herein may be prepared using methods described herein and/or available in the art; useful techniques are both readily apparent and accessible to one of ordinary skill in the art. The description below illustrates certain methods available for making the compounds described herein and is not intended to define the scope of reactions or reaction sequences that can be used to prepare the compounds described herein.

The present compounds can be generally prepared according to Schemes 1-7.

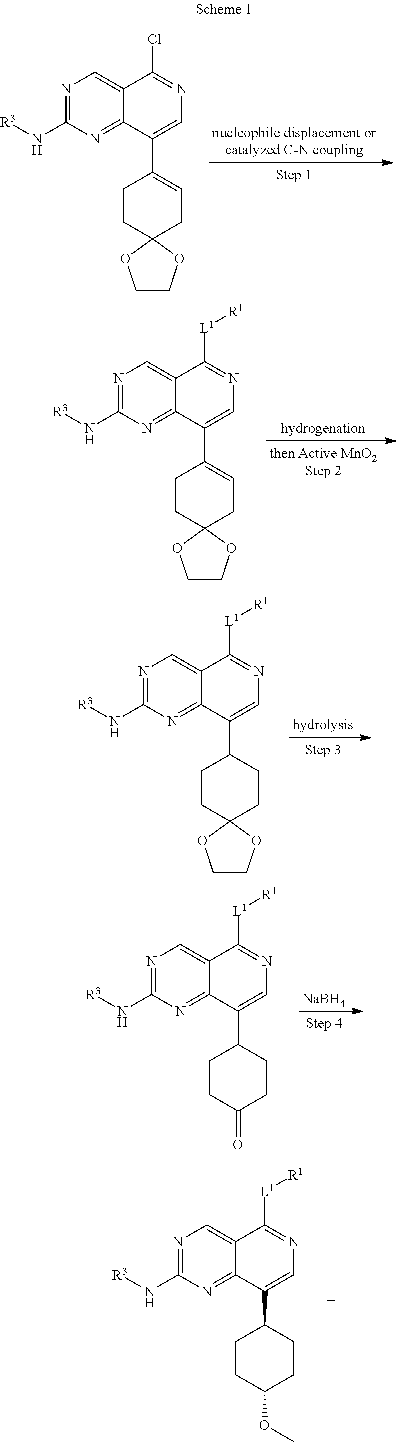

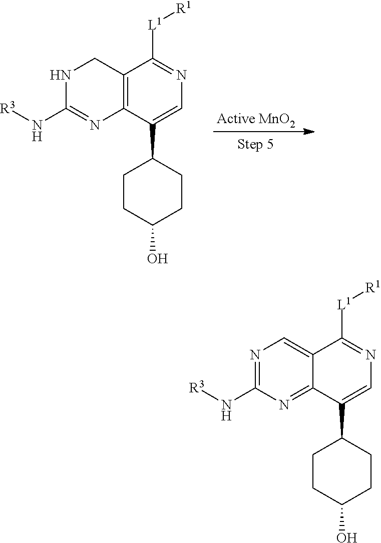

Each of the aforementioned synthetic steps may be performed sequentially, with isolation of each intermediate performed after each step, or each step (Step 1-Step 5 as depicted in Scheme 1), may be performed such that no isolation of one or more intermediates is performed. Furthermore, it will be readily apparent to one of ordinary skill in the art that additional steps may be performed to accomplish particular protection group and/or deprotection strategies.

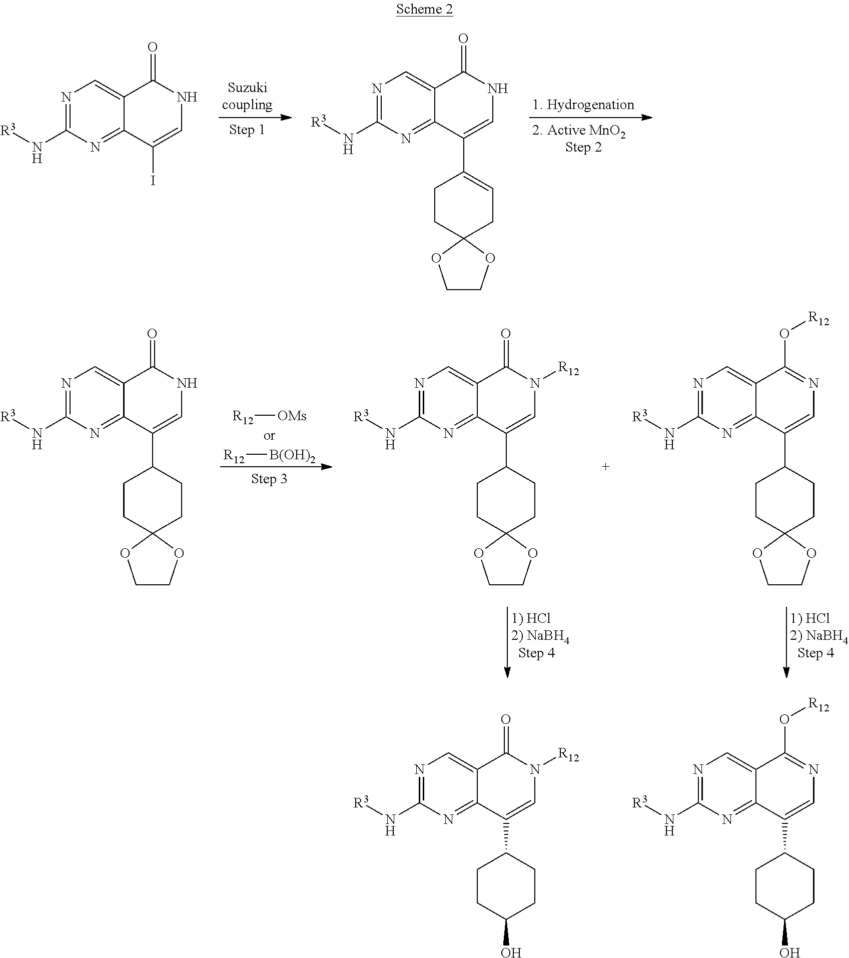

wherein R₁₂ is L⁴-R⁴ when bound to nitrogen and R¹ when bound to oxygen.

Each of the aforementioned synthetic steps may be performed sequentially, with isolation of each intermediate performed after each step, or each step (Step 1-Step 5 as depicted in Scheme 2), may be performed such that no isolation of one or more intermediates is performed. Furthermore, it will be readily apparent to one of ordinary skill in the art that additional steps may be performed to accomplish particular protection group and/or deprotection strategies.

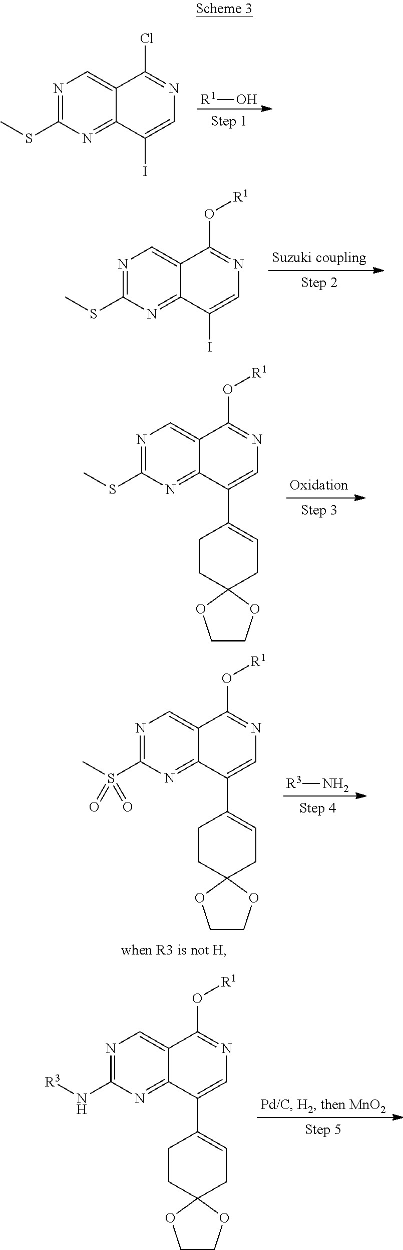

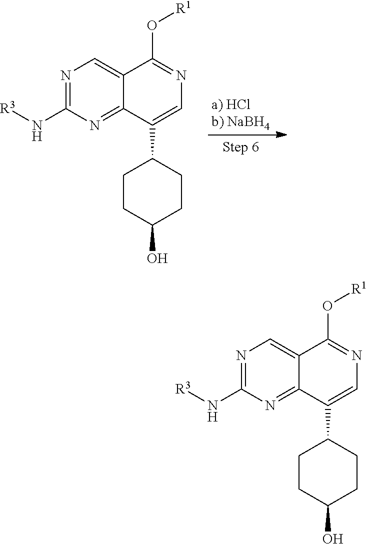

when R3 is H,

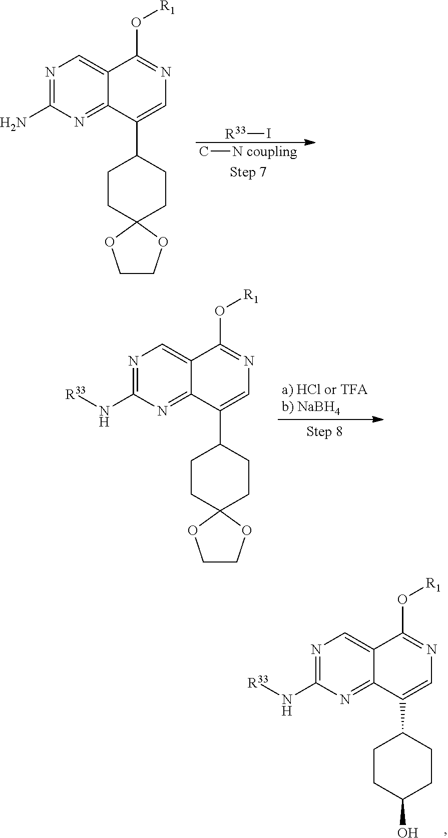

wherein R³³ is an optionally substituted aryl ring.

Each of the aforementioned synthetic steps may be performed sequentially, with isolation of each intermediate performed after each step, or each step (Step 1-Step 8 as depicted in Scheme 3), may be performed such that no isolation of one or more intermediates is performed. Furthermore, it will be readily apparent to one of ordinary skill in the art that additional steps may be performed to accomplish particular protection group and/or deprotection strategies.

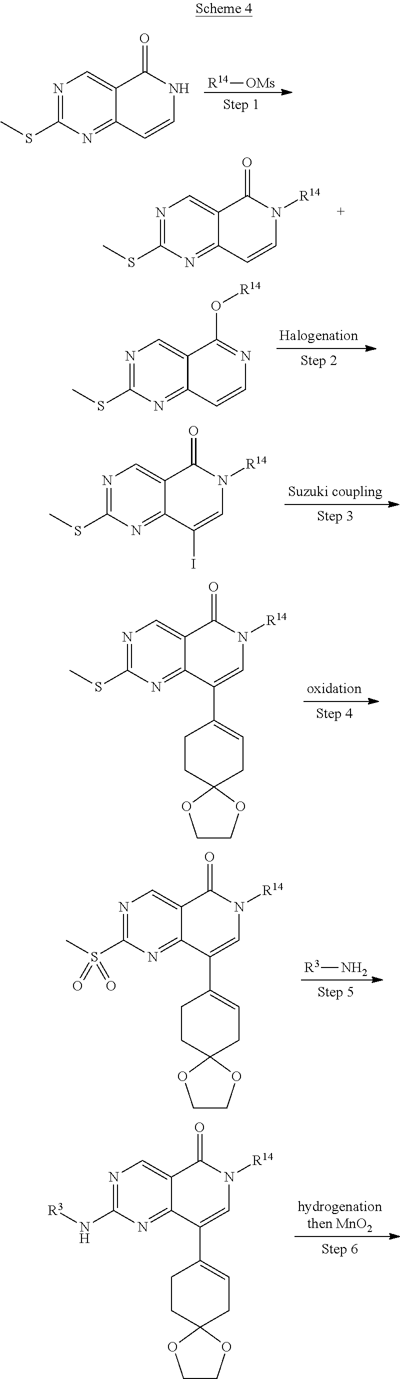

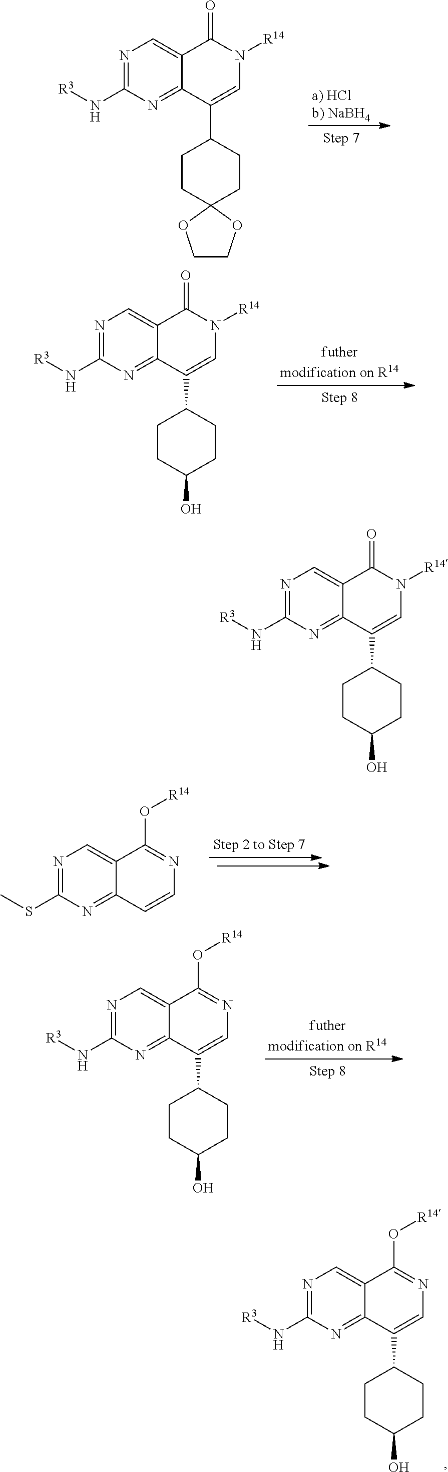

wherein R¹⁴ and R^{14′} is -L⁴-R⁴ when bound to the ring nitrogen; and —R¹ when bound to oxygen.

Each of the aforementioned synthetic steps may be performed sequentially, with isolation of each intermediate performed after each step, or each step (Step 1-Step 8 as depicted in Scheme 4), may be performed such that no isolation of one or more intermediates is performed. Furthermore, it will be readily apparent to one of ordinary skill in the art that additional steps may be performed to accomplish particular protection group and/or deprotection strategies.

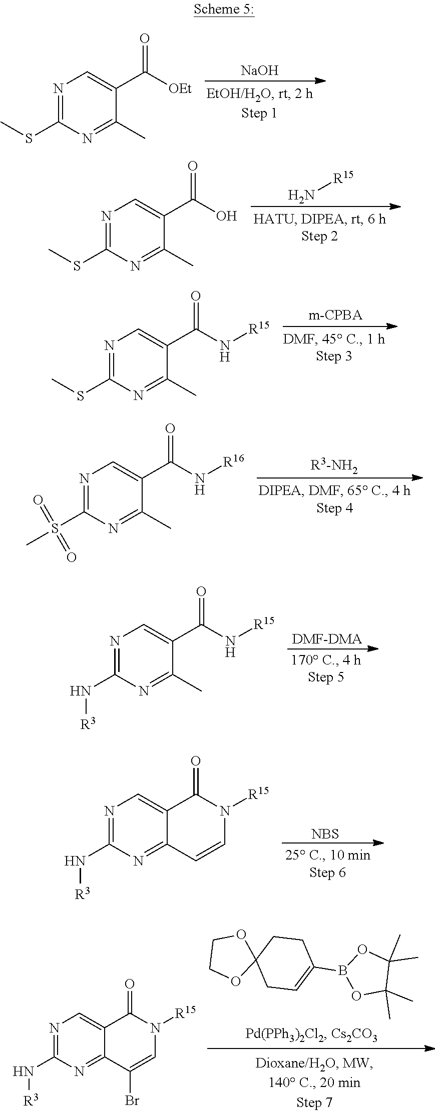

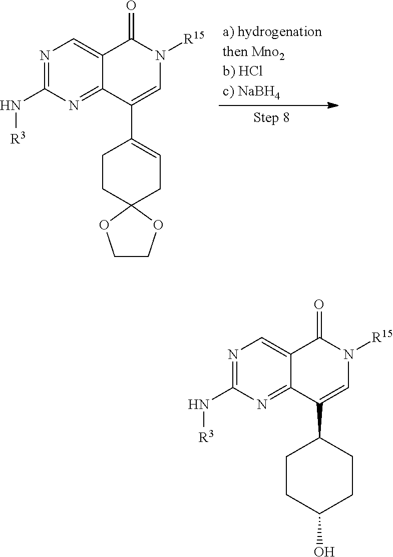

wherein R¹⁵ is L⁴-R⁴.

Each of the aforementioned synthetic steps may be performed sequentially, with isolation of each intermediate performed after each step, or each step (Step 1-Step 8 as depicted in Scheme 5), may be performed such that no isolation of one or more intermediates is performed. Furthermore, it will be readily apparent to one of ordinary skill in the art that additional steps may be performed to accomplish particular protection group and/or deprotection strategies.

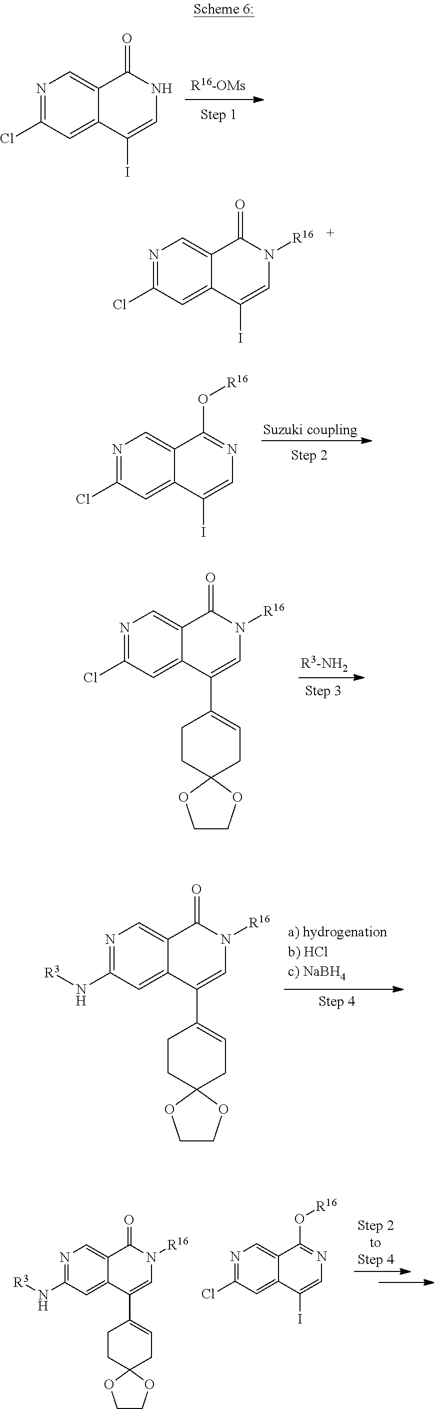

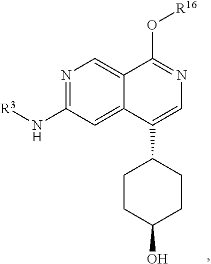

wherein R¹⁶ is -L⁴-R⁴ when bound to the ring nitrogen, or R¹ when bound to oxygen.

Each of the aforementioned synthetic steps may be performed sequentially, with isolation of each intermediate performed after each step, or each step (Step 1-Step 4 as depicted in Scheme 6), may be performed such that no isolation of one or more intermediates is performed. Furthermore, it will be readily apparent to one of ordinary skill in the art that additional steps may be performed to accomplish particular protection group and/or deprotection strategies.

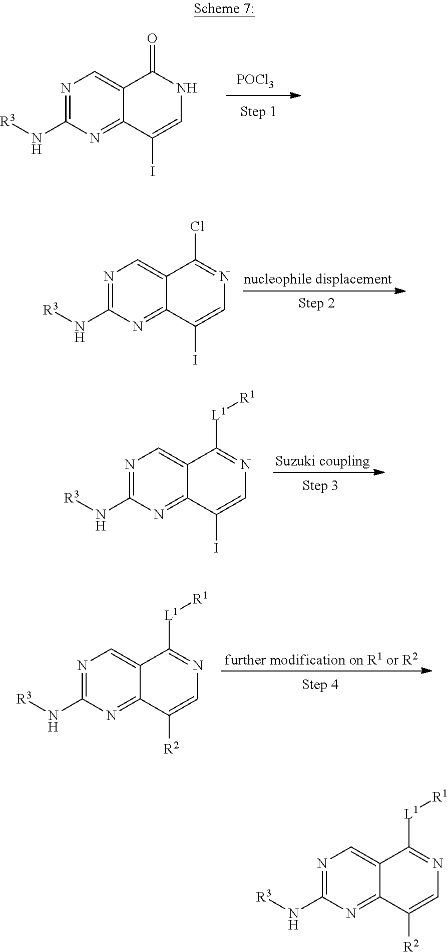

Each of the aforementioned synthetic steps may be performed sequentially, with isolation of each intermediate performed after each step, or each step (Step 1-Step 4 as depicted in Scheme 7), may be performed such that no isolation of one or more intermediates is performed. Furthermore, it will be readily apparent to one of ordinary skill in the art that additional steps may be performed to accomplish particular protection group and/or deprotection strategies.

In certain embodiments, any of the steps of the aforementioned syntheses may be performed to prepare the desired final product. In other embodiments, two, three, four, five, or more sequential steps may be performed to prepare an intermediate or the desired final product. Certain starting materials depicted in Schemes 1-7 may be readily interchanged with other starting materials or reagents to provide additional compounds of formula I. Such substitutions could be made with routine experimentation.

The term “specific,” when used herein with reference to an agent having an activity (e.g., a compound or composition described herein), means that the agent discriminates between potential target entities (e.g., kinases) or states. For example, an agent (e.g., compound) binds “specifically” to its target if it binds preferentially with that target in the presence of one or more competing alternative targets. Similarly, an agent “specifically” inhibits a target if it inhibits the expression or activity of that target preferentially in the presence of one or more competing alternative targets. It is to be understood that specificity need not be absolute and may be evaluated with regard to different reference points, some of which are discussed further below. For example, specificity may be evaluated relative to that of an agent for one or more other potential target entities (e.g., competitors); relative to that of a reference specific agent; or relative to that of a reference non-specific agent. In some embodiments, the agent does not detectably bind or otherwise inhibit the competing alternative target under conditions in which it binds or otherwise inhibits its own target entity. While the invention is not limited to compounds that exert their effect in any particular way, the compounds of the invention may exhibit, with respect to their target(s), a higher on-rate, lower off-rate, increased affinity, decreased dissociation, and/or increased stability compared with a competing alternative target(s).

Where specificity is generated by specific binding between a compound and its target (e.g., a TAM kinase), that binding can be assessed by detecting or determining the degree of association between the binding agent (e.g., a compound described herein) and its target (e.g. a TAM kinase); in some embodiments, specific binding is assessed by detecting or determining the degree of dissociation of the components in a compound-target complex; or by detecting or determining the ability of the compound to compete with an alternative interaction between its target and another entity. In some embodiments, specific binding is assessed by performing such detections or determinations across a range of concentrations.

In some embodiments, a provided compound or composition demonstrates specificity by virtue of its binding activity, inhibitory activity, ability to compete with an alternative ligand for binding (e.g., a reference compound or composition) to and/or other effect on the kinase. One of ordinary skill in the art will be familiar with techniques for assessing these activities and abilities. For example, they may be variously assessed as an IC₅₀, by competitive inhibition assays, by determining a inhibitory constant, or by determining kinase inhibition potency.

In various embodiments, a provided compound and/or composition shows a comparable level of activity against each of TYRO3, AXL and MERTK; shows activity above a particular reference level with respect to each of TYRO3, AXL and MERTK; shows specificity for one or more of TYRO3, AXL and MERTK; shows specificity for each of TYRO3, AXL and MERTK; shows more specificity for one or more TAM kinases relative to other kinases; shows more specificity for one or more of TYRO3, AXL and MERTK relative to other kinases; and/or shows more specificity for each of TYRO3, AXL and MERTK relative to other kinases.

A compound and/or composition described herein is considered to be specific for a given TAM kinase when it shows at least or about 2×, 5×, 10×, 20×, 30×, 40×, 50×, 60×, 70×, 80×, 90×, 100× or more activity for that kinase (e.g., ability to inhibit that kinase) than for one or more appropriate comparator kinase(s) (e.g., for one or more TAM kinases relative to one or more non-TAM kinases, for one or more of TYRO3, AXL and MERTK relative to one or more kinases other than TYRO3, AXL and MERTK, or for one or more of TYRO3, AXL, and MERTK relative to one another). The comparator kinase can be FLT3. In a more specific aspect of these embodiments, a provided compound and/or composition shows at least or about 2×, 5×, 10×, 20×, 30×, 40×, 50×, 60×, 70×, 80×, 90×, 100× or lower IC₅₀ against the TAM kinase member against which it is most active (e.g., has the lowest IC₅₀) than against a comparator kinase (e.g., FLT3). For example, a provided compound and/or composition can show at least or about 2×, 5×, 10×, 20×, 30×, 40×, 50×, 60×, 70×, 80×, 90×, 100× or lower IC₅₀ against MERTK than against a comparator kinase (e.g., FLT3).

In some embodiments, a provided compound and/or composition is considered to be specific for a given kinase or set of kinases when it shows at least or about a 2-fold, 3-fold, 4-fold, 5-fold, 10-fold, 15-fold, 20-fold, 50-fold, 100-fold or more activity for the specific kinase(s) than for one or more appropriate comparator kinase(s) (e.g., for one or more TAM kinases relative to one or more non-TAM kinases, for one or more of TYRO3, AXL and MERTK relative to one or more kinases other than TYRO3, AXL and MERTK, or for one or more of TYRO3, AXL, and MERTK relative to one another). As above, the comparator kinase can be FLT3. In a more specific aspect of these embodiments, a provided compound and/or composition shows at least or about a 2-fold, 3-fold, 4-fold, 5-fold, 10-fold, 15-fold, 20-fold, 50-fold, 100-fold or lower IC₅₀ against the TAM kinase member against which it is most active (e.g., has the lowest IC₅₀) than against FLT3. In some embodiments, a provided compound and/or composition shows at least or about a 2-fold, 3-fold, 4-fold, 5-fold, 10-fold, 15-fold, 20-fold, 50-fold, 100-fold or lower IC₅₀ against MERTK than against FLT3.

In some embodiments, a provided compound and/or composition is considered to be specific for a given kinase or set of kinases when it shows at least or about 101%, 105%, 110%, 120%, 130%, 140%, 150%, 200%, 300%, 400%, 500% or more activity for the specific kinase(s) than for one or more appropriate comparator kinase(s) (e.g., for one or more TAM kinases relative to one or more non-TAM kinases, for one or more of TYRO3, AXL and MERTK relative to one or more kinases other than TYRO3, AXL and MERTK, or for one or more of TYRO3, AXL, and MERTK relative to one another). In one specific aspect of these embodiments, a provided compound and/or composition is considered to be specific for a given kinase when it shows at least or about 101%, 105%, 110%, 120%, 130%, 140%, 150%, 200%, 300%, 400%, 500% or more activity for the specific kinase(s) than for FLT3. In a more specific aspect of these embodiments, a provided compound and/or composition shows at least 101%, 105%, 110%, 120%, 130%, 140%, 150%, 200%, 300%, 400%, 500% or lower IC₅₀ against the TAM kinase member against which it is most active (e.g., has the lowest IC₅₀) than against FLT3. In an even more specific aspect of these embodiments, a provided compound and/or composition shows at least or about 101%, 105%, 110%, 120%, 130%, 140%, 150%, 200%, 300%, 400%, 500% or lower IC₅₀ against MERTK than against FLT3.

Compounds and/or compositions provided herein have a variety of uses, including uses in research, analysis, medicine, clinical therapy and/or prophylactic care. The invention encompasses methods for inhibiting a TAM kinase (e.g., TYRO3, AXL, MERTK or any combination thereof), the methods include the step of contacting the TAM kinase with a compound or composition described herein under conditions and for a time sufficient to allow the kinase to be inhibited. In some embodiments, the kinase is TYRO3. In some embodiments, the kinase is AXL. In some embodiments, the kinase is MERTK.

In some embodiments, compounds and/or compositions described herein can be administered (e.g., to a patient) in methods of treating and/or preventing a disease (e.g., a disease associated with overexpression or increased activity of a TAM kinase and/or a disease that is responsive to inhibition of one or more of the TAM kinases (e.g., TYRO3, AXL and/or MERTK)). The disease can be any described herein, including cancer, a hematologic disorder, or a benign lesion. In some embodiments, compounds of the present disclosure provide methods of enhancing an effect, the methods comprising administering to a subject an amount of a provided compound, thereby treating or preventing the disease. The amount can be a therapeutically effective amount. In some embodiments, compounds of the present disclosure are useful in the treatment of cancer, such as leukemias, lymphomas, gastric cancer, prostate cancer, breast cancer, pituitary adenoma, NSCLC, melanoma, glioblastoma, ovarian cancer and rhabdomyosarcoma.

Each therapeutic or diagnostic method that employs a compound described herein (e.g., a TAM kinase inhibitor) and involves administration of the compound or a composition containing it to a patient may also be expressed in terms of use and vice versa. For example, the invention encompasses the use of a compound or composition described herein for the treatment of a disease described herein (e.g., cancer, a hematologic disorder, or a benign lesion); a compound or composition for use in diagnosing and/or treating or a disease (e.g., cancer, a hematologic disorder, or a benign lesion); and the use of the compound or composition for the preparation of a medicament for treating a disease described herein (e.g., cancer).

The methods of the invention that concern diagnosing and/or treating a disease described herein (e.g., a cancer, hematologic disorder, or benign lesion) may specifically exclude any one or more of the types of diseases (e.g., cancers) described herein. For example, the invention features methods of treating cancer by administering a compound as described herein (e.g., a compound of Formula I) with the proviso that the cancer is not a breast cancer; with the proviso that the cancer is not a breast cancer or a leukemia; with the proviso that the cancer is not a breast cancer, a leukemia, or an ovarian cancer; and so forth, with exclusions selected from any of the diseases listed herein and with the same notion of variable exclusion from lists of elements relevant to other aspects of the invention (e.g., chemical substituents of a compound described herein or components of kits and pharmaceutical compositions).

The present invention provides pharmaceutical compositions that include a compound described herein (e.g., a compound of formula I, a pharmaceutically acceptable salt thereof, or a prodrug thereof) and a pharmaceutically acceptable carrier (e.g., a diluent, excipient, or vehicle).

As noted, the pharmaceutical compositions can include optical isomers, diastereomers, or pharmaceutically acceptable salts of any one or more of the compounds disclosed herein. The compound, either in isolation or when included in the pharmaceutical composition may be covalently attached to a carrier moiety. Alternatively, the compound(s) included in the pharmaceutical composition is/are not covalently linked to a carrier moiety.

Compounds of the invention can be administered alone (i.e., as a first agent that is the sole active agent for treatment or prevention of a disease described herein) or can be co-administered to the subject (i.e., with another active agent (a second agent) intended to treat or prevent either a disease described herein or a distinct disease). Coadministration includes simultaneous or sequential administration of the compounds by the same or different routes of administration. Thus, the invention encompasses compositions (e.g., pharmaceutical compositions) that include a combination of compounds described herein (i.e., more than one of the compounds, pharmaceutically acceptable salts or prodrugs described herein) or a combination of a compounds described herein (or a pharmaceutically acceptable salt or prodrug thereof) and a second, distinct therapeutic agent. In some embodiments, the additional therapeutic agent includes a boron atom.

When a compound of the invention is used in combination with a second therapeutic agent that is active against the same disease, the dose of each compound (i.e., of either the first compound, the second compound, or both) may differ from that required or typically administered for efficacy when the compound is used alone. Appropriate and/or effective doses will be readily appreciated by one of ordinary skill in the art. It will be appreciated that the amount of a provided compound required for use in treatment or prophylactic methods will vary with the nature of the condition being treated and various attributes of the subject in question (e.g., the subject's age, gender, weight, and other conditions and physiological parameters) and will be ultimately determined at the discretion of the attendant physician or veterinarian.

Any compound described herein can be utilized in combination with (e.g., administered to subjects receiving) therapy (e.g., standard of care therapy) for the treatment of cancer. As noted above, the cancer can be a blood cancer, a bone cancer, a breast cancer (e.g., TNBC), an endocrine cancer (e.g., cancer of the thyroid or parathyroid gland), a gastrointestinal cancer (e.g., a gastric cancer or colorectal cancer), a genitourinary cancer (e.g., cancer of the bladder, kidney, prostate, cervix, or uterus (e.g., an endometrial cancer)), a head and neck cancer (e.g., cancer of the larynx), a liver cancer, a lung cancer (e.g., NSCLC), melanoma (e.g., a skin cancer or a cutaneous or intraocular melanoma), a nervous system or brain cancer (e.g., glioblastoma), oral cancer (e.g., a cancer of the mouth or throat), an ovarian cancer, a pancreatic cancer (e.g., pancreatic ductal adenocarcinoma), a plasma cell neoplasm or myeloma, or rhabdomyosarcoma. As noted, the blood cancer can be ALL; AML; CML; CLL; HL; NHL; follicular lymphoma; CLL/SLL; MCL; and others as set out herein. As noted above, any cancer or combination of cancers described herein can be excluded from the methods and uses of the invention.

Alternatively, or additionally, a compound described herein can be used in combination with (e.g., administered to subjects who have received, are receiving, or are scheduled to receive) immunotherapy. In some embodiments, such immunotherapy comprises or consists of checkpoint inhibitor therapy (e.g., PD-1 inhibitors (for example nivolumab and pembrolizumab), PD-L1 inhibitors (for example atezolizumab, avelumab and durvalumab), and CTLA4 inhibitors (for example ipilimumab), vaccine therapy (e.g., cancer vaccine therapy), and/or cell therapy (e.g., CAR-T therapy and/or CAR-NK therapy). In some embodiments, compounds are administered to subjects who have received, are receiving, or will receive antibody therapy, cell therapy (e.g., CAR-T therapy and/or CAR-NK therapy), chemotherapy, hormone therapy (e.g., a therapy that reduces the level of a relevant hormone and/or its receptor and/or inhibits hormone-receptor interaction or one or more downstream effects thereof), radiation therapy, and/or surgical therapy.

Compounds of the present disclosure can be prepared and administered in a wide variety of oral and parenteral forms. Thus, the compounds described herein can be administered by injection (e.g. intravenously, intramuscularly, intracutaneously, subcutaneously, intraduodenally, or intraperitoneally). The compounds can also be administered by inhalation (e.g., intranasally) or by insufflation. Additionally, the compounds described herein can be administered topically or transdermally.

For preparing pharmaceutical compositions including a compound described herein, pharmaceutically acceptable excipients can be added in either solid or liquid form or a combination thereof. Solid form preparations within the scope of the present invention include powders, tablets, pills, capsules, cachets, suppositories, and dispersible granules. A solid carrier can be a substance that may also act as a diluent, flavoring agent, binder, preservative, tablet disintegrating agent, or encapsulating material. In powders, the excipient (e.g., a carrier) is a finely divided solid in a mixture with the finely divided active component (e.g., a compound described herein). In tablets, the active component (e.g., a compound described herein) is mixed with the excipient having the necessary binding properties in suitable proportions and compacted in the shape and size desired. Pharmaceutical compositions, including those formulated as powders and tablets, can contain from 5% to 70% of the active compound (i.e., a compound described herein). Suitable excipients (e.g., carriers) are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation,” when used in connection with a pharmaceutical composition, is intended to include, but is not limited to, the formulation of an active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In some embodiments, suitable carriers for parenteral administration will be selected for human administration. In particular, carriers for parenteral administration include aqueous solutions of dextrose, saline, pure water, ethanol, glycerol, glycerol formal, polyethylene glycol, propylene glycol, peanut oil, sesame oil, polyoxyethylene-block polymers, pyrrolidine, N-methyl pyrrolidione, and the like. Ampoules are convenient unit dosages. The compounds of the present disclosure can also be incorporated into liposomes or administered via transdermal pumps or patches. Pharmaceutical admixtures suitable for use in the present disclosure include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical compositions can be in unit dosage forms. In such form, the preparation, specifically including any given preparation as described herein, is subdivided into unit doses containing quantities of the active component appropriate for administration to a patient and, optionally, instructions for storage, administration, or use. The unit dosage forms can be contained in packaged preparations, the package containing discrete quantities of prepared pharmaceutical compositions, such as packeted/packaged tablets, capsules, and powders in containers (e.g., vials or ampules). The unit dosage form can be a capsule, tablet, cachet, or lozenge itself, or it can be an appropriate number of any of these in packaged form.

The quantity of active component in a unit dose preparation can be varied from about 0.1 mg (milligram) to about 10000 mg, more typically 1.0 mg to 1000 mg, and most typically 10 mg to 500 mg (e.g., 50-450, 100-400, 200-300, 50-100, 100-200, 100-250, or 300-400 mg) according to the particular application and the potency of the active component. As noted, the compositions, including unit dosage forms, can contain (an) additional therapeutic agent(s).

Some compounds may have limited solubility in water and therefore may require a surfactant or other appropriate co-solvent (e.g., an emulsifier) in the composition. Such co-solvents include: Polysorbate 20, 60, and 80; PLURONIC® (polyoxyalkylene ether) F-68, F-84, and P-103; cyclodextrin; and polyoxyl 35 castor oil. Such co-solvents may be employed in a composition described herein at a level between about 0.01% and about 2% by weight.

Viscosity greater than that of simple aqueous solutions may be desirable to decrease variability in dispensing the formulations, to decrease physical separation of components of a suspension or emulsion of a formulation, and/or to otherwise improve the formulation. Such viscosity-building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Pharmaceutical compositions of the present invention may additionally include components to provide sustained release and/or comfort (e.g., high molecular weight, anionic mucomimetic polymers, gelling polysaccharides, and finely-divided drug carrier substrates). These components are described in greater detail in U.S. Pat. Nos. 4,911,920; 5,403,841; 5,212,162; and 4,861,760. The contents of these patents are incorporated herein by reference in their entirety for all purposes.

As noted, pharmaceutical compositions of the invention include those in which an active ingredient is contained/present in a therapeutically effective amount. The actual amount effective for a particular application will depend, inter alia, on the disease being treated.

The dosage of a compound or pharmaceutical composition described herein and the frequency and regimen of its administration (e.g., daily, weekly, twice per week, in single or multiple doses) is expected to vary depending upon a variety of factors, including the route of administration; size, age, sex, health, body weight, body mass index, and diet of the patient; the nature and extent of symptoms of the disease being treated; presence or absence of other diseases or other health-related problems; the kind of concurrent treatment, if any; and complications from any disease or treatment regimen.

For any compound or pharmaceutical composition described herein, the therapeutically effective amount can be initially determined from, or informed by data generated in, cell culture assays and/or animal models of disease. For example, a dose for humans can be formulated to achieve a concentration that has been found to be effective in animals. The dosage in humans can be adjusted by, for example, monitoring kinase inhibition, other markers, the signs and symptoms of the disease being treated, and side effects and subsequently adjusting the dosage upwards or downwards.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under a desired circumstance is reached. In some embodiments, the concentration of compound is about 0.001% to about 10% w/v (e.g., about 0.1% to about 5% w/v). Concentrations, dosage amounts, and intervals can be adjusted individually to provide levels of the administered compound effective for the particular disease being treated. This will provide a therapeutic regimen commensurate with the severity of the patient's disease.

### EXAMPLES

The following analytical instruments were used in the synthesis and analysis of the compounds of the invention. For liquid chromatography mass spectrometry (LCMS or LC-MS): Shimadzu UFLC MS: LCMS-2020; Agilent Technologies 1200 series MS: Agilent Technologies 6110; and Agilent Technologies 1200 series MS: LC/MSD VL. For nuclear magnetic resonance (NMR): BRUKER AVANCE III/400 MHz. For preparatory high performance liquid chromatography (prep-HPLC (high pressure liquid chromatography)): Gilson GX-281 systems: instruments GX-A, GX-B, GX-C, GX-D, GX-E, GX-F, GX-G and GX-H.

### Example 1: Synthesis of Intermediates

### A. 2-(methylthio)pyrido[4,3-d]pyrimidin-5(6H)-one

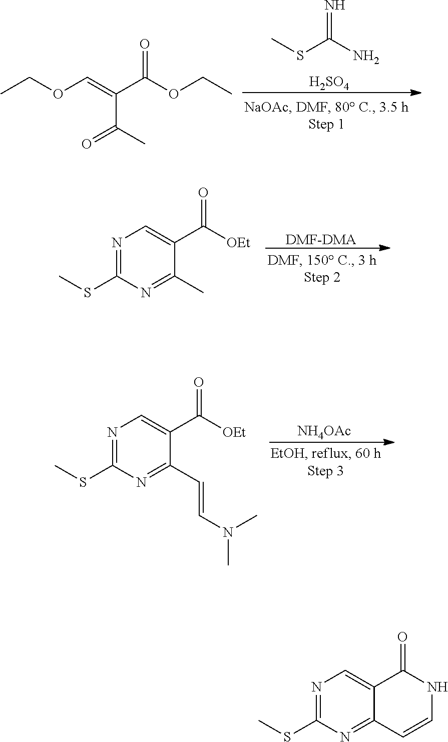

### Step 1: Ethyl 4-methyl-2-methylsulfanyl-pyrimidine-5-carboxylate

To a solution of ethyl (2E)-2-(ethoxymethylene)-3-oxo-butanoate (50 g, 269 mmol) in DMF (dimethylformamide; 270 mL) was added sodium acetate (22 g, 269 mmol) and 2-methylisothiourea sulfate (50 g, 268 mmol). The mixture was stirred at 80° C. for 3.5 hours. Upon completion, the mixture was cooled to room temperature (RT), then poured into ice water. Precipitation was formed, filtered and dried under reduced pressure to afford ethyl 4-methyl-2-methylsulfanyl-pyrimidine-5-carboxylate (47 g, crude) as a red solid, which was used in the next step without further purification.

### Step 2: Ethyl 4-[(E)-2-(dimethylamino)vinyl]-2-methylsulfanyl-pyrimidine-5-carboxylate

To a solution of ethyl 4-methyl-2-methylsulfanyl-pyrimidine-5-carboxylate (47 g, 221 mmol) in DMF (120 mL) was added DMF-DMA (dimethylformamide-dimethyl adipate; 59 mL, 443 mmol). The mixture was stirred at 150° C. for 3 hours. Upon completion, the mixture was poured into water (400 mL), and then extracted with ethyl acetate (400 mL×3). The organic layers were combined, washed with brine (200 mL×2), and then concentrated in vacuo to give ethyl 4-[(E)-2-(dimethylamino)vinyl]-2-methylsulfanyl-pyrimidine-5-carboxylate (60 g, crude) as a red solid, which was used in the next step directly.

### Step 3: 2-methylsulfanyl-6H-pyrido[4,3-d]pyrimidin-5-one

To a solution of ethyl 4-[(E)-2-(dimethylamino)vinyl]-2-methylsulfanyl-pyrimidine-5-carboxylate (60 g, 224 mmol) in EtOH (ethanol; 600 mL) was added NH₄OAc (173 g, 2.2 mol; Ac representing acetyl) under N₂. The mixture was stirred at 80° C. for 60 hours. Upon completion, the reaction was cooled to RT, then poured into ice water. Precipitation was formed, filtered and air-dried to generate 2-methylsulfanyl-6H-pyrido[4,3-d]pyrimidin-5-one (43 g, 99% yield) as a yellow solid.

### B. 5-chloro-8-iodo-2-(methylthio)pyrido[4,3-d]pyrimidine

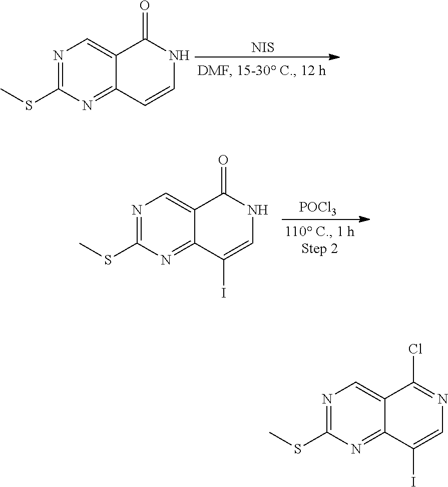

### Step 1: 8-iodo-2-(methylthio)pyrido[4,3-d]pyrimidin-5(6H)-one

A mixture of 2-methylsulfanyl-6H-pyrido[4,3-d]pyrimidin-5-one (5.0 g, 26 mmol) in DMF (50 mL) was added NIS (5.8 g, 26 mmol) at RT. The mixture was stirred at 30° C. for 2 hours then was added NIS (1.2 g, 5.2 mmol) and stirred at 30° C. for another 12 hours. Upon completion, the mixture was cooled to RT, then poured into water (500 mL). Yellow precipitation was formed, filtered and air-dried to afford 8-iodo-2-methylsulfanyl-6H-pyrido[4,3-d]pyrimidin-5-one (8.0 g, crude) as a yellow solid, which was used in the next step directly.

### Step 2: 5-chloro-8-iodo-2-(methylthio)pyrido[4,3-d]pyrimidine

To a mixture of 8-iodo-2-methylsulfanyl-6H-pyrido[4,3-d]pyrimidin-5-one (3.0 g, 9.4 mmol) in POCl₃ (66 g, 430 mmol, 40 mL) was heated at 110° C. for 1 hour. Upon completion, the mixture was cooled to RT and then rotovapped to remove most of POCl₃. The residue was then suspended in THF (tetrahydrofuran; 50 mL) and poured into water (200 mL). During this period, yellow precipitation was formed, filtered and air-dried to afford 5-chloro-8-iodo-2-methylsulfanyl-pyrido[4,3-d]pyrimidine (3.1 g, crude) as yellow solid, which was used in the next step without further purification.

### C. tetrahydropyran-4-yl methanesulfonate

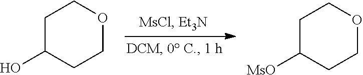 [SMILES: OC1CCOCC1 ; CS(=O)(=O)OC1CCOCC1]

To a solution of tetrahydropyran-4-ol (1.0 g, 9.8 mmol) in DCM (5 mL) was added Et₃N (triethylamine; 2.73 mL, 20 mmol). To the mixture was added a solution of methanesulfonyl chloride (0.91 mL, 12 mmol) in DCM (dichloromethane; 5 mL) at 0° C. The mixture was stirred at 0° C. for 1 hour. Upon completion, the reaction mixture was treated with water (5 mL), and extracted with DCM (10 mL×3). The organic layers were combined, washed with brine (10 mL×3), dried over Na₂SO₄, filtered and concentrated under reduced pressure to afford tetrahydropyran-4-yl methanesulfonate (1.3 g, crude) as a white solid, which was used directly in the next step.

### D. 5-Chloro-N-(pentan-2-yl)-8-(1,4-dioxaspiro[4.5]dec-7-en-8-yl)pyrido[4,3-d]pyrimidin-2-amine

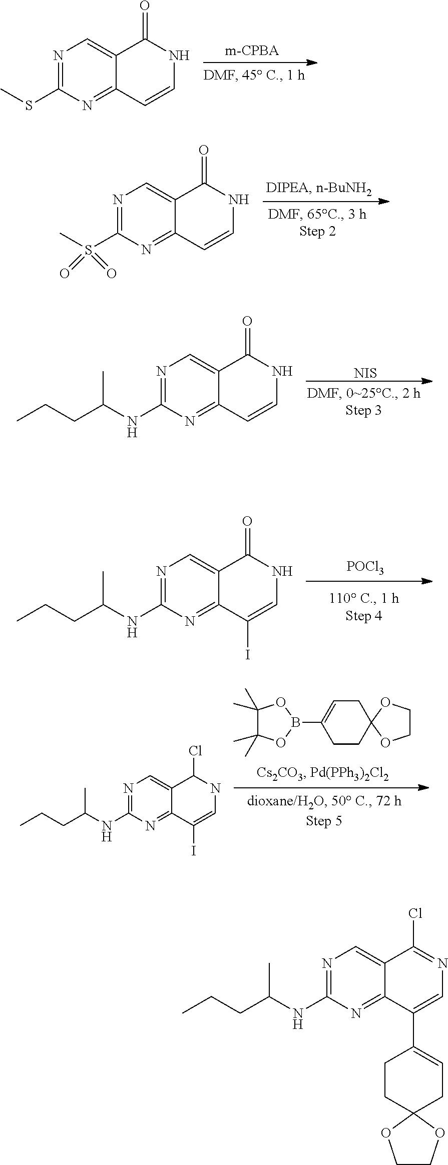

### Step 1 & Step 2: 2-(1-methylbutylamino)-6H-pyrido[4,3-d]pyrimidin-5-one

To a solution of 2-methylsulfanyl-6H-pyrido[4,3-d]pyrimidin-5-one (50 g, 259 mmol) in DMF (500 mL) was added m-CPBA (112 g, 518 mmol, 80% purity). The mixture was stirred at 45° C. for 1 hour. DIPEA (N,N-diisopropyl ethylamine 225 mL, 1.29 mol) and pentan-2-amine (23 g, 259 mmol) were then added to the mixture. The mixture was stirred at 65° C. for 3 hours then cooled to RT. The mixture was treated with Na₂SO₃ (100 mL) then extracted with EtOAc (ethyl acetate; 500 mL x 3). The organic layers were combined, washed with sat. (saturated) NaHCO₃(200 mL) and brined (300 mL) sequentially, dried over Na₂SO₄, filtered and concentrated under reduced pressure to afford 2-(1-methylbutylamino)-6H-pyrido[4,3-d]pyrimidin-5-one (60 g, crude) as a yellow solid, which was used in the next step without further purification.

### Step 3: 8-iodo-2-(1-methylbutylamino)-6H-pyrido[4,3-d]pyrimidin-5-one

To a solution of 2-(1-methylbutylamino)-6H-pyrido[4,3-d]pyrimidin-5-one (60 g, 258 mmol) in DMF (500 mL) was added NIS (58 g, 258 mmol) at 0° C. Then the mixture was stirred at RT for 12 hours. The mixture was poured into ice water (400 mL) and filtered to afford 8-iodo-2-(1-methylbutylamino)-6H-pyrido[4,3-d]pyrimidin-5-one (40 g, crude) as a yellow solid, which was used in the next step directly.

### Step 4: 5-chloro-8-iodo-N-(1-methylbutyl)pyrido[4,3-d]pyrimidin-2-amine

8-iodo-2-(1-methylbutylamino)-6H-pyrido[4,3-d]pyrimidin-5-one (50 g, 140 mmol) was added into POCl₃ (428 g, 2.8 mol, 259 mL). The resulting mixture was stirred at 110° C. for 1 hour then concentrated in vacuo to remove most of POCl₃. The residue was re-dissolved in THF (40 mL) and then poured into icy water. The mixture was filtered and the filtrate was collected and concentrated under reduced pressure. The residue was purified by prep-TLC (thin layer chromatography) to afford 5-chloro-8-iodo-N-(1-methylbutyl)pyrido[4,3-d]pyrimidin-2-amine (25 g, 48% yield) as a yellow solid.

### Step 5: 5-chloro-8-(1,4-dioxaspiro[4.5]dec-7-en-8-yl)-N-(1-methylbutyl)pyrido[4,3-d]pyrimidin-2-amine

To a solution of 5-chloro-8-iodo-N-(1-methylbutyl)pyrido[4,3-d]pyrimidin-2-amine (25 g, 66 mmol) in 1,4-dioxane (250 mL) and H₂O (50 mL) was added Cs₂CO₃ (43 g, 133 mmol), Pd(PPh₃)₂Cl₂ (4.7 g, 6.6 mmol) and 2-(1,4-dioxaspiro[4.5]dec-7-en-8-yl)-4,4,5,5-tetramethyl-1,3,2-dioxaborolane (18 g, 66 mmol). The mixture was stirred at 50° C. for 72 hours, then cooled to RT and extracted with ethyl acetate (500 mL×3). The organic layers were combined, washed with sat. NaHCO₃(200 mL) and brine (300 mL) sequentially, dried over Na₂SO₄, filtered and concentrated under reduced pressure to give a crude residue, which was purified by prep-TLC to afford 5-chloro-8-(1,4-dioxaspiro[4.5]dec-7-en-8-yl)-N-(1-methylbutyl)pyrido[4,3-d]pyrimidin-2-amine (19 g, 68% yield, 93% purity) as a yellow solid.

### E. 8-(1,4-dioxaspiro[4.5]decan-8-yl)-2-(1-methylbutylamino)-6H-pyrido[4,3-d]pyrimidin-5-one

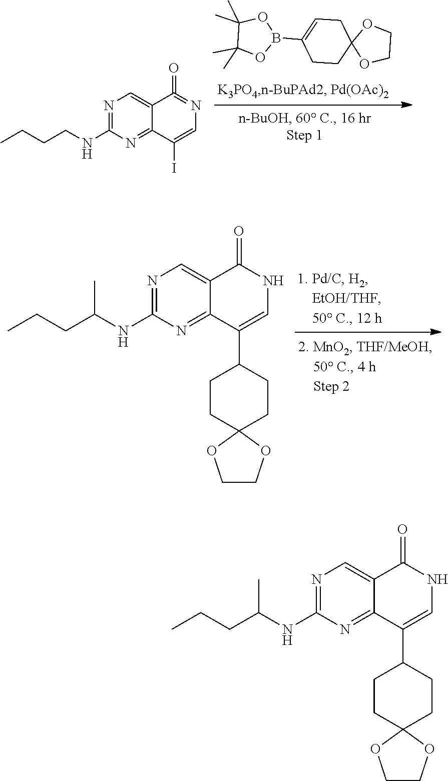

### Step 1: 8-(1,4-dioxaspiro[4.5]dec-7-en-8-yl)-2-(1-methylbutylamino)-6H-pyrido[4,3-d]pyrimidin-5-one

A mixture of 8-iodo-2-(1-methylbutylamino)-6H-pyrido[4,3-d]pyrimidin-5-one (15 g, 42 mmol), 2-(1,4-dioxaspiro[4.5]dec-7-en-8-yl)-4,4,5,5-tetramethyl-1,3,2-dioxaborolane (11 g, 42 mmol), K₃PO₄ (1.5 M, 84 mL), bis(1-adamantyl)-butyl-phosphane (1.5 g, 4.2 mmol) and Pd(OAc)₂ (940 mg, 4.2 mmol) in n-BuOH (250 mL) was degassed and purged with N₂ 3 times and the mixture was then stirred at 60° C. for 16 hours under N₂ atmosphere. Upon completion, the reaction mixture was cooled to RT, partitioned between water (300 mL) and EtOAc (600 mL). The organic phase was separated, washed with brine (100 mL×3), dried over anhydrous Na₂SO₄, filtered, and concentrated in vacuo. The residue was purified by column chromatography to afford 8-(1,4-dioxaspiro[4.5]dec-7-en-8-yl)-2-(1-methylbutylamino)-6H-pyrido[4,3-d]pyrimidin-5-one (13 g, 32.29 mmol, 77% yield, 92% purity) as a yellow solid.

### Step 2: 8-(1,4-dioxaspiro[4.5]decan-8-yl)-2-(1-methylbutylamino)-6H-pyrido[4,3-d]pyrimidin-5-one

To a solution of 8-(1,4-dioxaspiro[4.5]dec-7-en-8-yl)-2-(1-methylbutylamino)-6H-pyrido[4,3-d]pyrimidin-5-one (13 g, 35.09 mol) in THF (700 mL) and MeOH (methanol; 100 mL) was added 10% Pd-C (13 g) under N₂. The suspension was degassed and purged with H₂ several times, then stirred under H₂ (15 psi (pounds per square inch)) at 50° C. for 12 hours. Upon completion, the reaction mixture was filtered and the filtrate was concentrated in vacuo. The residue was suspended in THF (900 mL) and MeOH (100 mL) then treated with MnO₂ (12 g, 133 mmol). The mixture was stirred at 50° C. for 4 hours. Upon completion, the reaction mixture was filtered and concentrated in vacuo. The residue was purified by column chromatography to afford 8-(1,4-dioxaspiro[4.5]decan-8-yl)-2-(1-methylbutylamino)-6H-pyrido[4,3-d]pyrimidin-5-one (11 g, 81% yield, 91% purity) as a yellow solid.

### F. Tert-butyl 4-[8-(1,4-dioxaspiro[4.5]dec-7-en-8-yl)-2-methylsulfanyl-pyrido[4,3-d]pyrimidin-5-yl]oxypiperidine-1-carboxylate

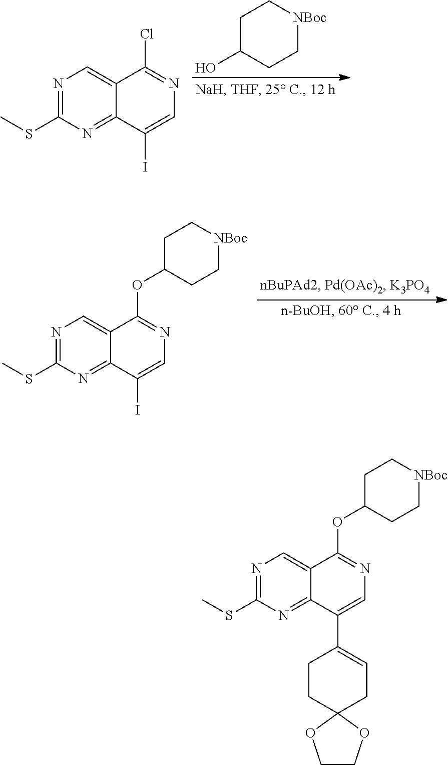

### Step 1: Tert-butyl 4-(8-iodo-2-methylsulfanyl-pyrido[4,3-d]pyrimidin-5-yl)oxypiperidine-1-carboxylate

To a solution of tert-butyl 4-hydroxypiperidine-1-carboxylate (12 g, 60 mmol) in THF (200 mL) was added NaH (2.5 g, 62 mmol, 60% purity) at 0° C. slowly under N₂. The mixture was stirred at RT for 1 hour before 5-chloro-8-iodo-2-methylsulfanyl-pyrido[4,3-d]pyrimidine (20 g, 59 mmol) solution in THF (200 mL) was added. The mixture was stirred at RT for an additional 11 hours then quenched with sat. NH₄Cl (500 mL) at 0° C. The mixture was diluted with water (150 mL) and extracted with DCM (300 mL×3). The organic layers were combined, washed with brine (100 mL), dried over Na₂SO₄, filtered and concentrated in vacuo. The residue was purified by column chromatography to afford the semi-pure product as solid. The solid was suspended in a mixed solvent of petroleum ether and ethyl acetate (300 mL, 1:1). After filtration, the filtrate was concentrated under reduced pressure to give tert-butyl 4-(8-iodo-2-methylsulfanyl-pyrido[4,3-d]pyrimidin-5-yl)oxypiperidine-1-carboxylate (19.8 g, 46% yield, 69% purity) as yellow solid.

### Step 2: Tert-butyl 4-[8-(1,4-dioxaspiro[4.5]dec-7-en-8-yl)-2-methylsulfanyl-pyrido[4,3-d]pyrimidin-5-yl]oxypiperidine-1-carboxylate

A mixture of tert-butyl 4-(8-iodo-2-methylsulfanyl-pyrido[4,3-d]pyrimidin-5-yl)oxy-piperidine-1-carboxylate (19 g, 39 mmol), 2-(1,4-dioxaspiro[4.5]dec-7-en-8-yl)-4,4,5,5-tetramethyl-1,3,2-dioxaborolane (10 g, 39 mmol) and K₃PO₄ solution (1.5 M, 77 mL), bis(1-adamantyl)-butyl-phosphane (1.4 g, 3.9 mmol), diacetoxypalladium (867 mg, 3.9 mmol) in n-BuOH (200 mL) was heated at 60° C. for 4 hours under N₂. The reaction mixture was quenched with brine (150 mL) at RT and then diluted with water (50 mL). The mixture was extracted with EtOAc (150 mL×3). The organic layers were combined, washed with brine (100 mL), dried over Na₂SO₄, filtered and concentrated in vacuo. The residue was purified by column chromate-graphy to afford tert-butyl 4-[8-(1,4-dioxaspiro[4.5]dec-7-en-8-yl)-2-methylsulfanyl-pyrido[4,3-d]pyrimidin-5-yl]oxypiperidine-1-carboxylate (10 g, 50% yield, 99% purity) as a yellow solid.

### G. N-butyl-5-chloro-8-(1,4-dioxaspiro[4.5]dec-7-en-8-yl)pyrido[4,3-d]pyrimidin-2-amine

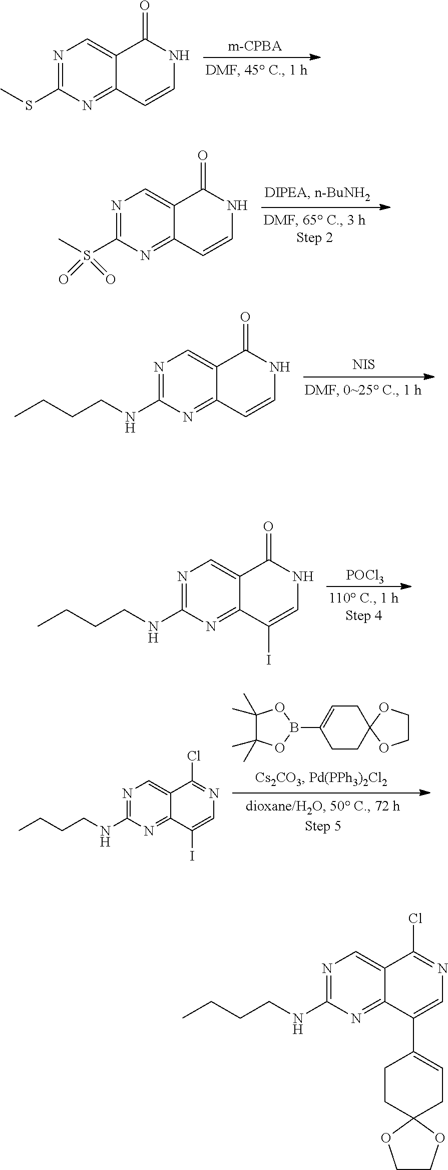

This synthesis was carried out similarly to Example 1, part D, substituting n-butylamine for pentan-2-amine.

### H. 6-chloro-4-iodo-2,7-naphthyridin-1(2H)-one

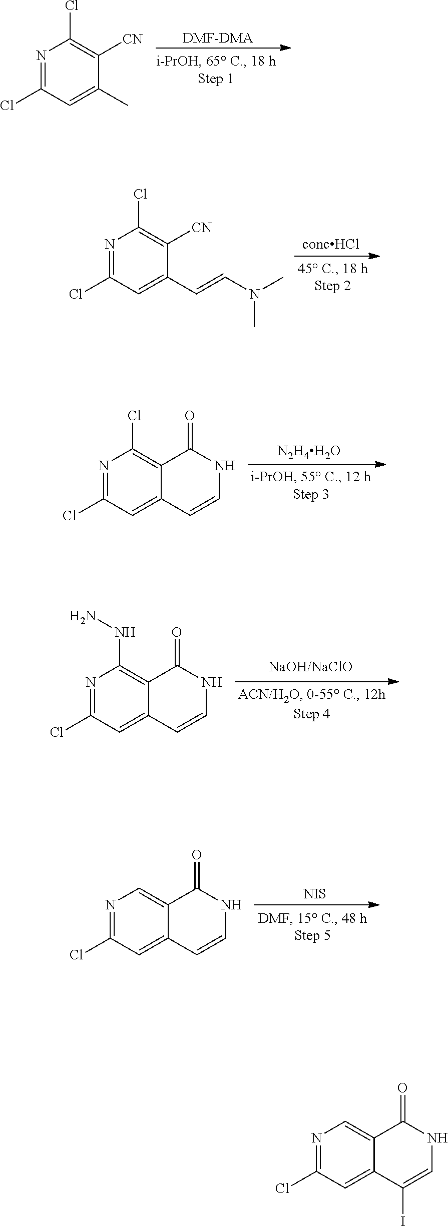

### Step 1: (E)-2,6-dichloro-4-(2-(dimethylamino)vinyl)nicotinonitrile

A solution of 2,6-dichloro-4-methyl-pyridine-3-carbonitrile (52 g, 278 mmol) in i-PrOH (460 mL) was added DMF-DMA (67 g, 564 mmol, 75 mL). The mixture was stirred at 65° C. for 18 hours then cooled to RT. White precipitate was formed and filtered. The filtered cake was washed with i-PrOH (30 mL×2), and air-dried to afford 2,6-dichloro-4-[(E)-2-(dimethylamino)-vinyl]pyridine-3-carbonitrile (22 g, 29% yield, 90% purity) as a yellow solid.

### Step 2: 6,8-dichloro-2,7-naphthyridin-1(2H)-one

A mixture of 2,6-dichloro-4-[(E)-2-(dimethylamino)vinyl]pyridine-3-carbonitrile (22 g, 90.9 mmol) and conc. (concentrated) aq. (aqueous) HCl (12 M, 110 mL) was heated at 45° C. for 18 hours, then cooled to RT and quenched by water (130 mL). Yellow precipitate was formed. The mixture was filtered and the filter cake was air-dried to afford 6,8-dichloro-2H-2,7-naphthyridin-1-one (10 g, crude) as a yellow solid, which was used in the next step without further purification.

### Step 3: 6-chloro-8-hydrazinyl-2,7-naphthyridin-1(2H)-one

A solution of 6,8-dichloro-2H-2,7-naphthyridin-1-one (10 g, 46.50 mmol) in i-PrOH (100 mL) was cooled to 0° C. To this was added NH₂NH₂.H₂O (23.3 g, 465 mmol, 22.6 mL) dropwise. The mixture was stirred at 0° C. for 0.5 hour, then heated at 55° C. for 11.5 hours. During this period, yellow precipitate was formed. The mixture was cooled to RT and filtered. The filter cake was washed with methanol (500 mL) and dried under vacuum to give 6-chloro-8-hydrazino-2H-2,7-naphthyridin-1-one (11.6 g, crude), which was used in the next step directly.

### Step 4: 6-chloro-2,7-naphthyridin-1(2H)-one

6-chloro-8-hydrazino-2H-2,7-naphthyridin-1-one (11.6 g, 55.1 mmol) was dissolved into MeCN (270 mL) to form a suspension. Aq. NaOH (1 M, 138 mL) was added, followed by water (320 mL). The mixture was then heated and stirred at 50° C. until becoming a clear solution. The mixture was then cooled to 0° C. and aq. 5% NaClO (169 mL, 137.69 mmol) was added dropwise. The reaction was stirred at RT for 12 hours. After that, the mixture was cooled to 0° C. and the pH was adjusted to 6 by aq. 1 M HCl. The mixture was extracted with ethyl acetate (200 mL×3), washed with brine (150 mL×2), dried with Na₂SO₄, filtered and concentrated under reduced pressure to afford 6-chloro-2H-2,7-naphthyridin-1-one (2 g, crude) as a red solid, which was confirmed by ¹H NMR and LCMS.

### Step 5: 6-chloro-4-iodo-2,7-naphthyridin-1(2H)-one

To a solution of 6-chloro-2H-2,7-naphthyridin-1-one (2 g, 11.1 mmol) in DMF (20 mL) was added NIS (2.5 g, 11.1 mmol) at RT. The mixture was stirred for 48 hours then poured into water (200 mL). Red precipitate was formed and filtered. The filter cake was air-dried to afford 6-chloro-4-iodo-2H-2,7-naphthyridin-1-one (3.5 g, crude) as a red solid.

### I. 1-(2-methoxyethyl)-2,2,6,6-tetramethyl-piperidin-4-ol

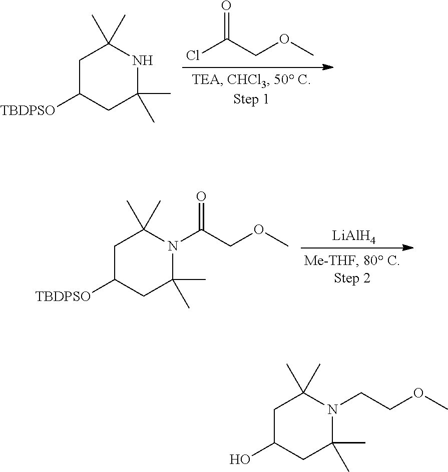

### Step 1: 1-[4-[tert-butyl(diphenyl)silyl]oxy-2,2,6,6-tetramethyl-1-piperidyl]-2-methoxy-ethanone

Tert-Butyl-diphenyl-[(2,2,6,6-tetramethyl-4-piperidyl)oxy]silane (8 g, 20 mmol, 1.0 eq) and CHCl₃ (100 mL) were charged into a one-necked flask. To this solution was added TEA (triethylanolamine; 8.4 mL, 61 mmol, 3.0 eq) and 2-methoxyacetyl chloride (4.4 g, 40 mmol, 3.7 mL, 2.0 eq). The reaction was stirred at 50° C. for 12 hrs. Upon completion, the mixture was diluted with ethyl acetate (500 mL) washed with H₂O (500 mL) and brine (500 mL), dried over Na₂SO₄, filtered and concentrated. The residue was purified by silica gel column chromatography (petroleum ether: Ethyl acetate=5:1, to give 1-[4-[tert-butyl(diphenyl)silyl]oxy-2,2,6,6-tetramethyl-1-piperidyl]-2-methoxy-ethanone (9.2 g, 97% yield) as white solid.

### Step 2: 1-(2-methoxyethyl)-2,2,6,6-tetramethyl-piperidin-4-ol

1-[4-[tert-butyl(diphenyl)silyl]oxy-2,2,6,6-tetramethyl-1-piperidyl]-2-methoxy-ethanone (9.2 g, 20 mmol, 1 eq) and 2-methyltetrahydrofuran (100 mL) were charged into a one-necked flask. To this solution was added LiAlH₄ (3.7 g, 98 mmol, 5 eq) in portions. The reaction was stirred at 80° C. for 60 hrs. Upon completion, the mixture was cooled and quenched with H₂O (3.7 mL) drop-wise. Then aq. 10% NaOH (3.7 mL) and H₂O (3.7 mL) were added. The precipitate was filtered and the filtrate was concentrated. The residue was purified by silica gel column chromatography (petroleum ether: ethyl acetate=1:1) to give 1-(2-methoxyethyl)-2,2,6,6-tetramethyl-piperidin-4-ol (2.7 g, 64% yield) as white solid.

### J. 1-(2-methoxyethyl)-2,2,6,6-tetramethyl-piperidin-4-ol

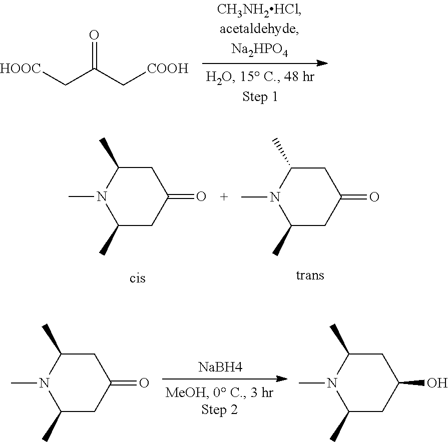

### Step 1: (2S,6R)-1,2,6-trimethylpiperidin-4-one

To a solution of methanamine (5 g, 74 mmol, 1.0 eq, HCl) in H₂O (100 mL) was added acetaldehyde (13 g, 118 mmol, 16.6 mL, 1.6 eq), Na₂HPO₄ (32 g, 180 mmol, 32 mL, 80% purity, 2.4 eq) and 3-oxopentanedioic acid (11 g, 74 mmol, 1 eq). The mixture was then stirred at 15° C. for 48 hrs. During the reaction, the pH rose to 7. The mixture was acidified with a solution of 1N HCl to pH 3 and stirred for 12 h. Solid K₂CO₃ was added portion-wise for neutralization (pH=9) and the solution was extracted with 2-Me-THF (400 mL×2). Combined extracts were dried and, after removal of the solvent, afforded the crude product (2.4 g). The residue was purified by flash silica gel chromatography (Eluent of EtOAcH/EtOH=100/1˜20/1) to give (cis)-1,2,6-trimethylpiperidin-4-one (505 mg, 4.8%) and (trans)-1,2,6-trimethylpiperidin-4-one (298 mg, 2.8% yield) as light yellow oil which was confirmed by LCMS and 1H NMR.

### Step 2: (2S,4r,6R)-1,2,6-trimethylpiperidin-4-ol

To a solution of (cis)-1,2,6-trimethylpiperidin-4-one (150 mg, 1.06 mmol, 1 eq) in MeOH (4 mL) was added NaBH₄ (40 mg, 1.06 mmol, 1 eq) at 0° C. The reaction was then stirred at 0° C. for 3 h. The reaction was quenched by addition of HCl (0.3 mL, 4 M in EtOAc), then it was concentrated under reduced pressure to give (cis)-1,2,6-trimethylpiperidin-4-ol (204 mg, crude) as a light yellow solid.

### K. Exo-8-((S)-tetrahydrofuran-3-yl)-8-azabicyclo[3.2.1]octan-3-ol

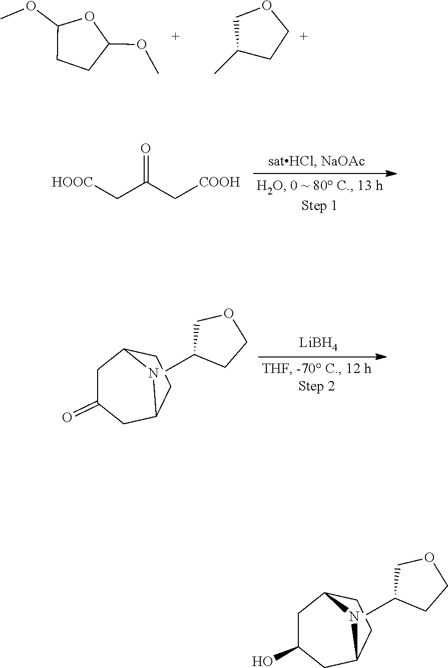

### Step 1: 8-((S)-tetrahydrofuran-3-yl)-8-azabicyclo[3.2.1]octan-3-one

To a solution of 2,5-dimethoxytetrahydrofuran (2.0 g, 15.1 mmol, 2.0 mL, 1.25 eq) in H₂O (6 mL) was added sat. HCl (203 mg, 2.06 mmol, 199 uL, 0.17 eq). The mixture was stirred at 80° C. for 0.5 hour. The solution was cooled to 0° C. In a separate flask, 3-oxopentanedioic acid (2.5 g, 17.0 mmol, 1.4 eq) was added to a solution of NaOAc (3.5 g, 42.4 mmol, 3.5 eq), (3S)-tetrahydrofuran-3-amine (1.50 g, 12.1 mmol, 1 eq. HCl) and sat. HCl (203 mg, 2.06 mmol, 199 uL, 0.17 eq) in H₂O (30 mL) at 5° C. The solution (furan) was then added and washed in with an additional 6 mL H₂O. The mixture was stirred at 5° C. for 15 min. and heated to 45° C. for 12.25 hours. On completion, the mixture was adjusted to a pH of 2 with 1 N HCl. The mixture was extracted with ethyl acetate (2×20 mL). The aqueous layer was adjusted to a pH of 10 with 2 M NaOH. Then the mixture was extracted with EtOAc: iPOH=10:1 (3×30 mL). The combined organic layer was washed with brine (30 mL), dried over Na₂SO₄ and concentrated in vacuo. The crude product was purified by reversed-phase chromatography (0.1% NH₃.H₂O, 10% ACN) and then lyophilized to give the title compound (1.3 g, 52% yield) as a yellow semisolid.

### Step 2: Exo-8-((S)-tetrahydrofuran-3-yl)-8-azabicyclo[3.2.1]octan-3-ol

To a solution of 8-[(3S)-tetrahydrofuran-3-yl]-8-azabicyclo[3.2.1]octan-3-one (0.6 g, 3.07 mmol, 1 eq) in THF (6 mL) was added LiBH₄ (67 mg, 3.07 mmol, 1 eq). The mixture was stirred at −70° C. for 12 hours. On completion, the mixture was quenched with 1 N HCl (2 mL) at 0° C., and then diluted with ethyl acetate (50 mL) dried over Na₂SO₄, filtered and concentrated under reduced pressure to give the title compound (550 mg, 90% yield) as a white solid.

### L. (S)-4,4-difluorobutan-2-amine hydrochloride

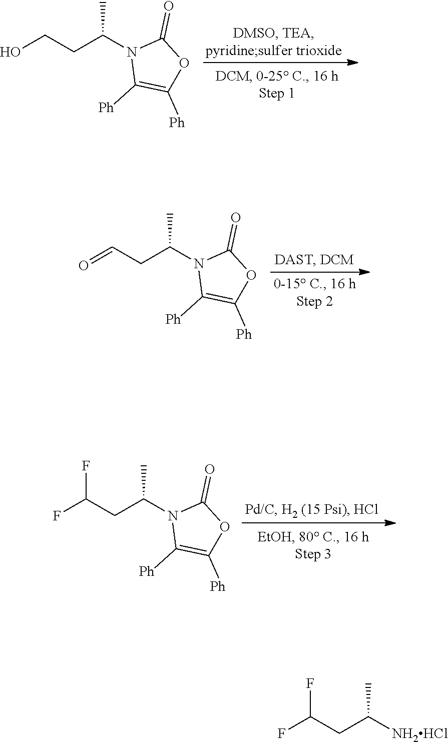

### Step 1: (S)-3-(2-oxo-4,5-diphenyloxazol-3(2H)-yl)butanal

To a solution of 3-[(1S)-3-hydroxy-1-methyl-propyl]-4,5-diphenyl-oxazol-2-one (5 g, 16.2 mmol, 1.0 eq) in DCM (50 mL) was added DMSO (3.8 mL, 48.5 mmol, 3 eq), TEA (6.8 mL, 48.5 mmol, 3 eq) and pyridine; sulfur trioxide (5.14 g, 32.3 mmol, 2 eq) at 0° C. Then the mixture was stirred at 25° C. for 16 hours. Upon completion, the mixture was concentrated in vacuo. The residue was diluted with water (50 mL) and extracted with ethyl acetate (30 mL×2). The combined organic layer was washed with brine (20 mL×2), dried over Na₂SO₄ and concentrated in vacuo. The residue was purified by column chromatography (SiO₂, petroleum ether/ethyl acetate=6:1) to give the title compound (2.7 g, 54% yield) as a white solid.

### Step 2: (S)-3-(4,4-difluorobutan-2-yl)-4,5-diphenyloxazol-2(3H)-one

To a solution of (3S)-3-(2-oxo-4,5-diphenyl-oxazol-3-yl)butanal (2.3 g, 7.5 mmol, 1 eq) in dry DCM (45 mL) was added DAST (diethylaminosulfur trifluoride; 12.1 g, 74.8 mmol, 9.89 mL, 10 eq) at 0° C. Then the mixture was stirred at 15° C. for 16 hours. Upon completion, the mixture was quenched by sat. NaHCO₃(50 mL), extracted with DCM (2×50 mL). The combined organic layer was washed with water (30 mL) and brine (30 mL), dried over Na₂SO₄ and concentrated in vacuo. The residue was purified by column chromatography (SiO₂, Petroleum ether/Ethyl acetate=20:1) to give the title compound (1.9 g, 76% yield) as a yellow oil.

### Step 3: (S)-4,4-difluorobutan-2-amine hydrochloride

To a solution of 3-[(1S)-3,3-difluoro-1-methyl-propyl]-4,5-diphenyl-oxazol-2-one (2.2 g, 6.7 mmol, 1 eq) in EtOH (80 mL) was added HCl (12 M, 2.78 mL, 5 eq) and Pd/C (2.83 g, 1.34 mmol, 5% purity, 0.2 eq) under N₂. The suspension was degassed under vacuum and purged with H₂ several times. The mixture was stirred under H₂ (15 psi) at 80° C. for 16 hours. Upon completion, the mixture was filtered, and the filtrated was concentrated in vacuo to give a crude product. To the residue was added 20 mL of petroleum ether/ethyl acetate (1:1) and 1 N HCl (30 mL). The aqueous layer was extracted with petroleum ether/ethyl acetate (1:1) (2×20 mL). The combined organic was washed with 1 N HCl (30 mL). The organic phase was discarded. The aqueous layer was combined and lyophilized to give the title compound (950 mg, 98% yield) as a white solid.

### Example 2: Synthesis of Compound 103

Compound 103 was synthesized according to General Scheme 1, above. The step numbers indicated below correspond to the steps shown in that scheme.

### Step 1: N2-butyl-8-(1,4-dioxaspiro[4.5]dec-7-en-8-yl)-N5-[(4-fluorophenyl)methyl]pyrido-[4,3-d]pyrimidine-2,5-diamine

A mixture of N-butyl-5-chloro-8-(1,4-dioxaspiro[4.5]dec-7-en-8-yl)pyrido[4,3-d]pyrimidin-2-amine (300 mg, 0.8 mmol), (4-fluorophenyl)methanamine (110 mg, 0.880 mmol, 0.10 mL), Pd₂(dba)₃ (tris(dibenzylideneacetone) dipalladium(O); 73 mg, 0.080 mmol), Xantphos (93 mg, 0.160 mmol) and Cs₂CO₃ (391 mg, 1.2 mmol) in dioxane (10 mL) was degassed and purged with N₂ 3 times, and then the mixture was stirred at 110° C. for 3 hours under N₂ atmosphere. Upon completion, the residue was diluted with water (10 mL) and extracted with EtOAc (10 mL×3). The organic layers were combined, washed with brine (15 mL), dried over Na₂SO₄, filtered and concentrated in vacuo. The residue was purified by prep-TLC to afford N₂-butyl-8-(1,4-dioxaspiro[4.5]dec-7-en-8-yl)-N₅-[(4-fluorophenyl)methyl]-pyrido[4,3-d]pyrimidine-2,5-diamine (190 mg, 34% yield, 67% purity) as a yellow solid.

### Step 2: N2-butyl-8-(1,4-dioxaspiro[4.5]decan-8-yl)-N5-[(4-fluorophenyl)methyl]-7,8-dihydropyrido[4,3-d]pyrimidine-2,5-diamine

To a solution of N2-butyl-8-(1,4-dioxaspiro[4.5]dec-7-en-8-yl)-N5-[(4-fluorophenyl)methyl]pyrido[4,3-d]pyrimidine-2,5-diamine (180 mg, 0.388 mmol) in THF (10 mL) was added 10% Pd/C (palladium on carbon; 180 mg) under N2. The suspension was degassed under vacuum and purged with H₂ several times then stirred under H₂ (15 psi) at 35° C. for 2 hours. Upon completion, the reaction mixture was filtered and concentrated under reduced pressure. The residue (180 mg, 0.385 mmol) was re-dissolved in THF (15 mL), then treated with MnO₂ (134 mg, 1.5 mmol). The mixture was stirred at 35° C. for 2 hours. Upon completion, the reaction mixture was filtered and concentrated under reduced pressure to afford N2-butyl-8-(1,4-dioxaspiro[4.5]decan-8-yl)-N5-[(4-fluorophenyl)methyl]pyrido[4,3-d]pyrimidine-2,5-diamine (140 mg, crude) as a yellow solid.

### Step 3: 4-[2-(butylamino)-5-[(4-fluorophenyl)methylamino]pyrido[4,3-d]pyrimidin-8-yl]cyclohexanone

To a solution of N2-butyl-8-(1,4-dioxaspiro[4.5]decan-8-yl)-N5-[(4-fluorophenyl)methyl]pyrido[4,3-d]pyrimidine-2,5-diamine (130 mg, 0.279 mmol) in MeCN (10 mL) was added aq. HCl (6 M, 10 mL). The mixture was stirred at RT for 1 hour. Upon completion, the reaction mixture was added sat. NaHCO₃ to adjust pH to 7. The mixture was extracted with EtOAc (10 mL×3). The organic layers were combined, washed with brine (10 mL×3), dried over Na₂SO₄, filtered and concentrated in vacuo to afford 4-[2-(butylamino)-5-[(4-fluorophenyl)methylamino]pyrido[4,3-d]pyrimidin-8-yl]cyclohexanone (112 mg, crude) as a yellow solid.

### Step 4: 4-[2-(butylamino)-5-[(4-fluorophenyl)methylamino]pyrido[4,3-d]pyrimidin-8-yl]cyclohexanol

To a solution of 4-[2-(butylamino)-5-[(4-fluorophenyl)methylamino]pyrido-[4,3-d]pyrimidin-8-yl]cyclohexanone (120 mg, 0.285 mmol) in MeOH (2 mL) was added NaBH₄ (sodium tetrahydroborate; 27 mg, 0.712 mmol). The mixture was stirred at 0° C. for 0.5 hour. Upon completion, to the reaction mixture was added sat. NH₄Cl (5 mL), and the mixture was extracted with EtOAc (3 mL×3). The organic layers were combined, washed with brine (3 mL×3), dried over Na₂SO₄, filtered and concentrated in vacuo. The residue was purified by prep-HPLC. Fractions were collected and concentrated under reduced pressure to remove MeCN. To the solution was added aq. HCl (0.2 M, 4 mL), then the residual aqueous solution was lyophilized to afford 4-[2-(butylamino)-5-[(4-fluorophenyl)methylamino]pyrido[4,3-d]pyrimidin-8-yl]cyclohexanol hydrochloride (18 mg, 13% yield, 94% purity) as a yellow solid. The structure was confirmed by LCMS and ¹H NMR.

### Example 3: Synthesis of Compound 104

Compound 104 was synthesized according to General Scheme 1, above. The step numbers indicated below correspond to the steps shown in that scheme.

### Step 1: N-butyl-8-(1,4-dioxaspiro[4.5]dec-7-en-8-yl)-5-tetrahydropyran-4-yloxy-pyrido[4,3-d]pyrimidin-2-amine

To a solution of N-butyl-5-chloro-8-(1,4-dioxaspiro[4.5]dec-7-en-8-yl)pyrido[4,3-d]pyrimidin-2-amine (300 mg, 0.800 mmol) in dioxane (10 mL) was added t-BuOK (potassium tert-butoxide; 269 mg, 2.4 mmol) and tetrahydropyran-4-ol (0.240 mL, 2.4 mmol). The mixture was stirred at 100° C. for 3 hours. Upon completion, the reaction mixture was cooled to RT. Water (10 mL) was then added prior to extract with EtOAc (15 mL×3). The organic layers were combined, washed with brine (10 mL×3), dried over Na₂SO₄, filtered and concentrated under reduced pressure to afford N-butyl-8-(1,4-dioxaspiro[4.5]dec-7-en-8-yl)-5-tetrahydropyran-4-yloxy-pyrido[4,3-d]pyrimidin-2-amine (310 mg, crude) as a black-brown solid, which was used in the next step without further purification.

### Step 2: N-butyl-8-(1,4-dioxaspiro[4.5]decan-8-yl)-5-((tetrahydro-2H-pyran-4-yl)oxy)pyrido[4,3-d]pyrimidin-2-amine

N-butyl-8-(1,4-dioxaspiro[4.5]dec-7-en-8-yl)-5-tetrahydropyran-4-yloxy-pyrido[4,3-d]pyrimidin-2-amine from the prior step was converted into the title compound by following the procedure set forth in Step 2 of Example 2.

### Step 3: 4-(2-(butylamino)-5-((tetrahydro-2H-pyran-4-yl)oxy)pyrido[4,3-d]pyrimidin-8-yl)cyclohexanone

N-butyl-8-(1,4-dioxaspiro[4.5]decan-8-yl)-5-((tetrahydro-2H-pyran-4-yl)oxy)pyrido[4,3-d]pyrimidin-2-amine from the prior step was converted into the title compound by following the procedure set forth in Step 3 of Example 2.

### Step 4: (1r,4r)-4-(2-(butylamino)-5-((tetrahydro-2H-pyran-4-yl)oxy)pyrido[4,3-d]pyrimidin-8-yl)cyclohexanol

4-(2-(butylamino)-5-((tetrahydro-2H-pyran-4-yl)oxy)pyrido[4,3-d]pyrimidin-8-yl)cyclohexanone from the prior step was converted into the title compound as a white solid by following the procedure set forth in Step 4 of Example 2. The structure was confirmed by LCMS, ¹H NMR, HSQC (heteronuclear single quantum coherence) and HMBC (heteronuclear multiple bond correlation).

### Example 4: Synthesis of Compound 106

Compound 106 was synthesized according to General Scheme 2, above. The step numbers indicated below correspond to the steps shown in that scheme.

### Step 1: 2-(butylamino)-8-(1,4-dioxaspiro[4.5]dec-7-en-8-yl)-6H-pyrido[4,3-d]pyrimidin-5-one

A mixture of 2-(butylamino)-8-iodo-6H-pyrido[4,3-d]pyrimidin-5-one (3.0 g, 8.7 mmol), 2-(1,4-dioxaspiro[4.5]dec-7-en-8-yl)-4,4,5,5-tetramethyl-1,3,2-dioxaborolane (3.5 g, 13 mmol), Pd(PPh₃)₂Cl₂ ([1,1′-bis(diphenylphosphino)ferrocene]dichloropalladium(II); 306 mg, 0.436 mmol) and Cs₂CO₃ (5.7 g, 17 mmol) in dioxane (90 mL) and H₂O (30 mL) was degassed and purged with N₂ 3 times, and then the mixture was stirred at 100° C. for 12 hours under N₂ atmosphere. The reaction mixture was diluted with ethyl acetate (500 mL) and extracted with water (50 mL×2). The organic layers were combined, washed with brine (100 mL×2), dried over anhydrous Na₂SO₄, and concentrated in vacuo. The residue was purified by prep-HPLC to afford 2-(butylamino)-8-(1,4-dioxaspiro[4.5]dec-7-en-8-yl)-6H-pyrido[4,3-d]pyrimidin-5-one (850 mg, 24% yield, 89% purity) as a white solid and the byproduct 2-(butylamino)pyrido[4,3-d]pyrimidin-5(6H)-one (1.2 g).

### Step 2: 2-(butylamino)-8-(1,4-dioxaspiro[4.5]decan-8-yl)pyrido[4,3-d]pyrimidin-5(6H)-one

To a solution of 2-(butylamino)-8-(1,4-dioxaspiro[4.5]dec-7-en-8-yl)-6H-pyrido[4,3-d]pyrimidin-5-one (650 mg, 1.8 mmol) in THF (20 mL) was added 10% Pd/C (650 mg) under N₂. The suspension was degassed under vacuum and purged with H₂ several times, then stirred under H₂ (15 psi) at 30° C. for 12 hours, later stirred at 40° C. for 12 hours, monitored by LCMS. Upon completion, the reaction mixture was filtered and concentrated under reduced pressure to give the title compound (600 mg, crude) as a yellow solid. The crude was re-dissolved in THF (10 mL), treated with MnO₂ (579 mg, 6.7 mmol) and stirred at 45° C. for 4 hours. Upon completion, the mixture was filtered and concentrated under reduced pressure to give the title compound (600 mg, crude) as a yellow solid.

### Step 3: 2-(butylamino)-8-(1,4-dioxaspiro[4.5]decan-8-yl)-6-tetrahydropyran-4-yl-pyrido[4,3-d]pyrimidin-5-one and N-butyl-8-(1,4-dioxaspiro[4.5]decan-8-yl)-5-tetrahydropyran-4-yloxy-pyrido[4,3-d]pyrimidin-2-amine

To a solution of 2-(butylamino)-8-(1,4-dioxaspiro[4.5]decan-8-yl)-6H-pyrido[4,3-d]pyrimidin-5-one (200 mg, 0.558 mmol) in DMF (20 mL) was added Cs₂CO₃ (363 mg, 1.1 mmol) and tetrahydropyran-4-yl methanesulfonate (101 mg, 0.558 mmol). The mixture was stirred at 80° C. for 24 hours. Upon completion, the reaction mixture was partitioned between EtOAc (50 mL) and water (20 mL). The organic phase was separated, washed with brine (15 mL×3), dried over Na₂SO₄, filtered and concentrated under reduced pressure. The residue was purified by prep-TLC to give N-butyl-8-(1,4-dioxaspiro[4.5]decan-8-yl)-5-tetrahydropyran-4-yloxy-pyrido[4,3-d]pyrimidin-2-amine (100 mg, 34% yield, 85% purity) as a yellow solid and 2-(butylamino)-8-(1,4-dioxaspiro[4.5]decan-8-yl)-6-tetrahydropyran-4-yl-pyrido[4,3-d]pyrimidin-5-one (80 mg, 22% yield, 67% purity) as a yellow solid.

### Step 4: 2-(butylamino)-8-(4-hydroxycyclohexyl)-6-tetrahydropyran-4-yl-pyrido[4,3-d]pyrimidin-5-one

To a solution of 2-(butylamino)-8-(1,4-dioxaspiro[4.5]decan-8-yl)-6-tetrahydropyran-4-yl-pyrido[4,3-d]pyrimidin-5-one (80 mg, 0.181 mmol) in DCM (3.0 mL) was added TFA (trifluoroacetic acid; 462 mg, 4.1 mmol, 0.3 mL). The mixture was stirred at 0° C. for 3 hours. Upon completion, the reaction mixture was partitioned between DCM (10 mL) and water (10 mL). The organic phase was separated, washed with brine (5 mL×3), dried over Na₂SO₄, filtered and concentrated under reduced pressure to give 2-(butylamino)-8-(4-oxocyclohexyl)-6-tetrahydropyran-4-yl-pyrido[4,3-d]pyrimidin-5-one (100 mg, crude) as a yellow solid. To a solution of 2-(butylamino)-8-(4-oxocyclohexyl)-6-tetrahydropyran-4-yl-pyrido[4,3-d]pyrimidin-5-one (100 mg, 0.251 mmol) in MeOH (1 mL) was added NaBH₄ (19 mg, 0.502 mmol) at 0° C., which was then stirred at 0° C. for 0.5 hour. Upon completion, the reaction mixture was quenched with sat. NH₄Cl (5 mL). The resulting mixture was extracted with EtOAc (3 mL×3). The organic layers were combined, washed with brine (3 mL×3), dried over Na₂SO₄, filtered and concentrated under reduced pressure. The residue was purified by prep-HPLC. After prep-HPLC, the mixture was concentrated under reduced pressure to remove MeCN and lyophilized to afford 2-(butylamino)-8-(4-hydroxycyclohexyl)-6-tetrahydropyran-4-yl-pyrido [4,3-d]pyrimidin-5-one (3 mg, 3% yield, 99% purity) as a white solid.

### Example 5: Synthesis of Compound 122

Compound 122 was synthesized according to General Scheme 3, above. The step numbers indicated below correspond to the steps shown in that scheme.

### Step 1: 8-iodo-2-(methylthio)-5-((tetrahydro-2H-pyran-4-yl)oxy)pyrido[4,3-d]pyrimidine

To a solution of tetrahydropyran-4-ol (1.42 mL, 14 mmol) in THF (50 mL) was added NaH (569 mg, 14 mmol, 60% purity) at RT under N₂. The mixture was stirred at RT for 0.5 hour. To the mixture was added 5-chloro-8-iodo-2-methylsulfanyl-pyrido[4,3-d]pyrimidine (4.8 g, 14 mmol), which was stirred for 12 hours. Upon completion, the reaction mixture was poured into sat. NH₄Cl (200 mL), extracted with ethyl acetate (200 mL×3). The organic layers were combined, washed with brine (150 mL), dried over Na₂SO₄, filtered and concentrated in vacuo. The residue was purified by column chromatography to give 8-iodo-2-methylsulfanyl-5-tetrahydropyran-4-yloxy-pyrido[4,3-d]pyrimidine (5.4 g, 8.4 mmol, 59% yield, 63% purity) as a white solid.

### Step 2: 2-(methylthio)-8-(1,4-dioxaspiro[4.5]dec-7-en-8-yl)-5-((tetrahydro-2H-pyran-4-yl)oxy)pyrido[4,3-d]pyrimidine

8-iodo-2-(methylthio)-5-((tetrahydro-2H-pyran-4-yl)oxy)pyrido[4,3-d]pyrimidine (5.3 g, 13.14 mmol), 2-(1,4-dioxaspiro[4.5]dec-7-en-8-yl)-4,4,5,5-tetramethyl-1,3,2-dioxaborolane (4.6 g, 17 mmol), Pd(PPh₃)₂C₁₂ (922 mg, 1.3 mmol) and Cs₂CO₃ (8.6 g, 26 mmol) in 1,4-dioxane (30 mL) and water (3 mL) was de-gassed and then refilled with N₂. The mixture was then heated to 50° C. for 48 hours under N₂. Upon completion, the residue was diluted with water (50 mL) and extracted with ethyl acetate (50 mL×3). The organic layers were combined, washed with brine (25 mL×2), dried over Na₂SO₄, filtered and concentrated in vacuo. The residue was purified by column chromatography to give 2-(methylthio)-8-(1,4-dioxaspiro[4.5]-dec-7-en-8-yl)-5-((tetrahydro-2H-pyran-4-yl)oxy)pyrido[4,3-d]pyrimidine (2.45 g, 41% yield, 93% purity) as a yellow solid.

### Step 3: 8-(1,4-dioxaspiro[4.5]dec-7-en-8-yl)-2-methylsulfonyl-5-tetrahydropyran-4-yloxy-pyrido[4,3-d]pyrimidine

To a solution of 8-(1,4-dioxaspiro[4.5]dec-7-en-8-yl)-2-methylsulfanyl-5-tetrahydropyran-4-yloxy-pyrido[4,3-d]pyrimidine (500 mg, 1.2 mmol) in DMF (10 mL) was added m-CPBA (519 mg, 2.4 mmol, 80% purity). The mixture was stirred at 45° C. for 2 hours. Upon completion, the mixture was used in the next step directly.

### Step 4: 8-(1,4-dioxaspiro[4.5]dec-7-en-8-yl)-N-[(1S)-2-methoxy-1-methyl-ethyl]-5-tetrahydropyran-4-yloxypyrido[4,3-d]pyrimidin-2-amine

To a solution of 8-(1,4-dioxaspiro[4.5]dec-7-en-8-yl)-2-methylsulfonyl-5-tetrahydropyran-4-yloxy-pyrido[4,3-d]pyrimidine (539 mg, 1.2 mmol) in DMF (5 mL) was added DIPEA (775 mg, 6.0 mmol, 1.0 mL) and (2S)-1-methoxypropan-2-amine (139 mg, 1.6 mmol). The reaction mixture was stirred at 65° C. for 3 hours. Upon completion, the reaction mixture was cooled to RT and treated with sat. Na₂S₂O₃ (150 mL). The mixture was extracted with EtOAc (100 mL×3). Organic layers were combined, washed with sat. NaHCO₃ (100 mL) and brine (50 mL×2), dried over Na₂SO₄, filtered and concentrated in vacuo. The residue was purified by column chromatography to give the title compound as a yellow solid.

### Step 5a: 4-[2-[[(1S)-2-methoxy-1-methyl-ethyl]amino]-5-tetrahydropyran-4-yloxy-pyrido[4,3-d]pyrimidin-8-yl]cyclohexanol

To a solution of 8-(1,4-dioxaspiro[4.5]dec-7-en-8-yl)-N-[(1S)-2-methoxy-1-methyl-ethyl]-5-tetrahydropyran-4-yloxypyrido[4,3-d]pyrimidin-2-amine (250 mg, 0.548 mmol) in MeOH (5 mL) was added 5% Pd/C (116 mg, 0.055 mmol). The mixture was stirred under H₂ (15 psi) at 45° C. for 2 hours. Upon completion, the reaction mixture was filtered and concentrated in vacuo. The residue was purified by prep-TLC to give an over-reduction byproduct (40 mg) and desired product (120 mg, 98% purity). The over-reduction byproduct was dissolved in THF (4 mL), treated with MnO₂ (75.50 mg, 0.869 mmol), and stirred at 50° C. for 16 hours. Upon completion, the mixture was filtered and the filtrate was concentrated in vacuo to afford 8-(1,4-dioxaspiro[4.5]decan-8-yl)-N-[(1S)-2-methoxy-1-methyl-ethyl]-5-tetrahydropyran-4-yloxy-pyrido[4,3-d]pyrimidin-2-amine (155 mg) as a white solid.

### Step 5b: 4-[2-[[(1S)-2-methoxy-1-methyl-ethyl]amino]-5-tetrahydropyran-4-yloxy-pyrido[4,3-d]pyrimidin-8-yl]cyclohexanone

To a solution of 8-(1,4-dioxaspiro[4.5]decan-8-yl)-N-[(1S)-2-methoxy-1-methyl-ethyl]-5-tetrahydropyran-4-yloxy-pyrido[4,3-d]pyrimidin-2-amine (155 mg, 0.338 mmol) in MeCN (2 mL) was added HCl (6 M, 2.0 mL). The mixture was stirred at RT for 1 hour. Upon completion, the reaction mixture was treated with sat. NaHCO₃(100 mL) to adjust pH to the range of 7˜10 and extracted with ethyl acetate (50 mL×3). The organic layers were combined, washed with brine (50 mL×2), dried over anhydrous Na₂SO₄, filtered and concentrated in vacuo to give 4-[2-[[(1S)-2-methoxy-1-methyl-ethyl]amino]-5-tetrahydropyran-4-yloxy-pyrido[4,3-d]pyrimidin-8-yl]cyclohexanone (138 mg, crude) as a yellow solid and used in the next step directly.

### Step 5c: (1S,4r)-4-(2-(((S)-1-methoxypropan-2-yl)amino)-5-((tetrahydro-2H-pyran-4-yl)oxy)pyrido[4,3-d]pyrimidin-8-yl)cyclohexan-1-ol

To a solution of 4-[2-[[(1S)-2-methoxy-1-methyl-ethyl]amino]-5-tetrahydropyran-4-yloxy-pyrido[4,3-d]pyrimidin-8-yl]cyclohexanone (138 mg, 0.333 mmol) in MeOH (3.0 mL) was added NaBH₄ (14 mg, 0.366 mmol) slowly. The mixture was stirred at 0° C. for 0.5 hour. Upon completion, the reaction mixture was added sat. NaHCO₃(100 mL) and extracted with EtOAc (50 mL×3). The organic layers were combined, washed with brine (50 mL×2) and dried over anhydrous Na₂SO₄, filtered and concentrated in vacuo to give the over reduced byproduct (139 mg, crude), which was dissolved in THF (5.0 mL), treated with MnO₂ (288 mg, 3.31 mmol) and stirred at 50° C. for 16 hours. Upon completion, the mixture was filtered and the filtrate was concentrated in vacuo. The residue was purified by prep-HPLC and the eluent was roto-vaped to remove organic solvent and the residual aqueous solution was lyophilized to give the title compound as a white solid. The structure was confirmed by LCMS and ¹H NMR.

### Example 6: Synthesis of Compound 134

Compound 134 was synthesized according to General Scheme 3, above. The step numbers indicated below correspond to the steps shown in that scheme.

### Step 3 & Step 4: 8-(1,4-dioxaspiro[4.5]dec-7-en-8-yl)-5-((tetrahydro-2H-pyran-4-yl)oxy)pyrido[4,3-d]pyrimidin-2-amine

To a solution of 8-(1,4-dioxaspiro[4.5]dec-7-en-8-yl)-2-methylsulfanyl-5-tetrahydro-pyran-4-yloxy-pyrido[4,3-d]pyrimidine (680 mg, 1.6 mmol) in DMF (10 mL) was added m-CPBA (883 mg, 4.1 mmol, 80% purity). The mixture was stirred at 40° C. for 2 hours, then added with 25% aq. ammonia (1.82 g, 13 mmol, 2 mL). The mixture was stirred at 40° C. for another 0.5 hour. Upon completion, the reaction mixture was partitioned between ethyl acetate (150 mL) and water (10 mL). The organic phase was separated, washed with brine (10 mL), dried over Na₂SO₄, filtered and concentrated under reduced pressure. The resulting residue was purified by prep-TLC to give 8-(1,4-dioxaspiro[4.5]dec-7-en-8-yl)-5-tetrahydro-pyran-4-yloxy-pyrido[4,3-d]pyrimidin-2-amine (160 mg, 20% yield, 80% purity) as a yellow solid.

### Step 5: 8-(1,4-dioxaspiro[4.5]decan-8-yl)-5-((tetrahydro-2H-pyran-4-yl)oxy)pyrido[4,3-d]pyrimidin-2-amine

To a solution of 8-(1,4-dioxaspiro[4.5]dec-7-en-8-yl)-5-tetrahydropyran-4-yloxy-pyrido[4,3-d]pyrimidin-2-amine (160 mg, 0.42 mmol) in THF (20 mL) was added 5% Pd/C (178 mg) under N₂. The suspension was degassed under vacuum and purged with H₂ several times. The mixture was stirred under H₂ (15 psi) at RT for 32 hours. Upon completion, the mixture was filtered and concentrated under reduced pressure. The resulting residue was dissolved in THF (30 mL), treated with MnO₂ (313 mg, 3.60 mmol), then stirred at 60° C. for 4 hours. Upon completion, the mixture was filtered and concentrated under reduced pressure. The residue was purified by prep-TLC to give 8-(1,4-dioxaspiro[4.5]decan-8-yl)-5-tetrahydro-pyran-4-yloxy-pyrido[4,3-d]pyrimidin-2-amine (90 mg, 51% yield, 79% purity) as a white solid.

### Step 7: N-(4-chlorophenyl)-8-(1,4-dioxaspiro[4.5]decan-8-yl)-5-tetrahydropyran-4-yloxy-pyrido[4,3-d]pyrimidin-2-amine

A mixture of 8-(1,4-dioxaspiro[4.5]decan-8-yl)-5-tetrahydropyran-4-yloxy-pyrido[4,3-d]pyrimidin-2-amine (60 mg, 0.16 mmol), NaOt-Bu (2 M in THF, 0.24 mL), 1-chloro-4-iodo-benzene (180 mg, 0.76 mmol), BrettPhos-Pd-G3 (14 mg, 0.02 mmol; with BrettPhos representing dicyclohexyl(2,4,6-triisopropyl-3,6-dimethoxy-[1,1-biphenyl]-2-yl)phosphine) in dioxane (6 mL) was degassed and refilled with nitrogen. The mixture was then heated at 80° C. for 12 hours under N₂. Upon completion, the reaction mixture was cooled to RT, quenched with sat. aq. NH₄Cl (40 mL). The mixture was diluted with water (10 mL) and extracted with ethyl acetate (20 mL×3). The organic layers were combined, washed with brine (30 mL), dried over Na₂SO₄, filtered and concentrated under reduced pressure. The residue was purified by prep-TLC to afford N-(4-chlorophenyl)-8-(1,4-dioxaspiro[4.5]decan-8-yl)-5-tetrahydropyran-4-yloxy-pyrido[4,3-d]pyrimidin-2-amine (35 mg, 0.06 mmol, 38% yield, 83% purity) as a white solid.

### Step 8a: 4-[2-(4-chloroanilino)-5-tetrahydropyran-4-yloxy-pyrido[4,3-d]pyrimidin-8-yl]-cyclohexanone

To a solution of N-(4-chlorophenyl)-8-(1,4-dioxaspiro[4.5]decan-8-yl)-5-tetrahydropyran-4-yloxy-pyrido[4,3-d]pyrimidin-2-amine (39 mg, 0.08 mmol) in MeCN (3 mL) was added HCl (6 M, 0.5 mL) at RT. The mixture was stirred for 0.5 hour. Upon completion, the reaction mixture was quenched by water (10 mL), then diluted with sat. aq. NaHCO₃(30 mL) and extracted with EtOAc (50 mL×3). The organic layers were combined, washed with addition sat. aq. NaHCO₃(30 mL), dried over Na₂SO₄, filtered and concentrated under reduced pressure to give 4-[2-(4-chloroanilino)-5-tetrahydropyran-4-yloxy-pyrido[4,3-d]pyrimidin-8-yl]-cyclohexanone (36 mg, crude).

### Step 8b: (1r,4r)-4-(2-((4-chlorophenyl)amino)-5-((tetrahydro-2H-pyran-4-yl)oxy)pyrido[4,3-d]pyrimidin-8-yl)cyclohexan-1-ol

A solution of 4-[2-(4-chloroanilino)-5-tetrahydropyran-4-yloxy-pyrido[4,3-d]pyrimidin-8-yl]-cyclohexanone (36 mg, 0.09 mmol) in MeOH (5 mL) was treated with NaBH₄ (3 mg, 0.08 mmol) at 0° C. The mixture was stirred at 0° C. for 10 mins, then quenched with sat. NH₄Cl (30 mL) at the same temperature. The resulted mixture was diluted with water (10 mL) and extracted with EtOAc (50 mL×3). The organic layers were combined, washed with brine (50 mL), dried over Na₂SO₄, and filtered and concentrated under reduced pressure. The residue was purified by prep-TLC to give the title compound (2.4 mg, 6.2% yield, 94% purity) as a white solid. The structure was confirmed by LCMS and ¹H NMR.

### Example 7: Synthesis of Compound 111

Compound 111 was synthesized according to General Scheme 4, above. The step numbers indicated below correspond to the steps shown in that scheme.

### Step 1: Tert-butyl 4-(2-methylsulfanyl-5-oxo-pyrido[4,3-d]pyrimidin-6-yl)piperidine-1-carboxylate

A solution of 2-methylsulfanyl-6H-pyrido[4,3-d]pyrimidin-5-one (5.0 g, 26 mmol) and tert-butyl 4-methylsulfonyloxypiperidine-1-carboxylate (11 g, 39 mmol) in DMF (100 mL) and DME (100 mL) was treated with Cs₂CO₃ (17 g, 52 mmol). The mixture was heated at 80° C. for 16 hours, cooled to RT and then poured into water (100 mL). The mixture was extracted with ethyl acetate (300 mL×2). The organic layers were combined, washed with brine (100 mL×2) and concentrated in vacuo. The residue was purified by column chromatography to afford tert-butyl 4-(2-methylsulfanyl-5-oxo-pyrido[4,3-d]pyrimidin-6-yl)piperidine-1-carboxylate (5.3 g, 52% yield, 95% purity) as a white solid and tert-butyl 4-(2-methylsulfanylpyrido[4,3-d]pyrimidin-5-yl)oxypiperidine-1-carboxylate (4.0 g, 40% yield, 98% purity) as a white solid. The product was confirmed by LCMS, 1H NMR and 2D NMR.

### Step 2: Tert-butyl 4-(8-iodo-2-methylsulfanyl-5-oxo-pyrido[4,3-d]pyrimidin-6-yl)piperidine-1-carboxylate

To a solution of tert-butyl 4-(2-methylsulfanyl-5-oxo-pyrido[4,3-d]pyrimidin-6-yl) -piperidine-1-carboxylate (2.0 g, 5.3 mmol) in dry DMF (10 mL) was added NIS (1.8 g, 8.0 mmol) at RT, which was then heated at 80° C. for 2 hours. Upon completion, the reaction solution was cooled to RT then poured into icy water (20 mL). The precipitated solid was filtered and dried in vacuo to afford the title compound (2.0 g, 68% yield, 91% purity) as a yellow solid, which was directly used in the next step.

### Step 3: Tert-butyl 4-[8-(1,4-dioxaspiro[4.5]dec-7-en-8-yl)-2-methylsulfanyl-5-oxo-pyrido[4,3-d]pyrimidin-6-yl]piperidine-1-carboxylate

To a solution of tert-butyl 4-(8-iodo-2-methylsulfanyl-5-oxo-pyrido[4,3-d]pyrimidin-6-yl)piperidine-1-carboxylate (2.0 g, 4.0 mmol) and 2-(1,4-dioxaspiro[4.5]dec-7-en-8-yl)-4,4,5,5-tetramethyl-1,3,2-dioxaborolane (2.1 g, 8.0 mmol) in a mixture of dioxane (12 mL) and H₂O (4 mL) was added Pd(PPh₃)₂Cl₂ (279 mg, 0.398 mmol), followed by addition of Cs₂CO₃ (2.6 g, 8.0 mmol) at RT. The suspension was degassed under vacuum and purged with N₂ several times, then heated at 50° C. for 12 hours. To the solution was added 4,4,5,5-tetramethyl-2-(1,4-dioxaspiro[4.5]dec-7-en-8-yl)-1,3,2-dioxaborolane (0.5 g), and the reaction was stirred at 50° C. for another 5 hours. The mixture was diluted with EtOAc (50 mL) and washed with brine (20 mL). The organic layer was separated, concentrated and purified by column chromatography to afford the title compound (1.6 g, 77% yield, 98% purity) as a yellow solid.

### Step 4 & Step 5: Tert-butyl 4-(2-((1-methoxypropan-2-yl)amino)-5-oxo-8-(1,4-dioxaspiro[4.5]dec-7-en-8-yl)pyrido[4,3-d]pyrimidin-6(5H)-yl)piperidine-1-carboxylate

To the solution of tert-butyl 4-[8-(1,4-dioxaspiro[4.5]dec-7-en-8-yl)-2-methylsulfanyl-5-oxo-pyrido[4,3-d]pyrimidin-6-yl]piperidine-1-carboxylate (0.3 g, 0.58 mmol) in dry N,N-dimethyl formamide (3 mL) was added metachloroperbenzoic (0.25 g, 1.2 mmol) at RT. The reaction was then heated at 50° C. for 1 hour. Upon completion, the reaction solution was cooled to RT. The resulting mixture composed of sulfoxide and sulfone was used directly in the next step. To the reaction solution was added diisopropylethylamine (0.41 mL, 2.3 mmol) and 1-methoxypropan-2-amine (0.12 mL, 1.2 mmol). The reaction mixture was then heated at 65° C. for 4 hours. Upon completion, the reaction solution was diluted with ethyl acetate (30 mL). The mixture was washed by saturated aqueous sodium sulfite (10 mL×3). The organic layer was dried over sodium sulfate, filtered and concentrated in vacuo. The residue was purified by silica gel chromatography to give the title compound (0.2 g, 53% yield) as a white solid.

### Step 6: tert-butyl 4-[8-(1,4-dioxaspiro[4.5]dec-7-en-8-yl)-2-[(2-methoxy-1-methyl-ethyl)amino]-5-oxo-pyrido[4,3-d]pyrimidin-6-yl]piperidine-1-carboxylate

To the solution of tert-butyl 4-[8-(1,4-dioxaspiro[4.5]dec-7-en-8-yl)-2-[(2-methoxy-1-methyl-ethyl)amino]-5-oxo-pyrido[4,3-d]pyrimidin-6-yl]piperidine-1-carboxylate (0.1 g, 0.18 mmol) in dry methanol (4 mL) was added Pd/C (10%, 10 mg, dry). The reaction mixture was stirred at 50° C. under hydrogen (50 psi) atmosphere for 4 hours. Upon completion, the reaction slurry was filtered, and the filtrate was concentrated to dryness. The residue was re-dissolved in dry dichloromethane (4 mL) and added manganese dioxide (0.16 g, 1.8 mmol). The mixture was stirred at 30° C. for 13 hours. Upon completion, the reaction slurry was filtered, and the filtrate was concentrated in vacuo. The residue was purified by silica gel chromatography to give tert-butyl 4-[8-(1,4-dioxaspiro[4.5]dec-7-en-8-yl)-2-[(2-methoxy-1-methyl-ethyl)amino]-5-oxo-pyrido[4,3-d]pyrimidin-6-yl]piperidine-1-carboxylate (54 mg, 52% yield) as a white solid.

### Step 7a: 2-[(2-methoxy-1-methyl-ethyl)amino]-8-(4-oxocyclohexyl)-6-(4-piperidyl)pyrido[4,3-d]pyrimidin-5-one

To a solution of tert-butyl 4-[8-(1,4-dioxaspiro[4.5]dec-7-en-8-yl)-2-[(2-methoxy-1-methyl-ethyl)-amino]-5-oxo-pyrido[4,3-d]pyrimidin-6-yl]piperidine-1-carboxylate (80 mg, 0.14 mmol) in dry dichloromethane (2 mL) was added trifluoroacetic acid (1 mL, 14 mmol) at 0° C. The reaction mixture was allowed to warm to RT and stirred for 12 hours. Upon completion, the volatile was removed and the residue was dissolved in sat. aq. sodium bicarbonate (8 mL). The mixture was extracted with ethyl acetate (10 mL×2). The organic layers were combined, dried over Na₂SO₄, filtered and concentrated in vacuo to give 2-[(2-methoxy-1-methyl-ethyl)amino]-8-(4-oxocyclohexyl)-6-(4-piperidyl)pyrido[4,3-d]pyrimidin-5-one (as crude) which was directly used in the next step.

### Step 7b: 8-((1r,4r)-4-hydroxycyclohexyl)-2-((1-methoxypropan-2-yl)amino)-6-(piperidin-4-yl)pyrido[4,3-d]pyrimidin-5(6H)-one

To a solution of 2-[(2-methoxy-1-methyl-ethyl)amino]-8-(4-oxocyclohexyl)-6-(4-piperidyl)pyrido[4,3-d]pyrimidin-5-one (60 mg, 0.15 mmol, crude) in dry methanol (2 mL) was added sodium borohydride (16 mg, 0.44 mmol) at 0° C. The mixture was stirred at the same temperature for 1 hour. Upon completion, the volatile was removed and the residue was dissolved in sat. aq. NH₄Cl (8 mL). The mixture was extracted with EtOAc (10 mL×2). The aqueous layer was separated and concentrated in vacuo. The residue was purified by prep-HPLC to give the title compound as a TFA salt, which was dissolved in water (10 mL), treated with 0.2 N HCl (0.2 mL), and then lyophilized to give the title compound (10 mg, 16% yield) as a yellow solid. The structure was confirmed by LCMS and ¹H NMR.

### Example 8: Synthesis of Compound 128

Compound 128 was synthesized according to General Scheme 4, above. The step numbers indicated below correspond to the steps shown in that scheme.

### Step 8a: (1r,4r)-4-(6-(1-acetylpiperidin-4-yl)-5-oxo-2-(pentan-2-ylamino)-5,6-dihydropyrido[4,3-d]pyrimidin-8-yl)cyclohexyl acetate

To a solution of Compound 112 (40 mg, 0.076 mmol) in dry DCM (0.5 mL) was added DIPEA (20 mg, 0.152 mmol), followed by addition of pyridine (6.0 mg, 0.076 mmol) then addition of acetyl chloride (11.9 mg, 0.152 mmol) at −20° C. The reaction was stirred at this temperature for 1 hour. Upon completion, the reaction solution was poured down to sat. aq. NaHCO₃(10 mL). The mixture was extracted with DCM (10 mL×3). The combined organic layers were dried, filtered and concentrated to dryness to give the title compound (43 mg, crude).

### Step 8b: 6-(1-acetylpiperidin-4-yl)-8-((1r,4r)-4-hydroxycyclohexyl)-2-(pentan-2-ylamino)pyrido[4,3-d]pyrimidin-5(6H)-one hydrochloride

To the solution of [4-[6-(1-acetyl-4-piperidyl)-2-(1-methylbutylamino)-5-oxo-pyrido[4,3-d]pyrimidin-8-yl]cyclohexyl] acetate (43 mg, crude) in MeOH (1 mL) was added NaOH (2 M, 0.086 mL) at 0° C. The reaction mixture was allowed to rise to 20° C., and it was stirred at this temperature for 1 hour. Upon completion, to the reaction solution was added 1N HCl (0.2 mL). The pH of the solution was 5, and the solution was filtered to give a residue. The residue was purified by prep-HPLC. After prep-HPLC purification, the eluent was concentrated to remove the organic solvent, adjusted with aq. HCl (0.2M, 0.2 mL), and then lyophilized to dryness to give the tittle compound (16 mg, 35% yield, 94% purity) as a yellow solid.

### Example 9: Synthesis of Compound 102

Compound 101 was synthesized according to General Scheme 5, above. The step numbers indicated below correspond to the steps shown in that scheme.

### Step 1: 4-methyl-2-(methylthio)pyrimidine-5-carboxylic acid

To a solution of ethyl 4-methyl-2-methylsulfanyl-pyrimidine-5-carboxylate (5.0 g, 24 mmol) in EtOH (50 mL) was added NaOH (1.9 g, 47 mmol) in H₂O (50 mL) at 0° C. The mixture was stirred at RT for 2 hours. Upon completion, the reaction mixture was quenched with conc. aq. HCl (4 mL) at 0° C., diluted with EtOAc (100 mL) and extracted with EtOAc (50 mL×2). The organic layers were combined, washed with brine (25 mL×2), dried over Na₂SO₄, filtered and concentrated under reduced pressure to give the tittle compound (4.2 g, 97% yield).

### Step 2: benzyl 4-(4-methyl-2-(methylthio)pyrimidine-5-carboxamido)piperidine-1-carboxylate

To a solution of 4-methyl-2-methylsulfanyl-pyrimidine-5-carboxylic acid (3.0 g, 16 mmol) in DMF (5 mL) was added HATU ((dimethylamino)-N,N-dimethyl(3H-[1,2,3]triazolo[4,5-b]yridine-3-yloxy)methaniminium hexafluorophosphate; 7.4 g, 20 mmol). The mixture was stirred at RT for 3 hours. Benzyl 4-aminopiperidine-1-carboxylate (4.2 g, 18 mmol) and DIPEA (8.4 g, 65 mmol, 11.4 mL) were then added in the mixture before stirring at RT for 3 hours. Upon completion, the reaction mixture was quenched with water (50 mL), diluted with EtOAc (30 mL) and extracted with EtOAc (30 mL×3). The organic layers were combined, washed with brine (10 mL×3), dried over anhydrous sodium sulfate, filtered and concentrated under reduced pressure. The residue was purified by column chromatography to give the tittle compound (6.0 g, 92% yield).

### Step 3: benzyl 4-(4-methyl-2-(methylsulfonyl)pyrimidine-5-carboxamido)piperidine-1-carboxylate

To a solution of benzyl 4-[(4-methyl-2-methylsulfanyl-pyrimidine-5-carbonyl)amino]piperidine-1-carboxylate (2.2 g, 5.5 mmol) in DMF (40 mL) was added m-CPBA (2.4 g, 11 mmol, 80% purity). The mixture was stirred at 45° C. for 1 hour then used in the next step directly.

### Step 4: benzyl 4-(2-(butylamino)-4-methylpyrimidine-5-carboxamido)piperidine-1-carboxylate

To a solution of benzyl 4-[(4-methyl-2-methyl sulfonyl-pyrimidine-5-carbonyl)amino]piperidine-1-carboxylate (2.4 g, 5.5 mmol) in DMF (40 mL) was added DIPEA (3.5 g, 27 mmol, 4.8 mL) and BuNH₂(521 mg, 7.1 mmol). The mixture was stirred at 65° C. for 4 hours. Upon completion, the reaction mixture was quenched by addition of saturated Na₂S₂O₃ (30 mL) and saturated NaHCO₃(30 mL), then diluted with EA (40 mL) and extracted with EA (40 mL×3) and water (30 mL). The organic layers were combined, washed with brine (30 mL×2), dried over anhydrous Na₂SO₄, filtered and concentrated under reduced pressure. The residue was washed with DCM (6 mL) and petroleum ether (150 mL) and then filtered. The filtrate was concentrated under reduced pressure to afford the title compound (2.1 g, 74% yield, 84% purity) as a white solid.

### Step 5: benzyl 4-(2-(butylamino)-5-oxopyrido[4,3-d]pyrimidin-6(5H)-yl)piperidine-1-carboxylate

Benzyl 4-[[2-(butylamino)-4-methyl-pyrimidine-5-carbonyl]amino]piperidine-1-carboxylate (1.0 g, 2.4 mmol) was taken up into a microwave tube in DMF-DMA (12 mL). The sealed tube was heated at 170° C. for 2 hours then heated at 170° C. for another 2 hours under microwave irradiation, monitored by LCMS. Upon completion, the reaction mixture was diluted with water (30 mL) and extracted with EA (30 mL×3). The organic layers were combined washed with brine (30 mL×3), dried over anhydrous Na₂SO₄, filtered and concentrated under reduced pressure. The residue was purified by column chromatography to give the title compound (295 mg, 20% yield, 70% purity) as a light yellow solid.

### Step 6: benzyl 4-(8-bromo-2-(butylamino)-5-oxopyrido[4,3-d]pyrimidin-6(5H)-yl)piperidine-1-carboxylate

To a solution of benzyl 4-[2-(butylamino)-5-oxo-pyrido[4,3-d]pyrimidin-6-yl]piperidine-1-carboxylate (140 mg, 0.322 mmol) in DMF (4 mL) was added NBS (69 mg, 0.386 mmol) at RT. The mixture was stirred for 10 minutes. Upon completion, the reaction mixture was diluted with water (30 mL) and extracted with EtOAc (30 mL×3). The organic layers were combined, washed with brine (30 mL×2), dried over anhydrous Na₂SO₄, filtered and concentrated under reduced pressure. The residue was purified by column chromatography, and concentrated in vacuo to give the tittle compound(140 mg, 73% yield, 86% purity) as a light yellow solid.

### Step 7: benzyl 4-(2-(butylamino)-5-oxo-8-(1,4-dioxaspiro[4.5]dec-7-en-8-yl)pyrido[4,3-d]pyrimidin-6(5H)-yl)piperidine-1-carboxylate

Benzyl 4-[8-bromo-2-(butylamino)-5-oxo-pyrido[4,3-d]pyrimidin-6-yl]piperidine-1-carboxylate (200 mg, 0.389 mmol), 8-(4,4,5,5-tetramethyl-1,3-dioxolan-2-yl)-1,4-dioxaspiro[4.5]dec-7-ene (125 mg, 0.467 mmol) and Pd(PPh₃)₂Cl₂ (27 mg, 0.039 mmol) were taken up into a microwave tube in dioxane (10 mL) and H₂O (3 mL). The sealed tube was heated at 140° C. for 20 minutes under microwave irradiation. Upon completion, the reaction mixture was diluted with ethyl acetate (20 mL) and extracted with ethyl acetate (20 mL×3) and water (20 mL). The organic layers were combined, washed with brine (20 mL×2), dried over anhydrous Na₂SO₄, filtered and concentrated in vacuo. The residue was purified by prep-TLC to give the tittle compound (200 mg, 75% yield, 84% purity) as a white solid.

### Step 8a: 2-(butylamino)-6-(1-methylpiperidin-4-yl)-8-(1,4-dioxaspiro[4.5]decan-8-yl)-3,4-dihydropyrido[4,3-d]pyrimidin-5(6H)-one

To a solution of benzyl 4-[2-(butylamino)-8-(1,4-dioxaspiro[4.5]dec-7-en-8-yl)-5-oxo-pyrido[4,3-d]pyrimidin-6-yl]piperidine-1-carboxylate (50 mg, 0.087 mmol) in MeOH (5 mL) was added 5% Pd/C (10 mg) under N₂. The suspension was degassed under vacuum and purged with H₂ several times, then stirred under H₂ (15 psi) at RT for 12 hours. Upon completion, the reaction mixture was filtered and concentrated under reduced pressure to give 2-(butylamino)-6-(1-methylpiperidin-4-yl)-8-(1,4-dioxaspiro[4.5]decan-8-yl)-3,4-dihydropyrido[4,3-d]pyrimidin-5(6H)-one (40 mg, crude).

### Step 8b: 2-(butylamino)-8-(1,4-dioxaspiro[4.5]decan-8-yl)-6-(1-methyl-4-piperidyl)pyrido[4,3-d]pyrimidin-5-one

To a solution of 2-(butylamino)-6-(1-methylpiperidin-4-yl)-8-(1,4-dioxaspiro[4.5]decan-8-yl)-3,4-dihydropyrido[4,3-d]pyrimidin-5(6H)-one (40 mg, 0.087 mmol) in acetonitrile (5 mL) was added dropwise MnO₂ (38 mg, 0.44 mmol) at 30° C. After addition, the mixture was stirred at this temperature for 12 hours. Upon completion, the reaction mixture was filtered and concentrated under reduced pressure to give the title compound (25 mg, crude).

### Step 8c: 2-(butylamino)-6-(1-methyl-4-piperidyl)-8-(4-oxocyclohexyl)pyrido[4,3-d]pyrimidin-5-one

To a solution of 2-(butylamino)-8-(1,4-dioxaspiro[4.5]decan-8-yl)-6-(1-methyl-4-piperidyl)pyrido[4,3-d]pyrimidin-5-one (25 mg, 0.055 mmol) in THF (5 mL) was added aq. HCl (6 M, 6.0 mL). The mixture was stirred at RT for 1 hour. Upon completion, to the reaction mixture was added NaHCO₃ to adjust pH to 6-7, and then diluted with water (20 mL), extracted with EtOAc (20 mL×3). The organic layers were combined, dried over anhydrous Na₂SO₄, filtered and concentrated under reduced pressure to give the title compound (30 mg, crude).

### Step 8d: 2-(butylamino)-8-((1r,4r)-4-hydroxycyclohexyl)-6-(1-methylpiperidin-4-yl)pyrido[4,3-d]pyrimidin-5(6H)-one

To a solution of 2-(butylamino)-6-(1-methyl-4-piperidyl)-8-(4-oxocyclohexyl)pyrido[4,3-d]pyrimidin-5-one (30 mg, crude) in MeOH (5 mL) was added NaBH₄ (11 mg, 0.292 mmol) at 0° C. The mixture was stirred at RT for 2 hours. Upon completion, the reaction mixture concentrated under reduced pressure. The residue was purified by prep-HPLC, adjusted by aq. HCl (0.2 M, 0.5 mL), and lyophilized to give the title compound as hydrochloride salt (5.3 mg, 16% yield overall) as a light yellow solid.

### Example 10: Synthesis of Compound 109

Compound 109 was synthesized according to General Scheme 6, above. The step numbers indicated below correspond to the steps shown in that scheme.

### Step 1: tert-butyl 4-(6-chloro-4-iodo-1-oxo-2,7-naphthyridin-2(1H)-yl)piperidine-1-carboxylate and tert-butyl 4-((6-chloro-4-iodo-2,7-naphthyridin-1-yl)oxy)piperidine-1-carboxylate

To a solution of 6-chloro-4-iodo-2H-2,7-naphthyridin-1-one (3.5 g, 11.4 mmol) in DMF (30 mL) was added Cs₂CO₃ (11.2 g, 34.3 mmol) and tert-butyl 4-methylsulfonyloxypiperidine-1-carboxylate (6.4 g, 22.8 mmol). The mixture was heated at 80° C. for 16 hours. The reaction mixture was quenched by water (5 mL) at RT, diluted with water (10 mL) and extracted with ethyl acetate (20 mL×3). The combined organic layers were washed with brine (25 mL×2), dried over Na₂SO₄, filtered and concentrated in vacuo. The residue was purified by column chromatography to afford tert-butyl 4-(6-chloro-4-iodo-1-oxo-2,7-naphthyridin-2-yl)piperidine-1-carboxylate (1.2 g, 18% yield, 86% purity) as a yellow solid and tert-butyl 4-[(6-chloro-4-iodo-2,7-naphthyridin-1-yl)oxy]piperidine-1-carboxylate (2 g, 30% yield, 84% purity) as a white solid.

### Step 2: tert-butyl 4-(6-chloro-1-oxo-4-(1,4-dioxaspiro[4.5]dec-7-en-8-yl)-2,7-naphthyridin-2(1H)-yl)piperidine-1-carboxylate

Tert-butyl 4-(6-chloro-4-iodo-1-oxo-2,7-naphthyridin-2-yl)piperidine-1-carboxylate (1.4 g, 2.9 mmol), 2-(1,4-dioxaspiro[4.5]dec-7-en-8-yl)-4,4,5,5-tetramethyl-1,3,2-dioxaborolane (760 mg, 2.9 mmol), Pd(PPh₃)₂Cl₂ (200 mg, 0.29 mmol) and Cs₂CO₃ (1.9 g, 5.7 mmol) in 1,4-dioxane (20 mL) and water (6 mL) were de-gassed then heated to 50° C. for 16 hours under N₂. The reaction mixture was concentrated under reduced pressure to remove 1,4-dioxane. The residue was diluted with water (5 mL) and extracted with ethyl acetate (10 mL×3). The combined organic layers were washed with brine (10 mL×2), dried over Na₂SO₄, filtered and concentrated in vacuo. The residue was purified by prep-TLC to give tert-butyl 4-[6-chloro-4-(1,4-dioxaspiro[4.5]dec-7-en-8-yl)-1-oxo-2,7-naphthyridin-2-yl]piperidine-1-carboxylate (0.9 g, 51% yield, 81% purity) as a black-brown solid.

### Step 3: tert-butyl 4-(6-(butylamino)-1-oxo-4-(1,4-dioxaspiro[4.5]dec-7-en-8-yl)-2,7-naphthyridin-2(1H)-yl)piperidine-1-carboxylate

A mixture of tert-butyl 4-[6-chloro-4-(1,4-dioxaspiro[4.5]dec-7-en-8-yl)-1-oxo-2,7-naphthyridin-2-yl]piperidine-1-carboxylate (900 mg, 1.8 mmol), butan-1-amine (393 mg, 5.4 mmol, 0.53 mL) and BrettPhos-Pd-G3 (162 mg, 0.18 mmol) and t-BuONa (2 M in THF, 0.89 mL) in 1,4-dioxane (5 mL) was de-gassed and then heated to 90° C. for 16 hours under N₂. The reaction mixture was quenched by addition of water (10 mL) at RT, and then extracted with ethyl acetate (60 mL×3). The combined organic layers were washed with brine (25 mL×2), dried over Na₂SO₄, filtered and concentrated in vacuo. The residue was purified by column chromatography to give tert-butyl 4-[6-(butylamino)-4-(1,4-dioxaspiro[4.5]dec-7-en-8-yl)-1-oxo-2,7-naphthyridin-2-yl]piperidine-1-carboxylate (600 mg, 54% yield, 87% purity) as a yellow solid.

### Step 4a: tert-butyl 4-(6-(butylamino)-1-oxo-4-(1,4-dioxaspiro[4.5]decan-8-yl)-2,7-naphthyridin-2(1H)-yl)piperidine-1-carboxylate

To a solution of tert-butyl 4-[6-(butylamino)-4-(1,4-dioxaspiro[4.5]dec-7-en-8-yl)-1-oxo-2,7-naphthyridin-2-yl]piperidine-1-carboxylate (600 mg, 1.1 mmol) in MeOH (50 mL) was added 10% Pd/C (100 mg) under N₂. The suspension was degassed under vacuum and purged with H₂ several times. The mixture was stirred under H₂ (15 psi) at 35° C. for 64 hours. The reaction mixture was filtered and concentrated under reduced pressure to give tert-butyl 4-[6-(butylamino)-4-(1,4-dioxaspiro[4.5]decan-8-yl)-1-oxo-2,7-naphthyridin-2-yl]piperidine-1-carboxylate (440 mg, crude) as a yellow solid.

### Step 4b: 6-(butylamino)-4-(4-oxocyclohexyl)-2-(piperidin-4-yl)-2,7-naphthyridin-1(2H)-one

To a solution of tert-butyl 4-[6-(butylamino)-4-(1,4-dioxaspiro[4.5]decan-8-yl)-1-oxo-2,7-naphthyridin-2-yl]piperidine-1-carboxylate (440 mg, 0.8 mmol) in DCM (2 mL) was added TFA (1.5 g, 13.5 mmol, 1 mL). The mixture was stirred at RT for 16 hours. The reaction mixture was concentrated under reduced pressure to remove DCM and TFA. The residue was partitioned between ethyl acetate (150 mL) and sat. aq. NaHCO₃(50 mL). The organic phase was separated, washed with brine (50 mL×2), dried over Na₂SO₄, filtered and concentrated in vacuo to give 6-(butylamino)-4-(4-oxocyclohexyl)-2-(4-piperidyl)-2,7-naphthyridin-1-one (300 mg, 67% yield, 72% purity) as a yellow solid.

### Step 4c: 6-(butylamino)-4-((1r,4r)-4-hydroxycyclohexyl)-2-(piperidin-4-yl)-2,7-naphthyridin-1(2H)-one hydrochloride

To a solution of 6-(butylamino)-4-(4-oxocyclohexyl)-2-(4-piperidyl)-2,7-naphthyridin-1-one (70 mg, 0.18 mmol) in MeOH (5 mL) was added NaBH₄ (13 mg, 0.35 mmol). The mixture was stirred at 0° C. for 1 hour. To the mixture was added 5 drops of 1 N HCl. The mixture was concentrated under reduced pressure to give a residue. The residue was purified by prep-HPLC. Fractions were combined and concentrated under reduced pressure to remove MeCN. The resulting mixture was treated with 0.2 N HCl (4 mL), then lyophilized to give 6-(butylamino)-4-(4-hydroxycyclohexyl)-2-(4-piperidyl)-2,7-naphthyridin-1-one hydrochloride (9.6 mg, 12% yield, 99% purity) as a yellow solid. The title compound was confirmed by ¹H NMR and LCMS.

### Example 11: Synthesis of Compound 307

Compound 307 was synthesized according to General Scheme 7, above. The step numbers indicated below correspond to the steps shown in that scheme.

### Step 1: (S)-5-chloro-8-iodo-N-(1-methoxypropan-2-yl)pyrido[4,3-d]pyrimidin-2-amine

A solution of 8-iodo-2-[[(1S)-2-methoxy-1-methyl-ethyl]amino]-6H-pyrido[4,3-d]pyrimidin-5-one (17 g, 47 mmol, 1 eq) in POCl₃ (56 g, 366 mmol, 34 mL) was stirred at 110° C. for 1 hour. On completion, the mixture was poured into ice/water (800 mL) and filtered. The solid was washed with water (2×50 mL) and dried in vacuo to give a pure product. The filtrate was adjusted to pH 8 with 2 N NaOH, extracted with ethyl acetate (2×100 mL). The organic layer was washed with water (50 mL) and brine (2×30 mL), dried over Na₂SO₄, and concentrated in vacuo to give a crude product. The crude product was purified by column chromatography (SiO₂, Petroleum ether/Ethyl acetate=40/1 to 20:1, 10% THF) to give the title compound (13.8 g, 74% yield) as a yellow solid.

### Step 2: (S)-8-iodo-N-(1-methoxypropan-2-yl)-5-((tetrahydro-2H-pyran-4-yl)oxy)pyrido[4,3-d]pyrimidin-2-amine

To a solution of 5-chloro-8-iodo-N-[(1S)-2-methoxy-1-methyl-ethyl]pyrido[4,3-d]pyrimidin-2-amine (10 g, 26 mmol, 1 eq) in 2-Me-THF (150 mL) was added tetrahydropyran-4-ol (5.4 g, 53 mmol, 5.3 mL, 2 eq) and t-BuOK (4.5 g, 40 mmol, 1.5 eq). The mixture was stirred at 90° C. for 1 hour. On completion, the mixture was diluted with ethyl acetate (300 mL) and washed with water (50 mL). The aqueous layer was extracted with ethyl acetate (2×30 mL). The combined organic layer was washed with sat. NH₄Cl (50 mL), brine (2×30 mL), dried over Na₂SO₄ and concentrated in vacuo. The residue was purified by column chromatography (SiO₂, Petroleum ether/Ethyl acetate=50:1/40:1, 10% THF) to give the title compound (10.2 g, 86% yield) as a yellow solid.

### Step 3: (S)-3-(2-((1-methoxypropan-2-yl)amino)-5-((tetrahydro-2H-pyran-4-yl)oxy)pyrido[4,3-d]pyrimidin-8-yl)-N,N-dimethylbenzenesulfonamide

A solution of 8-iodo-N-[(1S)-2-methoxy-1-methyl-ethyl]-5-tetrahydropyran-4-yloxy-pyrido [4,3-d]pyrimidin-2-amine (100 mg, 225 umol, 1 eq), N,N-dimethyl-3-(4,4,5,5-tetramethyl-1,3,2-dioxaborolan-2-yl)benzenesulfonamide (106 mg, crude), K₃PO₄ (143 mg, 675 umol, 3 eq), Pd(OAc)₂ (5 mg, 23 umol, 0.1 eq) and bis(1-adamantyl)-butylphosphane (8 mg, 23 umol, 0.1 eq) in n-BuOH (2 mL) was degassed and purged with N₂ 3 times, and then the mixture was stirred at 50° C. for 16 hours under N₂ atmosphere. On completion, the mixture was diluted with ethyl acetate (20 mL) and water (30 mL). The mixture was extracted with ethyl acetate (2×30 mL). The combined organic layer was washed with water (30 mL) and brine (2×30 mL), dried over Na₂SO₄ and concentrated in vacuo. The residue was purified by prep-HPLC. The eluent was concentrated to remove organic solvent, and the aqueous solution was lyophilized to give the title compound (33.6 mg, 30% yield) as a white solid. The title compound was confirmed by ¹H NMR and LCMS.

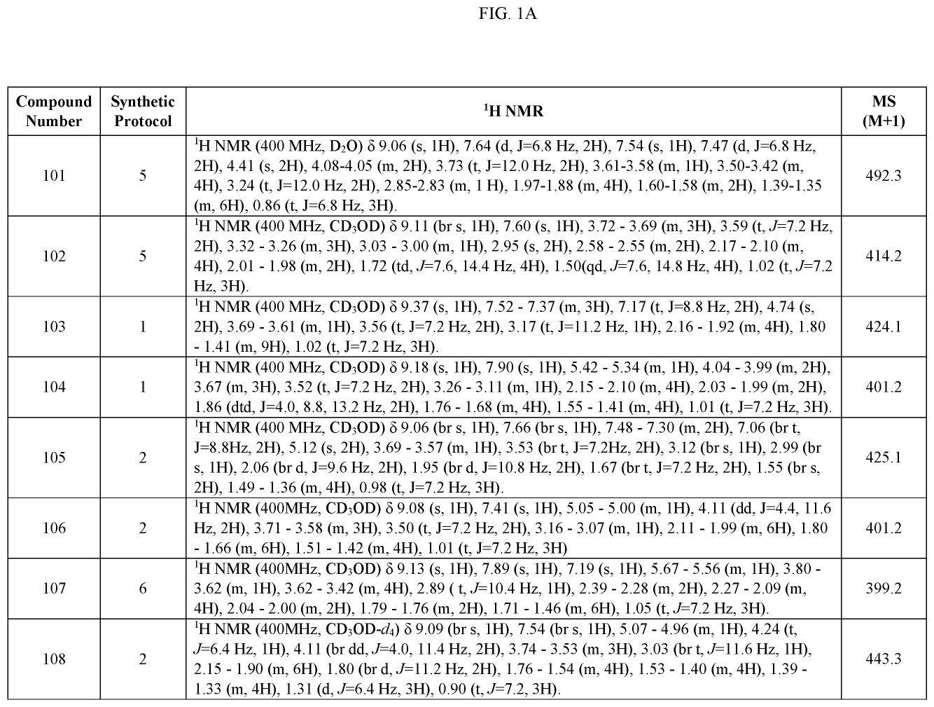

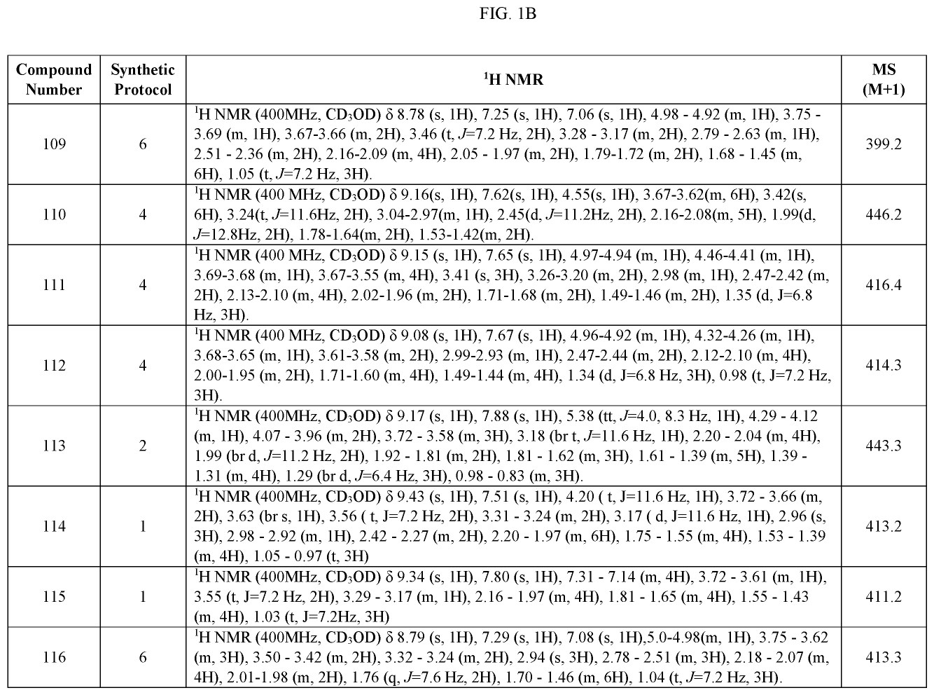

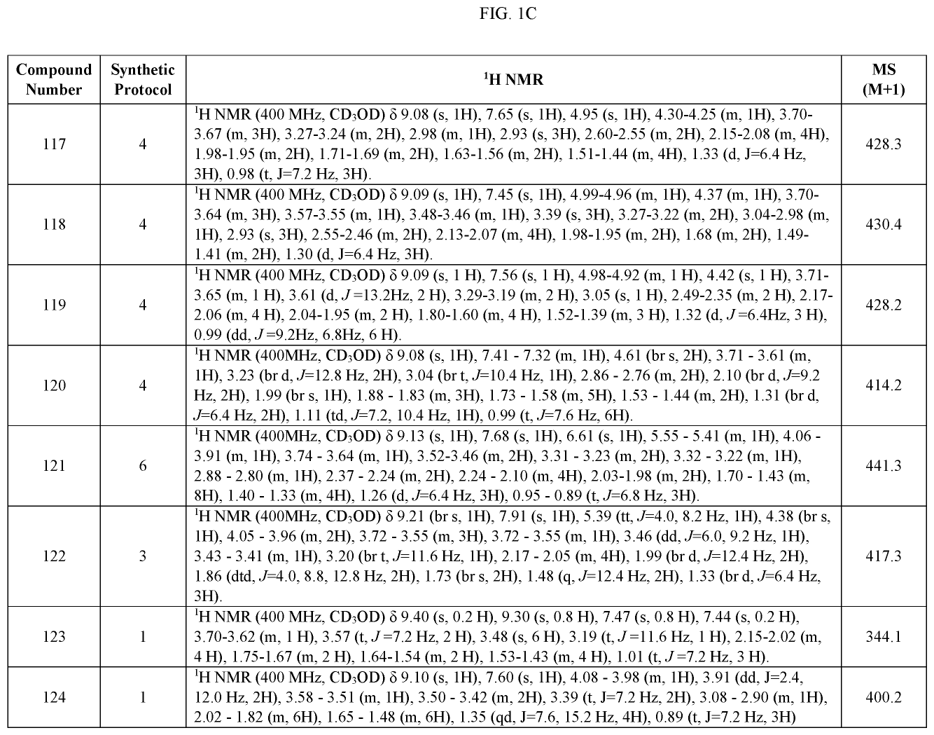

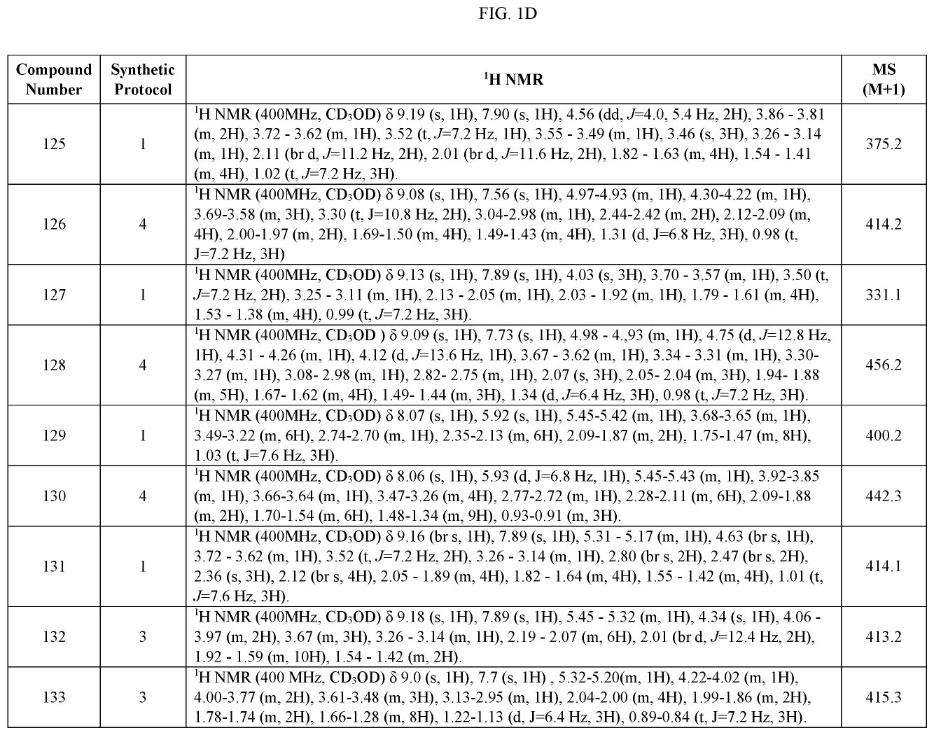

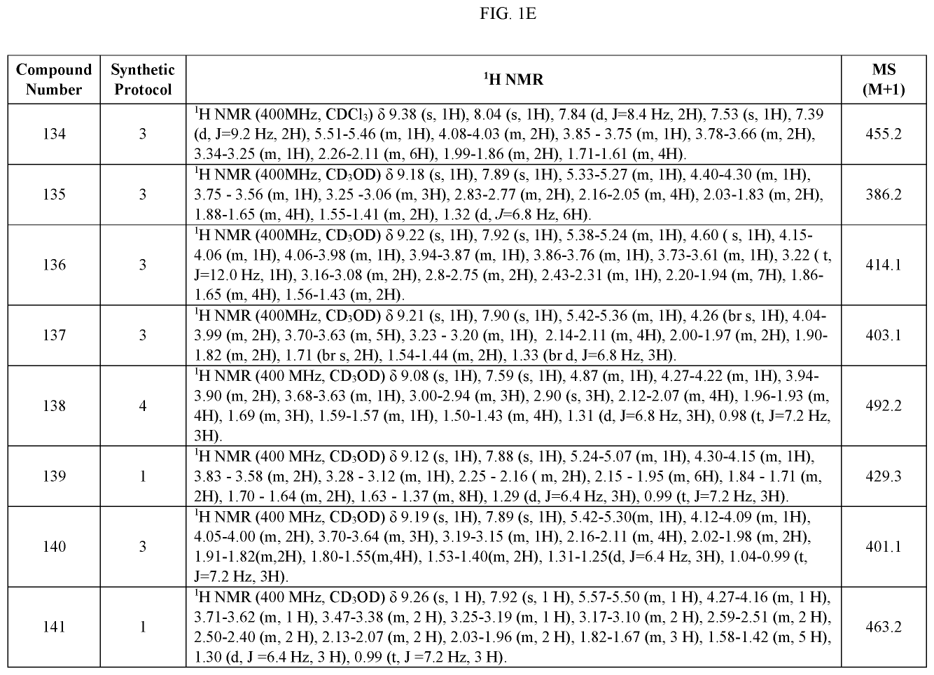

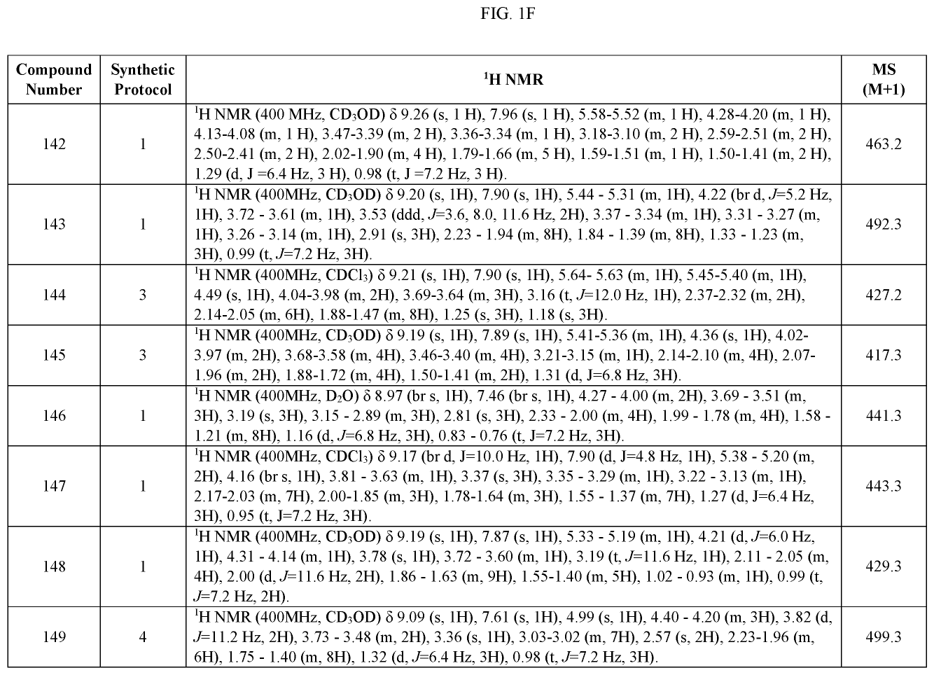

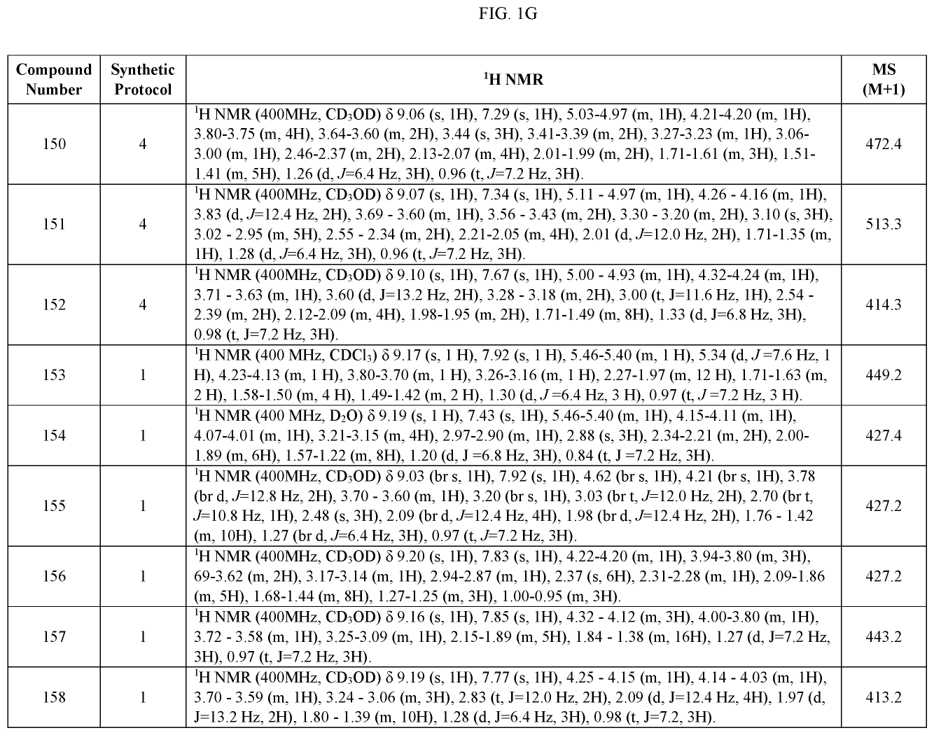

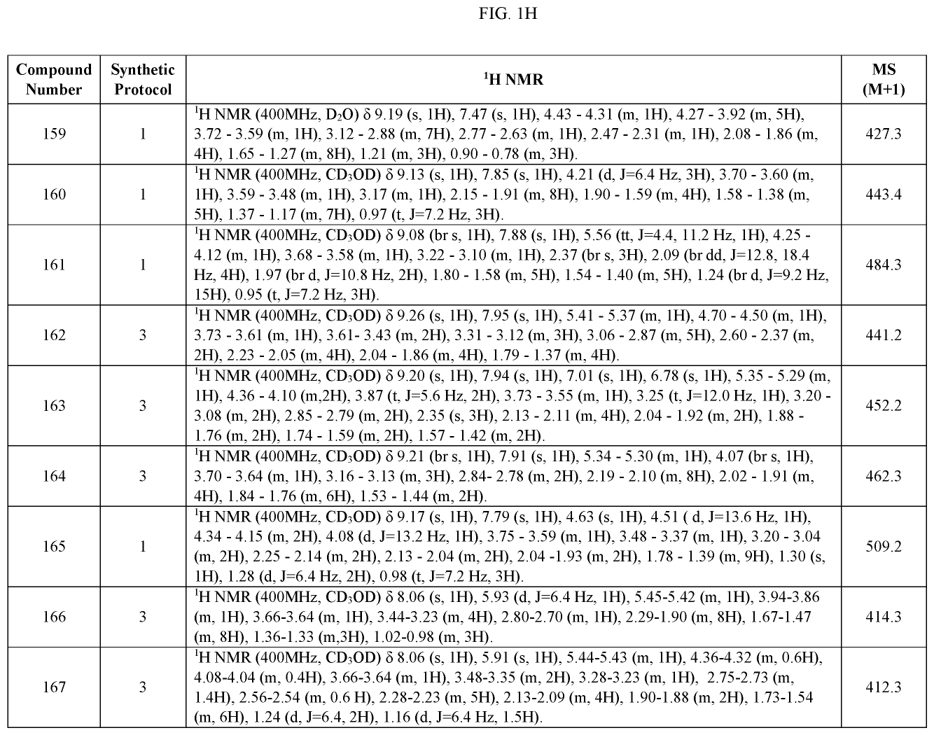

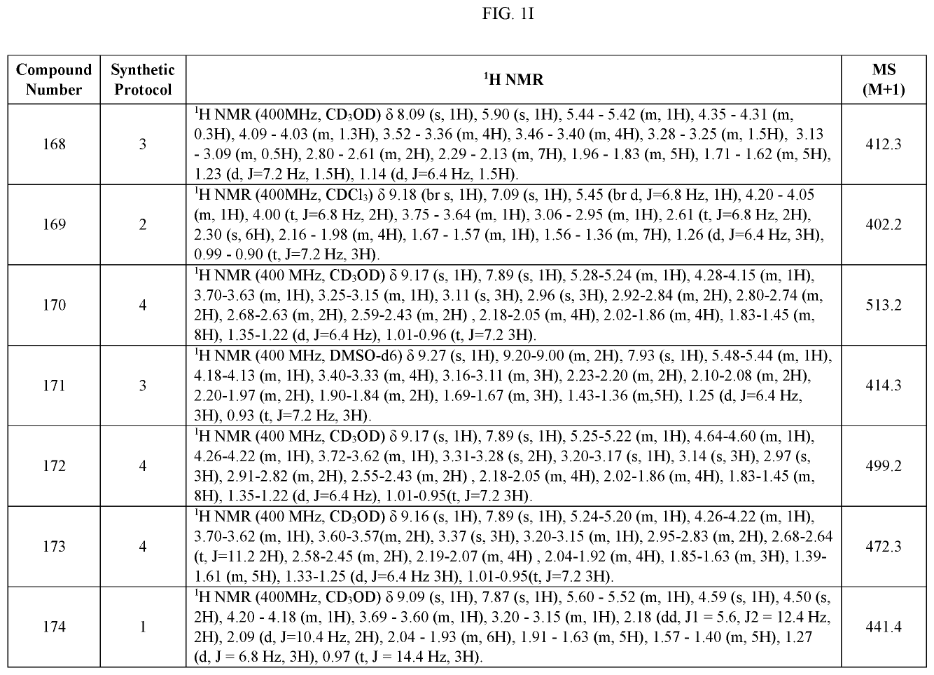

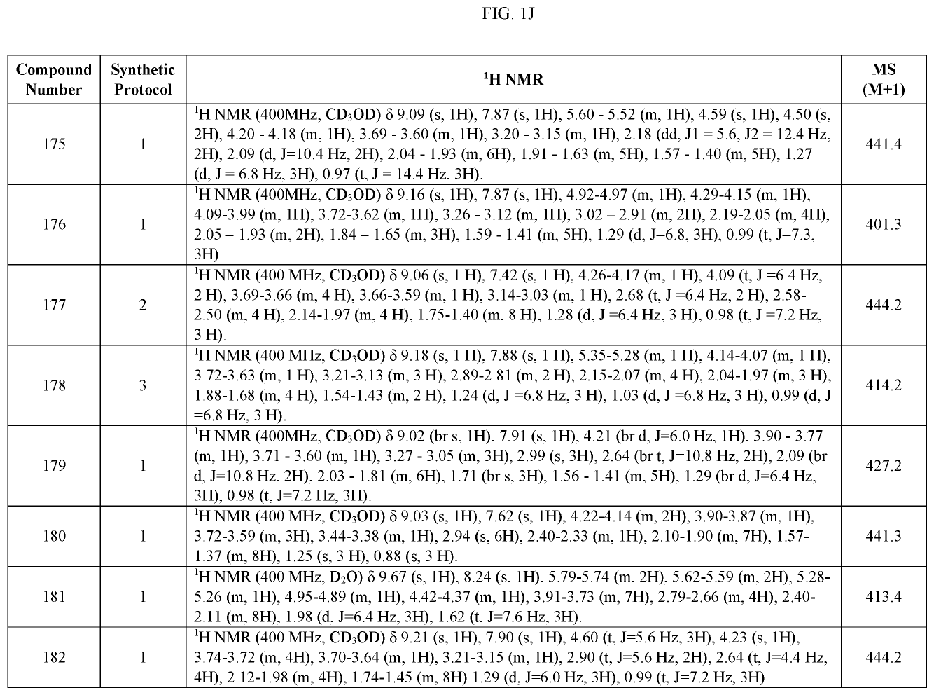

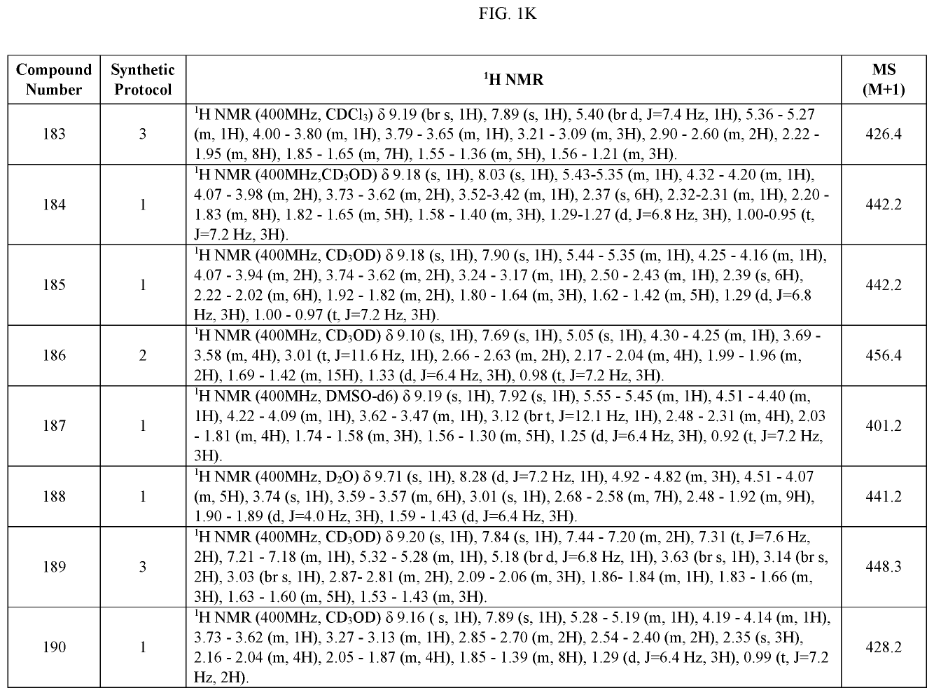

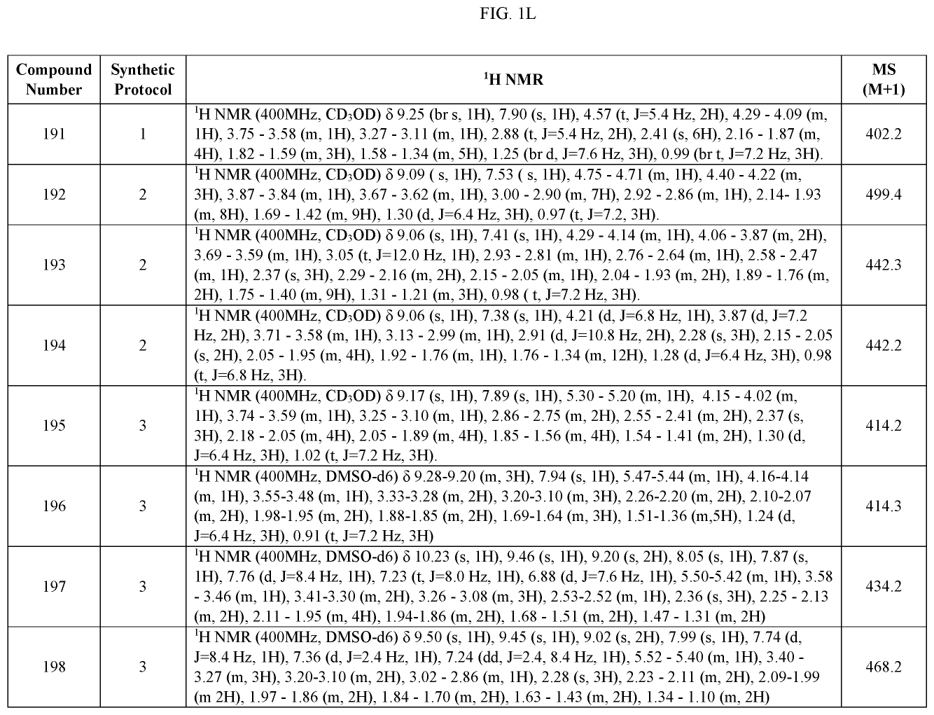

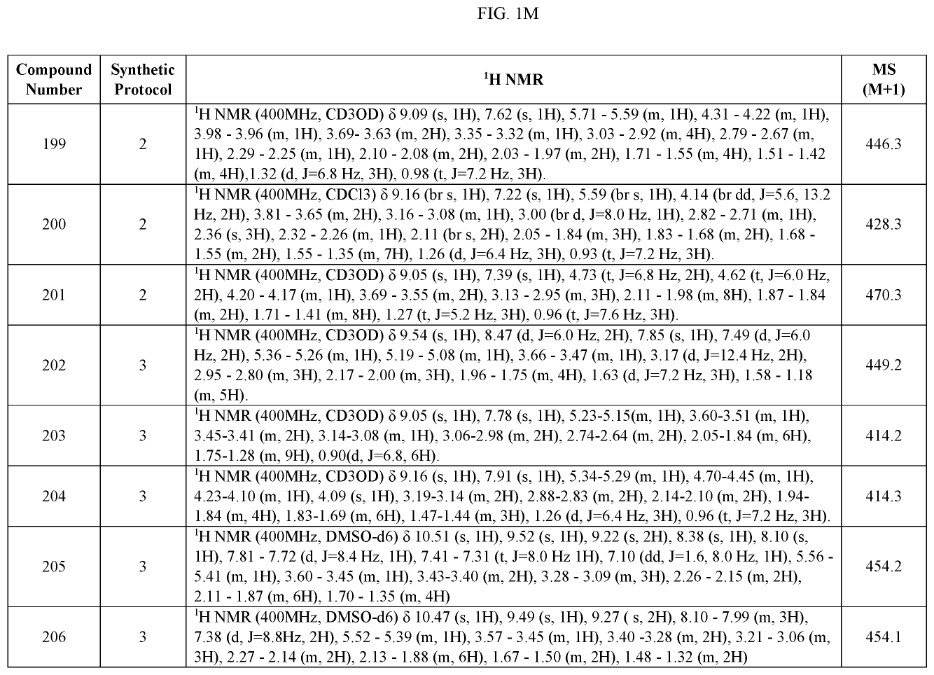

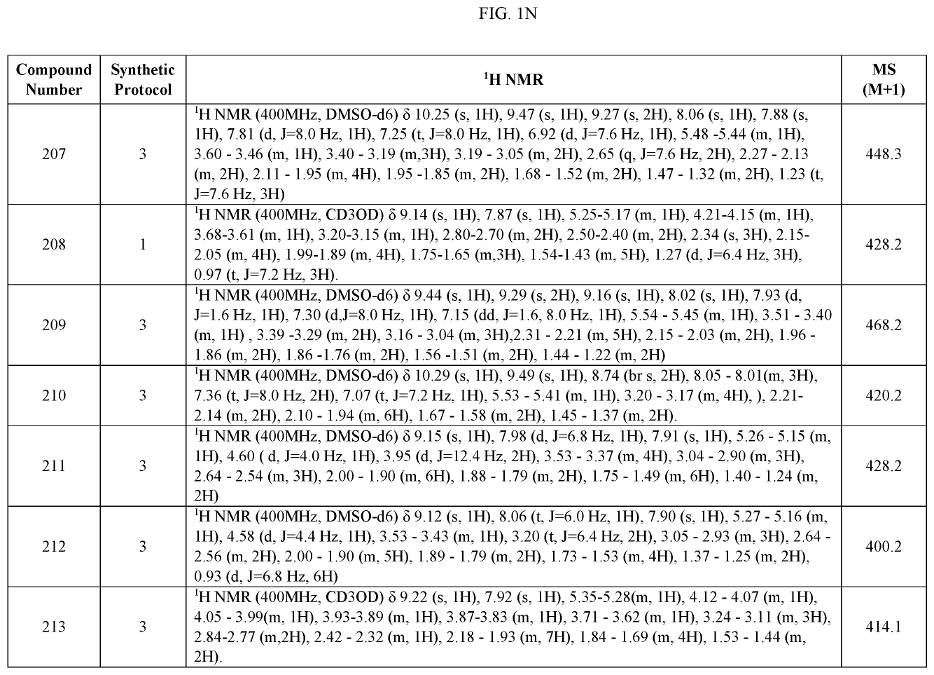

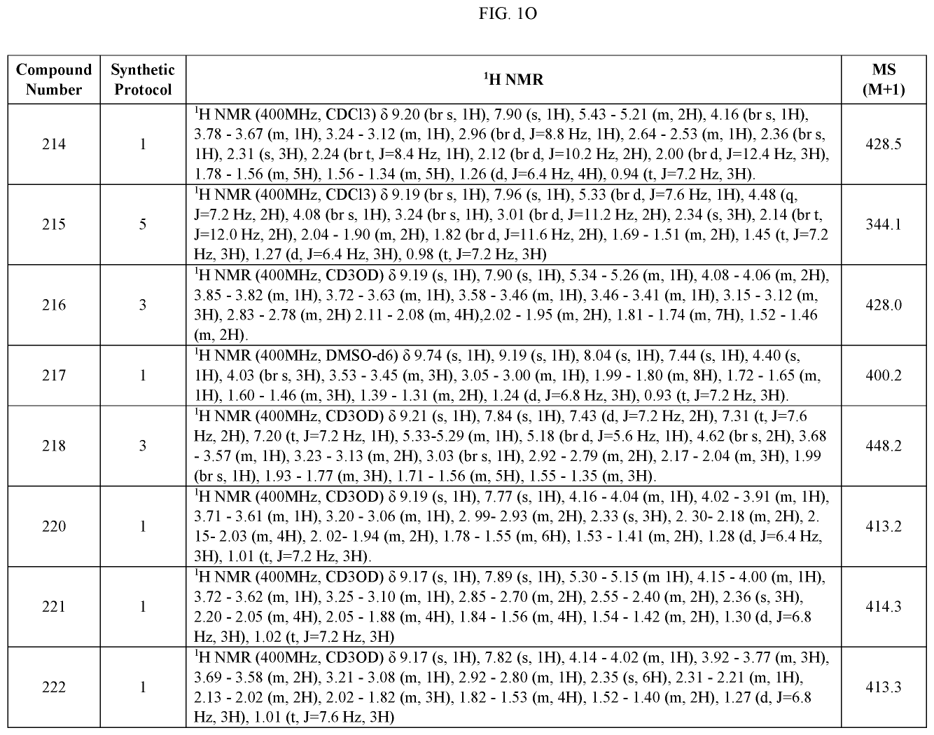

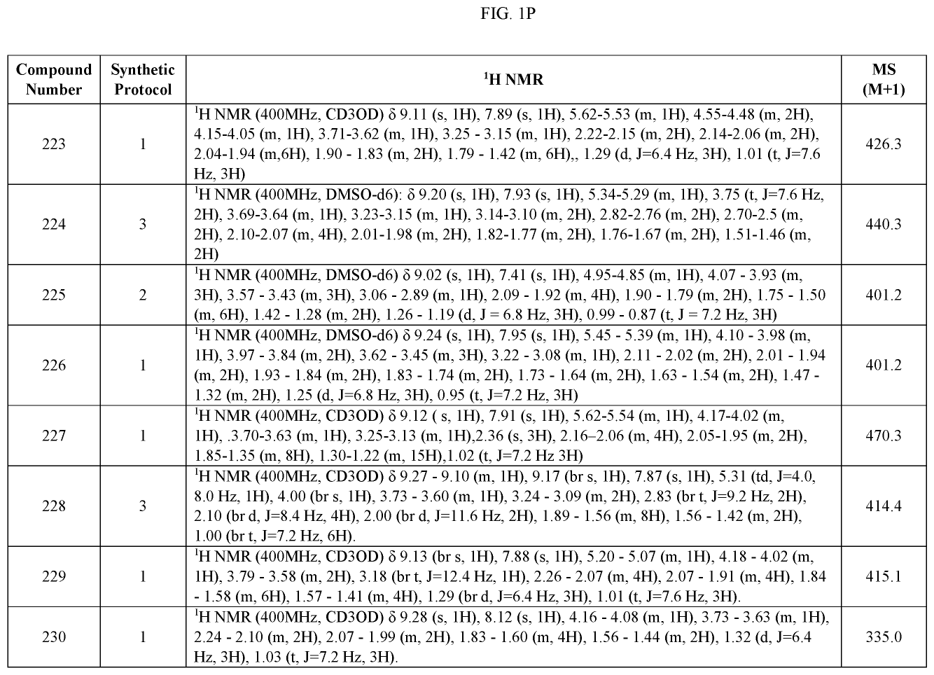

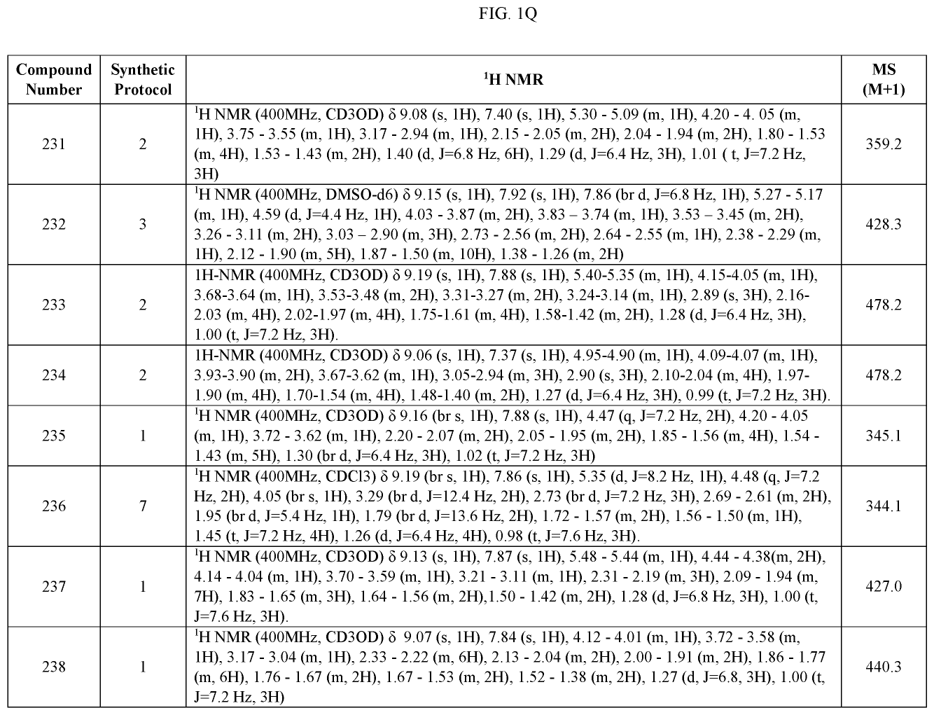

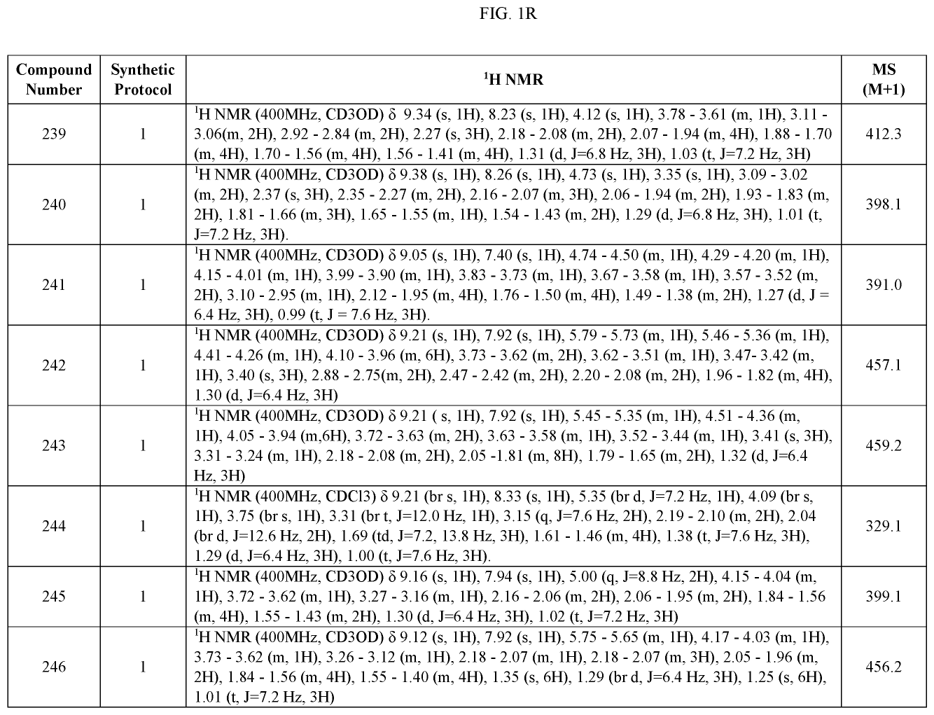

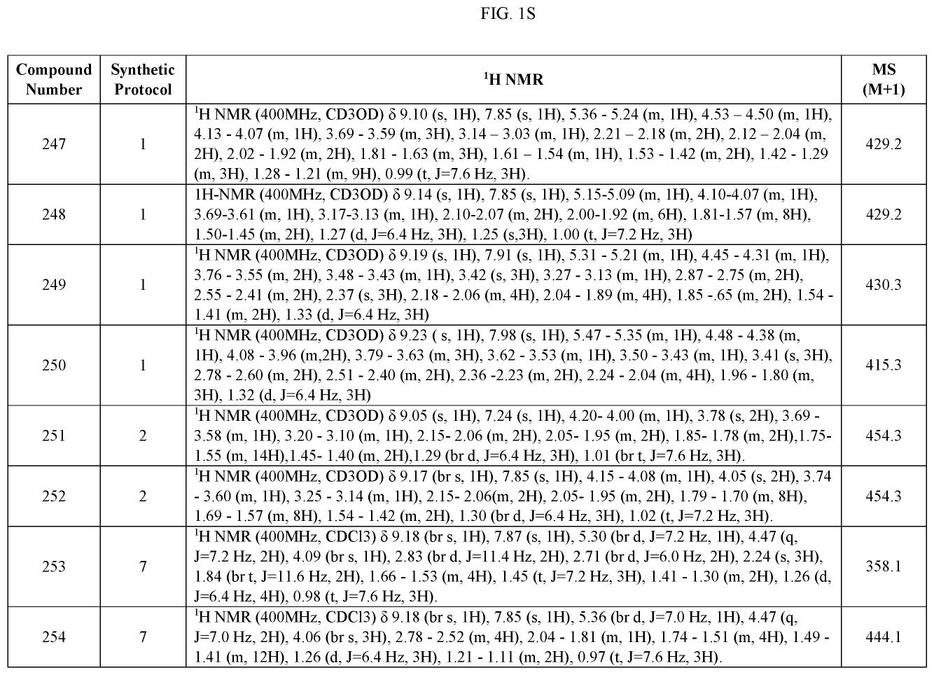

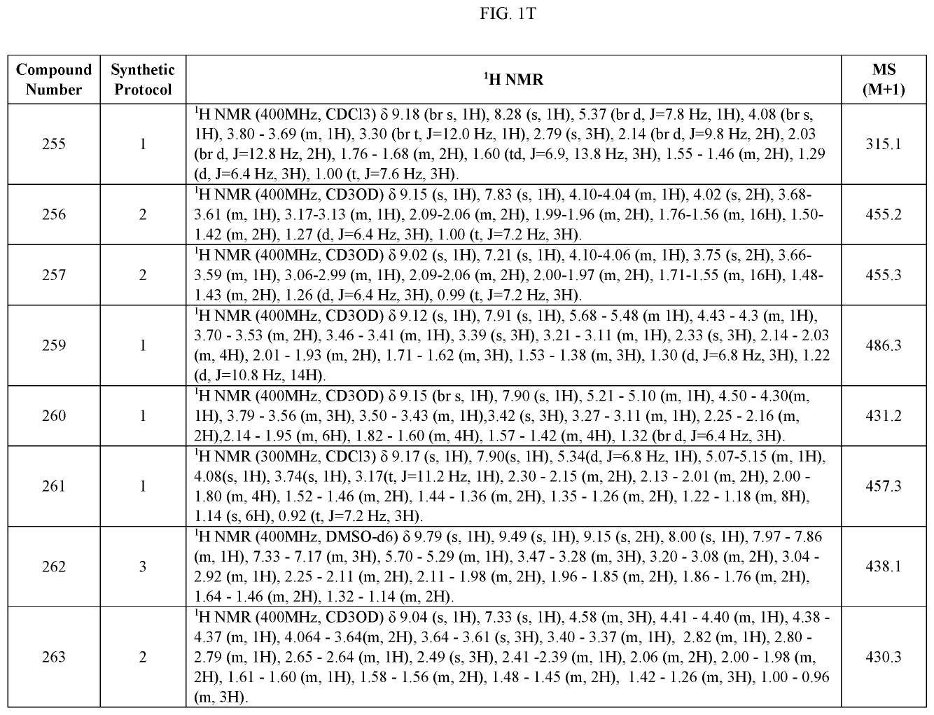

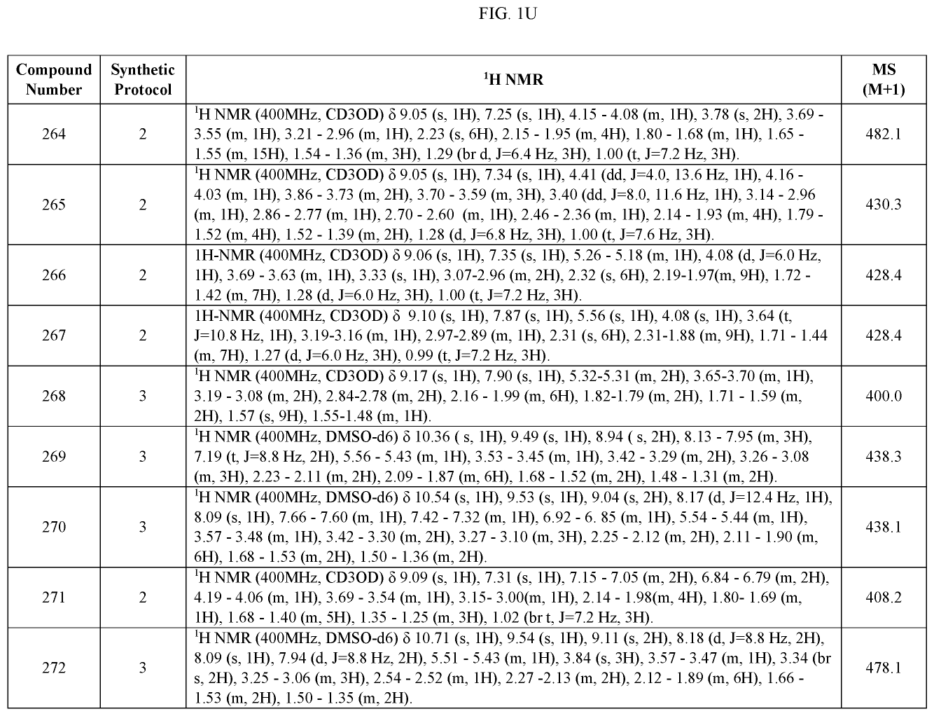

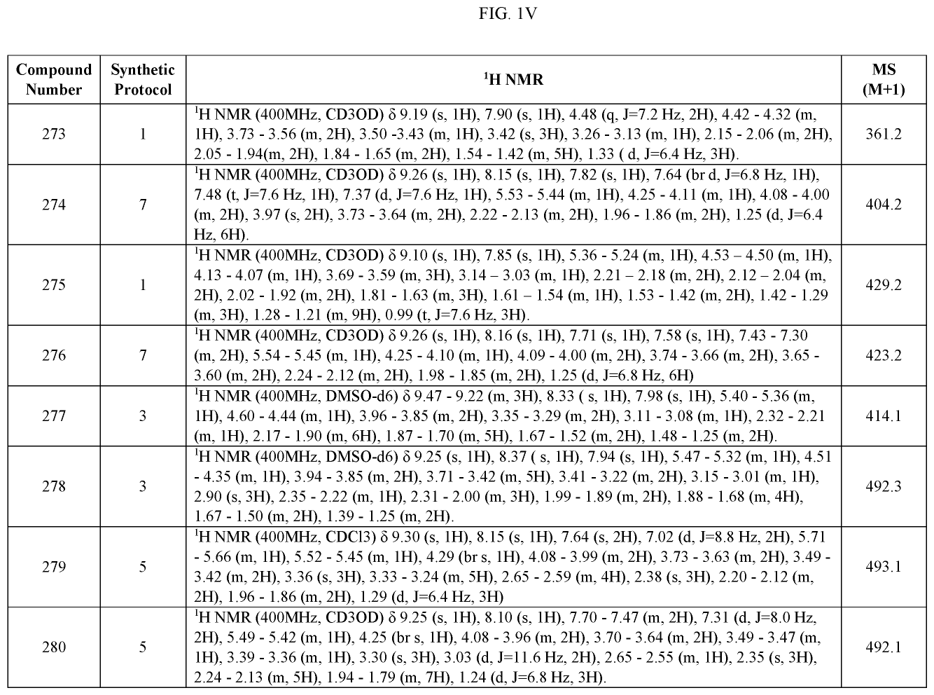

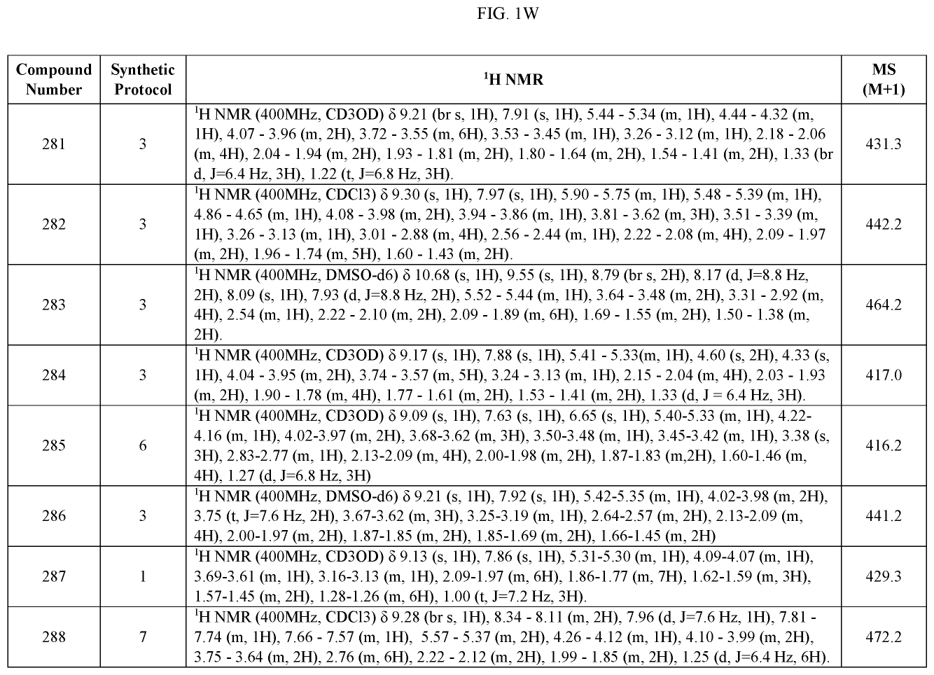

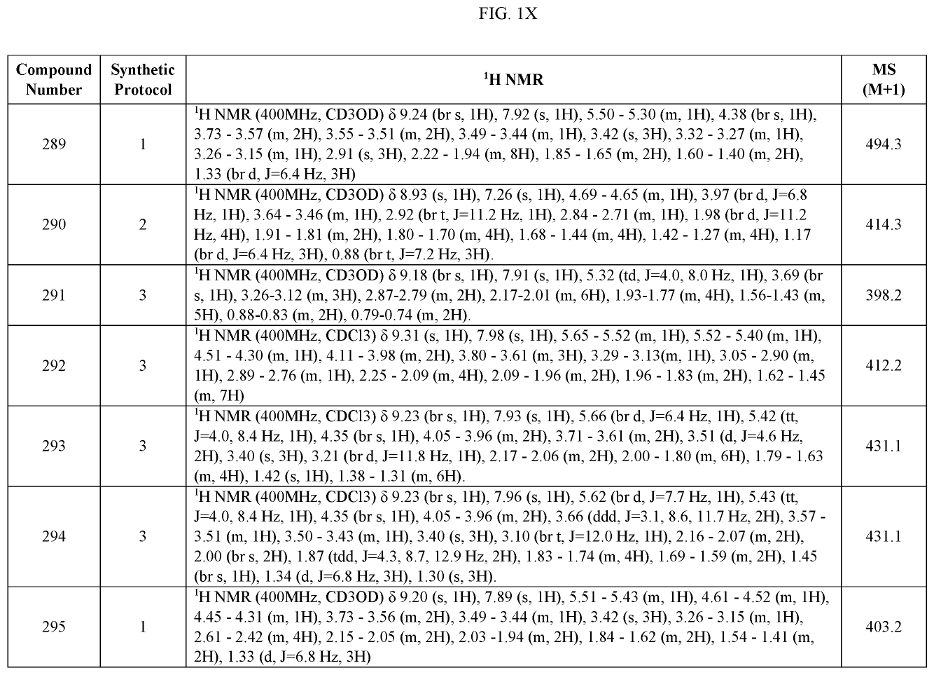

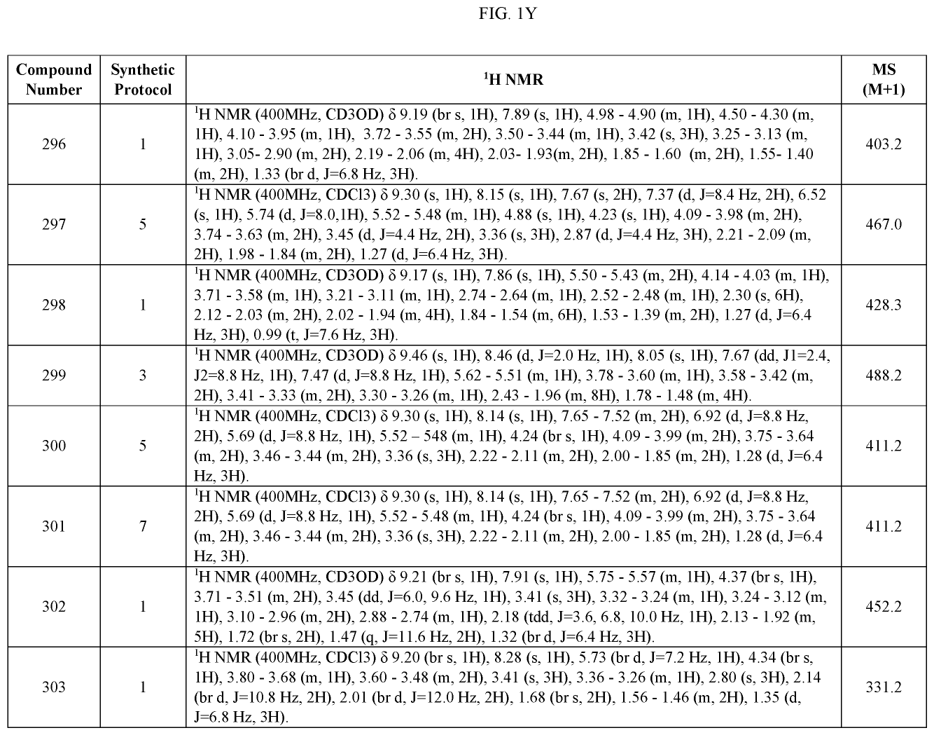

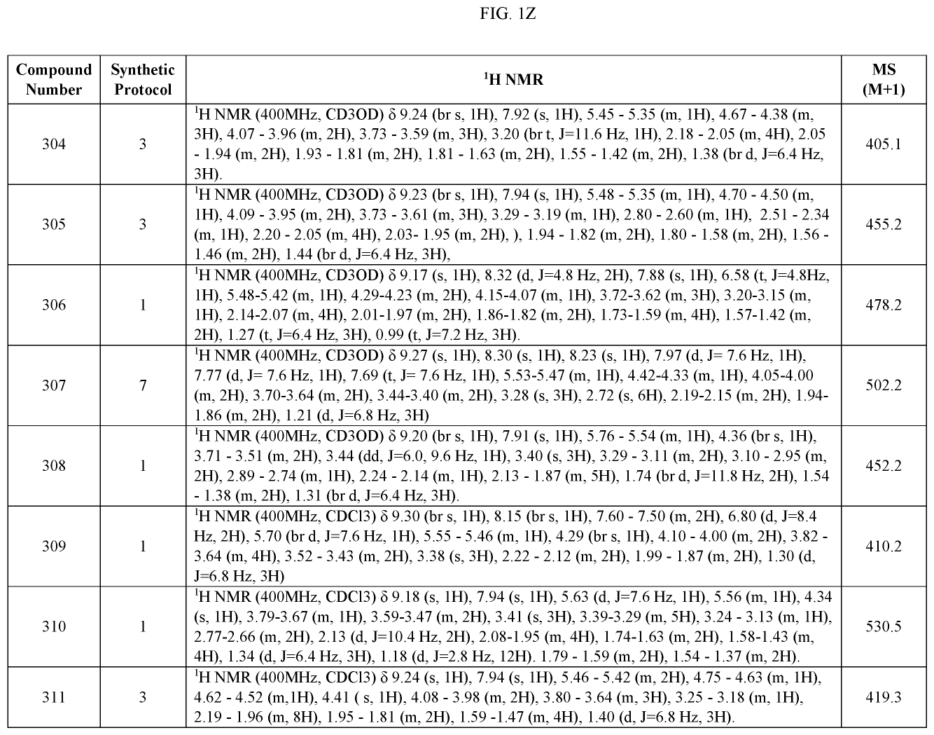

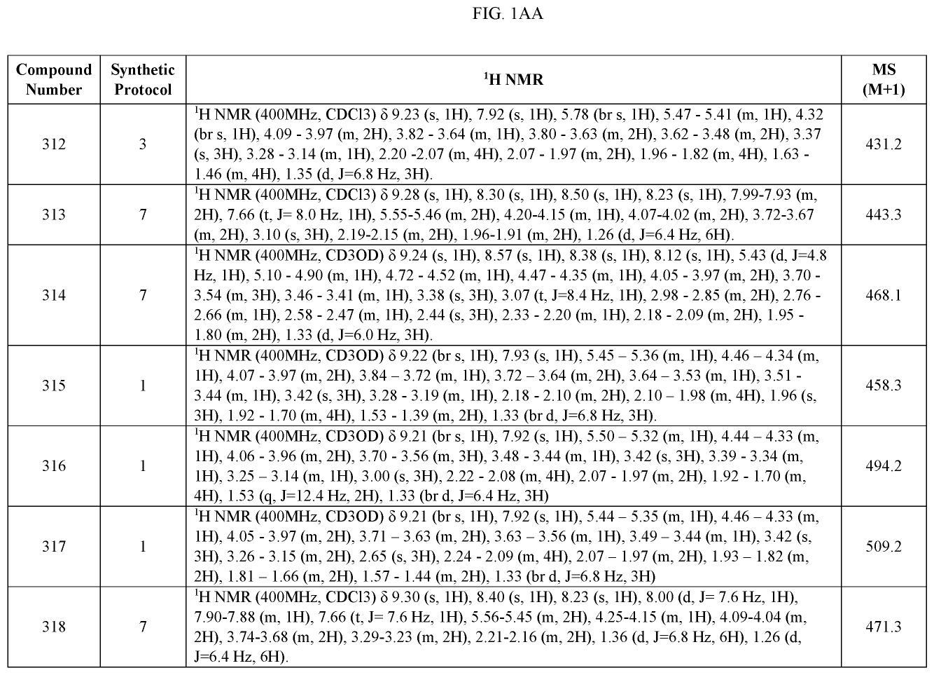

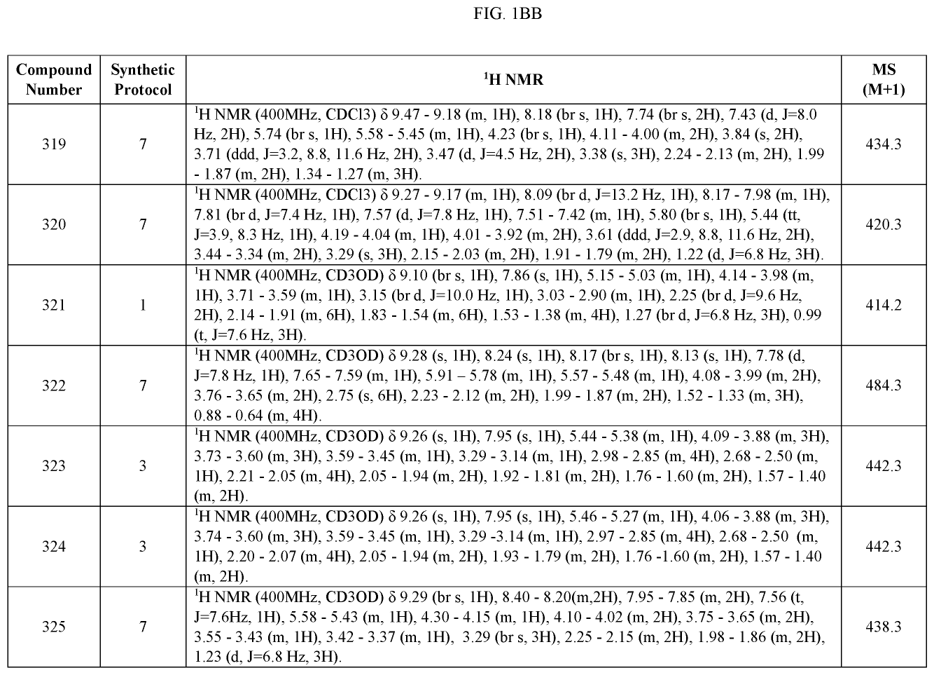

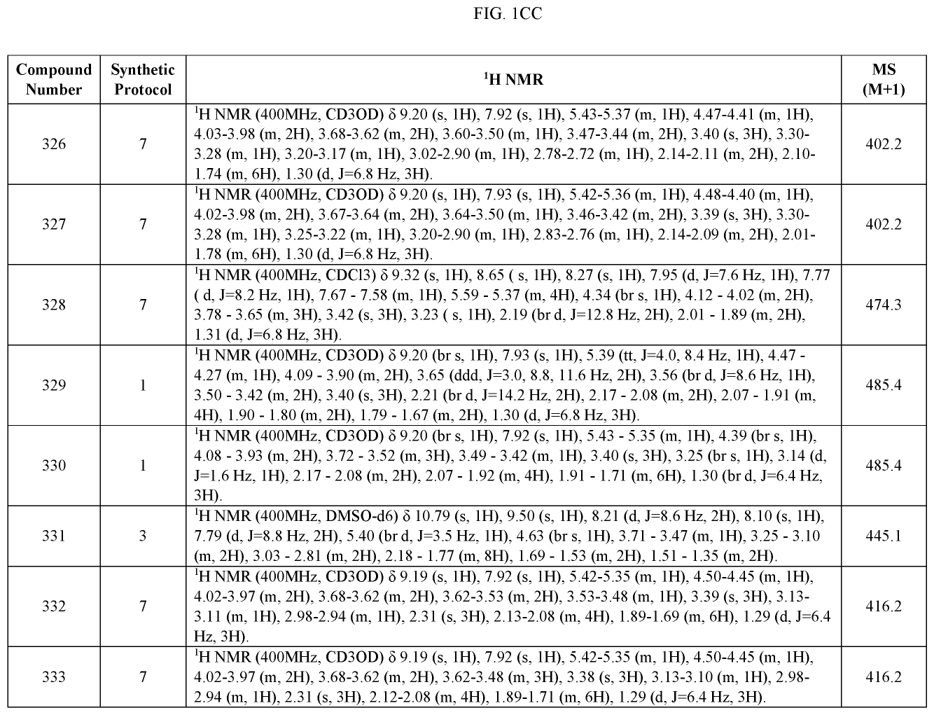

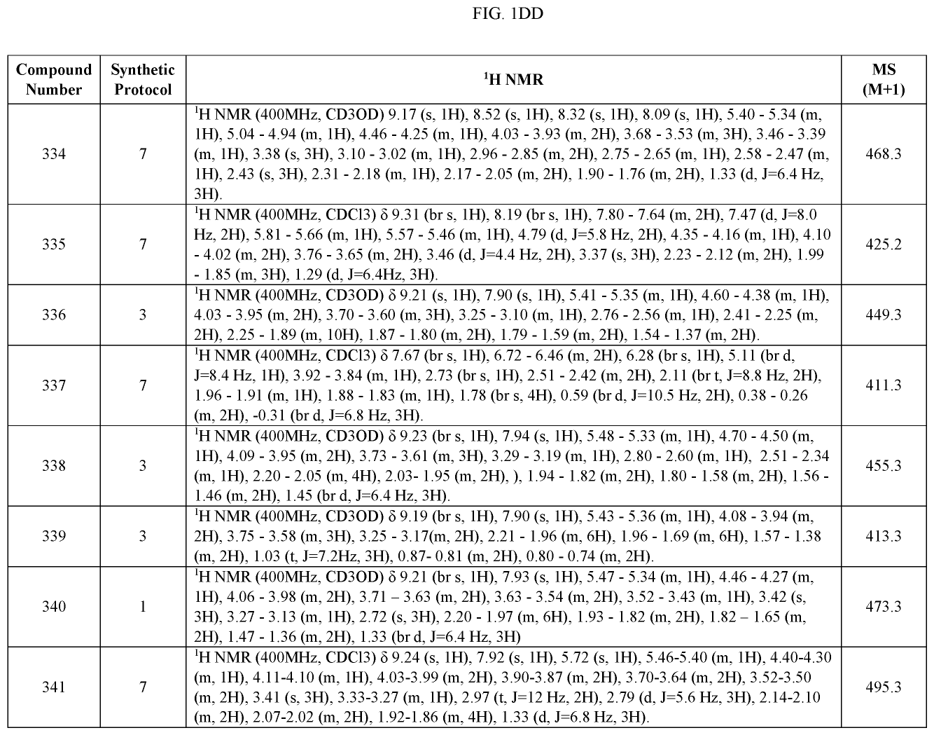

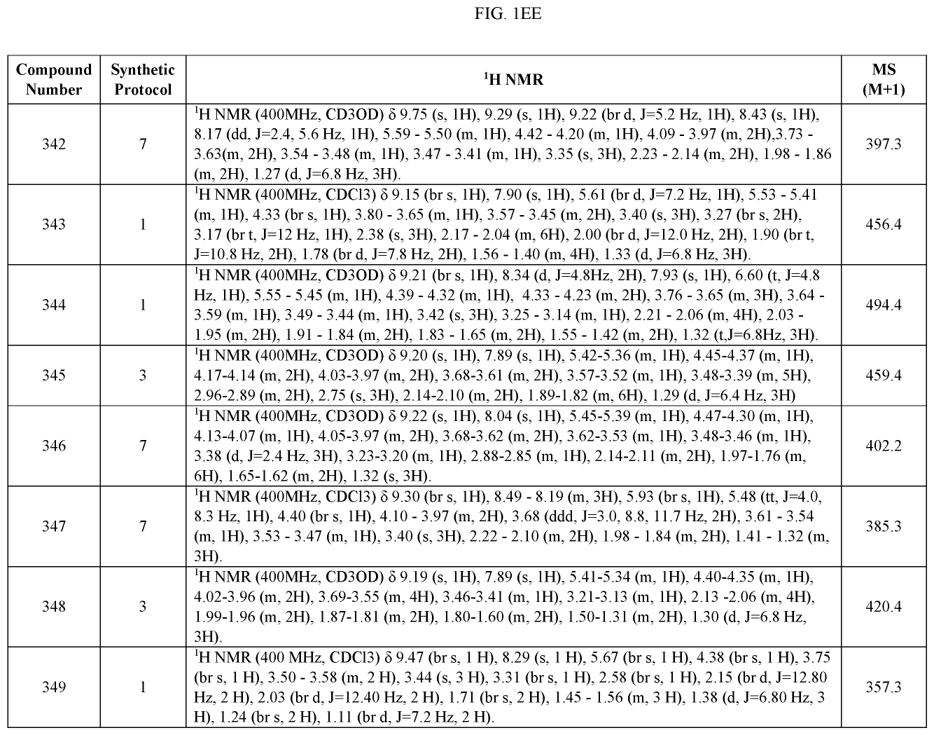

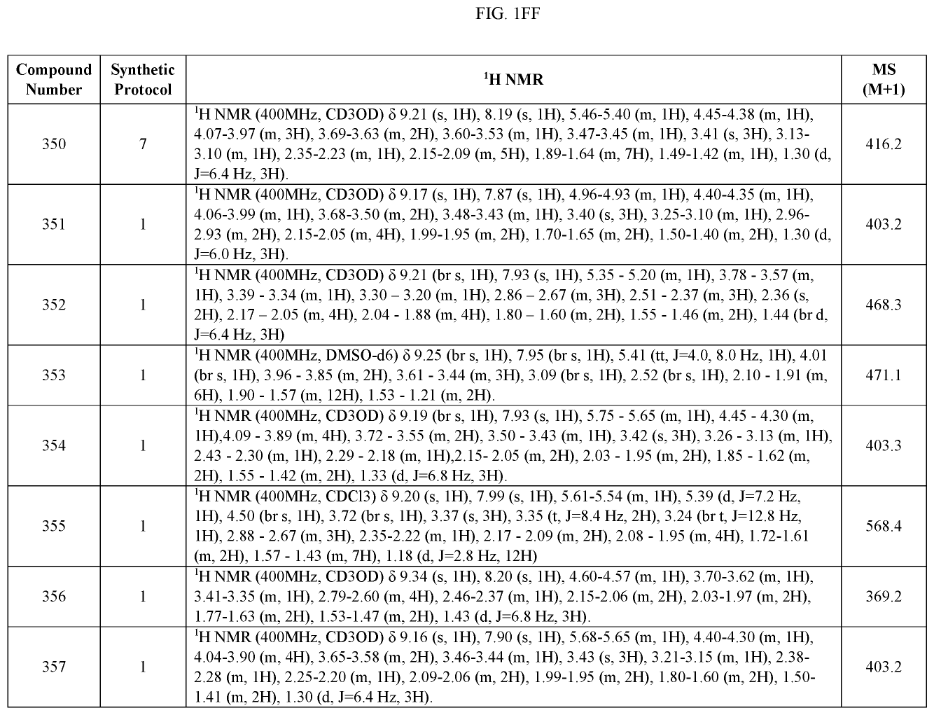

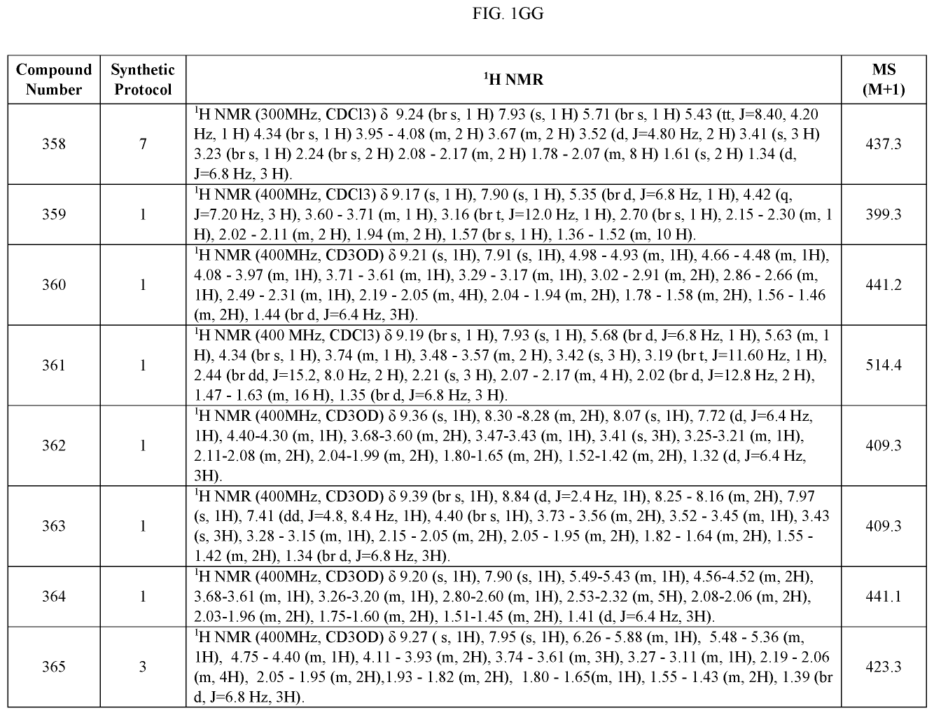

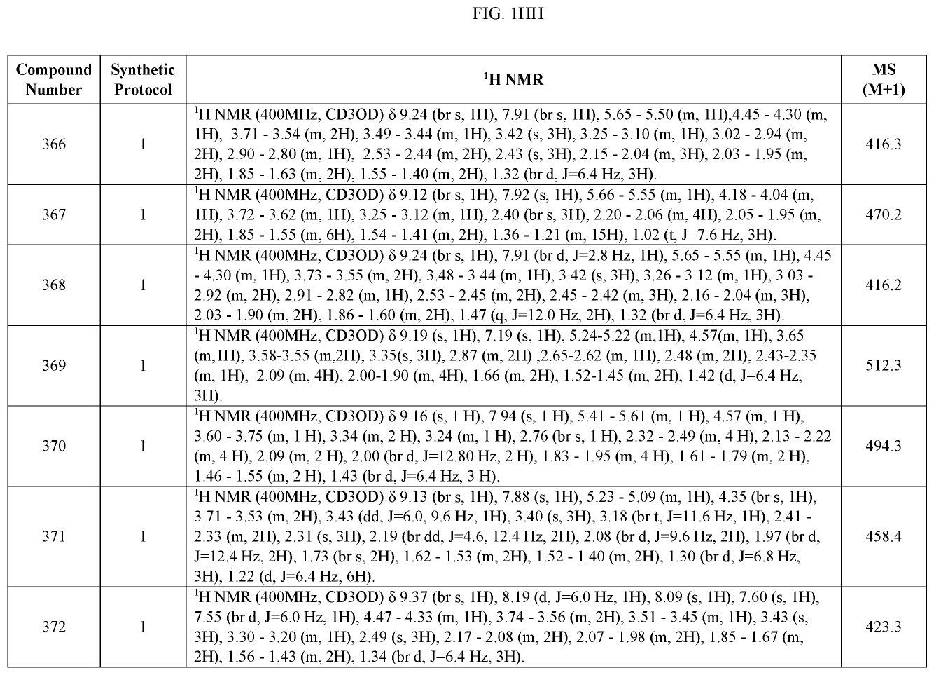

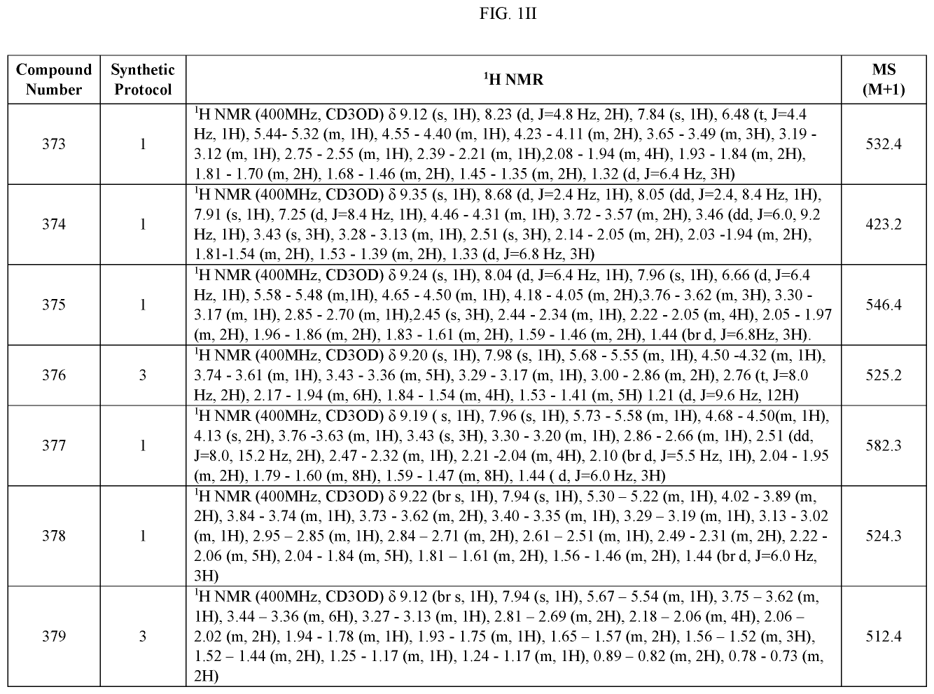

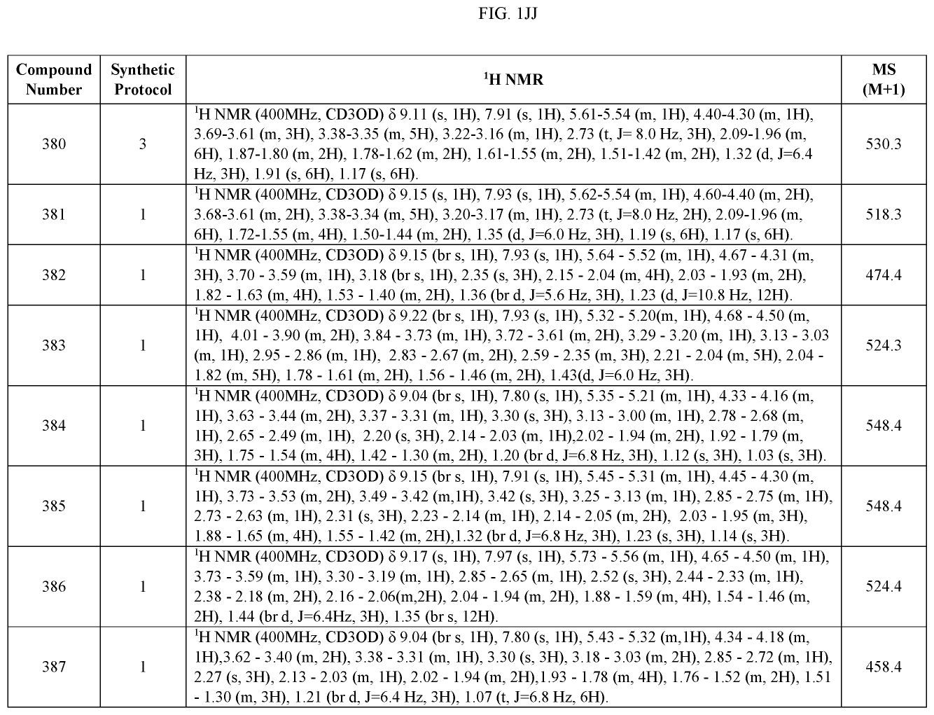

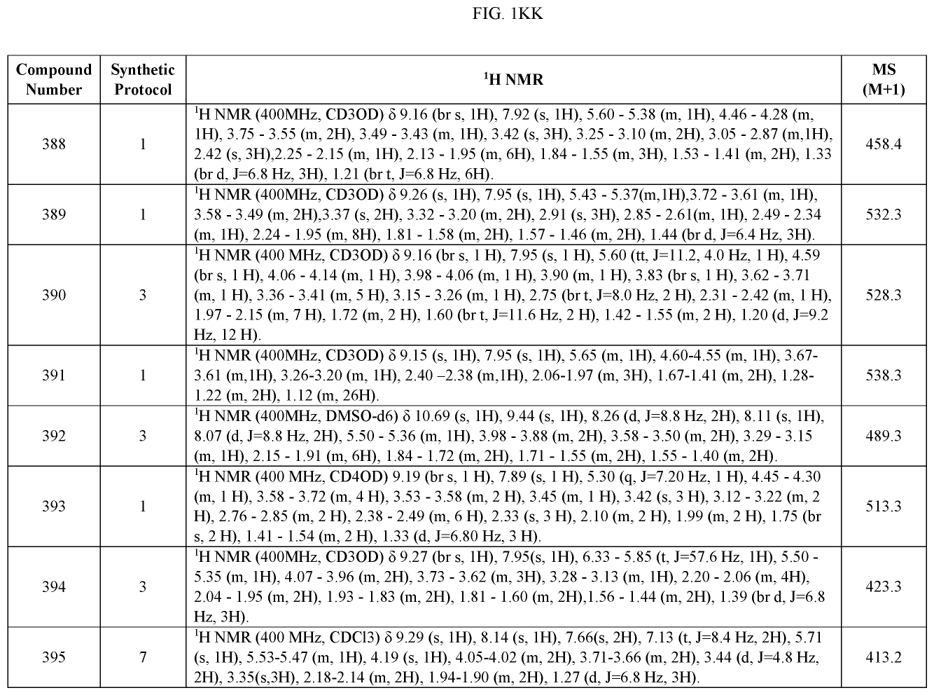

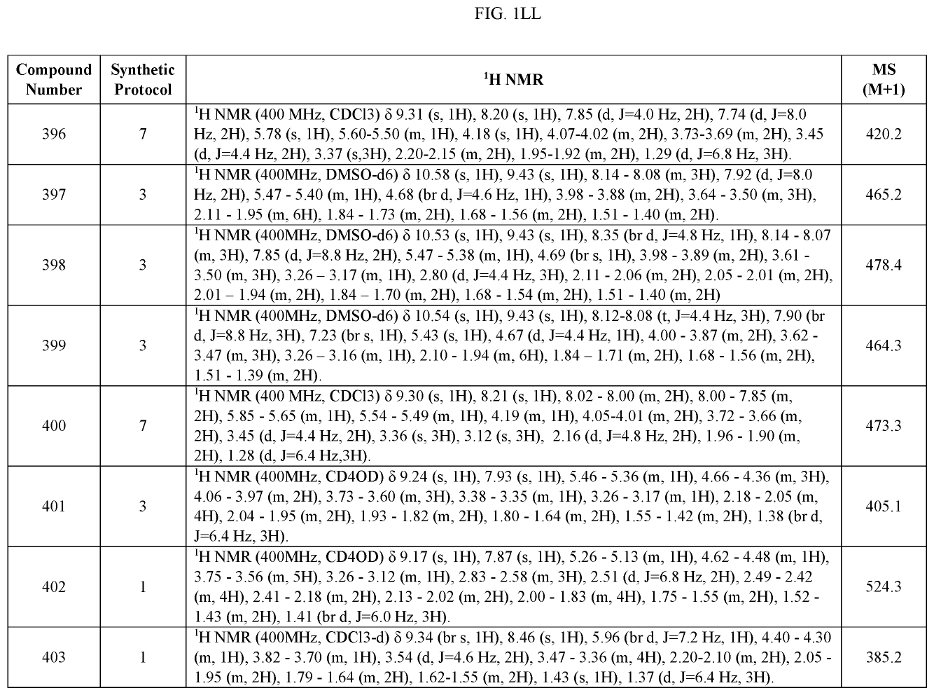

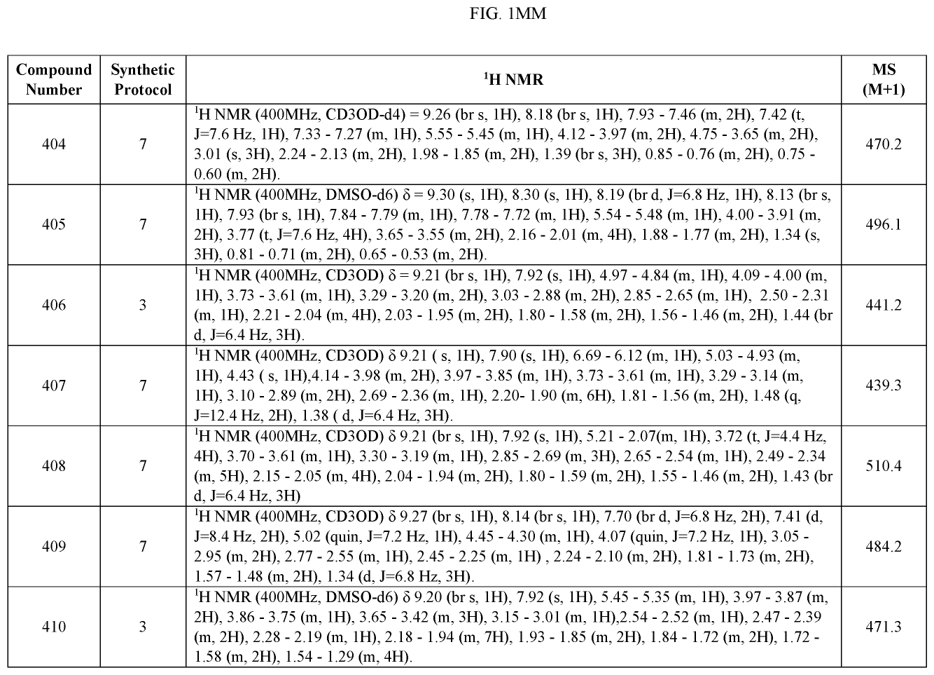

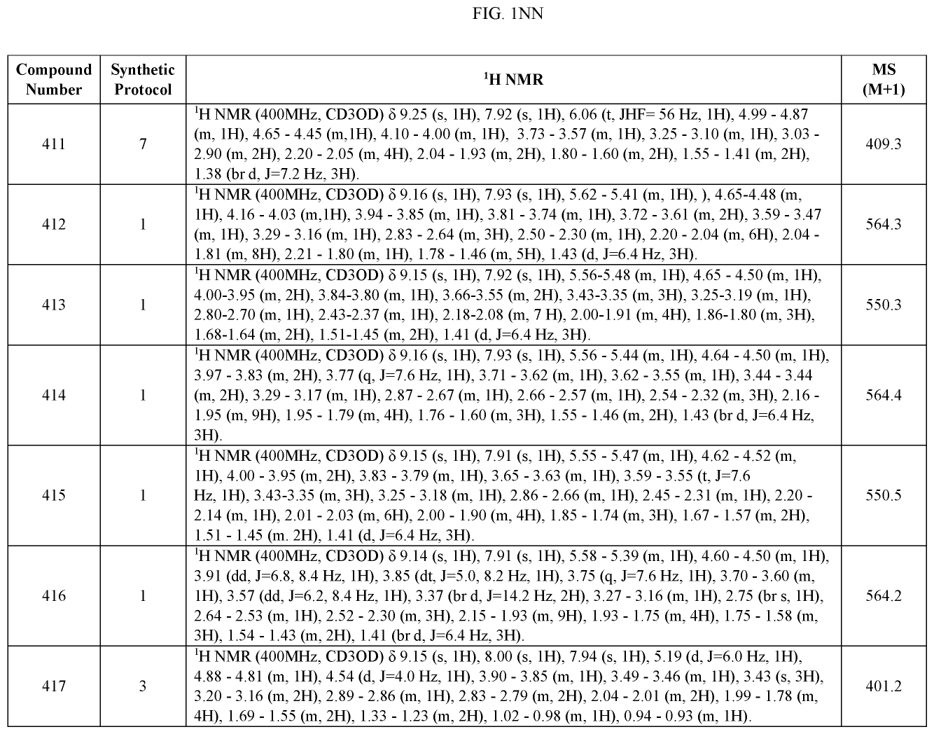

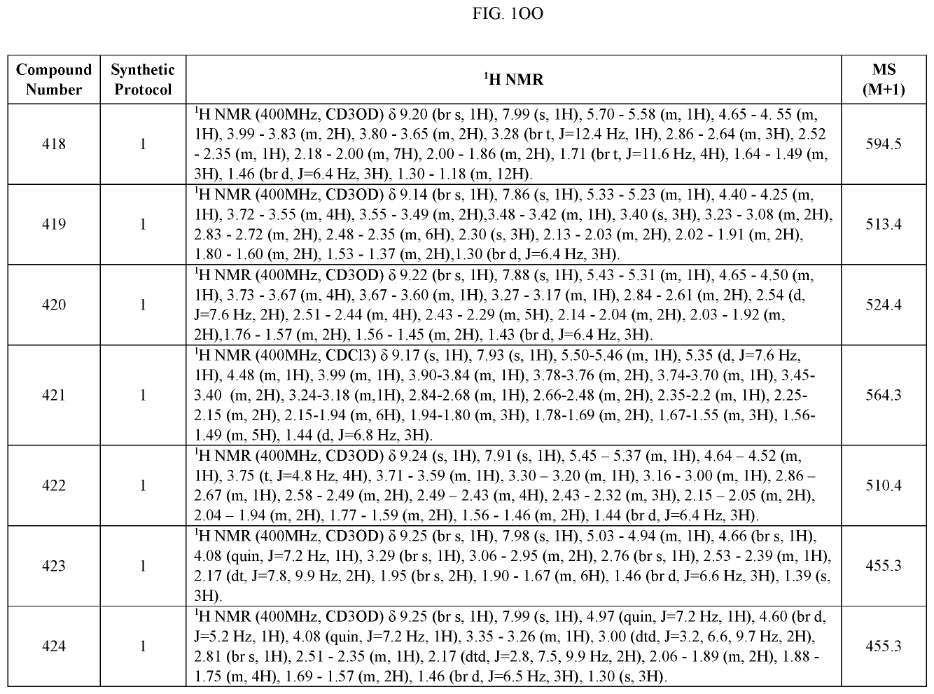

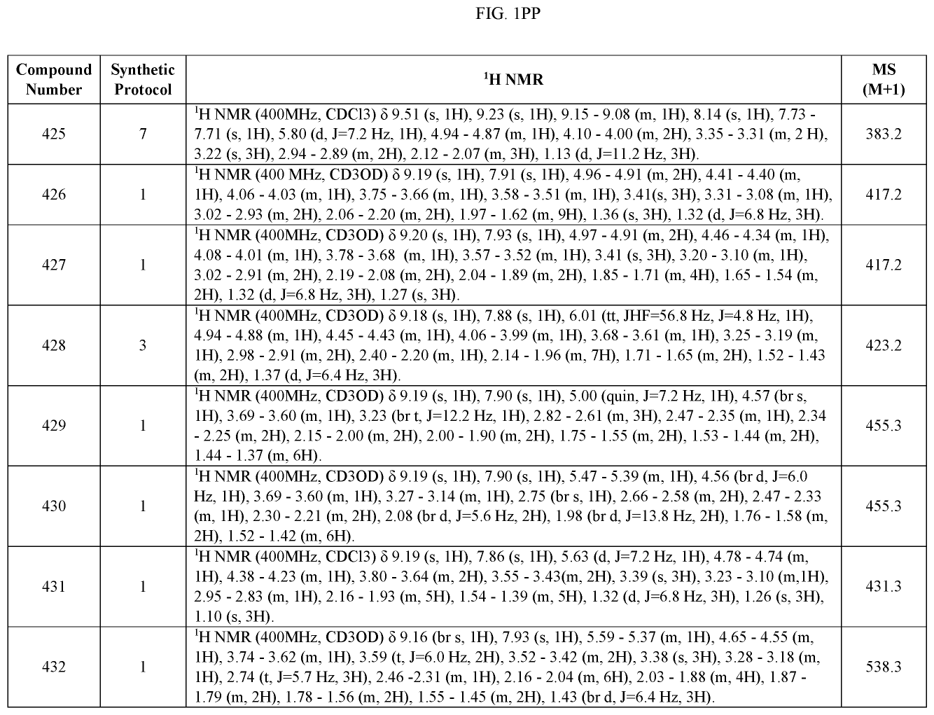

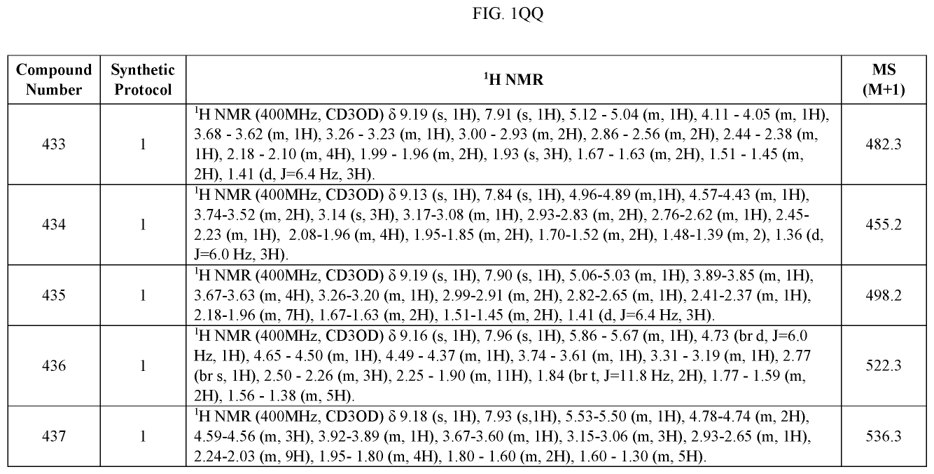

The synthetic protocols and intermediates described above were used to prepare other compounds disclosed herein as indicated below and further described in the Table of FIG. 1. The NMR and LCMS data obtained for these compounds are shown. One of ordinary skill in the art will be readily able to make other compounds of the invention based on the general synthesis schemes, intermediate synthesis protocols and specific compound synthesis protocols set forth above. The Table disclosed herein as FIG. 1, provides the physiochemical properties of exemplary compounds (Compound Nos. 101-218, 220-257, and 259-437) and the protocol number by which they were made.

### Example 12: Kinase Activity Assay

IC₅₀ values for compound inhibition of AXL, FLT3, MERTK and TYRO3 activity were determined using a TR-FRET activity assay. A peptide substrate with a fluorescent label is phosphorylated on a tyrosine by each enzyme, where the now phosphorylated product is bound by a Europium-labeled antibody specific to that phosphorylation site. The proximity between the antibody and the substrate gives a signal known as TR-FRET. As the enzyme is inhibited by compound, less phosphorylated peptide product is made, causing a decrease in overall signal.

Activity assays were performed in a 384-well, small volume, black microplates in an active enzyme volume of 10 uL and final developed volume of 20 uL. Final assay conditions are 50 mM HEPES pH 7.5, 10 mM MgCl₂, 1 mM EGTA, 0.01% Brij 35 and 1 mM DTT, all at RT. Enzyme, substrate-labeled peptide and compound are mixed, and then the reaction is initiated with the addition of ATP at Km concentration for each enzyme. The reaction is allowed to run 60 minutes and then quenched with a 12.5 mM EDTA (ethylenediamine tetraacetic acid) solution. The plate is then developed with the addition of Europium-labeled antibody for a final volume of 20 uL and is then incubated for 30 minutes in darkness. Plates are read in a PHERASTAR® plate reader. Data are analyzed in GraphPad PRISM.

TABLE 2
Assay conditions for TR-FRET Activity

| | Kinase | Labelled | ATP final |
|---|---|---|---|
| Kinase | [pM] | peptide [nM] | conc [μM] |
| AXL | 200 | 50 | 87 |
| FLT3 | 125 | 50 | 140 |
| MERTK | 20 | 50 | 19 |
| TYRO3 | 30 | 50 | 53 |

The results of these assays are shown in Table 3, below, where “A” represents a value of ≤100 nM; “B” a value of >100 nM and ≤1 μM; “C” a value of >1 μM and ≤10 μM; “D” a value of >10 μM; and “ND” is not determined. The ratio of Mer to FLT3 is calculated as follows: FLT3 IC₅₀/Mer IC₅₀. “+” represents a ratio of >2.0 and <10; “++” represents a ratio of between 10 and 100; “+++” represents a ratio of >100; and “NS” represent a ratio of ≤2.0.

TABLE 3
Activity of Exemplary Compounds against Various
Kinases and Mer:FLT3 Activity

| Compound | Axl | Flt3 | Mer | Tyro3 | Mer/Flt3 |
|---|---|---|---|---|---|
| 101 | B | B | A | B | + |
| 102 | B | A | A | A | + |
| 103 | B | A | A | A | + |
| 104 | A | A | A | A | + |
| 105 | B | B | A | ND | + |
| 106 | B | B | A | ND | + |
| 107 | A | A | A | A | ++ |
| 108 | B | C | A | A | +++ |
| 109 | B | B | A | A | ++ |
| 110 | C | C | C | ND | NS |
| 111 | B | C | A | ND | +++ |
| 112 | B | C | A | A | +++ |
| 113 | B | C | A | ND | +++ |
| 114 | B | A | A | ND | +++ |
| 115 | C | A | A | ND | + |
| 116 | B | B | A | A | + |
| 117 | B | C | A | A | +++ |
| 118 | B | C | B | ND | ++ |
| 119 | B | C | A | A | +++ |
| 120 | C | C | A | A | +++ |
| 121 | B | C | A | ND | +++ |
| 122 | B | C | A | B | +++ |
| 123 | B | A | C | ND | NS |
| 124 | A | A | A | A | + |
| 125 | B | A | A | C | NS |
| 126 | B | C | A | A | +++ |
| 127 | A | A | A | A | + |
| 128 | B | C | A | A | +++ |
| 129 | A | A | A | A | + |
| 130 | A | C | A | A | +++ |
| 131 | A | A | A | A | + |
| 132 | A | A | A | A | ++ |
| 133 | B | C | A | A | +++ |
| 134 | A | B | A | A | + |
| 135 | A | C | A | A | +++ |
| 136 | B | C | A | A | ++ |
| 137 | C | C | B | B | ++ |
| 138 | B | C | A | A | +++ |
| 139 | B | B | A | A | +++ |
| 140 | B | C | A | A | +++ |
| 141 | B | C | A | A | +++ |
| 142 | C | C | A | B | ++ |
| 143 | B | C | A | A | +++ |
| 144 | B | A | A | B | + |
| 145 | C | C | B | A | ++ |
| 146 | C | C | A | A | ++ |
| 147 | B | C | A | A | +++ |
| 148 | B | C | A | A | +++ |
| 149 | B | C | A | A | +++ |
| 150 | B | C | A | A | +++ |
| 151 | B | C | A | A | +++ |
| 152 | C | C | A | A | +++ |
| 153 | C | C | A | B | +++ |
| 154 | A | B | A | A | +++ |
| 155 | B | C | A | A | +++ |
| 156 | B | C | A | A | ++ |
| 157 | B | C | A | A | ++ |
| 158 | A | B | A | A | +++ |
| 159 | C | C | A | A | ++ |
| 160 | B | C | A | A | +++ |
| 161 | A | B | A | A | +++ |
| 162 | C | C | C | C | + |
| 163 | C | C | C | A | + |
| 164 | C | C | A | B | ++ |
| 165 | B | B | A | A | +++ |
| 166 | B | B | A | A | +++ |
| 167 | A | A | A | A | ++ |
| 168 | B | B | A | A | ++ |
| 169 | C | C | A | A | +++ |
| 170 | B | C | A | A | +++ |
| 171 | A | B | A | A | +++ |
| 172 | B | C | A | A | +++ |
| 173 | B | C | A | A | +++ |
| 174 | B | C | A | B | ++ |
| 175 | B | B | A | B | ++ |
| 176 | B | B | A | A | +++ |
| 177 | C | C | A | A | +++ |
| 178 | B | C | A | A | +++ |
| 179 | B | C | A | A | ++ |
| 180 | C | C | A | A | +++ |
| 181 | C | C | A | D | ++ |
| 182 | C | B | A | B | ++ |
| 183 | B | B | A | D | ++ |
| 184 | C | C | A | D | ++ |
| 185 | B | B | A | D | ++ |
| 186 | B | C | A | D | +++ |
| 187 | B | B | A | A | ++ |
| 188 | C | C | A | D | +++ |
| 189 | C | C | A | D | ++ |
| 190 | B | C | A | B | +++ |
| 191 | C | C | A | D | +++ |
| 192 | C | C | A | D | +++ |
| 193 | B | C | A | D | +++ |
| 194 | B | C | A | D | +++ |
| 195 | B | C | A | D | +++ |
| 196 | B | C | A | A | +++ |
| 197 | A | B | A | A | ++ |
| 198 | C | B | B | A | + |
| 199 | C | C | A | A | +++ |
| 200 | C | C | A | A | +++ |
| 201 | B | C | B | A | +++ |
| 202 | C | C | B | A | ++ |
| 203 | A | A | A | A | + |
| 204 | B | B | A | B | ++ |
| 205 | A | A | A | A | ++ |
| 206 | A | A | A | A | + |
| 207 | A | B | A | B | ++ |
| 208 | B | B | A | B | +++ |
| 209 | C | B | B | A | NS |
| 210 | A | A | A | A | + |
| 211 | B | B | A | B | ++ |
| 212 | B | B | A | A | ++ |
| 213 | B | C | A | B | ++ |
| 214 | C | C | A | A | ++ |
| 215 | C | C | B | A | ++ |
| 216 | C | C | B | B | ++ |
| 217 | C | C | A | A | +++ |
| 218 | C | C | B | B | + |
| 220 | A | B | A | B | +++ |
| 221 | B | C | A | B | +++ |
| 222 | B | C | A | A | ++ |
| 223 | B | B | A | B | ++ |
| 224 | A | A | A | B | ++ |
| 225 | B | C | A | A | ++ |
| 226 | B | B | A | B | +++ |
| 227 | A | B | A | A | +++ |
| 228 | B | C | A | B | ++ |
| 229 | B | B | A | B | ++ |
| 230 | B | C | A | B | +++ |
| 231 | B | C | A | A | +++ |
| 232 | B | B | B | B | + |
| 233 | A | B | A | A | +++ |
| 234 | B | C | A | A | +++ |
| 235 | A | B | A | A | +++ |
| 236 | B | B | A | B | + |
| 237 | C | C | A | C | +++ |
| 238 | B | B | A | C | ++ |
| 239 | B | C | A | A | +++ |
| 240 | C | C | A | B | +++ |
| 241 | C | C | B | A | ++ |
| 242 | C | C | B | B | ++ |
| 243 | C | C | A | C | +++ |
| 244 | B | C | A | C | +++ |
| 245 | B | B | A | A | +++ |
| 246 | B | C | A | A | +++ |
| 247 | C | C | B | B | ++ |
| 251 | B | C | A | A | +++ |
| 252 | B | C | A | V | ++ |
| 253 | C | C | C | B | ++ |
| 254 | C | C | C | C | + |
| 255 | A | C | A | A | +++ |
| 256 | C | C | A | B | +++ |
| 257 | A | C | A | A | +++ |
| 259 | A | C | A | A | +++ |
| 260 | B | C | A | A | +++ |
| 261 | B | B | A | B | ++ |
| 262 | B | B | A | A | ++ |
| 263 | B | C | A | B | +++ |
| 264 | A | C | A | A | +++ |
| 265 | C | C | A | B | +++ |
| 266 | B | C | A | A | +++ |
| 267 | A | B | A | A | +++ |
| 268 | B | C | A | B | +++ |
| 269 | A | A | A | A | + |
| 270 | A | A | A | A | + |
| 271 | C | C | A | B | +++ |
| 272 | A | B | A | A | + |
| 273 | B | C | A | B | +++ |
| 274 | B | C | A | B | ++ |
| 275 | C | C | A | B | +++ |
| 276 | C | C | C | B | ++ |
| 277 | C | C | C | C | NS |
| 278 | C | C | B | B | ++ |
| 279 | C | C | B | B | ++ |
| 280 | B | C | A | A | ++ |
| 281 | C | C | A | B | +++ |
| 282 | C | C | C | B | ++ |
| 283 | A | B | A | A | +++ |
| 284 | C | C | A | B | +++ |
| 285 | C | C | A | B | +++ |
| 286 | B | A | A | B | + |
| 287 | B | B | A | B | ++ |
| 288 | B | B | A | A | +++ |
| 289 | B | C | A | B | +++ |
| 290 | B | C | A | A | +++ |
| 291 | B | C | A | A | +++ |
| 292 | B | C | A | B | +++ |
| 293 | B | C | A | A | +++ |
| 294 | C | C | A | B | +++ |
| 295 | B | C | A | A | +++ |
| 296 | B | C | A | A | +++ |
| 297 | C | C | B | B | ++ |
| 298 | B | C | A | A | +++ |
| 299 | A | A | A | A | ++ |
| 300 | A | B | A | A | ++ |
| 301 | C | C | A | B | +++ |
| 302 | C | C | B | B | ++ |
| 303 | B | C | A | A | +++ |
| 304 | B | C | A | B | +++ |
| 305 | B | B | A | A | +++ |
| 306 | B | C | A | B | +++ |
| 307 | C | C | A | B | +++ |
| 308 | C | C | A | B | +++ |
| 309 | C | C | A | B | +++ |
| 310 | A | C | A | A | +++ |
| 311 | B | C | A | A | +++ |
| 312 | C | C | A | B | +++ |
| 313 | B | C | A | B | +++ |
| 314 | C | C | B | B | ++ |
| 315 | C | C | A | B | +++ |
| 316 | C | C | A | A | +++ |
| 317 | C | D | A | A | +++ |
| 318 | B | C | A | B | +++ |
| 319 | B | C | A | B | +++ |
| 320 | C | C | B | B | ++ |
| 321 | A | B | A | A | +++ |
| 322 | B | B | A | B | +++ |
| 323 | C | C | A | C | +++ |
| 324 | C | C | B | C | ++ |
| 325 | C | C | B | C | ++ |
| 326 | B | A | B | B | NS |
| 327 | C | C | B | C | ++ |
| 328 | C | C | B | B | ++ |
| 329 | B | C | A | B | +++ |
| 330 | C | C | B | B | ++ |
| 331 | A | A | A | A | + |
| 332 | C | C | B | B | + |
| 333 | C | C | C | C | + |
| 334 | C | C | A | B | ++ |
| 335 | C | C | A | B | +++ |
| 336 | B | B | A | B | ++ |
| 337 | C | C | A | B | +++ |
| 338 | C | C | A | B | +++ |
| 339 | B | C | A | B | +++ |
| 340 | C | C | A | B | +++ |
| 341 | C | C | A | B | +++ |
| 342 | C | C | C | C | + |
| 343 | B | C | A | A | +++ |
| 344 | B | C | A | B | +++ |
| 345 | C | C | B | C | ++ |
| 346 | C | C | B | C | + |
| 347 | C | B | B | B | + |
| 348 | ND | ND | ND | ND | ND |
| 349 | B | C | A | A | +++ |
| 350 | C | C | B | C | ++ |
| 351 | C | C | A | A | +++ |
| 352 | B | C | A | A | +++ |
| 353 | C | C | C | C | + |
| 354 | C | C | A | B | +++ |
| 355 | A | B | A | A | +++ |
| 356 | B | C | A | A | +++ |
| 357 | C | C | A | B | +++ |
| 358 | C | C | A | B | ++ |
| 359 | B | B | A | A | +++ |
| 360 | B | B | A | A | +++ |
| 361 | C | C | A | B | +++ |
| 362 | C | C | A | B | +++ |
| 363 | C | C | A | A | +++ |
| 364 | B | B | A | A | +++ |
| 365 | B | C | A | A | +++ |
| 366 | C | C | A | B | +++ |
| 367 | A | B | A | A | +++ |
| 368 | C | D | A | A | +++ |
| 369 | B | C | A | A | +++ |
| 370 | A | B | A | A | +++ |
| 371 | B | C | A | A | +++ |
| 372 | C | C | A | B | +++ |
| 373 | B | C | A | A | +++ |
| 374 | C | C | A | A | +++ |
| 375 | B | B | A | A | +++ |
| 376 | B | C | A | A | +++ |
| 377 | C | C | A | B | +++ |
| 378 | B | C | A | A | +++ |
| 379 | A | B | A | A | +++ |
| 380 | B | C | A | A | +++ |
| 381 | A | C | A | A | +++ |
| 382 | B | C | A | A | +++ |
| 383 | B | C | A | A | +++ |
| 384 | B | C | A | A | +++ |
| 385 | B | C | A | A | +++ |
| 386 | A | B | A | A | +++ |
| 387 | B | C | A | A | +++ |
| 388 | B | C | A | A | +++ |
| 389 | B | C | A | B | +++ |
| 390 | B | C | A | B | ++ |
| 391 | A | B | A | A | +++ |
| 392 | A | B | A | A | +++ |
| 393 | B | C | A | A | +++ |
| 394 | B | C | A | B | +++ |
| 395 | C | C | B | C | ++ |
| 396 | C | C | A | B | +++ |
| 397 | A | A | A | A | ++ |
| 398 | B | B | A | A | + |
| 399 | A | A | A | A | NS |
| 400 | C | C | B | B | ++ |
| 401 | B | C | A | B | +++ |
| 402 | A | B | A | A | +++ |
| 403 | C | C | B | C | ++ |
| 404 | B | C | A | B | ++ |
| 405 | B | C | A | B | +++ |
| 406 | C | C | A | A | +++ |
| 407 | B | C | A | A | +++ |
| 408 | B | B | A | A | +++ |
| 409 | C | B | A | A | ++ |
| 410 | C | C | A | B | +++ |
| 411 | B | C | A | A | +++ |
| 412 | A | A | A | A | +++ |
| 413 | A | A | A | A | +++ |
| 414 | A | A | A | A | +++ |
| 415 | A | B | A | A | +++ |
| 416 | A | A | A | A | +++ |
| 417 | A | B | A | A | +++ |
| 418 | A | A | A | A | +++ |
| 419 | B | C | A | A | +++ |
| 420 | A | B | A | A | +++ |
| 421 | A | A | A | A | +++ |
| 422 | A | B | A | A | +++ |
| 423 | A | B | A | A | +++ |
| 424 | A | B | A | A | +++ |
| 425 | C | C | B | C | ++ |
| 426 | B | C | A | A | +++ |
| 427 | B | D | A | A | +++ |
| 428 | A | B | A | A | +++ |
| 429 | A | B | A | A | +++ |
| 430 | B | B | A | A | +++ |
| 431 | C | C | A | A | +++ |
| 432 | A | B | A | A | +++ |
| 433 | A | B | A | A | +++ |
| 434 | B | B | A | A | +++ |
| 435 | A | A | A | A | +++ |
| 436 | A | B | A | A | +++ |
| 437 | A | B | A | A | +++ |

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists every possible subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

One of ordinary skill in the art will recognize, or be able to ascertain using no more than routine experimentation, many equivalents to the specific embodiments described and claimed herein. Such equivalents are intended to be encompassed by the following claims.

What is claimed is:

1. A compound of formula I: 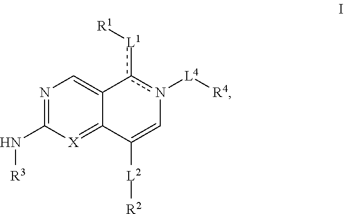
or a prodrug or pharmaceutically acceptable salt thereof, wherein:
each [symbol] represents a single or a double bond, wherein only one [symbol] is a single bond;
X is N or C(R);
R is hydrogen, deuterium, halogen, —CN, —S(O)₂R⁶, —S(O)₂N(R⁵)R⁶, —C(O)N(R⁵)R⁶, C(O)₂R⁵, P═O(R⁶)₂, N(R⁵)R⁶, OR⁵, optionally substituted —C₁-C₆ alkyl, optionally substituted aryl, optionally substituted carbocyclyl, optionally substituted heteroaryl, or optionally substituted heterocyclyl;
R⁵ is hydrogen, C₁-C₆ alkyl, aryl, C₃-C₅ cycloalkyl, heteroaryl, or heterocyclyl, wherein R⁵ is optionally substituted when other than hydrogen;
each R⁶ is independently C₁-C₆ alkyl, aryl, C₃-C₅ cycloalkyl, heteroaryl, or heterocycloalkyl, wherein R⁶ is optionally substituted; or
R⁵ and R⁶ are optionally taken together to form an optionally substituted heterocyclyl;
L¹ is a bond, ═O, —C₁-C₆ alkylene, —O—(C₀-C₆ alkylene)-†, —NH—(C₀-C₆ alkylene)-†, or —N(C₁-C₆ alkyl)-(C₀-C₆ alkylene-†, wherein “†” represents a portion of L¹ bound to R¹, and the alkylene portion of L¹ if present, is optionally substituted with one or more monovalent substituents;
when L¹ is ═O, R¹ is absent, or
when L¹ is a bond, —C₁-C₆ alkylene, —O—(C₀-C₆ alkylene)-†, —NH—(C₀-C₆ alkylene)-†, or —N—(C₀-C₆ alkylene)-(C₀-C₆ alkylene-†, R¹ is C₂-C₆ alkyl, aryl, heteroaryl, heterocyclyl, or carbocyclyl, wherein:
when R¹ is aryl, heteroaryl, heterocyclyl, or carbocyclyl, R¹ is optionally substituted with up to four different substituents;
when R¹ is C₂-C₆ alkyl, R¹ is optionally substituted with up to four different monovalent substituents, and
when L¹ is a bond R¹ is additionally selected from halo;
L² is a bond, —O—(C₀-C₆ alkylene)-*, —NH—(C₀-C₆ alkylene)-*, —N—(C₀-C₆ alkylene)-(C₀-C₆ alkylene)-*, wherein “*” represents a portion of L² bound to R², and the alkylene or alkyl portion of L² if present, is optionally substituted with one or more monovalent substituents;
R² is C₁-C₆ alkyl, aryl, heteroaryl, heterocyclyl or carbocyclyl, and R² is additionally hydrogen when L² is other than a bond, wherein:
when R² is aryl, heteroaryl, heterocyclyl or carbocyclyl, R² is optionally substituted with up to four different substituents;
when R² is C₁-C₆ alkyl, R² is optionally substituted with up to four different monovalent substituents; and
when L² is a bond, R² is other than trifluoromethyl-substituted 1H-indol-3-yl or cyano-substituted 1H-indol-3-yl;
R³ is optionally substituted —C₁-C₆ alkyl, —C₂-C₆ alkenyl, —C₂-C₆ alkynyl, —(C₂-C₆ alkylene)-O—(C₁-C₆ alkyl), —(C₂-C₆ alkylene)-O-aryl, —(C₂-C₆ alkylene)-O-heteroaryl, —(C₀-C₆ alkylene)-aryl, —(C₀-C₆ alkylene)-carbocyclyl, —(C₀-C₆ alkylene)-heterocyclyl, or —(C₀-C₆ alkylene)-heteroaryl, wherein each substituent on an alkyl or alkylene portion of R³ is a monovalent substituent, and wherein R³ is other than 2-aminocyclohexyl or 2-(t-butyloxycarbonylamino)cyclohexyl;
L⁴-R⁴ is absent when L¹ is other than ═O, or
L⁴ is a bond, or —(C₁-C₆ alkylene)-‡, wherein “‡” represents a portion of L⁴ bound to R⁴, and the alkylene portion of L⁴ if present, is optionally substituted; and
R⁴ is C₁-C₆ alkyl, —OR⁶, —N(R⁵)R⁶, —S(O)₂N(R⁵)R⁶, —S(O)₂—R⁶, —N(R⁵)—S(O)₂—R⁶, —N(R⁵)—C(O)—R⁶, —N(R⁵)—C(O)—N(R⁵)R⁶, —C(O)—N(R⁵)R⁶, aryl, heteroaryl, heterocyclyl, or carbocyclyl, wherein R⁴ is optionally substituted with up to four different substituents, and R⁴ is additionally hydrogen when L⁴ is other than a bond, wherein:
when X is N and L¹ is ═O, R³ is other than optionally substituted phenyl.

2. The compound of claim 1, wherein L¹, if present, is a bond, —O—, —O—CH₂-†, —O—CH₂—CH₂†, —NH—, —N(CH₃)—, or —NH—CH₂-†.

3. The compound of claim 1, wherein R¹, if present, is —Cl, —CH₃, —CF₃, —CH₂CHOCH₃, —CH₂CH₃, —CH₂CH₂N(CH₃)₂, —CH₂CF₃, —CH(CH₂OH)₂, morpholin-4-yl, 4,4-difluorocyclohexyl, 4-methoxycyclohexyl, 4-hydroxycyclohexyl, 4-hydroxycyclobutyl, tetrahydropyran-4-yl, piperidin-4-yl, 1-trifluoromethylcarbonylpiperidin-4-yl, 1-methylpiperidin-4-yl, 1-methyl sulfonylpiperidin-4-yl, 1-(2-dimethylaminocarbonylethyl)piperidin-4-yl, 1-(dimethylaminocarbonylmethyl)piperidin-4-yl, 1-(2-methoxyethyl)piperidin-4-yl, 1,1-dioxo-tetrahydro-2H-thiopyran-4-yl, 1,2,2,6,6-pentamethylpiperidin-4-yl, 8-oxabicyclo-[3.2.1]octan-3-yl, or 4-fluorophenyl, 1-(2-methoxyethyl)-2,2,6,6-tetrafluoro-piperidin-4-yl, 1-(2-methylpyrimidin-4-yl)piperidin-4-yl, 1-(2-methoxyethyl)-2,2,6,6-tetramethyl-piperidin-4-yl, 1-(methoxymethylcarbonyl)-2,2,6,6-tetramethylpiperidin-4-yl, 1-(pyrimidin-2-yl)piperidin-4-yl, 1-(tetrahydrofuran-2-ylmethyl)-2,2,6,6-tetramethylpiperidin-4-yl, 1,2,2-trimethylpiperidin-4-yl, 1,2,6-trimethylpiperidin-4-yl, 1-acetyl-2,2,6,6-tetramethyl-piperidin-4-yl, 1-ethyl-2,2,6,6-tetramethylpiperidin-4-yl, 1-methylpiperidin-3-yl, 1-methyl-pyridin-4-yl, 1-methylpyrrolidin-3-yl, 1-tetrahydrofuran-3-ylpiperidin-4-yl, 2-methylpyridin-4-yl, 2,2-dimethyl-3-hydroxycyclobutyl, 2,6-dimethyltetrahydrpyran-4-yl, 2,2,6,6-tetramethylpiperidin-4-yl, 3-(4-methylpiperazin-1-ylcarbonyl)cyclobutyl, 3-(methoxycarbonylamino)cyclobutyl, 3-(methylcarbonylamino)cyclobutyl, 3-(morpholin-4-yl)cyclobutyl, 3-(morpholin-4-ylmethyl)cyclobutyl, 3,3-difluoropiperidin-4-yl, 3-dimethylaminocyclopentyl, 3-hydroxycyclobutyl, 3-methoxycyclobutyl, 3-methyl-3-hydroxycyclobutyl, 4-aminocyclohexyl, 4-methyl-4-hydroxycyclohexyl, 4-(1-methyl-1-hydroxyethyl)cyclohexyl, 4-hydroxybicyclo[2.2.2]octanyl, 6-methylpyridin-3-yl, 8-methyl-8-azabicyclo[3.2.1]octan-3-yl, cyclopropyl, pyridin-3-yl, pyridin-4-yl, tetrahydrofuran-3-yl, tetrahydropyran-4-yl, 3-methoxycyclobutyl, 3-acetylaminocyclobutyl, 3-methoxycarbonyl-aminocyclobutyl, 3-(morpholin-4-yl)cyclobutyl, 8-(2-methoxyethyl)-8-azabicyclo[3.2.1]octan-3-yl, 8-(oxetan-3-yl)-8-azabicyclo[3.2.1]octan-3-yl, 8-methylcarbonyl-8-azabicyclo[3.2.1]octan-3-yl, 8-(tetrahydrofuran-2-ylmethyl)-8-azabicyclo[3.2.1]octan-3-yl, 8-(tetrahydrofuran-3-yl)-8-azabicyclo[3.2.1]octan-3-yl, 8-(tetrahydrofuran-3-ylmethyl)-8-azabicyclo[3.2.1]octan-3-yl, 8-(2,2,2-trifluoroethyl)-8-azabicyclo[3.2.1]octan-3-yl, and 1-(1-(morpholin-4-yl)carbonyl-1-methyl-ethyl)pyrazol-4-yl; or wherein L¹ and R¹ are taken together to form 3-dimethylamino-pyrrolidin-1-yl, 3-dimethylaminopiperidin-3-yl, 3-deimethylaminoazetidin-3-yl.

4. The compound of claim 1, wherein R, if present, is hydrogen or L⁴, if present, is a bond, —CH₂—, or —CH₂CH₂—.

5. The compound of claim 1, wherein R⁴, if present, is amino-substituted C₁-C₆ alkyl, optionally substituted phenyl or optionally substituted saturated heterocyclyl.

6. The compound of claim 1, wherein R³ is —C₃-C₆ alkyl, —(C₂-C₆ alkylene)-O—(C₁-C₆ alkyl), phenyl, C₃-C₆ cycloalkyl, saturated heterocyclyl, or —(C₁-C₂ alkylene)-heteroaryl, wherein each R³ is optionally substituted with 1-2 substituents independently selected from halo, —OH, —C₁-C₄ alkyl, and —O—C₁-C₄ alkyl.

7. The compound of claim 1, wherein L2 is a bond or —CH₂— or R² is phenyl, cyclohexyl, 1H-pyrazolyl, piperidinyl, pyridinyl, or pyridazinyl, wherein R² is optionally substituted with up to 3 substituents independently selected from halo, —OH, —NH₂, —NH(C₁-C₄ alkyl), —N—(C₁-C₄ alkyl)₂, —S(O)₂—C₁-C₄ alkyl, —C₁-C₄ alkyl optionally substituted with one or more substituent selected from —CN, —OH and halo, —C(O)—NH₂, —C(O)—NH(C₁-C₄ alkyl), —C(O)—N(C₁-C₄ alkyl)₂, —S(O)₂—NH₂, —S(O)₂—NH(C₁-C₄ alkyl), —S(O)₂—N(C₁-C₄ alkyl)₂, —NH—C(O)—C₁-C₄ alkyl, —NH—S(O)₂—C₁-C₄ alkyl, —NH—C(O)—NH—C₁-C₄ alkyl, —NH—S(O)₂—NH—C₁-C₄ alkyl, and optionally substituted pyrrolidinyl.

8. The compound of claim 1, wherein R³ is —C₃-C₆ alkyl, —(C₂-C₆ alkylene)-O—(C₁-C₆ alkyl), phenyl, C₃-C₆ cycloalkyl, saturated heterocyclyl, —(C₁-C₂ alkylene)-aryl or —(C₁-C₂ alkylene)-heteroaryl, wherein each R³ is optionally substituted with 1-2 substituents independently selected from deuterium, halo, —OH, —C₁-C₄ alkyl, —O—C₁-C₄ alkyl, —CN, —S(O)₂—C₁-C₄ alkyl, —C(O)OH, —C(O)O—C₁-C₄ alkyl, —C(O)NH₂, —C(O)NH—(C₁-C₄ alkyl), tetrazolyl, and oxo.

9. The compound of claim 1, having Formula Ia: 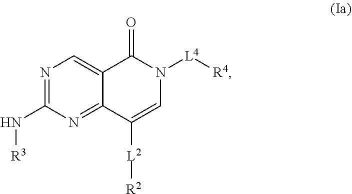
or a prodrug or a pharmaceutically acceptable salt thereof.

10. The compound of claim 9, wherein:
L⁴ is a bond; and
R⁴ is C₁-C₆ alkyl, heterocyclyl or carbocyclyl, wherein R⁴ is optionally substituted with up to four different substituents.

11. The compound of claim 1, having Formula Ic: 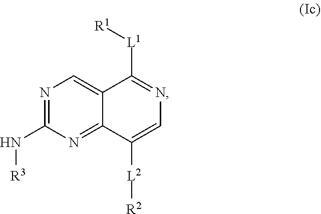
or a prodrug or a pharmaceutically acceptable salt thereof.

12. The compound of claim 11, having Formula Ic-1: 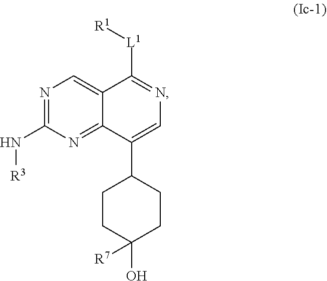
or a prodrug or a pharmaceutically acceptable salt thereof, wherein R⁷ is hydrogen, or C₁-C₃ alkyl.

13. The compound of claim 12, wherein:
L¹ is —O—;
R¹ is C₃-C₆, cycloalkyl or heterocyclyl, wherein R¹ is substituted with 1 to 5 substituents independently selected from —OH, —CH₃, —CH₂CH₃, —CH₂CH₂OCH₃, —NHC(O)CH₃, tetrahydrofuran-2-ylmethyl, tetrahydrofuran-3-ylmethyl, tetrahydrofuran-3-yl, tetrahydrofuran-2-yl, morpholin-4-yl, and morpholin-4-ylmethyl; and
R³ is a C₃-C₅ alkyl optionally substituted with up to 3 independently selected halo substituents.

14. The compound of claim 13, wherein:
R¹ is 3-hydroxycyclobutyl, 1-ethyl-2,2,6,6-tetramethylpiperidin-4-yl, 8-methyl-8-azabicyclo[3.2.1]octan-3-yl, 8-(2-methoxyethyl)-8-azabicyclo[3.2.1]octan-3-yl, 8-(oxetan-3yl)-8-azabicyclo[3.2.1]octan-3-yl, or 8-methylcarbonyl-8-azabicyclo[3.2.1]octan-3-yl; and
R³ is

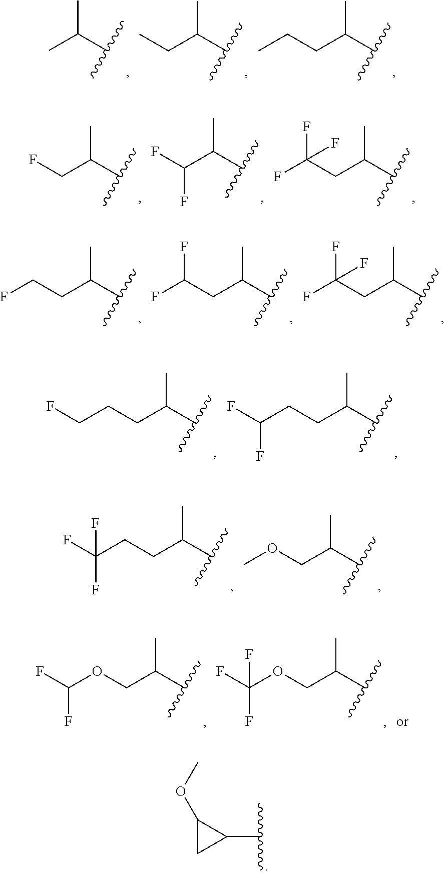

15. The compound of claim 12, wherein the compound has Formula Ic-2: 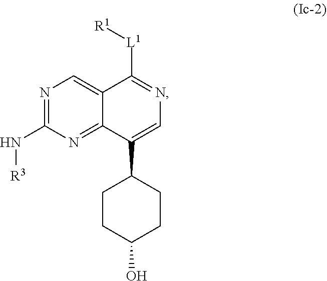
or a prodrug or a pharmaceutically acceptable salt thereof.

16. The compound of claim 15, wherein -L¹-R¹ is 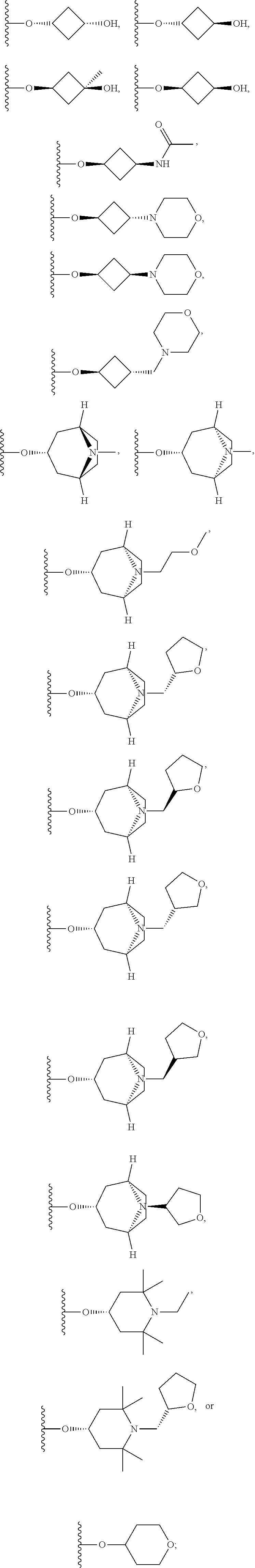
and
R³ is

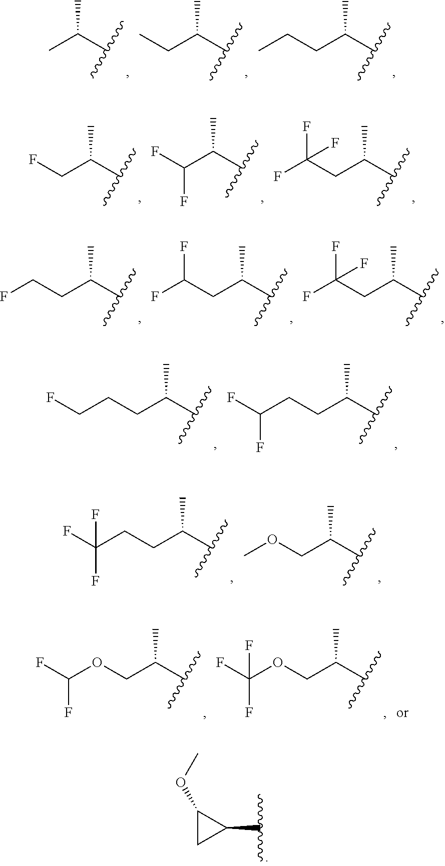

17. The compound of claim 12, wherein the compound has Formula Ic-3: 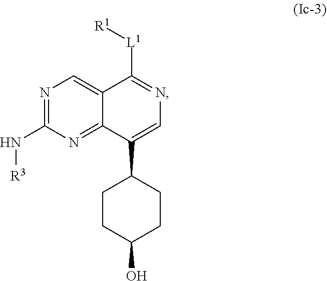
or a prodrug or a pharmaceutically acceptable salt thereof.

18. The compound of claim 17, wherein:
-L¹-R¹ is 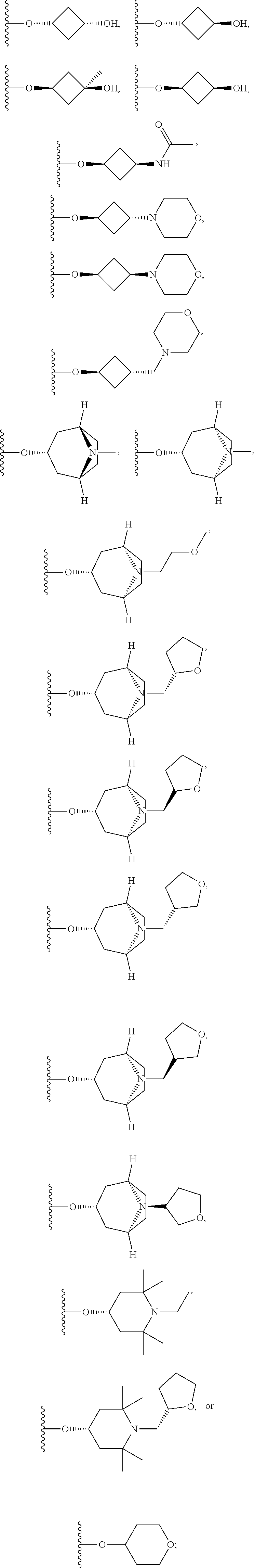
and
R³ is

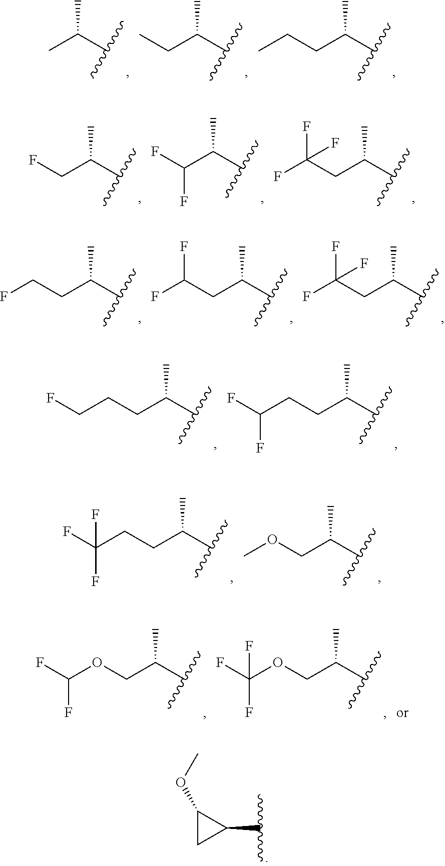

19. A pharmaceutical composition comprising a compound of claim 1, and a pharmaceutically acceptable carrier.

20. A method of treating a cancer associated with overexpression of or unwanted activity of a kinase selected from the group consisting of TYRO3, AXL and MERTK and combinations thereof, as compared to a reference cell, the method comprising a step of administering a compound or composition of claim 1 to a subject suffering from the cancer.
